SEQUENCE LISTING (258 sequences)

SEQ ID NO: 1 - protein, 472 aa, synthetic construct
  source 1..472: note = Human CD36
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 2 - protein, 472 aa, synthetic construct
  source 1..472: note = Cynomolgus monkey/Rhesus macaque CD36
MGCDRNCGLITGAVIGAVLAVFGGILMPVGDMLIQKTIKKEVVLEEGTIAFKNWVKTGTEIYRQFWIFDVQNPQEVMMNSSNIQVKQRGPYTYRVRFLAKENITQDPKDNTVSFLQPNGAIFEPSLSVGTEADNFTVLNLAVAAASHIYPNPFVQVVLNSLINKSKSSMFQVRTLRELLWGYTDPFLSLVPYPVSTRVGMFYPYNNTADGVYKVFNGKDSISKVAIIDTYKGKRNLSYWESYCDMINGTDAASFPPFVEKSQVLQFFSSDICRSIYAVFESDVNLKGIPVYRFVLPSKAFASPVQNPDNHCFCTEKIISKNCTSYGVLDISKCKEGKPVYISLPHFLYASPDVSETIDGLNPNEEEHRTYLDIEPITGFTLQFAKRLQVNLLVKPSNKIQVLKRLKRNYIVPILWLNETGTIGDEKAKMFRSQVTGKINLLGLIEMILLSVGVVMFVAFMISYCACRSKTIK

SEQ ID NO: 3 - protein, 472 aa, synthetic construct
  source 1..472: note = Murine CD36
MGCDRNCGLIAGAVIGAVLAVFGGILMPVGDMLIEKTIKREVVLEEGTTAFKNWVKTGTTVYRQFWIFDVQNPDDVAKNSSKIKVKQRGPYTYRVRYLAKENITQDPEDHTVSFVQPNGAIFEPSLSVGTEDDNFTVLNLAVAAAPHIYQNSFVQVVLNSLIKKSKSSMFQTRSLKELLWGYKDPFLSLVPYPISTTVGVFYPYNDTVDGVYKVFNGKDNISKVAIIESYKGKRNLSYWPSYCDMINGTDAASFPPFVEKSRTLRFFSSDICRSIYAVFGSEIDLKGIPVYRFVLPANAFASPLQNPDNHCFCTEKVISNNCTSYGVLDIGKCKEGKPVYISLPHFLHASPDVSEPIEGLHPNEDEHRTYLDVEPITGFTLQFAKRLQVNILVKPARKIEALKNLKRPYIVPILWLNETGTIGDEKAEMFKTQVTGKIKLLGMVEMALLGIGVVMFVAFMISYCACKSKNGK

SEQ ID NO: 4 - protein, 472 aa, synthetic construct
  source 1..472: note = Rat CD36
MGCDRNCGLITGAVIGAVLAVFGGILMPVGDLLIEKTIKREVVLEEGTIAFKNWVKTGTTVYRQFWIFDVQNPEEVAKNSSKIKVKQRGPYTYRVRYLAKENITQDPKDSTVSFVQPNGAIFEPSLSVGTENDNFTVLNLAVAAAPHIYTNSFVQGVLNSLIKKSKSSMFQTRSLKELLWGYKDPFLSLVPYPISTTVGVFYPYNNTVDGVYKVFNGKDNISKVAIIDTYKGKRNLSYWESYCDMINGTDAASFPPFVEKSQTLRFFSSDICRSIYAVFESEVNLKGIPVYRFVLPANAFASPLQNPDNHCFCTEKVISNNCTSYGVLDIGKCKEGKPVYISLPHFLHASPDVSEPIEGLNPNEDEHRTYLDVEPITGFTLQFAKRLQVNILVKPARKIEALKNLKRPYIVPILWLNETGTIGDEKAEMFRNQVTGKIKLLGLVEMVLLGVGVVMFVAFMISYCACRSKNGK

SEQ ID NO: 5 - protein, 462 aa, synthetic construct
  source 1..462: note = ONA-0-v1heavy chain
QVQLKQSGADLVRPGASVKLSCKASGYTFTDYYINWVKQRPGQGLEWIARIYPGSGNTYYNEKFKGKATLTAEKSSSTAYMQLSSLTSEDSAVYFCARGIGGGFGMDYWGQGTSVTVSSESARNPTIYPLTLPPVLCSDPVIIGCLIHDYFPFGTMNVTWGKSGKDITTVNFPPALASGGRYTMSSQLTLPAVECPEGESVKCSVQHDSNPVQELDVNCSPTPPPPITIPSCQPSLSLQRPALEDLLLGSDASITCTLNGLRNPEGAAFTWEPSTGKDAVQKKAAQNSCGCYSVSSVLPGCAERWNSGASFKCTVTHPESGTLTGTIAKVTVNTFPPQVHLLPPPSEELALNELLSLTCLVRAFNPKEVLVRWLHGNEELSPESYLVFEPLKEPGEGATTYLVTSVLRVSAETWKQGDQYSCMVGHEALPMNFTQKTIDRLSGKPTNVSVSVIMSEGDGICY

SEQ ID NO: 6 - protein, 214 aa, synthetic construct
  source 1..214: note = ONA-0-v1light chain
SIVMTQTPKFLLVSAGDRITITCKASQSVSDDVAWYQQKPGQSPKLLIYYASNRYTGVPDRFTGSGYGTDFTFTISTVQAEDLAVYFCQQDYSSPLTFGAGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 7 - protein, 119 aa, synthetic construct
  source 1..119: note = ONA-0-v1 VH
QVQLKQSGADLVRPGASVKLSCKASGYTFTDYYINWVKQRPGQGLEWIARIYPGSGNTYYNEKFKGKATLTAEKSSSTAYMQLSSLTSEDSAVYFCARGIGGGFGMDYWGQGTSVTVSS

SEQ ID NO: 8 - protein, 107 aa, synthetic construct
  source 1..107: note = ONA-0-v1 VL
SIVMTQTPKFLLVSAGDRITITCKASQSVSDDVAWYQQKPGQSPKLLIYYASNRYTGVPDRFTGSGYGTDFTFTISTVQAEDLAVYFCQQDYSSPLTFGAGTKLEIK

SEQ ID NO: 9 - protein, 449 aa, synthetic construct
  source 1..449: note = "1G04heavy chain"
QVQLKQSGADLVRPGASVKLSCKASGYTFTDYYINWVKQRPGQGLEWIARIYPGSGNTYYNEKFKGKATLTAEKSSSTAYMQLSSLTSEDSAVYFCARGIGGGFGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
  source 1..214: note = "1G04light chain"
SIVMTQTPKFLLVSAGDRITITCKASQSVSDDVAWYQQKPGQSPKLLIYYASNRYTGVPDRFTGSGYGTDFTFTISTVQAEDLAVYFCQQDYSSPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 449 aa, synthetic construct
  source 1..449: note = "1G06heavy chain"
QVQLKQSGADLVRPGASVKLSCKASGYTFTDYYINWVKQRPGQGLEWIARIYPGSGNTYYNEKFKGKATLTAEKSSSTAYMQLSSLTSEDSAVYFCARGIGGGFGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, synthetic construct; identical to SEQ ID NO: 10
  source 1..214: note = "1G06light chain"

SEQ ID NO: 13 - protein, 122 aa, synthetic construct
  source 1..122: note = 004G04 VH sequence
QVQLQQPGSELRSPGSSVKLSCKDFDSEVFPIAYMTWVRQKPGHGFEWIGDILPSIGGTIYGEKFEDKATLDADTVSNTAYLELNSLTSEDSAIYYCARDYYGSSYGYFDVWGAGTAVTVSS

SEQ ID NO: 14 - protein, 121 aa, synthetic construct
  source 1..121: note = 005G04 VH sequence
QVQLQQSGAELVMPGASVKMSCKASGYTFTDYWMHWVKQRPGQGLEWIGAIDTSDSYTSYNQKFKGKATLTVDESSSTAYMQLSSLTSEDSAVYYCARDYYGSSTAWFAYWGQGTLVTVSA

SEQ ID NO: 15 - protein, 120 aa, synthetic construct
  source 1..120: note = 006G04 VH sequence
QVQLQQSGAELVMPGASVKMSCKASGYTFTDYWMHWVKQRPGQGLEWIGSIDTSDSYTTYSQKFKGKATLTVDESSSTAYMELARLTSEDSAIYYCVRGEDYEGTWFAYWGQGTLVTVSA

SEQ ID NO: 16 - protein, 120 aa, synthetic construct
  source 1..120: note = 007G04 VH sequence
QVQMKESGAELVRPGASVKLSCKALGYTFTDYEIQWVKQTPVHGLEWIGGIHPGSSGIVYNQKFKGKATLTADKSSSTAYMELSSLTSEDSAVYYCTRGGGYDGAWFAYWGQGSLVTVSA

SEQ ID NO: 17 - protein, 120 aa, synthetic construct
  source 1..120: note = 009G04 VH sequence
QVQLQQSGAELVRPGASVTLSCKASGYTFTDYEMHWVKQTPVHGLEWIGTVDPETGGTAFNQKFKDKASLTADKSSSTVYMELRSLTSEDSAVYYCSRGYGNYGAWFAYWGQGTLVTVSA

SEQ ID NO: 18 - protein, 119 aa, synthetic construct
  source 1..119: note = 010G04 VH sequence
DVQLQESGPELVKPGASVKISCKASGYTFTDYNMHWVKQSHGKSLEWIGYIYPYNGGTGYNQKFKSKATLTVDNSSSTAYMELRSLTSEDSAVYYCARDGGYDVYFDYWGQGTTLTVSS

SEQ ID NO: 19 - protein, 115 aa, synthetic construct
  source 1..115: note = 011G04 VH sequence
EVQLQQSGAELVRPGASVTLSCKASGYRFSDYEMQWVKQTPVHGLEWIGGIDPETGGIAYNQKFKVKATLTADKSSSTAFMELRSLTSEDSAVYYCTRKLDFDYWGQGTTLTVSS

SEQ ID NO: 20 - protein, 123 aa, synthetic construct
  source 1..123: note = 012G04 VH sequence
EVQLVESGGGLVQPGESLKLSCESNEYEFPSHDMSWVRKTPEKRLELVAAINSDGGYTYYPDTMERRFIISRDNTKKTLYLQMSSLRSEDTALYYCARHGYDYDEEGAWFAYWGQGTLVTVSA

SEQ ID NO: 21 - protein, 122 aa, synthetic construct
  source 1..122: note = 013G04 VH sequence
QVQLQQSGAELVRPGASVKLSCKASGYTFTSYWMNWVKQRPGQGLEWIGMIDPSDNETHYNQMFKDKATLTVDKSSSTAYMQLSSLTSEDSAVYYCARSDYGNGYSFYLDVWGAGTTVTVSS

SEQ ID NO: 22 - protein, 119 aa, synthetic construct
  source 1..119: note = 014G04 VH sequence
QVQLQQSGAELVRSGASVRLSCTASGFNIKDYYIHWVKQRPEQGLEWIGWIDPENGDTEYAPRFQDKATMTADTSSNTAYLQLSSLTSEDTAVYYCNGWLLSGNGMDYWGQGTSVTVSS

SEQ ID NO: 23 - protein, 123 aa, synthetic construct
  source 1..123: note = 015G04 VH sequence
QVQLQQSGPELVKPGASVKISCKASGYTFSTSWMNWVKQRPGQGLEWIGRIYPGDGDTNYNGKFKGKATLTADTSSSTAYMQLSSLTSVDSAVYFCAREVYYGGYEDYGMDYWGQGTSVTVSS

SEQ ID NO: 24 - protein, 122 aa, synthetic construct
  source 1..122: note = 016G04 VH sequence
EVQLQQSGPELVKPGASVKISCKTSGYTFTEYTMHWVKQSHGKSLEWIGGINPYNGGTAYNQKFKNKATLTVDISSNTAFMEFRSLTSEDSAVYYCASDYFYGDANPWFAYWGQGTLVTVSA

SEQ ID NO: 25 - protein, 122 aa, synthetic construct
  source 1..122: note = 017G04 VH sequence
EVQLQQSGPELVKPGASVKISCKTSGYTFTEYTMHWVKQSHGKSLEWIGGIYPYNGGTAYNQKFKNKATLTVDISSNTAYMALRSLASDDSAVYYCASDYFYGDGYPWFTYWGQGTLVTVSA

SEQ ID NO: 26 - protein, 118 aa, synthetic construct
  source 1..118: note = 018G04 and 019G04 VH sequence
EVQLQQSGPELVKPGASVKISCETSGHTFTEYTMHWVKQSHGKSLEWIGGIYPNNGATKYNQKFKGKATLTVDKSSSTAYMELRSLTSEDSAVYYCARDRYDVWFAYWGQGTLVTVSA

SEQ ID NO: 27 - protein, 118 aa, synthetic construct
  source 1..118: note = 020G04 VH sequence
QIQLVQSGPELVKPGASVKISCETSGHTFTEYTMHWVKQSHGKSLEWIGGIYPNNGATKYNQKFKGKATLTVDKSSSTAYMELRSLTSEDSAVYYCARDRYDVWFAYWGQGTLVTVSA

SEQ ID NO: 28 - protein, 116 aa, synthetic construct
  source 1..116: note = 021G04 VH sequence
QVQLQQSGAELVKPGASVKLSCKASGYTFTSYWMHWVKQRPGQGLEWIGEIDPSDSHTNYNQKFKGKATLTVDKSSSTAYMQLSSLTSEDSAVYYCAPIYYGLDNWGQGTTLTVSS

SEQ ID NO: 29 - protein, 116 aa, synthetic construct
  source 1..116: note = 022G04 VH sequence
QVQLQQPGAELVKPGASVKLSCKASGYTFTSYWMHWVKQRPGQGLEWIGEIDPSDSHANYNQKFKGKATLTVDKSSSTAYMQLSSLTSEDSAVYYCAPIHYGLDNWGQGTTLTVSS

SEQ ID NO: 30 - protein, 116 aa, synthetic construct
  source 1..116: note = 023G04 VH sequence
QVQLQQPGTELVKPGASVKLSCKASGYTFTSYWMHWVKQRPGQGLEWIGEIDPSDSHTNYNQKFKGKATLTVDKSSSTAYMQLSSLTSEDSAVYYCAPIYYGLDNWGQGTTLTVSS

SEQ ID NO: 31 - protein, 116 aa, synthetic construct
  source 1..116: note = 024G04 VH sequence
QVQLQQSGAELVKPGASVKLSCKASGYTFTSYWMHWVKQRPGQGLEWIGEIDPSDSHANYNQKFKGKATLTVDKSSSTAYMQLSSLTSEDSAVYYCAPIYYGLDNWGQGTTLTVSS

SEQ ID NO: 32 - protein, 116 aa, synthetic construct
  source 1..116: note = 025G04 VH sequence
EVQLQQSGAELVKPGASVKLSCKASGYTFTSYWMHWVKQRPGQGLEWIGEIDPSDSHANYNQKFKGKATLTVDKSSSTAYMQLSSLTSEDSAVYYCAPIYYGLDNWGQGTTLTVSS

SEQ ID NO: 33 - protein, 120 aa, synthetic construct
  source 1..120: note = 026G04 VH sequence
QVQMKESGAELMKPGASVKISCKATGYIFSNYWIEWLKERPGHGLEWIGDFLPGSSSANYNEKFKGKATFTADTSSNTAYMQLSSLTSEDSAVYYCVPFTVEVTDAMDYWGQGTSVTVSS

SEQ ID NO: 34 - protein, 118 aa, synthetic construct
  source 1..118: note = 027G04 VH sequence
EVQLQQSGAELLKPGASVKISCKTTGYTFSNYWIEWVKQRPGHGLEWIGEILPGSGSPKYNEKFKGKATLTADTSSNTAFMQLSSLTSEDSAVYYCAPFILENYFDYWGQGTTLTVSS

SEQ ID NO: 35 - protein, 117 aa, synthetic construct
  source 1..117: note = 028G04 VH sequence
EVQLQQSGPELVKPGASVKISCKTSGYTFTEYTIHWVKQSHGKSLEWIGGIIPNNGGSSHKQNFKDKATLTVDKSSSTAYMELRSLTSEDSAVYYCARAGDYAFDYWGQGTTLTVSS

SEQ ID NO: 36 - protein, 116 aa, synthetic construct
  source 1..116: note = 029G04 VH sequence
QVQLQQSGTELVKPGASVKLSCKASGYTFTSYWMHWVKQRPGQGLEWIGEIDPSDSHTNYNQKFKGKATLTVDKSSSTAYMQLSSLTSEDSAVYYCAPIYYGLDNWGQGTTLTVSS

SEQ ID NO: 37 - protein, 119 aa, synthetic construct
  source 1..119: note = 030G04 VH sequence
EVQLVESGGGVVQPGESLKLSCESNEYEFPSHDMSWVRKTPERRLELVAAINSDGAITYYPDTMERRFIISRDNTKKTLYLQMSSLKSEDTAMYYCARHGQTGTWFAYWGQGTLVTVSA

SEQ ID NO: 38 - protein, 122 aa, synthetic construct
  source 1..122: note = 031G04 VH sequence
QVQLQQSGSELRSPGSSVKLSCKDFDSEVFPIAYMSWVRQKPGHGFEWIGDILPSIGSTIYGEKFEDKATLDADTVSNTAYLELNSLTSEDSAIYYCARDYYGSSYGYFDVWGAGTTVTVPS

SEQ ID NO: 39 - protein, 115 aa, synthetic construct
  source 1..115: note = 032G04 VH sequence
EEKLEESGGGLVQPGGSMKLSCVASGFTFSNYWMNWVRQSPEKGLEWVAEIRLKSNNYATHYAESVKGRFTISRDDSKSSVYLQMNNLRAEDTGIYYCTPLLLRWGQGTTLTVSS

SEQ ID NO: 40 - protein, 129 aa, synthetic construct
  source 1..129: note = 033G04 and 034G04 VH sequence
AVTLDESGGGLQTPGGALSLVCKASGFDFSSYAMGWVRQAPGKGLEWVAGKAKDGGGTAYGSAVKGRATISRDNGQSTVRLQLSNLRAEDTGVYYCAKSAYGDWFYGSSGPYADSIDAWGHGTEVIVSS

SEQ ID NO: 41 - protein, 124 aa, synthetic construct
  source 1..124: note = 035G04 VH sequence
AVTLDESGGGLQTPGGALSLVCKASGFTFSNYGMGWMRQAPGKGLEFVAKIYKDGGYTGYGAAVDGRATISRDDGQSTVRLQLNDLRAEDTATYFCAKAADSGYLYITDSIDAWGHGTEVIVSS

SEQ ID NO: 42 - protein, 128 aa, synthetic construct
  source 1..128: note = 036G04 VH sequence
AVTLDESGGGLQTPGGALSLVCKASGFIFSSYGMGWVRQAPGKGLEYIAAISNDGSKADYGAAVKGRATISRDNGQSTVRLQLNNLRAEDTATYYCAKSADTGYCSWSACIADSIDAWGHGTEVIVSS

SEQ ID NO: 43 - protein, 124 aa, synthetic construct
  source 1..124: note = 037G04 VH sequence
AVTLDESGGGLQTPGGALSLVCKASGFTFSDYDMLWVRQAPGKGLEFVAGINAASTYTDYGAAVKGRATISRDNGQSTVRLQVNNLRAEDTGTYYCAKSSYGGGWSDVGSIDAWGHGTEVIVSS

SEQ ID NO: 44 - protein, 118 aa, synthetic construct
  source 1..118: note = 038G04 VH sequence
AVTLDESGGGLQTPGRALSLVCKASGFTFSSYNMFWVRQAPGKGLEWVAGIDDDGSFTLYGAAVKGRATISRDNGQSTVRLQLNNLRAEDTGTYYCARDSGAVSIDAWGHGTEVIVSS

SEQ ID NO: 45 - protein, 107 aa, synthetic construct
  source 1..107: note = 004G04 VL sequence
DIVLTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIK

SEQ ID NO: 46 - protein, 107 aa, synthetic construct
  source 1..107: note = 005G04 VL sequence
ETTVTQSPASLSMAIGEKVTIRCITSTDIDDDMNWYQQKPGEPPKLLISEGNTLRPGVPSRFSSSGYGTDFVFTIDNMLSEDVADYYCLQSDNLPFTFGSGTKLEIK

SEQ ID NO: 47 - protein, 107 aa, synthetic construct
  source 1..107: note = 006G04 VL sequence
ETTVTQSPASLSVATGEKVTIRCITSTDIDDDMNWYQQKPGEPPKLLISEGNTLRPGVPSRFSSSGYGTDFVFTIENTLSEDVADYYCLQSDNMPLTFGAGTKLELK

SEQ ID NO: 48 - protein, 107 aa, synthetic construct
  source 1..107: note = 007G04 VL sequence
ETTVTQSPASLSMAIGEKVTIRCITSTDIDDDMNWYQQKPGEPPKLLISEGNTLRPGVPSRFSSSGYGTDFVFTIENMLSEDVADYYCLQSDNLPFTFGSGTKLEIK

SEQ ID NO: 49 - protein, 107 aa, synthetic construct
  source 1..107: note = 009G04 VL sequence
ETTVTQSPASLSVATGEKVTIRCITSTDIDDDMNWYQQKPGEPPKLLISEGNTLRPGVPSRFSSSGYGTDFVFTIENMLSEDVADYYCLQSDNLPFTFGSGTKLEIK

SEQ ID NO: 50 - protein, 112 aa, synthetic construct
  source 1..112: note = 010G04 VL sequence
DVVMTQTPLSLPVSLGDQASISCRSSQSLVHSNGNTYLHWYLQKPGQSPKLLIYKVSNRFSGVPDRFSGSGSGTDFTLKISRVEAEDLGVYYCFQGSHVPWTFGGGTKLEIK

SEQ ID NO: 51 - protein, 107 aa, synthetic construct
  source 1..107: note = 011G04 VL sequence
ETTVTQSPASLSVATGEKVTIRCITSTDIDDDMNWYQQKPGEPPKLLISEGNTLRPGVPSRFSSSGYGTDFVFTIENMLSEDVADYYCLQSDNLPLTFGAGTKLELK

SEQ ID NO: 52 - protein, 107 aa, synthetic construct
  source 1..107: note = 012G04 VL sequence
ETTVTQSPASLSMAIGEKVTIRCITSTDIDDDMNWYQQKPGEPPKLLISEGNTLRPGVPSRFSSTGYGTDFVFSIENMLSEDVADYYCLQSDNLPFTFGSGTKLEIK

SEQ ID NO: 53 - protein, 111 aa, synthetic construct
  source 1..111: note = 013G04 VL sequence
DIVLTQSPASLAVSLGQRATISCRASESVDSYGNSFMHWYQQKPGQPPKLLIYRASNLESGIPARFSGSGSRTDFTLTINPVEADDVATYYCQQSNEDPWTFGGGTKLEIK

SEQ ID NO: 54 - protein, 106 aa, synthetic construct
  source 1..106: note = 014G04 VL sequence
DIVMTQSHKFMSTSVGDRVSITCKASQDVGTAVAWYQQKPGQSPKLLIYWASTRHTGVPDRFTGSGSGTDFTLTISNVQSEDLADYFCQQYSSYPTFGGGTKLEIK

SEQ ID NO: 55 - protein, 112 aa, synthetic construct
  source 1..112: note = 015G04 VL sequence
DVVMTQTPLSLPVSLGDQASISCRSSQSLLHTNVNTYLHWYLQKPGQSPKLLIYKVSNRFSGVPDRFSGSGSGTDFTLKISRVEAEDLGVYFCSQSTHVPYTFGGGTKLEIK

SEQ ID NO: 56 - protein, 107 aa, synthetic construct
  source 1..107: note = 016G04 VL sequence
ETTVTQSPASLSMAIGEKVTIRCITSTDIDDDMNWYQQKPGEPPKLLISEGNTLRPGVPSRFSSSGYGTDFVFTIENMLSEDVADYYCLQSDNLPLTFGAGTKLELK

SEQ ID NO: 57 - protein, 107 aa, synthetic construct
  source 1..107: note = 017G04 VL sequence
ETTVTQSPASLSVATGEKVTIRCITSTDIDDDMNWYQQRPGEPPNLLISEGNTLRPGVPSRFSSSGYGTDFVFTIENTLSEDVADYYCLQSDNLPFTFGSGTRLEIK

SEQ ID NO: 58 - protein, 107 aa, synthetic construct
  source 1..107: note = 018G04 VL sequence
ETTVTQSPASLSMAIGEKVTIRCITSTDIDDDMNWYQQKPGEPPKLLISEGNTLRPGVPSRFSSSGYGTDFVFTIENLLSEDVADYYCLQSDNLPLTFGGGTKLEIK

SEQ ID NO: 59 - protein, 107 aa, synthetic construct
  source 1..107: note = 019G04 VL sequence
ETTVTQSPASLSMAIGEKVTIRCITSTDIDDDMNWYQQKPGEPPKLLISEGNTLRPGVPSRFSSSGYGTDFIFTIENMLSEDVADYYCLQSDNLPLTFGAGTKLELK

SEQ ID NO: 60 - protein, 107 aa, synthetic construct
  source 1..107: note = 020G04 VL sequence
ETTVTQSPASLSVATGEKVTIRCITSTDIDDDMNWYQQKPGEPPKLLISEGNTLRPGVPSRFSSGGYGTDFVFTVENMLSEDVADYYCLQSDNLPLTFGAGTKLELK

SEQ ID NO: 61 - protein, 107 aa, synthetic construct
  source 1..107: note = 021G04 VL sequence
ETTVTQSQKFMSTSVGDRVSVTCKASQNVGTNVAWYQQKPGQSPKALIYSASYRYSGVPDRFTGSGSGTDFTLTISNVQSEDLAEYFCQQYNSYPFTFGSGTKLEIK

SEQ ID NO: 62 - protein, 107 aa, synthetic construct
  source 1..107: note = 022G04 VL sequence
DIVLTQSQKFMSTSVGDRVSVTCKASQNVGTNVAWYQQKPGQSPKALIYSASYRYSGVPDRFTGSGSGTDFTLTISNVQSEDLAEYFCQQYNSYPLTFGAGTKLELK

SEQ ID NO: 63 - protein, 107 aa, synthetic construct; identical to SEQ ID NO: 45
  source 1..107: note = 023G04 VL sequence

SEQ ID NO: 64 - protein, 107 aa, synthetic construct
  source 1..107: note = 024G04 VL sequence
DIVMTQSQKFLSTSVGDRVSVTCKASQNVGTNVAWYQQKPGQSPKVLIYSASYRYSGVPDRFTGSGSGTDFTLTVSNVQSEDLAEYFCQQYNNYPLTFGAGTKLELK

SEQ ID NO: 65 - protein, 107 aa, synthetic construct
  source 1..107: note = 025G04 VL sequence
DIVLTQSQKFMSTSVGDRVSVTCKASQNVGTNVAWYQQKPGQSPKALIYSASYRYSGVPDRFTGSGSGTDFTLTISNVQPEDLAEYFCQQYNSYPLTFGGGTKLEIK

SEQ ID NO: 66 - protein, 107 aa, synthetic construct
  source 1..107: note = 026G04 VL sequence
DIVLTQSHKFMSTSVGDRVSITCKASQDVSTAVAWYQQKPGQSPKLLIYSASYRYTGVPDRFTGSGSGTDFTFTISSVQAEDLAVYYCQQHYSTPRTFGGGTKLEIK

SEQ ID NO: 67 - protein, 107 aa, synthetic construct
  source 1..107: note = 027G04 VL sequence
DIQMTQSHKFMSTSVGDRVSITCKASQDVSTAVAWYQQKPGQSPKLLIYSASYRYTGVPDRFTGSGSGTDFTFTISSVQAEDLAVYYCQQHYSTPRTFGGGTKLEIK

SEQ ID NO: 68 - protein, 107 aa, synthetic construct
  source 1..107: note = 028G04 VL sequence
ETTVTQSPASLSMAIGEKVTIRCITSTDIDDDMNWYQQKPGEPPKLLISEGNTLRPGVPSRFSSSGYGTDFVFTIENMLSEDVADYYCLQSGTLPFTFGSGTKLEIK

SEQ ID NO: 69 - protein, 107 aa, synthetic construct; identical to SEQ ID NO: 62
  source 1..107: note = 029G04 VL sequence

SEQ ID NO: 70 - protein, 107 aa, synthetic construct; identical to SEQ ID NO: 56
  source 1..107: note = 030G04 VL sequence

SEQ ID NO: 71 - protein, 111 aa, synthetic construct
  source 1..111: note = 031G04 VL sequence
DIVLTQSPASLAVSLGQRATISCRASESVDSYGNSFMHWYQQKPGQPPKLLIYRASNLESGIPARFSGSGSRTDFTLTINPVEADDVATYYCQQSNEDPYTFGGGTKLEIK

SEQ ID NO: 72 - protein, 108 aa, synthetic construct
  source 1..108: note = 032G04 VL sequence
ENVLTQSPAIMSASPGEKVTMTCRASSSLSSTYLHWYQQKSGASPKLWIYSTSNLASGVPARFSGSGSGTSYSLTISSVEAEDAATYYCQQYSGYPLTFGSGTKLEIK

SEQ ID NO: 73 - protein, 105 aa, synthetic construct
  source 1..105: note = 033G04 VL sequence
ALTQPSSVSASPGETVKITCSGSSDSWYGWYQQKSPGSALVTVIYDSTNRPSSIPSRFSGSTSGSTGTLTITGVRADDEAVYFCGSFDSSTDSTAFGAGTTLTVL

SEQ ID NO: 74 - protein, 109 aa, synthetic construct
  source 1..109: note = 034G04 VL sequence
ALTQPSSVSANPGETVKITCSGGDNYAGSYYYGWYQQKSPGSAPVTVIYDDTNRPADIPSRFSGSTSGSTNTLTITGVRAEDEAVYFCGGWDSINDRNIFGAGTTLTVL

SEQ ID NO: 75 - protein, 104 aa, synthetic construct
  source 1..104: note = 035G04 VL sequence
ALTQPSSVSANPGETVKITCSGGSGSYGWFQQKSPGNAPVTVIYHNDQRPSDIPSRFSGSKSGSTATLTITEVQADDEAVYVCGGYDNDNDAGIFGAGTTLTVL

SEQ ID NO: 76 - protein, 103 aa, synthetic construct
  source 1..103: note = 036G04 VL sequence
ALTQPSSVSAIPGETVKITCSGGGSDYGWYQQKSPGSAPVTVIYWSDNRPSNIPSRFSGSESGSTATLTITGVRAEDEAVYFCGGYDSSGSGMFGAGTTLTVL

SEQ ID NO: 77 - protein, 105 aa, synthetic construct
  source 1..105: note = 037G04 VL sequence
ALTQPSSVSANPGETVKITCSGDRSYYGYGWYQQKAPGSAPVTVIYWDDKRPSDIPSRFSGSTSGSTATLTITGVQAEDEAVYFCGNMDSSYVGMFGAGTTLTVL

SEQ ID NO: 78 - protein, 104 aa, synthetic construct
  source 1..104: note = 038G04 VL sequence
ALTQPSSVSANPGETVKITCSGGDSDYGWYQQKSPGSAPVTVIYQNDKRPSDIPSRFSGSASGSTATFTITGVQAEDEAVYYCGGWDGSYDGAVFGAGTTLTVL

SEQ ID NO: 79 - protein, 5 aa, synthetic construct
  source 1..5: note = ONA-0-v1 HDCR1
DYYIN

SEQ ID NO: 80 - protein, 17 aa, synthetic construct
  source 1..17: note = ONA-0-v1 HDCR2
RIYPGSGNTYYNEKFKG

SEQ ID NO: 81 - protein, 10 aa, synthetic construct
  source 1..10: note = ONA-0-v1 HDCR3
GIGGGFGMDY

SEQ ID NO: 82 - protein, 11 aa, synthetic construct
  source 1..11: note = ONA-0-v1 LDCR1
KASQSVSDDVA

SEQ ID NO: 83 - protein, 7 aa, synthetic construct
  source 1..7: note = ONA-0-v1 LDCR2
YASNRYT

SEQ ID NO: 84 - protein, 9 aa, synthetic construct
  source 1..9: note = ONA-0-v1 LDCR3
QQDYSSPLT

SEQ ID NO: 85 - protein, 6 aa, synthetic construct
  source 1..6: note = HCDR1
PIAYMT

SEQ ID NO: 86 - protein, 5 aa, synthetic construct
  source 1..5: note = HCDR1
DYWMH

SEQ ID NO: 87 - protein, 5 aa, synthetic construct
  source 1..5: note = HCDR1
DYEIQ

SEQ ID NO: 88 - protein, 5 aa, synthetic construct
  source 1..5: note = HCDR1
DYEMH

SEQ ID NO: 89 - protein, 5 aa, synthetic construct
  source 1..5: note = HCDR1
DYNMH

SEQ ID NO: 90 - protein, 5 aa, synthetic construct
  source 1..5: note = HCDR1
DYEMQ

SEQ ID NO: 91 - protein, 5 aa, synthetic construct
  source 1..5: note = HCDR1
SHDMS

SEQ ID NO: 92 - protein, 5 aa, synthetic construct
  source 1..5: note = HCDR1
SYWMN

SEQ ID NO: 93 - protein, 5 aa, synthetic construct
  source 1..5: note = HCDR1
DYYIH

SEQ ID NO: 94 - protein, 5 aa, synthetic construct
  source 1..5: note = HCDR1
TSWMN

SEQ ID NO: 95 - protein, 5 aa, synthetic construct
  source 1..5: note = HCDR1
EYTMH

SEQ ID NO: 96 - protein, 5 aa, synthetic construct
  source 1..5: note = HCDR1
SYWMH

SEQ ID NO: 97 - protein, 5 aa, synthetic construct
  source 1..5: note = HCDR1
NYWIE

SEQ ID NO: 98 - protein, 5 aa, synthetic construct
  source 1..5: note = HCDR1
EYTIH

SEQ ID NO: 99 - protein, 6 aa, synthetic construct
  source 1..6: note = HCDR1
PIAYMS

SEQ ID NO: 100 - protein, 5 aa, synthetic construct
  source 1..5: note = HCDR1
NYWMN

SEQ ID NO: 101 - protein, 5 aa, synthetic construct
  source 1..5: note = HCDR1
SYAMG

SEQ ID NO: 102 - protein, 5 aa, synthetic construct
  source 1..5: note = HCDR1
NYGMG

SEQ ID NO: 103 - protein, 5 aa, synthetic construct
  source 1..5: note = HCDR1
SYGMG

SEQ ID NO: 104 - protein, 5 aa, synthetic construct
  source 1..5: note = HCDR1
DYDML

SEQ ID NO: 105 - protein, 5 aa, synthetic construct
  source 1..5: note = HCDR1
SYNMF

SEQ ID NO: 106 - protein, 17 aa, synthetic construct
  source 1..17: note = HCDR2
DILPSIGGTIYGEKFED

SEQ ID NO: 107 - protein, 17 aa, synthetic construct
  source 1..17: note = HCDR2
AIDTSDSYTSYNQKFKG

SEQ ID NO: 108 - protein, 17 aa, synthetic construct
  source 1..17: note = HCDR2
SIDTSDSYTTYSQKFKG

SEQ ID NO: 109 - protein, 17 aa, synthetic construct
  source 1..17: note = HCDR2
GIHPGSSGIVYNQKFKG

SEQ ID NO: 110 - protein, 17 aa, synthetic construct
  source 1..17: note = HCDR2
TVDPETGGTAFNQKFKD

SEQ ID NO: 111 - protein, 17 aa, synthetic construct
  source 1..17: note = HCDR2
YIYPYNGGTGYNQKFKS

SEQ ID NO: 112 - protein, 17 aa, synthetic construct
  source 1..17: note = HCDR2
GIDPETGGIAYNQKFKV

SEQ ID NO: 113 - protein, 17 aa, synthetic construct
  source 1..17: note = HCDR2
AINSDGGYTYYPDTMER

SEQ ID NO: 114 - protein, 17 aa, synthetic construct
  source 1..17: note = HCDR2
MIDPSDNETHYNQMFKD

SEQ ID NO: 115 - protein, 17 aa, synthetic construct
  source 1..17: note = HCDR2
WIDPENGDTEYAPRFQD

SEQ ID NO: 116 - protein, 17 aa, synthetic construct
  source 1..17: note = HCDR2
RIYPGDGDTNYNGKFKG

SEQ ID NO: 117 - protein, 17 aa, synthetic construct
  source 1..17: note = HCDR2
GINPYNGGTAYNQKFKN

SEQ ID NO: 118 - protein, 17 aa, synthetic construct
  source 1..17: note = HCDR2
GIYPYNGGTAYNQKFKN

SEQ ID NO: 119 - protein, 17 aa, synthetic construct
  source 1..17: note = HCDR2
GIYPNNGATKYNQKFKG

SEQ ID NO: 120 - protein, 17 aa, synthetic construct
  source 1..17: note = HCDR2
EIDPSDSHTNYNQKFKG

SEQ ID NO: 121 - protein, 17 aa, synthetic construct
  source 1..17: note = HCDR2
EIDPSDSHANYNQKFKG

SEQ ID NO: 122 - protein, 17 aa, synthetic construct
  source 1..17: note = HCDR2
DFLPGSSSANYNEKFKG

SEQ ID NO: 123 - protein, 17 aa, synthetic construct
  source 1..17: note = HCDR2
EILPGSGSPKYNEKFKG

SEQ ID NO: 124 - protein, 17 aa, synthetic construct
  source 1..17: note = HCDR2
GIIPNNGGSSHKQNFKD

SEQ ID NO: 125 - protein, 17 aa, synthetic construct
  source 1..17: note = HCDR2
AINSDGAITYYPDTMER

SEQ ID NO: 126 - protein, 17 aa, synthetic construct
  source 1..17: note = HCDR2
DILPSIGSTIYGEKFED

SEQ ID NO: 127 - protein, 19 aa, synthetic construct
  source 1..19: note = HCDR2
EIRLKSNNYATHYAESVKG

SEQ ID NO: 128 - protein, 17 aa, synthetic construct
  source 1..17: note = HCDR2
GKAKDGGGTAYGSAVKG

SEQ ID NO: 129 - protein, 17 aa, synthetic construct
  source 1..17: note = HCDR2
KIYKDGGYTGYGAAVDG

SEQ ID NO: 130 - protein, 17 aa, synthetic construct
  source 1..17: note = HCDR2
AISNDGSKADYGAAVKG

SEQ ID NO: 131 - protein, 17 aa, synthetic construct
  source 1..17: note = HCDR2
GINAASTYTDYGAAVKG

SEQ ID NO: 132 - protein, 17 aa, synthetic construct
  source 1..17: note = HCDR2
GIDDDGSFTLYGAAVKG

SEQ ID NO: 133 - protein, 12 aa, synthetic construct
  source 1..12: note = HCDR3
DYYGSSYGYFDV

SEQ ID NO: 134 - protein, 12 aa, synthetic construct
  source 1..12: note = HCDR3
DYYGSSTAWFAY

SEQ ID NO: 135 - protein, 11 aa, synthetic construct
  source 1..11: note = HCDR3
GEDYEGTWFAY

SEQ ID NO: 136 - protein, 11 aa, synthetic construct
  source 1..11: note = HCDR3
GGGYDGAWFAY

SEQ ID NO: 137 - protein, 11 aa, synthetic construct
  source 1..11: note = HCDR3
GYGNYGAWFAY

SEQ ID NO: 138 - protein, 10 aa, synthetic construct
  source 1..10: note = HCDR3
DGGYDVYFDY

SEQ ID NO: 139 - protein, 6 aa, synthetic construct
  source 1..6: note = HCDR3
KLDFDY

SEQ ID NO: 140 - protein, 14 aa, synthetic construct
  source 1..14: note = HCDR3
HGYDYDEEGAWFAY

SEQ ID NO: 141 - protein, 13 aa, synthetic construct
  source 1..13: note = HCDR3
SDYGNGYSFYLDV

SEQ ID NO: 142 - protein, 10 aa, synthetic construct
  source 1..10: note = HCDR3
WLLSGNGMDY

SEQ ID NO: 143 - protein, 14 aa, synthetic construct
  source 1..14: note = HCDR3
EVYYGGYEDYGMDY

SEQ ID NO: 144 - protein, 13 aa, synthetic construct
  source 1..13: note = HCDR3
DYFYGDANPWFAY

SEQ ID NO: 145 - protein, 13 aa, synthetic construct
  source 1..13: note = HCDR3
DYFYGDGYPWFTY

SEQ ID NO: 146 - protein, 9 aa, synthetic construct
  source 1..9: note = HCDR3
DRYDVWFAY

SEQ ID NO: 147 - protein, 7 aa, synthetic construct
  source 1..7: note = HCDR3
IYYGLDN

SEQ ID NO: 148 - protein, 7 aa, synthetic construct
  source 1..7: note = HCDR3
IHYGLDN

SEQ ID NO: 149 - protein, 11 aa, synthetic construct
  source 1..11: note = HCDR3
FTVEVTDAMDY

SEQ ID NO: 150 - protein, 9 aa, synthetic construct
  source 1..9: note = HCDR3
FILENYFDY

SEQ ID NO: 151 - protein, 8 aa, synthetic construct
  source 1..8: note = HCDR3
AGDYAFDY

SEQ ID NO: 152 - protein, 10 aa, synthetic construct
  source 1..10: note = HCDR3
HGQTGTWFAY

SEQ ID NO: 153 - protein, 4 aa, synthetic construct
  source 1..4: note = HCDR3
LLLR

SEQ ID NO: 154 - protein, 20 aa, synthetic construct
  source 1..20: note = HCDR3
SAYGDWFYGSSGPYADSIDA

SEQ ID NO: 155 - protein, 15 aa, synthetic construct
  source 1..15: note = HCDR3
AADSGYLYITDSIDA

SEQ ID NO: 156 - protein, 19 aa, synthetic construct
  source 1..19: note = HCDR3
SADTGYCSWSACIADSIDA

SEQ ID NO: 157 - protein, 15 aa, synthetic construct
  source 1..15: note = HCDR3
SSYGGGWSDVGSIDA

SEQ ID NO: 158 - protein, 9 aa, synthetic construct
  source 1..9: note = HCDR3
DSGAVSIDA

SEQ ID NO: 159 - protein, 11 aa, synthetic construct
  source 1..11: note = LCDR1
KASQNVGTNVA

SEQ ID NO: 160 - protein, 11 aa, synthetic construct
  source 1..11: note = LCDR1
ITSTDIDDDMN

SEQ ID NO: 161 - protein, 16 aa, synthetic construct
  source 1..16: note = LCDR1
RSSQSLVHSNGNTYLH

SEQ ID NO: 162 - protein, 15 aa, synthetic construct
  source 1..15: note = LCDR1
RASESVDSYGNSFMH

SEQ ID NO: 163 - protein, 11 aa, synthetic construct
  source 1..11: note = LCDR1
KASQDVGTAVA

SEQ ID NO: 164 - protein, 16 aa, synthetic construct
  source 1..16: note = LCDR1
RSSQSLLHTNVNTYLH

SEQ ID NO: 165 - protein, 11 aa, synthetic construct
  source 1..11: note = LCDR1
KASQDVSTAVA

SEQ ID NO: 166 - protein, 12 aa, synthetic construct
  source 1..12: note = LCDR1
RASSSLSSTYLH

SEQ ID NO: 167 - protein, 9 aa, synthetic construct
  source 1..9: note = LCDR1
SGSSDSWYG

SEQ ID NO: 168 - protein, 13 aa, synthetic construct
  source 1..13: note = LCDR1
SGGDNYAGSYYYG

SEQ ID NO: 169 - protein, 8 aa, synthetic construct
  source 1..8: note = LCDR1
SGGSGSYG

SEQ ID NO: 170 - protein, 8 aa, synthetic construct
  source 1..8: note = LCDR1
SGGGSDYG

SEQ ID NO: 171 - protein, 10 aa, synthetic construct
  source 1..10: note = LCDR1
SGDRSYYGYG

SEQ ID NO: 172 - protein, 8 aa, synthetic construct
  source 1..8: note = LCDR1
SGGDSDYG

SEQ ID NO: 173 - protein, 7 aa, synthetic construct
  source 1..7: note = LCDR2
SASYRYS

SEQ ID NO: 174 - protein, 7 aa, synthetic construct
  source 1..7: note = LCDR2
EGNTLRP

SEQ ID NO: 175 - protein, 7 aa, synthetic construct
  source 1..7: note = LCDR2
KVSNRFS

SEQ ID NO: 176 - protein, 7 aa, synthetic construct
  source 1..7: note = LCDR2
RASNLES

SEQ ID NO: 177 - protein, 7 aa, synthetic construct
  source 1..7: note = LCDR2
WASTRHT

SEQ ID NO: 178 - protein, 7 aa, synthetic construct
  source 1..7: note = LCDR2
SASYRYT

SEQ ID NO: 179 - protein, 7 aa, synthetic construct
  source 1..7: note = LCDR2
STSNLAS

SEQ ID NO: 180 - protein, 7 aa, synthetic construct
  source 1..7: note = LCDR2
DSTNRPS

SEQ ID NO: 181 - protein, 7 aa, synthetic construct
  source 1..7: note = LCDR2
DDTNRPA

SEQ ID NO: 182 - protein, 7 aa, synthetic construct
  source 1..7: note = LCDR2
HNDQRPS

SEQ ID NO: 183 - protein, 7 aa, synthetic construct
  source 1..7: note = LCDR2
WSDNRPS

SEQ ID NO: 184 - protein, 7 aa, synthetic construct
  source 1..7: note = LCDR2
WDDKRPS

SEQ ID NO: 185 - protein, 7 aa, synthetic construct
  source 1..7: note = LCDR2
QNDKRPS

SEQ ID NO: 186 - protein, 9 aa, synthetic construct
  source 1..9: note = LCDR3
QQYNSYPYT

SEQ ID NO: 187 - protein, 9 aa, synthetic construct
  source 1..9: note = LCDR3
LQSDNLPFT

SEQ ID NO: 188 - protein, 9 aa, synthetic construct
  source 1..9: note = LCDR3
LQSDNMPLT

SEQ ID NO: 189 - protein, 9 aa, synthetic construct
  source 1..9: note = LCDR3
FQGSHVPWT

SEQ ID NO: 190 - protein, 9 aa, synthetic construct
  source 1..9: note = LCDR3
LQSDNLPLT

SEQ ID NO: 191 - protein, 9 aa, synthetic construct
  source 1..9: note = LCDR3
QQSNEDPWT

SEQ ID NO: 192 - protein, 8 aa, synthetic construct
  source 1..8: note = LCDR3
QQYSSYPT

SEQ ID NO: 193 - protein, 9 aa, synthetic construct
  source 1..9: note = LCDR3
SQSTHVPYT

SEQ ID NO: 194 - protein, 9 aa, synthetic construct
  source 1..9: note = LCDR3
QQYNSYPFT

SEQ ID NO: 195 - protein, 9 aa, synthetic construct
  source 1..9: note = LCDR3
QQYNSYPLT

SEQ ID NO: 196 - protein, 9 aa, synthetic construct
  source 1..9: note = LCDR3
QQYNNYPLT

SEQ ID NO: 197 - protein, 9 aa, synthetic construct
  source 1..9: note = LCDR3
QQHYSTPRT

SEQ ID NO: 198 - protein, 9 aa, synthetic construct
  source 1..9: note = LCDR3
LQSGTLPFT

SEQ ID NO: 199 - protein, 9 aa, synthetic construct
  source 1..9: note = LCDR3
QQSNEDPYT

SEQ ID NO: 200 - protein, 9 aa, synthetic construct
  source 1..9: note = LCDR3
QQYSGYPLT

SEQ ID NO: 201 - protein, 11 aa, synthetic construct
  source 1..11: note = LCDR3
GSFDSSTDSTA

SEQ ID NO: 202 - protein, 11 aa, synthetic construct
  source 1..11: note = LCDR3
GGWDSINDRNI

SEQ ID NO: 203 - protein, 11 aa, synthetic construct
  source 1..11: note = LCDR3
GGYDNDNDAGI

SEQ ID NO: 204 - protein, 10 aa, synthetic construct
  source 1..10: note = LCDR3
GGYDSSGSGM

SEQ ID NO: 205 - protein, 10 aa, synthetic construct
  source 1..10: note = LCDR3
GNMDSSYVGM

SEQ ID NO: 206 - protein, 11 aa, synthetic construct
  source 1..11: note = LCDR3
GGWDGSYDGAV

SEQ ID NO: 207 - DNA, 1386 bp, synthetic construct
  source 1..1386: mol_type = other DNA; note = ONA-0 heavy chain
caagtgcagctgaagcagtccggagctgatctggtgagacccggagccagcgtgaagctgagctgcaaggccagcggctacaccttcaccgactactacatcaactgggtgaagcagaggcccggccaaggactggagtggatcgctagaatctaccccggctccggcaatacatactacaacgagaagttcaaaggcaaggccacactgaccgccgagaagagcagcagcaccgcctacatgcagctgagctctctgacctccgaggacagcgccgtgtacttttgcgccagaggcatcggaggcggattcggcatggattactggggccaaggcacctccgtgaccgtctcgagcgaatcggccagaaaccccactatctaccctctgaccctgcctcctgtcctgtgttccgaccccgtgatcatcggatgcctgatccacgactacttccctttcggcaccatgaacgtgacctgggggaagtcgggaaaggacattactaccgtgaacttcccaccggccctggcgtcggggggtcgctacaccatgtccagccagcttactctgcccgctgtggagtgccccgaaggagagtcagtgaagtgctccgtgcaacatgactccaacccggtccaggaattggacgtcaattgctccccgactccgcctccgcctatcacgatcccaagctgccagccctccctgagcctccagcggccagccctggaggatcttctgctgggctccgacgcctccattacatgcactctgaacggcctgagaaacccggaaggggcggcctttacttgggagccctccaccgggaaggatgcggtccagaagaaggcagcccaaaattcctgcggatgctactcagtgtctagcgtgctgcctggttgtgccgaacggtggaactccggagcgtcattcaagtgtaccgtgacccaccctgagtccggaactctgaccggcaccatcgccaaggtcaccgtgaacacctttccgccacaagtgcacctcctgccgccgccgtcggaggaactcgctctgaacgagttgctctcgctgacttgtctcgtgcgcgccttcaaccctaaggaggtgctcgtgcgctggctgcatggcaacgaagaactgtcccccgaatcgtacctggtgttcgaaccgctgaaagagcccggagagggtgcaaccacctaccttgtgacgagcgtgctccgggtgtccgccgaaacctggaagcagggcgaccagtacagctgcatggtcggccacgaggccctccccatgaacttcactcagaaaaccattgataggttgtccggaaagcccaccaacgtgtcagtgtccgtgattatgagcgaaggagatggaatctgctat

SEQ ID NO: 208 - DNA, 642 bp, synthetic construct
  source 1..642: mol_type = other DNA; note = ONA-0-v1 light chain
tccatcgtgatgacccagacccccaagtttctgctggtgtccgccggagacagaatcaccatcacatgcaaggccagccagagcgtgagcgatgacgtggcttggtaccagcagaagcccggccagagccctaagctgctgatctactacgccagcaatagatacaccggagtgcccgatagattcaccggcagcggctacggcaccgacttcaccttcacaatctccaccgtgcaagccgaggatctggccgtgtacttctgtcagcaagactactccagccctctgaccttcggagccggcaccaagctcgagatcaagcgcgcagatgctgctcctaccgtgagcatcttcccgccgtccagcgaacaactcactagcggaggcgcgtcagtggtctgcttccttaacaatttctaccctaaggacatcaacgtcaagtggaagattgacggatcggaacgccagaacggagtgctgaactcatggactgatcaggattccaaagactcgacttactccatgtccagcaccctgaccctgaccaaagacgagtacgaaaggcacaactcgtacacgtgcgaagccacccacaagacttccacctcgcccatcgtgaagtccttcaatcgcaatgagtgc

SEQ ID NO: 209 - DNA, 357 bp, synthetic construct
  source 1..357: mol_type = other DNA; note = ONA-0-v1 VH
caagtgcagctgaagcagtccggagctgatctggtgagacccggagccagcgtgaagctgagctgcaaggccagcggctacaccttcaccgactactacatcaactgggtgaagcagaggcccggccaaggactggagtggatcgctagaatctaccccggctccggcaatacatactacaacgagaagttcaaaggcaaggccacactgaccgccgagaagagcagcagcaccgcctacatgcagctgagctctctgacctccgaggacagcgccgtgtacttttgcgccagaggcatcggaggcggattcggcatggattactggggccaaggcacctccgtgaccgtctcgagc

SEQ ID NO: 210 - DNA, 321 bp, synthetic construct
  source 1..321: mol_type = other DNA; note = ONA-0-v1 VL
tccatcgtgatgacccagacccccaagtttctgctggtgtccgccggagacagaatcaccatcacatgcaaggccagccagagcgtgagcgatgacgtggcttggtaccagcagaagcccggccagagccctaagctgctgatctactacgccagcaatagatacaccggagtgcccgatagattcaccggcagcggctacggcaccgacttcaccttcacaatctccaccgtgcaagccgaggatctggccgtgtacttctgtcagcaagactactccagccctctgaccttcggagccggcaccaagctcgagatcaag

SEQ ID NO: 211 - DNA, 1347 bp, synthetic construct
  source 1..1347: mol_type = other DNA; note = 1G04 heavy chain
caagtgcagctgaagcagtccggagctgatctggtgagacccggagccagcgtgaagctgagctgcaaggccagcggctacaccttcaccgactactacatcaactgggtgaagcagaggcccggccaaggactggagtggatcgctagaatctaccccggctccggcaatacatactacaacgagaagttcaaaggcaaggccacactgaccgccgagaagagcagcagcaccgcctacatgcagctgagctctctgacctccgaggacagcgccgtgtacttttgcgccagaggcatcggaggcggattcggcatggattactggggccaaggcacctccgtgaccgtctcgagcgccagcaccaaaggtccatccgtgtttccgctcgccccgtcctcaaagtcgacctccggaggcactgccgccctgggctgccttgtcaaggactatttccccgaacctgtcacggtgtcctggaacagcggcgctctgacttccggagtgcacaccttccccgccgtcctgcaatccagcggcctgtactcactgtcatccgttgtgactgtcccgtcgtccagcctgggaacccaaacctacatttgcaacgtgaatcacaaaccatcgaataccaaggtcgataagaaagtcgagccgaagtcatgcgacaagactcacacctgtccgccttgcccggcgccagaagcggccggcggcccttcggtgtttttgtttccgccgaagccgaaggacactctgatgatctcacgcactccagaggtgacttgcgtggtggtcgatgtttcgcacgaggacccggaagtgaaattcaactggtatgtcgacggggtggaagtgcataatgccaagacgaagccgagggaggaacagtacaactccacctacagagtggtttcagtccttaccgtcctccatcaagattggctgaacggaaaggagtacaaatgtaaggtgtcgaacaaagcgttgccggcccctatcgaaaagactatcagcaaggccaaaggacagccgcgggagccgcaagtgtacaccctcccgccttcgcgggacgagctgaccaagaatcaggtgtcccttacttgcctggtgaagggattctacccctcggatatcgcagtcgaatgggaatcgaatggacagccagaaaacaactacaagaccactcccccggtgctcgactccgacggttccttcttcctgtactcgaagctgaccgtggacaaatcacgctggcagcagggaaacgtgtttagctgcagcgtgatgcatgaggcgctgcataatcactacacccagaagtcactctcgctcagcccagggaag

SEQ ID NO: 212 - DNA, 642 bp, synthetic construct
  source 1..642: mol_type = other DNA; note = 1G04 light chain
tccatcgtgatgacccagacccccaagtttctgctggtgtccgccggagacagaatcaccatcacatgcaaggccagccagagcgtgagcgatgacgtggcttggtaccagcagaagcccggccagagccctaagctgctgatctactacgccagcaatagatacaccggagtgcccgatagattcaccggcagcggctacggcaccgacttcaccttcacaatctccaccgtgcaagccgaggatctggccgtgtacttctgtcagcaagactactccagccctctgaccttcggagccggcaccaagctcgagatcaagagaactgtggccgcgccgtcagtgtttatcttccctccatcggatgaacagcttaagtccggcacggcgtctgtggtctgcctgctcaataacttttaccctagggaagctaaagtccaatggaaagtggataacgccctgcagtcaggaaacagccaggaatcggttaccgaacaggacagcaaggacagcacttactccttgtcgtcgactcttactctgagcaaggccgattacgagaagcacaaggtctacgcctgcgaggtcacccatcagggactctcgtccccggtgaccaaatccttcaatagaggcgaatgc

SEQ ID NO: 213 - protein, 483 aa, synthetic construct
  source 1..483: note = Flag-tagged human CD36
MDYKDDDDKSSGGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 214 - protein, 483 aa, synthetic construct
  source 1..483: note = CD36-Vlm1
MDYKDDDDKSSGGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFEADVALKGIPVARFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 215 - protein, 483 aa, synthetic construct
  source 1..483: note = CD36-Vlm2
MDYKDDDDKSSGGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVAAAQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 216 - protein, 483 aa, synthetic construct
  source 1..483: note = CD36-Vlm3
MDYKDDDDKSSGGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSAAFASPVENPANYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 217 - protein, 483 aa, synthetic construct
  source 1..483: note = CD36-Vlm4
MDYKDDDDKSSGGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPAAAEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 218 - protein, 15 aa, synthetic construct
  source 1..15: note = CD36 278-292
VFESDVNLKGIPVYR

SEQ ID NO: 219 - protein, 15 aa, synthetic construct
  source 1..15: note = CD36-V1m1 278-292
VFEADVALKGIPVAR

SEQ ID NO: 220 - protein, 15 aa, synthetic construct
  source 1..15: note = CD36 249-263
TDAASFPPFVEKSQV

SEQ ID NO: 221 - protein, 15 aa, synthetic construct
  source 1..15: note = CD36-V1m2 249-263
TDAASFPPFVAAAQV

SEQ ID NO: 222 - protein, 15 aa, synthetic construct
  source 1..15: note = CD36 295-309
LPSKAFASPVENPDN

SEQ ID NO: 223 - protein, 15 aa, synthetic construct
  source 1..15: note = CD36-V1m3 295-309
LPSAAFASPVENPAN

SEQ ID NO: 224 - protein, 15 aa, synthetic construct
  source 1..15: note = CD36 354-368
SEPIDGLNPNEEEHR

SEQ ID NO: 225 - protein, 15 aa, synthetic construct
  source 1..15: note = CD36-V1m4 354-368
SEPIDGLNPAAAEHR

SEQ ID NO: 226 - DNA, 360 bp, synthetic construct
  source 1..360: mol_type = other DNA; note = 6G04 VH
caagtgcagctgcagcagagcggcgctgaactggtcatgcccggcgctagcgtgaagatgagctgcaaggccagcggctacactttcactgactactggatgcactgggtgaagcagaggcccggccaaggactggagtggatcggcagcatcgacacttccgacagctacacaacttactcccagaaattcaagggcaaggccacactgacagtggacgagagcagcagcacagcctacatggagctggctaggctgactagcgaggatagcgccatctactactgtgtgaggggcgaggattacgagggaacatggttcgcctactggggccaaggcacactggtgactgtgagcgcc

SEQ ID NO: 227 - DNA, 321 bp, synthetic construct
  source 1..321: mol_type = other DNA; note = 6G04 VL
gagactacagtgactcagagcccagcctctctgagcgtcgctactggcgagaaggtcacaattaggtgcatcactagcacagacatcgacgacgacatgaactggtatcagcagaagcccggcgagcctcctaagctgctgatctccgagggcaacacactgaggcccggcgtgccttctaggttcagcagcagcggctacggcacagacttcgtcttcactatcgagaacactctgagcgaggacgtggccgattactactgtctgcagagcgacaacatgcctctgactttcggcgccggcacaaagctggagctgaaa

SEQ ID NO: 228 - DNA, 360 bp, synthetic construct
  source 1..360: mol_type = other DNA; note = 7G04 VH
caagtgcagatgaaggaaagcggcgctgagctggtgagacccggcgctagcgtgaagctgagctgtaaggctctgggctacacattcacagactacgagatccagtgggtgaagcagacaccagtgcacggactggaatggatcggaggcatccaccccggcagctccggcatcgtgtacaaccagaagttcaagggcaaggccactctgactgctgacaagtccagcagcactgcctacatggagctgagctctctgactagcgaggatagcgccgtgtactactgtacaaggggcggcggctacgatggagcttggttcgcctattggggacaaggctctctggtcactgtgagcgcc

SEQ ID NO: 229 - DNA, 321 bp, synthetic construct
  source 1..321: mol_type = other DNA; note = 7G04 VL
gagacaactgtgactcagagcccagcctctctgtccatggctatcggcgagaaggtgacaattaggtgcatcactagcacagacatcgacgacgacatgaactggtatcagcaaaagcccggcgagcctcctaagctgctgatcagcgagggcaatacactgaggcccggcgtgccttctaggttcagcagcagcggctacggcacagacttcgtgttcactatcgagaacatgctgagcgaggacgtggctgattactactgtctgcagagcgacaatctgcctttcacattcggcagcggcacaaagctggagatcaag

SEQ ID NO: 230 - DNA, 345 bp, synthetic construct
  source 1..345: mol_type = other DNA; note = 11G04 VH
gaggtgcagctccagcagagcggcgccgaactggtgagacccggcgccagcgtgacactgagctgtaaggccagcggctataggttctccgattacgagatgcagtgggtgaagcagactccagtccacggactggaatggatcggcggcatcgatccagagactggcggcatcgcctacaaccagaagttcaaggtcaaggccactctcactgccgacaagagctcctccacagccttcatggagctgaggtctctgacaagcgaggatagcgccgtctactactgcactaggaagctggacttcgactactggggccaaggcactacactgactgtgagcagc

SEQ ID NO: 231 - DNA, 321 bp, synthetic construct
  source 1..321: mol_type = other DNA; note = 11G04 VL
gagacaactgtgacacagagcccagcctctctgagcgtcgctactggcgagaaggtgactattaggtgcatcacaagcacagacatcgacgacgacatgaactggtatcagcagaagcccggcgagcctcctaagctgctgatcagcgagggaaacactctgaggcccggcgtgccatctaggttcagcagcagcggctacggcactgacttcgtgttcacaatcgagaacatgctgagcgaggacgtggccgactactactgtctgcagagcgacaatctgcctctgactttcggcgccggcacaaagctggagctgaag

SEQ ID NO: 232 - DNA, 366 bp, synthetic construct
  source 1..366: mol_type = other DNA; note = 13G04 VH
caagtgcagctgcagcagagcggcgctgaactggtgagacccggcgccagcgtcaagctgagctgcaaggcctccggctacactttcacaagctactggatgaactgggtgaagcagagacccggccaaggactggagtggatcggcatgatcgaccctagcgacaacgagacacactacaaccagatgttcaaggacaaggccacactgactgtcgacaagagctcctccacagcctacatgcagctgagctctctgactagcgaggatagcgccgtgtactactgcgctaggagcgattacggcaacggctatagcttctatctggacgtctggggcgctggcacaactgtgactgtgagcagc

SEQ ID NO: 233 - DNA, 333 bp, synthetic construct
  source 1..333: mol_type = other DNA; note = 13G04 VL
gatatcgtgctgacacagagcccagcctctctggctgtgtctctgggacagagagccacaatcagctgtagggccagcgagagcgtggatagctacggcaactccttcatgcactggtatcagcagaagcccggccagcctcctaagctgctgatctatagggccagcaatctggagagcggcatcccagctaggtttagcggctccggctctaggactgacttcactctgactatcaacccagtggaggccgacgatgtggccacttactactgccagcagagcaacgaggatccttggacattcggcggcggcactaagctggagatcaag

SEQ ID NO: 234 - DNA, 357 bp, synthetic construct
  source 1..357: mol_type = other DNA; note = 14G04 VH
caagtgcagctgcagcagagcggcgccgaactggtgagaagcggagctagcgtgaggctgagctgtacagcctccggcttcaacatcaaggactactacatccactgggtgaagcagaggccagagcaaggactggagtggatcggctggatcgatccagagaatggcgacacagagtacgctccaaggttccaagataaggccactatgactgccgacactagcagcaacacagcctatctgcagctgagctctctgacaagcgaggacacagctgtgtactactgcaatggctggctgctgagcggcaatggcatggattactggggccaaggcacatccgtgacagtgagctcc

SEQ ID NO: 235 - DNA, 318 bp, synthetic construct
  source 1..318: mol_type = other DNA; note = 14G04 VL
gacatcgtgatgacacagagccacaagttcatgagcacaagcgtgggcgatagggtgagcatcacttgcaaggctagccaagatgtgggcacagctgtggcttggtatcagcagaagcccggccagagcccaaagctgctgatctactgggccagcactaggcacactggcgtgccagataggttcactggcagcggcagcggcacagacttcactctgactatcagcaacgtgcagagcgaggatctggccgattacttctgccagcagtactccagctaccctacattcggcggcggcacaaagctggagatcaag

SEQ ID NO: 236 - DNA, 351 bp, synthetic construct
  source 1..351: mol_type = other DNA; note = 28G04 VH
gaggtgcagctgcagcagagcggaccagagctggtgaagcccggcgccagcgtcaagatcagctgcaagactagcggctacactttcactgagtacactatccactgggtgaagcagagccacggcaagtctctggagtggatcggcggcatcatccctaacaatggcggcagcagccacaagcagaacttcaaggacaaggccactctgacagtggataagagcagcagcacagcctacatggagctgaggtctctgacaagcgaggattccgccgtctactactgtgctagggccggcgactacgccttcgattactggggccaaggcactacactgacagtcagcagc

SEQ ID NO: 237 - DNA, 321 bp, synthetic construct
  source 1..321: mol_type = other DNA; note = 28G04 VL
gagacaactgtgacacagagcccagcttctctgagcatggccatcggcgagaaggtcacaattaggtgcatcacttccacagacatcgacgacgacatgaactggtatcagcagaagcccggcgagcctcctaagctgctgatcagcgagggaaacactctgaggcccggcgtgccatctaggttcagcagcagcggctacggcacagatttcgtgttcactatcgagaacatgctgagcgaggacgtggccgactactactgtctgcagagcggaactctgccattcactttcggctccggcactaagctggagatcaag

SEQ ID NO: 238 - protein, 8 aa, synthetic construct
  source 1..8: note = Flag tag
DYKDDDDK

SEQ ID NO: 239 - protein, 6 aa, synthetic construct
  source 1..6: note = B7 binding motif
MYPPPY

SEQ ID NO: 240 - protein, 214 aa, synthetic construct
  source 1..214: note = 11G06 VL 1
ETTVTQSPAFMSATTGDKVTISCITSTDIDDDMNWYQQKPGEPPKLLISEGNTLRPGVPSRFSSSGYGTDFTFTINNIESEDAAYYYCLQSDNLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 241 - protein, 445 aa, synthetic construct
  source 1..445: note = 11G06 VH 1
EVQLVQSGAEVKKPGSSVKVSCKASGYRFSDYEMQWVRQAPGQGLEWMGGIDPETGGIAYAQKFQGRVTLTADKSTSTAYMELSSLRSEDTAVYYCTRKLDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 214 aa, synthetic construct
  source 1..214: note = 11G06 VL 2
ETTVTQSPAFMSATPGDKVTISCITSTDIDDDMNWYQQKPGEPPKLLISEGNTLRPGVPSRFSSSGYGTDFTFTINNIESEDAAYYYCLQSDNLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - protein, 445 aa, synthetic construct
  source 1..445: note = 11G06 VH 2
QVQLVQSGAEVKKPGSSVKVSCKASGYRFSDYEMQWVRQAPGQGLEWMGGIDPETGGIAYAQKFQGRVTLTADKSTSTAYMELSSLRSEDTAVYYCTRKLDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 214 aa, synthetic construct
  source 1..214: note = 11G06 VL 3
ETTVTQSPAFMSATTGDKVTISCITSTDIDDDMNWYQQKPGEPPKLLISEGSTLRPGVPSRFSSSGYGTDFTFTINNIESEDAAYYYCLQSDNLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - protein, 214 aa, synthetic construct
  source 1..214: note = 11G06 VL 4
ETTVTQSPAFMSATPGDKVTISCITSTDIDDDMNWYQQKPGEPPKLLISEGSTLRPGVPSRFSSSGYGTDFTFTINNIESEDAAYYYCLQSDNLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 7 aa, synthetic construct
  source 1..7: note = 11G06 VL_3-4 CDR2
EGSTLRP

SEQ ID NO: 247 - protein, 9 aa, synthetic construct
  source 1..9: note = 11G06 VL_1-4 CDR3
LQSGNSQES

SEQ ID NO: 248 - protein, 17 aa, synthetic construct
  source 1..17: note = 11G06 VH_1-2 CDR2
GIDPETGGIAYAQKFQG

SEQ ID NO: 249 - protein, 727 aa, synthetic construct
  source 1..727: note = 11G06_VL
AAGCTTGCCGCCACCATGTCTGTGCCTACACAGGTTCTGGGACTGCTGCTGCTGTGGCTGACCGACGCTAGATGCGAGACAACCGTGACTCAGTCCCCTGCCTCTCTGTCTGTGGCTACCGGCGAGAAAGTGACCATCCGGTGCATCACCTCCACCGACATCGACGACGACATGAACTGGTATCAGCAGAAGCCCGGCGAGCCTCCTAAGCTGCTGATCTCTGAGGGCAACACACTGAGGCCTGGCGTGCCCTCCAGATTCTCCTCTTCTGGCTACGGCACCGACTTCGTGTTCACCATCGAGAACATGCTGTCCGAGGACGTGGCCGACTACTACTGCCTGCAGTCTGACAACCTGCCTCTGACCTTTGGCGCTGGCACCAAGCTGGAACTGAAGAGAACAGTGGCCGCTCCTTCCGTGTTCATCTTCCCACCTTCCGACGAGCAGCTGAAGTCTGGCACAGCCTCTGTCGTGTGCCTGCTGAACAACTTCTACCCTCGGGAAGCCAAGGTGCAGTGGAAGGTGGACAACGCTCTGCAGTCCGGCAACTCCCAAGAGTCTGTGACCGAGCAGGACTCCAAGGACAGCACCTACAGCCTGTCCTCCACACTGACCCTGTCCAAGGCCGATTACGAGAAGCACAAGGTGTACGCCTGCGAAGTGACCCATCAGGGCCTGTCTAGCCCTGTGACCAAGTCTTTCAACCGGGGCGAGTGCTGATGAATTC

SEQ ID NO: 250 - protein, 1417 aa, synthetic construct
  source 1..1417: note = 11G06_VH
AAGCTTGCCGCCACCATGGAATGGTCCTGGGTGTTCCTGTTCTTCCTGTCCGTGACCACCGGCGTGCACTCTGAAGTTCAGTTGCAGCAGTCTGGCGCCGAACTCGTTAGACCTGGCGCTTCTGTGACCCTGTCCTGCAAGGCTTCCGGCTACCGGTTCTCCGACTACGAGATGCAGTGGGTCAAGCAGACCCCTGTGCATGGCCTGGAATGGATCGGCGGCATCGATCCTGAGACAGGCGGAATCGCCTACAACCAGAAGTTCAAAGTCAAGGCTACCCTGACCGCCGACAAGTCCTCTTCCACCGCCTTCATGGAACTGCGGTCCCTGACCTCTGAGGACTCCGCCGTGTACTACTGCACCCGGAAGCTGGACTTCGACTATTGGGGCCAGGGCACCACACTGACCGTGTCCTCTGCTTCTACCAAGGGACCCAGCGTGTTCCCTCTGGCTCCTTCCAGCAAGTCTACCTCTGGCGGAACAGCTGCTCTGGGCTGCCTGGTCAAGGACTACTTTCCTGAGCCTGTGACAGTGTCCTGGAACTCTGGCGCTCTGACATCTGGCGTGCACACCTTTCCAGCTGTGCTGCAGTCCTCCGGCCTGTACTCTCTGTCCTCTGTCGTGACCGTGCCTTCCAGCTCTCTGGGAACCCAGACCTACATCTGCAATGTGAACCACAAGCCTTCCAACACCAAGGTGGACAAGAAGGTGGAACCCAAGTCCTGCGACAAGACCCACACCTGTCCTCCATGTCCTGCTCCAGAGTCCACCAGAGGACCCTCCGTGTTTCTGTTCCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTACAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGACATGCCTCGTGAAGGGCTTCTACCCCTCCGATATCGCCGTGGAATGGGAGTCTAATGGCCAGCCTGAGAACAACTACAAGACAACCCCTCCTGTGCTGGACTCCGACGGCTCATTCTTTCTGTACTCCAAGCTGACAGTGGACAAGTCCAGATGGCAGCAGGGCAACGTGTTCTCCTGCAGCGTGATGCACGAGGCCCTGCACAATCACTATACCCAGAAGTCCCTGTCTCTGTCCCCTGGCAAGTGATGAATTC

SEQ ID NO: 251 - protein, 234 aa, synthetic construct
  source 1..234: note = 11G06 VL AA
MSVPTQVLGLLLLWLTDARCETTVTQSPASLSVATGEKVTIRCITSTDIDDDMNWYQQKPGEPPKLLISEGNTLRPGVPSRFSSSGYGTDFVFTIENMLSEDVADYYCLQSDNLPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 252 - protein, 464 aa, synthetic construct
  source 1..464: note = 11G06 HL AA
MEWSWVFLFFLSVTTGVHSEVQLQQSGAELVRPGASVTLSCKASGYRFSDYEMQWVKQTPVHGLEWIGGIDPETGGIAYNQKFKVKATLTADKSSSTAFMELRSLTSEDSAVYYCTRKLDFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 253 - DNA, 727 bp, synthetic construct
  source 1..727: mol_type = other DNA; note = 11G06 _VL 1
aagcttgccgccaccatgtctgtgcctacacaggttctgggactgctgctgctgtggctgaccgacgctagatgcgagacaaccgtgactcagtcccctgccttcatgtctgctaccaccggcgacaaagtgaccatctcctgcatcacctccaccgacatcgacgacgacatgaactggtatcagcagaagcccggcgagcctcctaagctgctgatctctgagggcaacacactgaggcctggcgtgccctccagattctcctcttctggctacggcaccgacttcaccttcaccatcaacaacatcgagtccgaggacgccgcctactactactgcctgcagtctgacaacctgcctctgacctttggccagggcaccaagctggaaatcaagcggacagtggccgctccttccgtgttcatcttcccaccttccgacgagcagctgaagtctggcacagcctctgtcgtgtgcctgctgaacaacttctaccctcgggaagccaaggtgcagtggaaggtggacaacgctctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctgatgaattc

SEQ ID NO: 254 - DNA, 727 bp, synthetic construct
  source 1..727: mol_type = other DNA; note = 11G06_VL 2
aagcttgccgccaccatgtctgtgcctacacaggttctgggactgctgctgctgtggctgaccgacgctagatgcgagacaaccgtgactcagtcccctgccttcatgtctgctacccctggcgacaaagtgaccatctcctgcatcacctccaccgacatcgacgacgacatgaactggtatcagcagaagcccggcgagcctcctaagctgctgatctctgagggcaacacactgaggcctggcgtgccctccagattctcctcttctggctacggcaccgacttcaccttcaccatcaacaacatcgagtccgaggacgccgcctactactactgcctgcagtctgacaacctgcctctgacctttggccagggcaccaagctggaaatcaagcggacagtggccgctccttccgtgttcatcttcccaccttccgacgagcagctgaagtctggcacagcctctgtcgtgtgcctgctgaacaacttctaccctcgggaagccaaggtgcagtggaaggtggacaacgctctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctgatgaattc

SEQ ID NO: 255 - DNA, 727 bp, synthetic construct
  source 1..727: mol_type = other DNA; note = 11G06_VL 3
aagcttgccgccaccatgtctgtgcctacacaggttctgggactgctgctgctgtggctgaccgacgctagatgcgagacaaccgtgactcagtcccctgccttcatgtctgctaccaccggcgacaaagtgaccatctcctgcatcacctccaccgacatcgacgacgacatgaactggtatcagcagaagcccggcgagcctcctaagctgctgatctctgagggctctacactgaggcctggcgtgccctccagattctcctcttctggctacggcaccgacttcaccttcaccatcaacaacatcgagtccgaggacgccgcctactactactgcctgcagtctgacaacctgcctctgacctttggccagggcaccaagctggaaatcaagcggacagtggccgctccttccgtgttcatcttcccaccttccgacgagcagctgaagtctggcacagcctctgtcgtgtgcctgctgaacaacttctaccctcgggaagccaaggtgcagtggaaggtggacaacgctctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctgatgaattc

SEQ ID NO: 256 - DNA, 727 bp, synthetic construct
  source 1..727: mol_type = other DNA; note = 11G06_VL 4
aagcttgccgccaccatgtctgtgcctacacaggttctgggactgctgctgctgtggctgaccgacgctagatgcgagacaaccgtgactcagtcccctgccttcatgtctgctacccctggcgacaaagtgaccatctcctgcatcacctccaccgacatcgacgacgacatgaactggtatcagcagaagcccggcgagcctcctaagctgctgatctctgagggctctacactgaggcctggcgtgccctccagattctcctcttctggctacggcaccgacttcaccttcaccatcaacaacatcgagtccgaggacgccgcctactactactgcctgcagtctgacaacctgcctctgacctttggccagggcaccaagctggaaatcaagcggacagtggccgctccttccgtgttcatcttcccaccttccgacgagcagctgaagtctggcacagcctctgtcgtgtgcctgctgaacaacttctaccctcgggaagccaaggtgcagtggaaggtggacaacgctctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctgatgaattc

SEQ ID NO: 257 - DNA, 1417 bp, synthetic construct
  source 1..1417: mol_type = other DNA; note = 11G06_VH 1
aagcttgccgccaccatggaatggtcctgggtgttcctgttcttcctgtccgtgaccaccggcgtgcactctgaagttcagctggttcagtctggcgccgaagtgaagaaacctggctcctccgtgaaggtgtcctgcaaggcttccggctaccggttctccgactacgagatgcagtgggtccgacaggctcctggacaaggcttggaatggatgggcggcatcgatcctgagacaggcggaatcgcttacgcccagaaattccagggcagagtgaccctgaccgccgacaagtctacctccaccgcctacatggaactgtccagcctgagatctgaggacaccgccgtgtactactgcacccggaagctggacttcgactattggggccagggcaccctggtcacagtgtcctctgcttctaccaagggacccagcgtgttccctctggctcctagctctaagtccacctctggcggaacagctgctctgggctgtctggtcaaggactacttccctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagagtccaccagaggaccctccgtgtttctgttccctccaaagcctaaggacaccctgtacatcacccgcgagcctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgatatcgccgtggaatgggagtctaatggccagccagagaacaactacaagacaacccctcctgtgctggactccgacggctcattctttctgtactccaagctgacagtggacaagtccagatggcagcagggcaacgtgttctcctgcagcgtgatgcacgaggccctgcacaatcactacacacagaagtccctgtctctgtcccctggcaagtgatgaattc

SEQ ID NO: 258 - DNA, 1417 bp, synthetic construct
  source 1..1417: mol_type = other DNA; note = 11G06_VH 2
aagcttgccgccaccatggaatggtcctgggtgttcctgttcttcctgtccgtgaccaccggcgtgcactctcaggttcagttggttcagtctggcgccgaagtgaagaaacccggctcctctgtgaaggtgtcctgcaaggcttctggctaccggttctccgactacgagatgcagtgggtccgacaggctcctggacaaggcttggaatggatgggcggcatcgatcctgagacaggcggaatcgcttacgcccagaaattccagggcagagtgaccctgaccgccgacaagtctacctccaccgcctacatggaactgtccagcctgagatctgaggacaccgccgtgtactactgcacccggaagctggacttcgactattggggccagggcaccctggtcacagtgtcctctgcttctaccaagggacccagcgtgttccctctggctcctagctctaagtccacctctggcggaacagctgctctgggctgtctggtcaaggactacttccctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagagtccaccagaggaccctccgtgtttctgttccctccaaagcctaaggacaccctgtacatcacccgcgagcctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgatatcgccgtggaatgggagtctaatggccagccagagaacaactacaagacaacccctcctgtgctggactccgacggctcattctttctgtactccaagctgacagtggacaagtccagatggcagcagggcaacgtgttctcctgcagcgtgatgcacgaggccctgcacaatcactacacacagaagtccctgtctctgtcccctggcaagtgatgaattc

(19) United States
(12) Patent Application Publication
(10) Pub. No.: US 2025/0206836 A1
(43) Pub. Date: Jun. 26, 2025

(54) ANTI-CD36 ANTIBODIES AND THEIR USE TO TREAT CANCER

(71) Applicant: ONA THERAPEUTICS S.L., Barcelona (ES)

(72) Inventors: Salvador AZNAR BENITAH, Barcelona (ES); Valerie VANHOOREN, Barcelona (ES); Mercè DE FRIAS SÁNCHEZ, Barcelona (ES); Beatriz MORANCHO ARMISEN, Barcelona (ES); Salvador GUARDIOLA BAGÁN, Barcelona (ES); Geoffrey HALE, Oxford (GB); Ian WILKINSON, Oxford (GB)

(21) Appl. No.: 18/293,581

(22) PCT Filed: Jul. 29, 2022
(86) PCT No.: PCT/IB2022/057098

§ 371 Date: Jan. 30, 2024

Related U.S. Application Data
(60) Provisional application No. 63/227,806, filed on Jul. 30, 2021.

(51) Int. Cl. C07K 16/28; A61K 39/00; A61P 35/00

(52) U.S. Cl. CPC C07K 16/2896; A61P 35/00; A61K 2039/505; C07K 2317/24; C07K 2317/76; C07K 2317/92

(57) ABSTRACT

The claimed invention relates to treating cancer by targeting CD36, a fatty acid receptor. The claimed invention also relates to treating cancer metastases by targeting CD36. The invention involves using anti-CD36 antibodies as blockers or inhibitors of CD36 activity to treat or prevent cancer and/or metastatic cancer in a subject.

### CROSS-REFERENCE TO RELATED APPLICATION

This application is a 35 U.S.C. § 371 National Phase Application of International Application No. PCT/IB2022/057098, filed Jul. 29, 2022, which claims the priority benefit of U.S. Provisional Application No. 63/227,806, filed Jul. 30, 2021, which are incorporated herein by reference in their entireties.

### REFERENCE TO SEQUENCE LISTING SUBMITTED ELECTRONICALLY

The content of the electronically submitted sequence listing in XML format (Name: 4427_0110001_Seglisting_ST26.xml; Size: 302,213 bytes; and Date of Creation: Feb. 7, 2025) filed with the application is incorporated herein by reference in its entirety.

### TECHNICAL FIELD

The disclosure relates to the treatment of cancer, particularly cancer metastases, and the control of said disease. More specifically, the disclosure relates to the use of anti-CD36 antibodies for the treatment of cancer. The disclosure also relates to the use of anti-CD36 antibodies for the treatment of primary cancers, cancer metastases, or both. The treatments relate to the use of both full-length antibodies and fragments thereof.

### BACKGROUND

CD36 (HGNC:1663, EntrezGene:948, Ensembl:ENSG00000135218, OMIM: 173510, UniProtKB: P16671) is a receptor protein with several different known functions, as it is indicated by the different alternative names that it receives: it is known, among others, as cluster determinant 36, thrombospondin receptor, collagen type I receptor, leukocyte differentiation antigen CD36, platelet glycoprotein 4 or fatty acid translocase. The Entrez Gene and UniProt/SwissProt Summaries for CD36 gene, as recapitulated by GeneCards (http://www.genecards.org/cgi-bin/carddisp.pl?gene=CD36) describe the protein as the fourth major glycoprotein of the platelet surface that serves as a receptor for thrombospondin in platelets and various cell lines. Since thrombospondins are widely distributed proteins involved in a variety of adhesive processes, this protein is implicated as a cell adhesion molecule. It binds to collagen and thrombospondin, mediating the antiangiogenic effect of the latter, as well as to anionic phospholipids and oxidized LDL. It directly mediates cytoadherence of Plasmodium falciparum parasitized erythrocytes and it binds long chain fatty acids. It is a co-receptor for the TLR4-TLR6 heterodimer that promotes inflammation in monocytes/macrophages. When CD36 binds a ligand such as oxidized LDL (“oxLDL”) or amyloid-beta 42, CD36 rapidly induces the formation of a heterodimer of TLR4 and TLR6. The TLR4-TLR6 heterodimer is internalized and triggers an inflammatory response that leads to NF-kappa-B-dependent production of CXCL1, CXCL2 and CCL9 cytokines (via the MYD88 signalling pathway), production of CCL5 cytokine (via the TICAM1 signalling pathway), and IL1b secretion. CD36 is also at the top of the signalling cascade that uptakes lipids from the extracellular environment and triggers their beta-oxidation to obtain energy in the form of ATP (Coburn, C. T. et al., J. Biol. Chem. 275(42):32523-9 (2000); Ibrahimi, A. et al., J. Biol. Chem. 274(38):26761-6 (1999); Pepino, M. Y. et al., Annu. Rev. Nutr. 34:281-303 (2014).

CD36 has been previously linked to cancer, but its implication for therapy and mechanism of action were not clear. WO 03/032813 discloses assays where it is shown that CD36 is one of the genes upregulated in renal cell carcinoma. Squamous cell carcinoma (SCC) is mentioned as one of the possible cancer types where the treatment with CD36 antibodies, or antagonists such as antisense RNA, can be of use, but without providing any evidence of changes of CD36 expression in SCC or, particularly, of the efficacy of CD36 antibodies or other antagonists for preventing or treating either primary tumors or metastases. Spontaneous animal tumors are proposed for testing the efficacy of antibodies specifically binding the proteins that are overexpressed in renal cell carcinoma according to the assays shown in WO 03/032813, and, given that it is a highly invasive and malignant tumor, feline oral SCC is proposed as a suitable model. However, again, such proposal is done without providing examples of the actual utility of said approach and moreover, without showing any evidence that any of the genes overexpressed in renal cell carcinoma are also overexpressed in feline oral SCC and, particularly, not showing either any data about changes (increase or decrease) in the level of expression of CD36 in feline oral SCC or any evidence about a possible involvement of CD36 in the initiation, development or spread of metastasis in such type of cancer. Moreover, it is commented that feline oral SCC exhibits low incidence of metastasis, but also mentioning that this might be due to the short survival times of cats with this tumor.

With regard to metastasis, it has been previously shown that inhibition of CD36 (both by antibodies neutralizing its activity or by shRNAs) has a dramatic effect regarding metastasis initiation and progression, decreasing metastatic penetrance and growth of all cell lines and patient-derived tumours tested. See, U.S. Publ. No. 2019-0106503, which is incorporated herein by reference in its entirety. Moreover, anti-CD36 antibodies useful in the treatment of cancer and cancer metastases have been disclosed in PCT/IB2021/051881, which is incorporated by reference in its entirety. The current invention provides new and improved anti-CD36 antibodies and methods of using such antibodies in the treatment of cancer and in the prevention/treatment of cancer metastasis.

### SUMMARY

The disclosure of this application is directed to anti-CD36 antibodies, and the use of such antibodies for the treatment of cancer. In some embodiments, the anti-CD36 antibodies are used to treat cancer metastases. In some embodiments, the anti-CD36 antibodies are used to treat both primary tumors and cancer metastases.

In some embodiments, the anti-CD36 antibody is an isolated antibody comprising one or more particular complementarity determining region (CDR) sequences. In some embodiments, the heavy chain CDR1 region comprises a sequence selected from the group consisting of SEQ ID NOs: 85-105. In some embodiments, the heavy chain CDR2 region comprises a sequence selected from the group consisting of SEQ ID NOs: 106-132, and 248. In some embodiments, the heavy chain CDR3 region comprises a sequence selected from the group consisting of SEQ ID NOs: 133-158. In some embodiments, the light chain CDR1 region comprises a sequence selected from the group consisting of SEQ ID NOs: 159-172. In some embodiments, the light chain CDR2 region comprises a sequence selected from the group consisting of SEQ ID NOs: 173-185, and 246. In some embodiments, the light chain CDR3 region comprises a sequence selected from the group consisting of SEQ ID NOs: 186-206, and 247. And in some embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region selected from SEQ ID NOs: 85-206 and 246-248. In some embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region selected from the particular combinations of CDR sequences listed in Table 2. In some embodiments, the anti-CD36 antibody is a chimeric antibody. In some embodiments, the anti-CD36 antibody is a humanized antibody. In some embodiments, the anti-CD36 antibody is a fully human antibody. In some embodiments, the anti-CD36 antibody is an isolated antibody comprising a heavy chain variable region and a light chain variable region. In some embodiments, the heavy chain variable region has at least 80%, at least 85%, at least 90%, at least 95%, at least 96%, at least 97%, at least 98%, at least 99%, or 100% identity with the amino acid sequence of at least one of SEQ ID NOs: 13-44, 241, and 243. In some embodiments, the light chain variable region has at least 80%, at least 85%, at least 90%, at least 95%, at least 96%, at least 97%, at least 98%, at least 99%, or 100% identity with the amino acid sequence of at least one of SEQ ID NOs: 45-78, 240, 242, 244, and 245. In some embodiments, the anti-CD36 antibody is an isolated antibody comprising a heavy chain variable region and a light chain variable region, wherein said heavy chain variable region and light chain variable region are as described in the above embodiments. In some embodiments, the heavy chain variable region and the light chain variable region, respectively, are SEQ ID NOs: 13 and 45, SEQ ID NOs: 14 and 46, SEQ ID NOs: 15 and 47, SEQ ID NOs: 16 and 48, SEQ ID NOs: 17 and 49, SEQ ID NOs: 18 and 50, SEQ ID NOs: 19 and 51, SEQ ID NOs: 20 and 52, SEQ ID NOs: 21 and 53, SEQ ID NOs: 22 and 54, SEQ ID NOs: 23 and 55, SEQ ID NOs: 24 and 56, SEQ ID NOs: 25 and 57, SEQ ID NOs: 26 and 58, SEQ ID NOs: 26 and 59, SEQ ID NOs: 27 and 60, SEQ ID NOs: 28 and 61, SEQ ID NOs: 29 and 62, SEQ ID NOs: 30 and 63, SEQ ID NOs: 31 and 64, SEQ ID NOs: 32 and 65, SEQ ID NOs: 33 and 66, SEQ ID NOs: 34 and 67, SEQ ID NOs: 35 and 68, SEQ ID NOs: 36 and 69 SEQ ID NOs: 37 and 70, SEQ ID NOs: 38 and 71, SEQ ID NOs: 39 and 72, SEQ ID NOs: 40 and 73, SEQ ID NOs: 40 and 74, SEQ ID NOs: 41 and 75, SEQ ID NOs: 42 and 76, SEQ ID NOs: 43 and 77, and SEQ ID NOs: 44 and 78.

In certain embodiments, the anti-CD36 antibody binds to human CD36. In some embodiments, the anti-CD36 antibody specifically binds to human CD36. In other embodiments, the anti-CD36 antibody has cross-reactivity for human CD36 and non-human CD36. In some embodiments, the antibody has cross-reactivity for human CD36 and non-human primate CD36. In some embodiments, the anti-CD36 antibody has cross-reactivity for human CD36, non-human primate CD36, and rodent CD36. In some embodiments the non-human primate CD36 is cynomolgus monkey CD36 or rhesus macaque CD36. In some embodiments, the rodent CD36 is rat CD36 or mouse CD36.

In some embodiments, the antibody binds to an epitope in human CD36 comprising at least one amino acid selected from the group consisting of, 145A, 146S, 147H, 148I, 149Y, 150Q, 151N, 152Q, 153F, 154V, 155Q, 156M, 1571, 158L, 159N, 160S, 185P, 186F, 187L, 188S, 189L, 190V, 191P, 192Y, 193P, 194V, 195T, 196T, 197T, 198V, 199G and 398K, 399I, 400Q, 401V, 402L, 403K, 404N, 405L, 406K, 407R, 408N, 409Y, 4101, 411V, 412P, 4131, and 414L. In another embodiment, the antibody binds to an epitope in human CD36 comprising or consisting of all of these amino acids in human CD36. In other embodiments the antibody binds to an epitope in human CD36 comprising at least one amino acid selected from the group consisting of 280E, 281S, 282D, 283V, 284N, 285L, 286K, 287G, 2881, 289P, 290V, 291Y, 292R, 293F, 294V, 295L, 296P, 297S, 298K, 341I, 342S, 343L, 344P, 345H, 346F, 347L, 348Y, 349A, 350S, 351P, 352D, 353V, 354S, 355E, 356P, 3571, 358D, 359G, 360L, 361N 362P, 363N, 364E, 365E. In another embodiment, the antibody binds to an epitope comprising or consisting of all of these amino acids in human CD36. Binding to a particular epitope can be determined, for example, by using techniques such as alanine scanning mutagenesis. In some embodiments, the antibody binds to an epitope in human CD36 comprising at least one amino acid, preferably comprising or consisting of all the amino acids, selected from the group consisting of 149Y, 150Q, 151N, 152Q, 153F, 154V, 155Q, 156M, 188S, 189L, 190V, 191P, 192Y, 193P, 194V, 195T, 196T, 400Q, 401V, 402L, 403K. In some embodiments, the antibody binds to an epitope comprising or consisting of 149Y, 150Q, 151N, 152Q, 153F, 154V, 155Q and 156M. In some embodiments, the antibody binds to an epitope comprising or consisting of 188S, 189L, 190V, 191P, 192Y, 193P, 194V, 195T and 196T. In some embodiments, the antibody binds to an epitope comprising or consisting of 400Q, 401V, 402L and 403K. In other embodiments, the antibody binds to an epitope in human CD36 comprising at least one amino acid, preferably comprising or consisting of all the amino acids, selected from the group consisting of 286K, 287G, 2881, 289P, 290V, 291Y, 292R, 341I, 342S, 343L, 344P, 345H, 346F, 347L, 348Y, 349A and 350S. In some embodiments, the antibody binds to an epitope in human CD36 comprising at least one amino acid, preferably comprising or consisting of all the amino acids, selected from the group consisting of 286K, 287G, 2881, 289P, 290V, 291Y, and 292R. In some embodiments, the antibody binds to an epitope in human CD36 as defined in any of the embodiments described herein above wherein the epitope is identified by hydrogen-deuterium exchange mass spectrometry.

In some embodiments, the anti-CD36 antibody binds to human CD36 with an affinity of greater than 20 nM, as measured using surface plasmon resonance with a bivalent model. In some embodiments, the anti-CD36 antibody binds to human CD36 with an affinity of greater than 10 nM, as measured using surface plasmon resonance with a bivalent model.

In certain embodiments, the anti-CD36 antibody further comprises a heavy chain constant region. In some embodiments, the antibody comprises an IgA or IgG heavy chain constant region. In some embodiments, the heavy chain constant region is selected from the group consisting of human immunoglobulin IgA1, IgA2, IgG1, IgG2, IgG3, or IgG4 heavy chain constant regions. In some embodiments, the heavy chain constant region comprises a constant region containing one or more mutations at amino acid positions E233, L234, L235, G236, N297, P331 and P329. In some embodiments, the heavy chain constant region comprises an IgG constant region containing a LALA mutation—which consists of leucine to alanine alterations at amino acid positions 234 and 235.

In some embodiments, the heavy chain constant region comprises an IgG constant region containing mutations at amino acid positions L234, L235, and/or G236. In some embodiments, the heavy chain constant region comprises an IgG constant region containing a set of mutations selected from the group consisting of L234A, L235S, and G236R; L234G, L235S, and G236R; L234Q, L235S, and G236R; L234S, L235G, and G236R; L234S, L235T, and G236R; L234S, L235V, and G236R; L234T, L235Q, and G236R; L234T, L235S, and G236R; L234T, L235T, and G236R; L234A and L235A; L234A, L235A, and P329G; G236R and L328R; L234A and G237A; L234A, L235A, and G237A; L234A and L235E; L235V, F243L, R292P, Y300L, and P396L; D265A and P329A; L234A, L235A, and K322A; L234F, L235E, and P331S; L234F, L235Q, and K322Q; L234A, L235A, G237A, P238S, H268A, A330S, and P331S; E233P, L234V, L235A, G236A, A327G, A330S, and P331S; L235A and G236R; L235S and G236R; G236R; L234Q and L235S; L235G and G236R; L234Q, L235S, and G236R; L234Q and L235S; L234Q, L235S, and G236R; L234Q, L235S, and G236R; L234Q, L235S, and G236R; L234Q, L235S, and G236R; L234Q, L235S, G236R, M252Y, S254T, and T256E; and L234Q, L235S, G236R, T250Q, and M428L. In some embodiments, the heavy chain constant region comprises an IgG constant region containing the L234G, L235S, and G236R mutations. In some embodiments, the heavy chain constant region comprises an IgG constant region containing the L234S, L235T, and G236R mutations. In some embodiments, the heavy chain constant region comprises an IgG constant region containing the L234S, L235V, and G236R mutations. In some embodiments, the heavy chain constant region comprises an IgG constant region containing the L234T, L235Q, and G236R mutations. In some embodiments, the heavy chain constant region comprises an IgG constant region containing the L234T, L235T, and G236R mutations. In some embodiments, the heavy chain constant region comprises an IgG constant region containing the L234A, L235S, and G236R mutations. In some embodiments, the heavy chain constant region comprises an IgG constant region containing the L234Q, L235S, and G236R mutations. In some embodiments, the heavy chain constant region comprises an IgG constant region containing the L234S, L235G, and G236R mutations. In some embodiments, the heavy chain constant region comprises an IgG constant region containing the L234T, L235S, and G236R mutations. In some embodiments, the heavy chain constant region comprises an IgG constant region containing the L234Q, L235S, and G236R mutations. In some embodiments, the heavy chain constant region comprises an IgG constant region containing the L234A and L235A mutations. In some embodiments, the heavy chain constant region comprises an IgG constant region containing the L234A, L235A, and P329G mutations.

In certain embodiments, the anti-CD36 antibody further comprises a light chain constant region. In some embodiments, the light chain constant region is selected from the group consisting of human immunoglobulins kappa (κ) and lambda (λ) light chain constant regions. In some embodiments, the antibody comprises a heavy chain constant region and a light chain constant region, wherein the heavy chain constant region is a human IgG1 heavy chain constant region, and wherein the light chain constant region is a human κ light chain constant region.

In certain embodiments, the antibody is an antigen-binding fragment. In some embodiments, the antigen binding fragment comprises a Fab, Fab′, F(ab′)₂, single chain Fv (scFv), disulfide linked Fv, V-NAR domain, IgNar, intrabody, IgGΔCH2, minibody, F(ab′)₃, tetrabody, triabody, diabody, single-domain antibody, DVD-Ig, Fcab, mAb2, (scFv)₂, or scFv-Fc.

In certain embodiments, the anti-CD36 antibody is a bispecific antibody. In some embodiments, the anti-CD36 bispecific antibody comprises a first antigen-binding domain that specifically binds to mammalian, e.g., human, CD36 and a second antigen-binding domain that specifically binds to a second antigen. In some embodiments the second antigen is a human immune cell antigen or tumor-specific antigen.

In certain embodiments, the anti-CD36 antibody is a biparatopic antibody, which binds at least two distinct epitopes on CD36. In some embodiments, the biparatopic anti-CD36 antibody binds to a first and second epitope on CD36. In some embodiments, the biparatopic anti-CD36 antibody comprises a first antigen-binding domain that specifically binds to a first epitope on CD36 and a second antigen-binding domain that specifically binds to a second epitope on CD36. In one embodiment, the biparatopic antibody comprises the antigen-binding domain of 1G04 and the antigen-binding domain of 10G04, 11G04, 19G04, 20G04 or 30G04. In certain embodiments, the biparatopic antibody binds the epitope of 1G04 and binds the epitope of 10G04, 11G04, 19G04, 20G04 or 30G04.

Certain embodiments are pharmaceutical compositions comprising an anti-CD36 antibody described herein and a pharmaceutically acceptable excipient. In some embodiments, at least 95% of the antibodies in the pharmaceutical composition are afucosylated. In some embodiments, the pharmaceutical composition further comprises one or more other therapeutic agents. In some embodiments, the pharmaceutical composition further comprises a PD-1 inhibitor. Suitable PD-1 inhibitors include the anti-PD-1 antibodies pembrolizumab, pidilizumab, or nivolumab. In some embodiments, the pharmaceutical composition further comprises a PD-L1 inhibitor such as the anti-PD-L1 antibodies atezolizumab, durvalumab, avelumab, or BMS-936559. In some embodiments, the pharmaceutical composition further comprises a CTLA-4 inhibitor such as the anti-CTLA-4 antibody ipilimumab. In some embodiments, the pharmaceutical composition further comprises a chemotherapeutic agent such as cisplatin.

Certain embodiments are methods of administering the anti-CD36 antibodies and pharmaceutical compositions containing anti-CD36 antibodies described herein. In some embodiments, the anti-CD36 antibody inhibits fatty acid uptake in HEK 293 cells expressing CD36 with an IC50 of less than 250 nM, less than 200 nM, less than 150 nM, less than 100 nM, less than 50 nM, less than 20 nM, less than 10 nM, less than 5 nM, less than 2 nM, or less than 1 nM as measured by FACS assay. In some embodiments, the anti-CD36 antibody inhibits oxLDL uptake with an IC₅₀ of less than 250 nM, less than 200 nM, less than 150 nM, less than 100 nM, less than 50 nM, less than 20 nM, less than 10 nM, less than 5 nM, less than 2 nM, or less than 1 nM, as measured by the anti-CD36 antibody's ability to inhibit uptake of oxLDL linked to a fluorophore (e.g., DiI) into SCC cells stably expressing human CD36. Some embodiments are directed to methods of treating cancer in a patient comprising administering to a subject in need thereof a therapeutically effective amount of an antibody disclosed herein, or a therapeutically effective amount of a pharmaceutical composition disclosed herein. In some embodiments, the cancer is oral squamous cell carcinoma, head and neck cancer, esophageal cancer, gastric cancer, ovarian cancer, cervical cancer, lung cancer, breast cancer, colon cancer, renal cancer, prostate cancer, sarcoma, e.g., liposarcoma, melanoma, leukemia, or lymphoma. Some embodiments are methods of treating one or more metastatic tumors in a patient comprising administering to a subject in need thereof a therapeutically effective amount of an antibody disclosed herein or a therapeutically effective amount of the pharmaceutical composition disclosed herein. Related embodiments are directed to an antibody disclosed herein for use in a method of treating cancer. Further related embodiments are directed to the use of an antibody disclosed herein in the manufacture of a medicament for the treatment of cancer. In some embodiments, the metastatic tumors are metastatic oral squamous cell carcinoma, metastatic head and neck cancer, metastatic esophageal cancer, metastatic gastric cancer, metastatic ovarian cancer, metastatic cervical cancer, metastatic lung cancer, metastatic breast cancer, metastatic colon cancer, metastatic renal cancer, metastatic prostate cancer, metastatic sarcoma, e.g., liposarcoma, metastatic melanoma, metastatic leukemia, or metastatic lymphoma. In some embodiments, the metastatic tumors are in the cervical lymph nodes, liver, lung, spleen, kidney, or peritoneal wall. In some embodiments, the treatment reduces the size of metastatic tumors, as measured by IVIS imaging or H&E staining. In some embodiments, the treatment reduces the size of the metastatic tumors in the cervical lymph nodes, liver, lung, spleen, kidney, or peritoneal wall. In some embodiments, the treatment prevents or inhibits the formation or development of metastatic tumors, as measured by IVIS imaging or H&E staining. In some embodiments, the treatment prevents or inhibits the formation or development of metastatic tumors in the cervical lymph nodes, liver, lung, spleen, kidney, or peritoneal wall. In some embodiments, the treatment reduces the number of metastatic tumors. In some embodiments, the patient is a human patient. In some embodiments, the treatment is effective in treating both a primary tumor and a metastatic tumor.

In certain embodiments, the method includes administering an anti-CD36 antibody that is a full length antibody, a single chain antibody, a scFv, a Fab fragment, or a F(ab′)₂ fragment. In some embodiments, the method includes administering an anti-CD36 antibody that is a full length antibody.

In certain embodiments, the method of treating a subject with cancer comprises administering a bispecific antibody comprising a first antigen-binding domain that binds human CD36 and a second antigen-binding domain that binds a human immune cell antigen selected from the group consisting of: PD-1, PD-L1, CTLA-4, CD3, LAG3, OX40, CD28, B7H3, CD47, TIM3, ICOS, LGR5, 4-1BB, CD40, CD40-L and TIGIT.

In certain embodiments, the method of treating a subject with cancer comprises administering a bispecific antibody comprising a first antigen-binding domain that binds human CD36 and a second antigen-binding domain that binds a human tumor antigen selected from the group consisting of: HER2, HER3, EGFR, VEGF, IGF-1, IGF-2, ANG2, DLL1, IGF-1R, cMET, DLL4, FAP, DR5, IL15, IL15Ra, CD3, CEA, EpCAM, PSMA, PMEL and GPC3.

In certain embodiments, the method includes administering a second therapy in addition to the anti-CD36 antibody. In some embodiments, the second therapy administered is an immunotherapy. In some embodiments, the administered immunotherapy is a PD-1 inhibitor such as the anti-PD-1 antibodies pembrolizumab, pidilizumab, or nivolumab. In some embodiments, the administered immunotherapy is a PD-L1 inhibitor such as the anti-PD-L1 antibodies atezolizumab, durvalumab, avelumab, or BMS-936559. In some embodiments, the administered immunotherapy is a CTLA-4 inhibitor such as the anti-CTLA-4 antibody ipilimumab. In some embodiments, the second therapy is a chemotherapeutic agent. In some embodiments, the administered chemotherapeutic agent is cisplatin.

In certain embodiments, metastasis is reduced or inhibited in the subject. In some embodiments, metastasis to the cervical lymph nodes, liver, lung, spleen, kidney, or peritoneal wall is reduced or inhibited in the subject. In some embodiments in which the method involves administering a second therapy in addition to the anti-CD36 antibody, the two therapies are administered sequentially. In some embodiments in which the method involves administering a second therapy in addition to the anti-CD36 antibody, the two therapies are administered simultaneously.

Certain embodiments are isolated polynucleotides that encode the antibodies disclosed herein. In some embodiments, the isolated polynucleotide encodes a heavy chain selected from the group consisting of SEQ ID NOs: 226, 228, 230, 232, 234, 236, 250, 257, and 258. In some embodiments, the isolated polynucleotide encodes a light chain selected from the group consisting of SEQ ID NOs: 227, 229, 231, 233, 235, 237, 249, and 253-256. In some embodiments, the isolated polynucleotide comprises SEQ ID NOs: 226 and 227, SEQ ID NOs: 228 and 229, SEQ ID NOs: 230 and 231, SEQ ID NOs: 232 and 233, SEQ ID NOs: 234 and 235, SEQ ID NOs: 236 and 237, SEQ ID NOs: 253 and 257, SEQ ID NOs: 253 and 258, SEQ ID NOs: 254 and 257, SEQ ID NOs: 254 and 258, SEQ ID NOs: 255 and 257, SEQ ID NOs: 255 and 258, SEQ ID NOs: 256 and 257, or SEQ ID NOs: 256 and 258.

Certain embodiments are vectors comprising the isolated polynucleotides disclosed herein. Certain other embodiments are cells comprising the isolated polynucleotides or vectors disclosed herein. In some embodiments, the cell is selected from the group consisting of E. coli, Pseudomonas, Bacillus, Streptomyces, yeast, CHO, YB/20, NS0, PER-C6, HEK 293, HEK 293T, NIH 3T3, HeLa, BHK, Hep G2, SP2/0, R1.1, B-W, L-M, COS 1, COS 7, BSC1, BSC40, BMT10 cell, plant cell, insect cell, and human cell in tissue culture. In some embodiments, the cell lacks a functional alpha-1,6-fucosyltransferase gene (FUT8) gene.

Certain embodiments are methods of making the antibodies disclosed herein. In some embodiments, the method of making an antibody comprises expressing the antibody using cells comprising the isolated polynucleotides or vectors disclosed herein. In some embodiments, the method of making an antibody comprises culturing a cell containing an isolated polynucleotide or vector disclosed herein under conditions suitable for expression of the antibody and isolating the antibody expressed therein.

### BRIEF DESCRIPTION OF THE FIGURES

FIG. 1A is a schematic showing an experimental overview of a study of the effects of a commercial anti-CD36 antibody in a mouse model of oral cancer metastasis using Detroit-562 cells, both with and without cisplatin. FIG. 1B details the study groups tested in that study, particularly the therapeutics and doses given to each group.

FIGS. 2A-2C provide results relating to the effects of an anti-CD36 antibody and/or cisplatin on the primary tumor in the Detroit-562 mouse model of oral cancer metastasis. FIG. 2A shows the quantitation of IVIS imaging of the primary tumor during the course of treatment with the anti-CD36 antibody and/or cisplatin. FIG. 2B shows a representative image of an H&E stained primary tumor from the tongue of an orthotopically-injected mouse. And FIG. 2C presents the surface area of the primary tumors in at the end of the treatment regimen. These figures illustrate that the tested anti-CD36 Ab had at least additive anti-tumor activity with cisplatin on suppressing the growth of a primary tumor in oral cancer.

FIG. 3 contains representative H&E stained images of lung metastases at the end of the course of treatment with an anti-CD36 antibody and/or cisplatin in the Detroit-562 mouse model of oral cancer metastasis. This figure illustrates that mice treated with cisplatin (top right), anti-CD36 antibody (bottom left), or cisplatin and anti-CD36 antibody (bottom right) have fewer and smaller metastases than control treated mice (top left).

FIGS. 4A and 4B contain quantitation of the number and size of lung metastasis, respectively, in the Detroit-562 mouse model of oral cancer metastasis. These figures illustrate that mice treated with an anti-CD36 antibody alone had smaller and fewer metastases than control mice. Mice treated with cisplatin alone had similar numbers of metastases to control mice, though cisplatin did reduce the size of the metastatic tumors. Treatment with both anti-CD36 antibody and cisplatin resulted in mice with similar numbers of metastases than treatment with anti-CD36 antibody alone. However, treatment with both the anti-CD36 antibody and cisplatin resulted in reduction of metastatic tumor size to a greater extent than either the anti-CD36 antibody or cisplatin alone.

FIG. 5A is a schematic showing an experimental overview of a study of the effects of the ONA-0-v1 anti-CD36 antibody in a mouse model of oral cancer metastasis using FaDu cells, both with and without cisplatin. FIG. 5B details the study groups tested in that study, particularly the therapeutics and doses given to each group.

FIGS. 6A and 6B show the results of IVIS imaging (FIG. 6A) and H&E staining (FIG. 6B) of primary tumors from the study of the effects of the ONA-0-v1 anti-CD36 antibody in a mouse model of oral cancer metastasis using FaDu cells. In both assays, while cisplatin inhibited tumor growth, treatment with the administered dose of ONA-0-v1 did not have a statistically significant effect on the primary tumor relative to treatment with an isotype control antibody in this model.

FIGS. 7A and 7B show the results of IVIS imaging of metastases from the study of the effects of the ONA-0-v1 anti-CD36 antibody in a mouse model of oral cancer metastasis using FaDu cells. These results show that treatment with ONA-0-v1 was able to inhibit growth of metastases.

FIG. 8 and FIG. 9 show the results of IVIS imaging of lymph node metastases from the study of the effects of the ONA-0-v1 anti-CD36 antibody in a mouse model of oral cancer metastasis using FaDu cells. Treatment with ONA-0-v1 antibody inhibited metastatic tumor growth by greater than 50% relative to the IgA isotype control, and addition of ONA-0-v1 to cisplatin enhanced cisplatin's ability to inhibit metastatic tumor growth.

FIG. 10 shows the results of IVIS imaging of lymph node metastases from the study of the effects of the ONA-0-v1 anti-CD36 antibody in a mouse model of oral cancer metastasis using FaDu cells. Treatment with either cisplatin or ONA-0-v1 reduced metastasis into the lymph nodes, and ONA-0-v1's inhibition of penetrance was synergistic with that of cisplatin.

FIGS. 11A and 11B contain measurements of body weight and platelet count during the course of treatment with ONA-0-v1 and/or cisplatin. These data show that, unlike cisplatin, ONA-0-v1 treatment alone did not have any effects on mouse body weight or platelet count relative to isotype control-treated mice.

FIG. 12A is a schematic showing an experimental overview of a study of the effects of the ONA-0-v1 anti-CD36 antibody in a mouse model of ovarian cancer using OVCAR-3 cells. FIG. 12B is an image of the primary tumors excised from mice tested in this model, with tumors from vehicle-injected mice on the top row and tumors from mice injected with ONA-0-v1 on the bottom row. FIG. 12C presents the quantification of the weight of these primary tumors, and shows that treatment with ONA-0-v1 resulted in a relative decrease in the weight of the primary tumors (** indicates unpaired t test p=0.033). FIG. 12D and FIG. 12E show the results of histological analysis of the OVCAR-3 primary tumors for percent necrosis and fibrosis/collagen, respectively (* indicates unpaired t test p=0.0287). FIGS. 12D and 12E show that treatment with ONA-0-v1 results in increased necrosis and fibrosis occur in the analyzed tumors.

FIGS. 13A and 13B show representative images of metastases formed in the mouse model of ovarian cancer using OVCAR-3 cells. FIG. 13A shows exemplary metastases in the peritoneal wall, and FIG. 13B shows exemplary liver metastases. Each image includes a centimeter-marked ruler for scale, and white arrows that point to the metastases.

FIGS. 14A-14C depict the quantification of the number and size of metastases in the OVCAR-3 mouse model of ovarian cancer in control-treated mice and mice treated with ONA-0-v1. FIG. 14A shows the total number of macroscopic metastases that were observed in any organ in control (“vehicle”) mice (sum from all vehicle mice; n=9) and in mice treated with ONA-0-v1 (sum from all treated mice; n=8), and that treatment with ONA-0-v1 reduced the number of metastases by more than 50%. FIGS. 14B and 14C show the macroscopic quantification of the size of metastases in the peritoneal wall and liver, respectively. Collectively, FIGS. 14A, 14B, and 14C show that treating with ONA-0-v1 decreases the size and number of metastases in the OVCAR-3 mouse model of ovarian cancer.

FIGS. 15A-15G show the results of testing the effects of the ONA-0-v1 and 1G04 anti-CD36 antibodies in the OVCAR-3 mouse model of ovarian cancer, relative to control-treated mice. FIG. 15A is a schematic showing an experimental overview of this study. FIG. 15B depicts the change in body weight of treated mice over time. FIGS. 15C-15G show that both ONA-0-v1 and 1G04 reduce both the number and size of metastases in the treated mice.

FIGS. 16A-16G show the effects of 1G04 treatment on the number of HCT-116 cells in particular organs (i.e., in metastases) in the mouse model of colon cancer, as measured by ex vivo analysis of luciferase luminescence. FIG. 16A is a schematic showing an experimental overview of this study. FIG. 16B depicts the change in body weight of treated mice over time. FIG. 16C shows that 1G04 reduces overall cancer cell burden in treated mice, and FIGS. 16D-16G show that treating with ONA-0-v1 resulted in decreased luminescence in the liver (FIG. 16D), lungs (FIG. 16E), spleen (FIG. 16F), and kidney (FIG. 16G).

FIGS. 17A-17E show the results of testing the 1G04 anti-CD36 antibody in the A549 model of metastatic lung cancer, relative to vehicle-treated mice. FIG. 17A is a schematic showing an experimental overview of this study. FIG. 17B details the study groups tested in that study, particularly the therapeutics and dose given to each group. FIG. 17C shows that 1G04 reduces overall cancer cell burden in treated mice, as measured by luminescence. FIGS. 17D and 17E show that lung weight and lung luminescence ex vivo, respectively, are decreased after treatment with 1G04.

FIGS. 18A-18E show the effect of 1G04 treatment in the MC38 syngeneic colon cancer model. FIG. 18A is a schematic showing an experimental overview of this study. FIG. 18B details the study groups tested in that study, particularly the therapeutics and dose given to each group. FIG. 18C shows that 1G04 reduces overall cancer cell burden in treated mice, as measured by luminescence. FIG. 18D shows that liver luminescence is reduced after 1G04 treatment, indicating a reduced level of metastasis in the liver. Similarly, FIG. 18E shows that lung luminescence is reduced after 1G04 treatment, indicating a reduced level of metastasis in the lung.

FIGS. 19A-19C show the effect of treating mice bearing 4T1 breast cancer tumors with 1G04 anti-CD36 antibody. FIG. 19A is a schematic showing an experimental overview of this study. FIG. 19B details the study groups tested in that study, particularly the therapeutics and dose given to each group. FIG. 19C shows that luminescence in the lung is decreased after 1G04 treatment compared to vehicle treatment, indicating a reduced level of metastasis in the lung.

FIG. 20 shows the results of an assay in SCC25 cells testing the ability of anti-CD36 antibodies to affect CD36-mediated fatty acid uptake. Each anti-CD36 antibody tested inhibited fatty acid uptake relative to the uptake observed after treatment with an isotype control antibody.

FIGS. 21A-21J shows an assay in HEK 293 cells testing the ability of anti-CD36 antibodies to inhibit CD36-mediated uptake of palmitic acid linked to a fluorophore (BODIPY FL C16). The basic experimental protocol is provided in FIG. 21A. The results of this assay are plotted in FIGS. 21B-21J, which show that each of 6G04 (FIG. 21D), 7G04 (FIG. 21E), 9G04 (FIG. 21F), 11G04 (FIG. 21G), 13G04 (FIG. 21H), 14G04 (FIG. 21I), and 28G04 (FIG. 21J) inhibited palmitic acid uptake more effectively than an isotype control (FIG. 21B) or 1G04 (FIG. 21C).

FIGS. 22A and 22B show a test of the ability of anti-CD36 antibodies to inhibit CD36-mediated oxLDL uptake at a range of different antibody concentrations. The basic experimental protocol is provided in FIG. 22A. FIG. 22B shows titration curves for each of 1G04, 6G04, 7G04, 11G04, 13G04, 14G04, and 28G04, which indicate that these antibodies all have similar ability to inhibit oxLDL uptake into SCC25 cells.

FIGS. 23A and 23B show a test of whether anti-CD36 antibodies interfere with the CD36's interaction with TSP1 in an SPR competition experiment. An exemplary plot of the data obtained using 1G04, with the protocol steps annotated, is provided in FIG. 23A. As shown in FIG. 23B, none of the tested antibodies had a significant effect on the TSP-1/CD36 interaction.

FIGS. 24A-24D show the results of testing antibodies engineered to have various constant regions, each of which contained a different Fc silencing alteration. FIGS. 24A and 24B show ELISA assays demonstrating that none of the Fc alterations disrupted binding to human CD36 (FIG. 24A) or mouse CD36 (FIG. 24B). FIG. 24C shows that the different Fc-formatted anti-CD36 antibodies bound equivalently to SCC25 cells stably expressing human CD36 (SEQ ID NO: 1) in a FACS assay. And FIG. 24D shows that each of the different Fc variants inhibited palmitic acid uptake into SCC25 cells at similar levels.

FIG. 25A shows testing to determine the ability of anti-CD36 antibodies with Fc silencing mutations to bind to various FcγRs, and indicates that the tested Fc alterations reduced interaction with human and cynomolgus FcγRs. FIGS. 25B-25D show that anti-CD36 antibodies with the Fc silencing mutations did not induce ADCC, ADCP, or CDC complement response.

FIG. 26 shows testing to determine the ability of anti-CD36 antibodies with Fc silencing mutations to bind to FcRn, and indicates that the tested Fc alterations did not affect FcRn binding.

FIG. 27 shows an ELISA assay testing binding of anti-CD36 antibodies with Fc silencing mutations to complement C1q protein, which detected no binding by any of the Fc-engineered samples.

FIGS. 28A and 28B show an in vitro human platelet aggregation assay testing different Fc formatted anti-CD36 antibodies, and show that the Fc alterations eliminated antibody-induced platelet aggregation with or without the platelet aggregation agonist ADP.

FIG. 29A shows the sequence of human CD36 (SEQ ID NO: 1), with residues identified as constituting the main 1G04 binding epitope shown underlined in bold, as identified by hydrogen-deuterium exchange experiments. FIG. 29B shows both space-filling and ribbon models of CD36 structure, with the residues that form the 1G04 epitope (as defined by CD36 peptides with >0.5 D difference in deuteration between unbound and bound state) highlighted in red.

FIG. 30 shows the differential deuterium uptake plots (ΔD) for a selection of peptides along the sequence of CD36 (SEQ ID NO: 1). The comparison of deuterium incorporation of the unbound versus the antibody-bound protein state allows the identification of sites in the target protein involved in the binding of 1G04.

FIG. 31A shows the sequence of human CD36 (SEQ ID NO: 1), with residues identified as constituting the main 11G04 binding epitope shown underlined in bold, as identified by hydrogen-deuterium exchange experiments. FIG. 31B shows both space-filling and ribbon models of CD36 structure, with the residues that form the 11G04 epitope (as defined by CD36 peptides with >0.5 D difference in deuteration between unbound and bound state) highlighted in red. FIG. 31C show the the differential deuterium uptake plots (ΔD) for a selection of peptides along the sequence of CD36 (SEQ ID NO: 1). The comparison of deuterium incorporation of the unbound versus the antibody-bound protein state allows the identification of sites in the target protein involved in the binding of 11G04.

FIGS. 32A-32C show the results of testing anti-CD36 antibodies in the HCT-116 model of metastatic colorectal cancer, relative to vehicle-treated mice. FIG. 32A is a schematic showing an experimental overview of this study and details the study groups tested in that study, particularly the therapeutics and dose given to each group. FIG. 32B shows that anti-CD36 antibodies reduce overall cancer cell burden in treated mice, as measured by luminescence. FIG. 32C shows that liver luminescence ex vivo is decreased after treatment with anti-CD36 antibodies.

### DETAILED DESCRIPTION

The present disclosure related to anti-CD36 antibodies, nucleotides encoding anti-CD36 antibodies, pharmaceutical compositions comprising anti-CD36 antibodies, and methods of treating (e.g., reducing and/or inhibiting) cancer, particularly cancer metastases, using anti-CD36 antibodies. The anti-CD36 antibodies disclosed include both IgA and IgG antibodies, both of which are effective in the disclosed methods of treating cancer. The disclosed anti-CD36 antibodies are effective at treating primary tumors, metastatic cancer, or both primary tumors and metastatic cancer.

### Definitions of General Terms and Expressions

In order that the present disclosure can be more readily understood, certain terms are first defined. As used in this application, except as otherwise expressly provided herein, each of the following terms shall have the meaning set forth below. Additional definitions are set forth throughout the application.

The term “antibody” means an immunoglobulin molecule that recognizes and specifically binds to a target, such as a protein, polypeptide, peptide, carbohydrate, polynucleotide, lipid, or combinations of the foregoing. As used herein, the term “antibody” encompasses polyclonal antibodies, monoclonal antibodies, chimeric antibodies, humanized antibodies, fully human antibodies, recombinant antibodies, bispecific antibodies, biparatopic antibodies, fusion proteins comprising a full length antibody or fragments thereof, antigen-binding fragments of such antibodies, and any other modified immunoglobulin molecule so long as it exhibits the desired biological activity, e.g., antigen binding. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain the antigenic determining regions of an intact antibody (e.g., the complementarity determining regions (CDR)). Examples of antigen-binding fragments of antibodies include, but are not limited to Fab, Fab′, F(ab′)₂, and Fv fragments, linear antibodies, and single chain antibodies. An antigen-binding fragment of an antibody can be derived from any animal species, such as rodents (e.g., mouse, rat, or hamster) and humans or can be artificially produced.

The terms “anti-CD36 antibody,” “CD36 antibody” and “antibody that binds to CD36” refer to an antibody that is capable of specifically binding CD36, e.g., human CD36, with sufficient affinity such that the antibody is useful as a diagnostic and/or therapeutic agent in targeting CD36. The extent of binding of an anti-CD36 antibody to an unrelated, non-CD36 protein can be less than about 10% of the binding of the antibody to CD36 as measured, e.g., by a radioimmunoassay (RIA).

The terms “anti-PD-1 antibody,” “PD-1 antibody” and “antibody that binds to PD-1” refer to an antibody that is capable of binding PD-1 with sufficient affinity such that the antibody is useful as a diagnostic and/or therapeutic agent in targeting PD-1. The extent of binding of an anti-PD-1 antibody to an unrelated, non-PD-1 protein can be less than about 10% of the binding of the antibody to PD-1 as measured, e.g., by a radioimmunoassay (RIA).

An “isolated antibody” refers to an antibody population that comprises a single species of antibody. For example, a particular isolated anti-CD36 antibody consists of an antibody population having a single heavy chain amino acid sequence and a single light chain amino acid sequence, which binds to a single CD36 epitope. An isolated antibody that binds specifically to CD36 can, however, have cross-reactivity to other antigens, such as CD36 molecules from different species. Also, a population of antibodies may still be an “isolated antibody” when contaminated by small amounts of other antibody species. In particular, an isolated antibody may contain less than 5%, less than 4%, less than 3%, less than 2%, less than 1%, or no other antibody species.

A “monoclonal antibody” refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody encompasses intact and full length monoclonal antibodies, as well as antibody fragments (such as Fab, Fab′, F(ab′)₂, Fv), single chain (scFv) mutants, fusion proteins comprising an antibody portion, and any other modified immunoglobulin molecule comprising an antigen recognition site. Furthermore, “monoclonal” antibody refers to such antibodies and antigen-binding fragments thereof made in any number of manners including but not limited to by hybridoma, phage selection, recombinant expression, and transgenic animals.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody or an antigen-binding fragment thereof. In certain aspects, CDRs can be determined according to the Kabat numbering system (see, e.g., Kabat E A & Wu T T (1971) Ann. NY Acad. Sci. 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In a specific embodiment, the CDRs of the antibodies described herein have been determined according to the Kabat numbering scheme.

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

The AbM hypervariable regions represent a compromise between the Kabat CDRs and Chothia structural loops, and are used by Oxford Molecular's AbM antibody modeling software. In specific embodiments, the CDRs of the antibodies described herein have been determined according to the Chothia numbering scheme or the AbM numbering scheme.

TABLE 1
CDR Numbering

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

In some aspects, the CDR regions CDRs can be determined according to the IMGT numbering system (see, e.g., Guidicelli et al., Nucl. Acids Res. 34:D781-D784 (2006)). This numbering scheme unifies numbering across antibody lambda and kappa light chains, heavy chains and T-cell receptor chains.

As used herein, the terms “constant region” and “constant domain” are interchangeable and have their common meaning in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In certain aspects, an antibody or antigen-binding fragment comprises a constant region or portion thereof that is sufficient for antibody-dependent cell-mediated cytotoxicity (ADCC).

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG (e.g., IgG1, IgG2, IgG3, and IgG4) and subclasses of IgA (e.g., IgA1 and IgA2). Heavy chain amino acid sequences are well known in the art. In specific embodiments, the heavy chain is a human heavy chain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

The term “chimeric antibody” refers to a full length antibody or an antigen-binding fragment thereof wherein the amino acid sequence is derived from two or more species. Typically, the variable region of both light and heavy chains corresponds to the variable region of antibodies derived from one species of mammals (e.g. mouse, rat, rabbit, etc.) with the desired specificity, affinity, and capability while the constant regions are homologous to the sequences in derived from another (usually human) to avoid eliciting an immune response in that species.

A “humanized antibody” refers to a chimeric antibody, or antigen-binding fragment thereof, comprising amino acid residues from non-human CDRs and amino acid residues from human framework regions and constant regions. In certain embodiments, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Typically, humanized antibodies are human immunoglobulins in which residues from the CDRs are replaced by residues from the CDRs of a non-human species (e.g. mouse, rat, rabbit, hamster) that have the desired specificity, affinity, and capability (Jones et al., Nature 321:522-525 (1986); Riechmann et al., Nature 332:323-327 (1988); Verhoeyen et al., Science 239:1534-1536 (1988)). Accordingly, humanized antibodies are also referred to as “CDR grafted” antibodies. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996).

A “human antibody” refers to a full length antibody or fragment thereof having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

An “afucosylated” antibody or antigen-binding fragment thereof, or an antibody or antigen-binding fragment thereof “lacking fucose,” refers to an IgG1 or IgG3 isotype antibody or antigen-binding fragment thereof that lacks any fucose residues in the constant region glycosylation on at least 50% of the antibody population. Glycosylation of human IgG1 or IgG3 occurs at Asn297 as core fucosylated biantennary complex oligosaccharide glycosylation terminated with up to 2 Gal residues. In some embodiments, an afucosylated antibody lacks fucose at Asn297. These structures are designated as G0, G1 (a 1.6 or a 1.3), or G2 glycan residues, depending on the amount of terminal Gal residues. See, e.g., Raju, T. S., BioProcess Int. 1: 44-53 (2003). CHO type glycosylation of antibody Fc is described, e.g., in Routier, F. FL, Glycoconjugate J. 14: 201-207 (1997).

Methods of measuring fucose include any methods known in the art. For purposes herein, fucose can be detected by the method described in Example 1 of WO2015/017600, which is herein incorporated by reference in its entirety. Briefly, glycan analysis can be performed by releasing glycans from the antibody (e.g., by enzymatic release), labeling the glycans with anthranilic acid (2-AA), and then purifying the labeled glycans. Normal phase HPLC with fluorescent detection is used to separate the glycans and measure the relative amount of each glycan in the antibody. The glycans may be positively identified as lacking or including fucose by mass spectrometry. In some embodiments, fucose is undetectable in a composition comprising a plurality of afucosylated antibodies. In some embodiments, an afucosylated antibody has enhanced ADCC activity, which may be measured by the assay provided in Example 13 herein. In some embodiments, an afucosylated antibody has enhanced affinity for Fc gamma RIIIA. In some embodiments, an afucosylated antibody has enhanced affinity for Fc gamma RIIIA(V158). In some embodiments, an afucosylated antibody has enhanced affinity for Fe gamma RIIIA(F158). Affinity for Fe gamma RIIIA or its alleles may be measured by the assay provided in Example 13 herein.

An “Fc silencing mutation” refers to a mutation in the Fc domain of an antibody which decreases, partially or wholly, binding to one or more cell surface Fc receptors, thereby reducing or dampening, and in some embodiments abrogating substantially completely, one or more Fc-mediated antibody effector functions, such as ADCC, ADCP, and CDC complement response. (See, e.g., Kang and Jung, Experimental & Molecular Medicine (2019) 51:138). Fc silencing mutations have been described in the art, including amino acid substitutions at one of more of positions E233, L234, L235, G236, N297, P331 and P329 (see e.g. U.S. Pat. Nos. 6,737,056, 7,332,581; WO 2004/056312, WO2021/234402 (the contents of each of which are hereby incorporated by reference in their entirety) and Shields, R. L. et al., J. Biol. Chem. 276 (2001) 6591-6604).

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody to an antigen, and k_{off} refers to the dissociation of, e.g., an antibody from an antigen. The kₒₙ and k_{off} can be determined by techniques known to one of ordinary skill in the art, such as BIAcore® or KinExA.

As used herein, an “epitope” refers to a localized region of an antigen, e.g., human CD36, to which an antibody, e.g., an anti-CD36 antibody described herein, specifically binds. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody binds can be determined by, e.g., computational docking methods, NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping such as alanine scanning mutagenesis mapping). Computational docking-based epitope analysis may be accomplished using methods known in the art (e.g., Bourquard T et al., J. Immunol. 201(10):3096-3105 (2018); Weitzner B D et al., Nat. Protocols 12(2):401-416 (2017)). For X-ray crystallography, crystallization may be accomplished using any of the known methods in the art (e.g., Giegé R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Crystals of an antibody bound to antigen can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323). Mutagenesis mapping studies can be accomplished using any method known to one of skill in the art. See, e.g., Champe M et al., (1995) J Biol Chem 270: 1388-1394 and Cunningham B C & Wells J A (1989) Science 244: 1081-1085 for a description of mutagenesis techniques, including alanine scanning mutagenesis techniques.

A CD36 antibody that “binds to the same epitope” as a reference CD36 antibody refers to an antibody that binds to the same CD36 amino acid residues as the reference CD36 antibody. The ability of a CD36 antibody to bind to the same epitope as a reference CD36 antibody can be determined by epitope binning in a competition assay (e.g., the AlphaScreen® assay disclosed in Bembenek M E et al., Analytical Bioch. 408(2):321-327 (2011)), a hydrogen/deuterium exchange coupled with mass spectrometry, referred also herein as a hydrogen/deuterium exchange assay (see Coales et al., Rapid Commun. Mass Spectrom. 2009; 23: 639-647), FACS analysis combined with alanine scanning, crosslinking-coupled mass spectrometry (XL-MS), peptide scanning, or mutagenesis.

As used herein, the terms “immunospecifically binds,” “immunospecifically recognizes,” “specifically binds,” and “specifically recognizes” are analogous terms in the context of antibodies. These terms indicate that the antibody binds to an epitope via its antigen-binding domain and that the binding entails some complementarity between the antigen binding domain and the epitope. Accordingly, an antibody that “specifically binds” to human CD36 (SEQ ID NO: 1) may also bind to CD36 from other species (e.g., non-human primate, mouse, and/or rat CD36) and/or CD36 proteins produced from other human alleles, but the extent of binding to an un-related, non-CD36 protein is less than about 10% of the binding of the antibody to CD36 as measured, e.g., by a radioimmunoassay (RIA).

In a specific embodiment, provided herein is an antibody that binds to human, cynomolgus monkey, rhesus macaque, mouse, and rat CD36.

An antibody is said to “competitively inhibit” binding of a reference antibody to a given epitope if it preferentially binds to that epitope or an overlapping epitope to the extent that it blocks, to some degree, binding of the reference antibody to the epitope. Competitive inhibition may be determined by any method known in the art, for example, competition ELISA assays or competition FACS. An antibody may be said to competitively inhibit binding of the reference antibody to a given epitope by at least 90%, at least 80%, at least 70%, at least 60%, or at least 50%.

As used herein, the characteristic of being “substantially free” of a substance refers to a near complete or complete lack of that substance. For example, a pharmaceutical composition that is substantially free of a particular antibody species has a near-complete or complete lack of that antibody species in the pharmaceutical composition in question. In this context, substantially free can refer to having less than 5%, less than 4%, less than 3%, less than 2%, less than 1%, or none of the antibody in the pharmaceutical composition be the antibody species in question. Moreover, “substantially free” of contaminants can refer to being purified such that it contains little other cellular material and/or chemicals (e.g., less than 5%, less than 4%, less than 3%, less than 2%, less than 1%, or no other cellular material and/or chemicals).

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

“Percent identity” refers to the extent of identity between two sequences (e.g., amino acid sequences or nucleic acid sequences). Percent identity can be determined by aligning two sequences, introducing gaps to maximize identity between the sequences. Alignments can be generated using programs known in the art. For purposes herein, alignment of nucleotide sequences can be performed with the blastn program set at default parameters, and alignment of amino acid sequences can be performed with the blastp program set at default parameters (see National Center for Biotechnology Information (NCBI) on the worldwide web, ncbi.nlm.nih.gov).

As used herein, the term “host cell” can be any type of cell, e.g., a primary cell, a cell in culture, or a cell from a cell line. In specific embodiments, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

The terms “pharmaceutical composition” and “pharmaceutical formulation” refer to a preparation which is in such form as to permit the biological activity of the active ingredient to be therapeutically effective, and which contains no additional components which are unacceptably toxic to a subject to which the composition or formulation would be administered. The composition or formulation can be sterile.

The terms “administer”, “administering”, “administration”, and the like, as used herein, refer to methods that may be used to enable delivery of a drug, e.g., an anti-CD36 antibody, to the desired site of biological action. Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa. Administration refers to the physical introduction of a composition comprising a therapeutic agent to a subject, using any of the various methods and delivery systems known to those skilled in the art. Preferred routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, preferably orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

Administration “in combination with” one or more further therapeutic agents includes simultaneous (concurrent) or consecutive administration in any order.

The combination therapy can provide “synergy,” i.e., the effect achieved when the active agents used together is greater than the sum of the effects that result from using the active agents separately. A synergistic effect can be attained when the active agents are: (1) co-formulated and administered or delivered simultaneously in a combined, unit dosage formulation; (2) delivered serially, by alternation, or in parallel as separate formulations; or (3) by some other regimen. When delivered in alternation therapy, a synergistic effect can be attained when the active agents are administered or delivered sequentially, e.g., by different injections in separate syringes. A “synergistic combination” produces an effect that is greater than the sum of the effects of the individual active agents of the combination.

The combination therapy can provide an “additive” effect, i.e., the effect achieved when the active agents used together is equal to the sum of the effects the result from using the active agents separately.

As used herein, the terms “subject” and “patient” are used interchangeably. The subject can be an animal. In some embodiments, the subject is a mammal such as a non-human animal (e.g., cow, pig, horse, cat, dog, rat, mouse, monkey or other primate, etc.). In some embodiments, the subject is a cynomolgus monkey. In some embodiments, the subject is a human.

The term “therapeutically effective amount” refers to an amount of a drug, e.g., an anti-CD36 antibody, effective to achieve the desired therapeutic or prophylactic result. In some instances, the desired result is treating a disease or disorder in a subject. In the case of cancer, the therapeutically effective amount of the drug can reduce the number of cancer cells; reduce the tumor size or burden; inhibit (i.e., slow to some extent and in a certain embodiment, stop) cancer cell infiltration into peripheral organs; inhibit (i.e., slow to some extent and in a certain embodiment, stop) tumor metastasis; inhibit, to some extent, tumor growth; relieve to some extent one or more of the symptoms associated with the cancer; and/or result in a favorable response such as increased progression-free survival (PFS), disease-free survival (DFS), or overall survival (OS), complete response (CR), partial response (PR), or, in some cases, stable disease (SD), a decrease in progressive disease (PD), a reduced time to progression (TTP), or any combination thereof. To the extent the drug can prevent growth and/or kill existing cancer cells, it can be cytostatic and/or cytotoxic.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. In certain embodiments, a subject is successfully “treated” for cancer according to the methods of the present invention if the patient shows one or more of the following: a reduction in the number of or complete absence of cancer cells; a reduction in the tumor size; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; increased progression-free survival (PFS), disease-free survival (DFS), or overall survival (OS), complete response (CR), partial response (PR), stable disease (SD), a decrease in progressive disease (PD), a reduced time to progression (TTP), or any combination thereof. In the context of metastatic cancer, treatment also refers to preventing the development of new metastatic tumors, reducing the size of metastatic tumors, or eliminating existing metastatic tumors.

A “cancer” refers a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. Such distal tumors are also referred to as “metastatic tumors” or “metastases.”

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of” and/or “consisting essentially of” are also provided. In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

Any compositions or methods provided herein can be combined with one or more of any of the other compositions and methods provided herein.

### Anti-CD36 Antibodies

In a specific aspect, provided herein are full length antibodies (e.g., monoclonal antibodies, such as chimeric, humanized, or human antibodies) and antigen-binding fragments thereof which specifically bind to CD36 (e.g., human CD36, non-human primate CD36, and rodent CD36). The amino acid sequences for human, cynomolgus monkey, rhesus macaque, murine, and rat CD36 are known in the art and are also provided herein as represented by SEQ ID NOs: 1-4, as shown below.

### Human CD36 (SEQ ID NO: 1; UNIPROT P16671):

MGCDRNCGLIAGAVIGAVLAVFGGILMPVGDLLIQKTIKKQVVLEEGTI
AFKNWVKTGTEVYRQFWIFDVQNPQEVMMNSSNIQVKQRGPYTYRVRFL
AKENVTQDAEDNTVSFLQPNGAIFEPSLSVGTEADNFTVLNLAVAAASH
IYQNQFVQMILNSLINKSKSSMFQVRTLRELLWGYRDPFLSLVPYPVTT
TVGLFYPYNNTADGVYKVFNGKDNISKVAIIDTYKGKRNLSYWESHCDM
INGTDAASFPPFVEKSQVLQFFSSDICRSIYAVFESDVNLKGIPVYRFV
LPSKAFASPVENPDNYCFCTEKIISKNCTSYGVLDISKCKEGRPVYISL
PHFLYASPDVSEPIDGLNPNEEEHRTYLDIEPITGFTLQFAKRLQVNLL
VKPSEKIQVLKNLKRNYIVPILWLNETGTIGDEKANMFRSQVTGKINLL
GLIEMILLSVGVVMFVAFMISYCACRSKTIK

Cynomolgus Monkey/Rhesus Macaque CD36 (SEQ ID NO: 2; UNIPROT Q4R6B4 and Q6J512, respectively):

MGCDRNCGLITGAVIGAVLAVFGGILMPVGDMLIQKTIKKEVVLEEGTI
AFKNWVKTGTEIYRQFWIFDVQNPQEVMMNSSNIQVKQRGPYTYRVRFL
AKENITQDPKDNTVSFLQPNGAIFEPSLSVGTEADNFTVLNLAVAAASH
IYPNPFVQVVLNSLINKSKSSMFQVRTLRELLWGYTDPFLSLVPYPVST
RVGMFYPYNNTADGVYKVFNGKDSISKVAIIDTYKGKRNLSYWESYCDM
INGTDAASFPPFVEKSQVLQFFSSDICRSIYAVFESDVNLKGIPVYRFV
LPSKAFASPVQNPDNHCFCTEKIISKNCTSYGVLDISKCKEGKPVYISL
PHFLYASPDVSETIDGLNPNEEEHRTYLDIEPITGFTLQFAKRLQVNLL
VKPSNKIQVLKRLKRNYIVPILWLNETGTIGDEKAKMFRSQVTGKINLL
GLIEMILLSVGVVMFVAFMISYCACRSKTIK

### Murine CD36 (SEQ ID NO: 3; UNIPROT Q08857):

MGCDRNCGLIAGAVIGAVLAVFGGILMPVGDMLIEKTIKREVVLEEGTT
AFKNWVKTGTTVYRQFWIFDVQNPDDVAKNSSKIKVKQRGPYTYRVRYL
AKENITQDPEDHTVSFVQPNGAIFEPSLSVGTEDDNFTVLNLAVAAAPH
IYQNSFVQVVLNSLIKKSKSSMFQTRSLKELLWGYKDPFLSLVPYPIST
TVGVFYPYNDTVDGVYKVENGKDNISKVAIIESYKGKRNLSYWPSYCDM
INGTDAASFPPFVEKSRTLRFFSSDICRSIYAVFGSEIDLKGIPVYRFV
LPANAFASPLQNPDNHCFCTEKVISNNCTSYGVLDIGKCKEGKPVYISL
PHFLHASPDVSEPIEGLHPNEDEHRTYLDVEPITGFTLQFAKRLQVNIL
VKPARKIEALKNLKRPYIVPILWLNETGTIGDEKAEMFKTQVTGKIKLL
GMVEMALLGIGVVMFVAFMISYCACKSKNGK

### Rat CD36 (SEQ ID NO: 4; UNIPROT Q07969):

MGCDRNCGLITGAVIGAVLAVFGGILMPVGDLLIEKTIKREVVLEEGTI
AFKNWVKTGTTVYRQFWIFDVQNPEEVAKNSSKIKVKQRGPYTYRVRYL
AKENITQDPKDSTVSFVQPNGAIFEPSLSVGTENDNFTVLNLAVAAAPH
IYTNSFVQGVLNSLIKKSKSSMFQTRSLKELLWGYKDPFLSLVPYPIST
TVGVFYPYNNTVDGVYKVFNGKDNISKVAIIDTYKGKRNLSYWESYCDM
INGTDAASFPPFVEKSQTLRFFSSDICRSIYAVFESEVNLKGIPVYRFV
LPANAFASPLQNPDNHCFCTEKVISNNCTSYGVLDIGKCKEGKPVYISL
PHFLHASPDVSEPIEGLNPNEDEHRTYLDVEPITGFTLQFAKRLQVNIL
VKPARKIEALKNLKRPYIVPILWLNETGTIGDEKAEMFRNQVTGKIKLL
GLVEMVLLGVGVVMFVAFMISYCACRSKNGK

In certain embodiments, an antibody described herein binds to human CD36. In certain embodiments, an antibody binds to human and cynomolgus monkey CD36. In certain embodiments, an antibody binds to human and murine CD36. In certain embodiments, an antibody binds to human, murine, and rat CD36. In certain embodiments, an antibody binds to human, cynomolgus monkey, rhesus macaque, murine, and rat CD36.

In some embodiments, an antibody described herein has cross-reactivity to human CD36 and non-human CD36. In some embodiments, the antibody has cross-reactivity to human CD36 and non-human primate CD36. In some embodiments, said antibody is selected from the group consisting of 4G04, 5G04, 6G04, 7G04, 9G04, 10G04, 11G04, 12G04, 13G04, 14G04, 15G04, 16G04, 17G04, 18G04, 19G04, 20G04, 21G04, 22G04, 23G04, 24G04, 25G04, 26G04, 27G04, 28G04, 29G04, 30G04, 31G04, 32G04, 33G04, 34G04, 35G04, 36G04, 37G04, and 38G04; or antibodies comprising the same six CDRs or the same VH and VL sequences. In some embodiments the non-human primate CD36 is cynomolgus monkey CD36 or rhesus macaque CD36. In some embodiments, the non-human primate CD36 is cynomolgus monkey CD36. In some embodiments, said antibody is selected from the group consisting of 4G04, 5G04, 6G04, 7G04, 9G04, 10G04, 11G04, 12G04, 13G04, 14G04, 15G04, 16G04, 17G04, 18G04, 19G04, 20G04, 21G04, 22G04, 23G04, 24G04, 25G04, 27G04, 28G04, 29G04, 30G04, 32G04, 33G04, 34G04, 35G04, 36G04, 37G04, and 38G04; or antibodies comprising the same six CDRs or the same VH and VL sequences. In some embodiments, the non-human primate CD36 is rhesus macaque CD36.

In some embodiments, the antibody has cross-reactivity to human CD36 and rodent CD36. In some embodiments, said antibody is selected from the group consisting of 4G04, 5G04, 6G04, 7G04, 9G04, 10G04, 11G04, 12G04, 13G04, 14G04, 16G04, 17G04, 18G04, 19G04, 20G04, 28G04, 29G04, 30G04, 32G04, 33G04, 34G04, 35G04, 36G04, 37G04, and 38G04; or antibodies comprising the same six CDRs or the same VH and VL sequences. In some embodiments, the rodent CD36 is mouse CD36. In preferred embodiments, said antibody is selected from the group consisting of 4G04, 5G04, 6G04, 7G04, 9G04, 10G04, 11G04, 12G04, 13G04, 14G04, 16G04, 17G04, 18G04, 19G04, 20G04, 28G04, 29G04, 30G04, 32G04, 33G04, 34G04, 35G04, 36G04, 37G04, and 38G04; or antibodies comprising the same six CDRs or the same VH and VL sequences. In some embodiments, the rodent CD36 is rat CD36.

In some embodiments, the antibody has cross-reactivity to human CD36, non-human primate CD36, and rodent CD36 (e.g., 4G04, 5G04, 6G04, 7G04, 9G04, 10G04, 11G04, 12G04, 13G04, 14G04, 16G04, 17G04, 18G04, 19G04, 20G04, 28G04, 29G04, 30G04, 32G04, 33G04, 34G04, 35G04, 36G04, 37G04, and 38G04). In other embodiments, the antibody is specific for human CD36.

Anti-CD36 antibodies of the invention include a full length antibody, a single chain antibody, and a scFv, Fab or F(ab′)₂ fragment. In some embodiments, the anti-CD-36 antibody is a full length antibody. In some embodiments, the anti-CD36 antibody is a humanized antibody. In some embodiments, the anti-CD36 antibody is a human antibody. In some embodiments, the anti-CD36 antibody is ONA-0-v1, 1G03, 1G04, 1G05, 1G06, 1G07, 4G04, 5G04, 6G04, 7G04, 9G04, 10G04, 11G04, 12G04, 13G04, 14G04, 15G04, 16G04, 17G04, 18G04, 19G04, 20G04, 21G04, 22G04, 23G04, 24G04, 25G04, 26G04, 27G04, 28G04, 29G04, 30G04, 31G04, 32G04, 33G04, 34G04, 35G04, 36G04, 37G04, 38G04, 73G06, 74G06, 75G06, 76G06, 77G06, 78G06, 79G06, or 80G06. In some embodiments, the anti-CD36 antibody is an antibody containing one or more of the CDR sequences from ONA-0-v1, 1G03, 1G04, 1G05, 1G06, 1G07, 4G04, 5G04, 6G04, 7G04, 9G04, 10G04, 11G04, 12G04, 13G04, 14G04, 15G04, 16G04, 17G04, 18G04, 19G04, 20G04, 21G04, 22G04, 23G04, 24G04, 25G04, 26G04, 27G04, 28G04, 29G04, 30G04, 31G04, 32G04, 33G04, 34G04, 35G04, 36G04, 37G04, 38G04, 73G06, 74G06, 75G06, 76G06, 77G06, 78G06, 79G06, or 80G06.

Embodiments of the invention also include antibody fragments derived from the anti-CD36 antibodies disclosed herein, including but not limited to Fab, Fab′, F(ab′)₂, single chain Fv (scFv), disulfide linked Fv, V-NAR domain, IgNar, intrabody, IgGΔCH2, minibody, F(ab′)₃, tetrabody, triabody, diabody, single-domain antibody, DVD-Ig, Fcab, mAb2, (scFv)₂, or scFv-Fc. An antibody fragment can be produced by any technique known to those of skill in the art. In certain embodiments, the antibody fragment further comprises a moiety that extends the half-life of the antibody in vivo. The moiety is also termed a “half-life extending moiety.” Any moiety known to those of skill in the art for extending the half-life of an antibody fragment in vivo can be used. For example, the half-life extending moiety can include a Fc region, a polymer, an albumin, or an albumin binding protein or compound. The polymer can include a natural or synthetic, optionally substituted straight or branched chain polyalkylene, polyalkenylene, polyoxylalkylene, polysaccharide, polyethylene glycol, polypropylene glycol, polyvinyl alcohol, methoxypolyethylene glycol, lactose, amylose, dextran, glycogen, or derivative thereof. Substituents can include one or more hydroxy, methyl, or methoxy groups. In certain embodiments, the Fab, Fab′, F(ab′)₂, or scFv can be modified by the addition of one or more C-terminal amino acids for attachment of the half-life extending moiety. In certain embodiments the half-life extending moiety is polyethylene glycol or human serum albumin. In certain embodiments, the Fab, Fab′, F(ab′)₂, or scFv is fused to a Fc region.

In some embodiments, the antibody thereof is a humanized antibody comprising one or more of the CDRs of the antibodies disclosed herein, as identified by the Chothia, Kabat, or IMGT antibody numbering schemes. Exemplary embodiments of antibodies comprising one or more of the CDRs of the antibodies disclosed herein (as identified according to the Kabat numbering scheme) are provided in Table 2 below. In some embodiments, the anti-CD36 antibody comprises the CDR combinations provided in Table 2. In some embodiments, the anti-CD36 antibody is a chimeric antibody comprising the CDR combinations provided in Table 2. In some embodiments, the anti-CD36 antibody is a humanized antibody comprising the CDR combinations provided in Table 2.

TABLE 2
CDRs in Anti-CD36 Antibodies

| | CDR-H1 | CDR-H2 | CDR-H3 | CDR-L1 | CDR-L2 | CDR-L3 |
|---|---|---|---|---|---|---|
| | SEQ ID | SEQ ID | SEQ ID | SEQ ID | SEQ ID | SEQ ID |
| Antibody | NO | NO | NO | NO | NO | NO |
| ONA-0-v1 | 79 | 80 | 81 | 82 | 83 | 84 |
| 1G04 | 79 | 80 | 81 | 82 | 83 | 84 |
| 1G06 | 79 | 80 | 81 | 82 | 83 | 84 |
| 4G04 | 85 | 106 | 133 | 159 | 173 | 186 |
| 5G04 | 86 | 107 | 134 | 160 | 174 | 187 |
| 6G04 | 86 | 108 | 135 | 160 | 174 | 188 |
| 7G04 | 87 | 109 | 136 | 160 | 174 | 187 |
| 9G04 | 88 | 110 | 137 | 160 | 174 | 187 |
| 10G04 | 89 | 111 | 138 | 161 | 175 | 189 |
| 11G04 | 90 | 112 | 139 | 160 | 174 | 190 |
| 12G04 | 91 | 113 | 140 | 160 | 174 | 187 |
| 13G04 | 92 | 114 | 141 | 162 | 176 | 191 |
| 14G04 | 93 | 115 | 142 | 163 | 177 | 192 |
| 15G04 | 94 | 116 | 143 | 164 | 175 | 193 |
| 16G04 | 95 | 117 | 144 | 160 | 174 | 190 |
| 17G04 | 95 | 118 | 145 | 160 | 174 | 187 |
| 18G04 | 95 | 119 | 146 | 160 | 174 | 190 |
| 19G04 | 95 | 119 | 146 | 160 | 174 | 190 |
| 20G04 | 95 | 119 | 146 | 160 | 174 | 190 |
| 21G04 | 96 | 120 | 147 | 159 | 173 | 194 |
| 22G04 | 96 | 121 | 148 | 159 | 173 | 195 |
| 23G04 | 96 | 120 | 147 | 159 | 173 | 186 |
| 24G04 | 96 | 121 | 147 | 159 | 173 | 196 |
| 25G04 | 96 | 121 | 147 | 159 | 173 | 195 |
| 26G04 | 97 | 122 | 149 | 165 | 178 | 197 |
| 27G04 | 97 | 123 | 150 | 165 | 178 | 197 |
| 28G04 | 98 | 124 | 151 | 160 | 174 | 198 |
| 29G04 | 96 | 120 | 147 | 159 | 173 | 195 |
| 30G04 | 91 | 125 | 152 | 160 | 174 | 190 |
| 31G04 | 99 | 126 | 133 | 162 | 176 | 199 |
| 32G04 | 100 | 127 | 153 | 166 | 179 | 200 |
| 33G04 | 101 | 128 | 154 | 167 | 180 | 201 |
| 34G04 | 101 | 128 | 154 | 168 | 181 | 202 |
| 35G04 | 102 | 129 | 155 | 169 | 182 | 203 |
| 36G04 | 103 | 130 | 156 | 170 | 183 | 204 |
| 37G04 | 104 | 131 | 157 | 171 | 184 | 205 |
| 38G04 | 105 | 132 | 158 | 172 | 185 | 206 |
| 73G06 | 90 | 248 | 139 | 160 | 174 | 247 |
| 74G06 | 90 | 248 | 139 | 160 | 174 | 247 |
| 75G06 | 90 | 248 | 139 | 160 | 174 | 247 |
| 76G06 | 90 | 248 | 139 | 160 | 174 | 247 |
| 77G06 | 90 | 248 | 139 | 160 | 246 | 247 |
| 78G06 | 90 | 248 | 139 | 160 | 246 | 247 |
| 79G06 | 90 | 248 | 139 | 160 | 246 | 247 |
| 80G06 | 90 | 248 | 139 | 160 | 246 | 247 |

ONA-0-v1 has an Fc region corresponding to human IgG1 wild type sequence; Antibody name code: the prefix refers to the variable region (e.g., 1G04 and 1G06 have the variable region of ONA-0-v1); the suffix refers to the Fc region: the antibodies ending in “G04” have the Fc region of human IgG1 with the L234A and L235A (“LALA”) alteration; and the antibodies ending in “G06” have human IgG1 with the amino acid mutations L234S, L235T, and G236R (“STR”).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises a sequence selected from the group consisting of SEQ ID NOs: 85-105; the heavy chain CDR2 region comprises a sequence selected from the group consisting of SEQ ID NOs: 106-132, and 248; the heavy chain CDR3 region comprises a sequence selected from the group consisting of SEQ ID NOs: 133-158; the light chain CDR1 region comprises a sequence selected from the group consisting of SEQ ID NOs: 159-172; the light chain CDR2 region comprises a sequence selected from the group consisting of SEQ ID NOs: 173-185, and 246; and the light chain CDR3 region comprises a sequence selected from the group consisting of SEQ ID NOs: 186-206, and 247. In some embodiments, the anti-CD36 antibody is a chimeric antibody comprising the CDR a combination of these CDR regions. In some embodiments, the anti-CD36 antibody is a humanized antibody comprising a combination of these CDR regions.

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 79; the heavy chain CDR2 region comprises SEQ ID NO: 80; the heavy chain CDR3 region comprises SEQ ID NO:81; the light chain CDR1 region comprises SEQ ID NO: 82; the light chain CDR2 region comprises SEQ ID NO: 83; and the light chain CDR3 region comprises SEQ ID NO:84 (e.g. ONA-0-v1, 1G04, and 1G06).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 85; the heavy chain CDR2 region comprises SEQ ID NO:106; the heavy chain CDR3 region comprises SEQ ID NO:133; the light chain CDR1 region comprises SEQ ID NO: 159; the light chain CDR2 region comprises SEQ ID NO: 173; and the light chain CDR3 region comprises SEQ ID NO: 186 (e.g., 4G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 86; the heavy chain CDR2 region comprises SEQ ID NO:107; the heavy chain CDR3 region comprises SEQ ID NO:134; the light chain CDR1 region comprises SEQ ID NO: 160; the light chain CDR2 region comprises SEQ ID NO: 174; and the light chain CDR3 region comprises SEQ ID NO: 187 (e.g., 5G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 86; the heavy chain CDR2 region comprises SEQ ID NO: 108; the heavy chain CDR3 region comprises SEQ ID NO:135; the light chain CDR1 region comprises SEQ ID NO: 160; the light chain CDR2 region comprises SEQ ID NO: 174; and the light chain CDR3 region comprises SEQ ID NO: 188 (e.g., 6G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 87; the heavy chain CDR2 region comprises SEQ ID NO: 109; the heavy chain CDR3 region comprises SEQ ID NO: 136; the light chain CDR1 region comprises SEQ ID NO: 160; the light chain CDR2 region comprises SEQ ID NO: 174; and the light chain CDR3 region comprises SEQ ID NO: 187 (e.g., 7G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 88; the heavy chain CDR2 region comprises SEQ ID NO: 110; the heavy chain CDR3 region comprises SEQ ID NO: 137; the light chain CDR1 region comprises SEQ ID NO: 160; the light chain CDR2 region comprises SEQ ID NO: 174; and the light chain CDR3 region comprises SEQ ID NO: 187 (e.g., 9G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 89; the heavy chain CDR2 region comprises SEQ ID NO: 111; the heavy chain CDR3 region comprises SEQ ID NO: 138; the light chain CDR1 region comprises SEQ ID NO: 161; the light chain CDR2 region comprises SEQ ID NO: 175; and the light chain CDR3 region comprises SEQ ID NO: 189 (e.g., 10G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 90; the heavy chain CDR2 region comprises SEQ ID NO: 112; the heavy chain CDR3 region comprises SEQ ID NO: 139; the light chain CDR1 region comprises SEQ ID NO: 160; the light chain CDR2 region comprises SEQ ID NO: 174; and the light chain CDR3 region comprises SEQ ID NO: 190 (e.g., 11G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 91; the heavy chain CDR2 region comprises SEQ ID NO: 113; the heavy chain CDR3 region comprises SEQ ID NO: 140; the light chain CDR1 region comprises SEQ ID NO: 160; the light chain CDR2 region comprises SEQ ID NO: 174; and the light chain CDR3 region comprises SEQ ID NO: 187 (e.g., 12G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 92; the heavy chain CDR2 region comprises SEQ ID NO: 114; the heavy chain CDR3 region comprises SEQ ID NO: 141; the light chain CDR1 region comprises SEQ ID NO: 162; the light chain CDR2 region comprises SEQ ID NO: 176; and the light chain CDR3 region comprises SEQ ID NO: 191 (e.g., 13G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 93; the heavy chain CDR2 region comprises SEQ ID NO: 115; the heavy chain CDR3 region comprises SEQ ID NO: 142; the light chain CDR1 region comprises SEQ ID NO: 163; the light chain CDR2 region comprises SEQ ID NO: 177; and the light chain CDR3 region comprises SEQ ID NO: 192 (e.g., 14G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 94; the heavy chain CDR2 region comprises SEQ ID NO: 116; the heavy chain CDR3 region comprises SEQ ID NO: 143; the light chain CDR1 region comprises SEQ ID NO: 164; the light chain CDR2 region comprises SEQ ID NO:175; and the light chain CDR3 region comprises SEQ ID NO: 193 (e.g., 15G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 95; the heavy chain CDR2 region comprises SEQ ID NO: 117; the heavy chain CDR3 region comprises SEQ ID NO: 144; the light chain CDR1 region comprises SEQ ID NO: 160; the light chain CDR2 region comprises SEQ ID NO: 174; and the light chain CDR3 region comprises SEQ ID NO: 190 (e.g., 16G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 95; the heavy chain CDR2 region comprises SEQ ID NO: 118; the heavy chain CDR3 region comprises SEQ ID NO: 145; the light chain CDR1 region comprises SEQ ID NO: 160; the light chain CDR2 region comprises SEQ ID NO: 174; and the light chain CDR3 region comprises SEQ ID NO: 187 (e.g., 17G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 95; the heavy chain CDR2 region comprises SEQ ID NO: 119; the heavy chain CDR3 region comprises SEQ ID NO: 146; the light chain CDR1 region comprises SEQ ID NO: 160; the light chain CDR2 region comprises SEQ ID NO: 174; and the light chain CDR3 region comprises SEQ ID NO: 190 (e.g., 18G04, 19G04, and 20G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 96; the heavy chain CDR2 region comprises SEQ ID NO: 120; the heavy chain CDR3 region comprises SEQ ID NO: 147; the light chain CDR1 region comprises SEQ ID NO: 159; the light chain CDR2 region comprises SEQ ID NO: 173; and the light chain CDR3 region comprises SEQ ID NO: 194 (e.g., 21G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 96; the heavy chain CDR2 region comprises SEQ ID NO: 121; the heavy chain CDR3 region comprises SEQ ID NO: 148; the light chain CDR1 region comprises SEQ ID NO: 159; the light chain CDR2 region comprises SEQ ID NO: 173; and the light chain CDR3 region comprises SEQ ID NO: 195 (e.g., 22G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 96; the heavy chain CDR2 region comprises SEQ ID NO: 120; the heavy chain CDR3 region comprises SEQ ID NO: 147; the light chain CDR1 region comprises SEQ ID NO: 159; the light chain CDR2 region comprises SEQ ID NO: 173; and the light chain CDR3 region comprises SEQ ID NO: 186 (e.g., 23G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 96; the heavy chain CDR2 region comprises SEQ ID NO: 121; the heavy chain CDR3 region comprises SEQ ID NO: 147; the light chain CDR1 region comprises SEQ ID NO: 159; the light chain CDR2 region comprises SEQ ID NO: 173; and the light chain CDR3 region comprises SEQ ID NO: 196 (e.g., 24G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 96; the heavy chain CDR2 region comprises SEQ ID NO: 121; the heavy chain CDR3 region comprises SEQ ID NO: 147; the light chain CDR1 region comprises SEQ ID NO: 159; the light chain CDR2 region comprises SEQ ID NO: 173; and the light chain CDR3 region comprises SEQ ID NO: 195 (e.g., 25G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 97; the heavy chain CDR2 region comprises SEQ ID NO: 122; the heavy chain CDR3 region comprises SEQ ID NO: 149; the light chain CDR1 region comprises SEQ ID NO: 165; the light chain CDR2 region comprises SEQ ID NO: 178; and the light chain CDR3 region comprises SEQ ID NO: 197 (e.g., 26G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 97; the heavy chain CDR2 region comprises SEQ ID NO: 123; the heavy chain CDR3 region comprises SEQ ID NO: 150; the light chain CDR1 region comprises SEQ ID NO: 165; the light chain CDR2 region comprises SEQ ID NO: 178; and the light chain CDR3 region comprises SEQ ID NO: 197 (e.g., 27G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 98; the heavy chain CDR2 region comprises SEQ ID NO: 124; the heavy chain CDR3 region comprises SEQ ID NO: 151; the light chain CDR1 region comprises SEQ ID NO: 160; the light chain CDR2 region comprises SEQ ID NO: 174; and the light chain CDR3 region comprises SEQ ID NO: 198 (e.g., 28G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 96; the heavy chain CDR2 region comprises SEQ ID NO: 120; the heavy chain CDR3 region comprises SEQ ID NO: 147; the light chain CDR1 region comprises SEQ ID NO: 159; the light chain CDR2 region comprises SEQ ID NO: 173; and the light chain CDR3 region comprises SEQ ID NO: 195 (e.g., 29G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 91; the heavy chain CDR2 region comprises SEQ ID NO: 125; the heavy chain CDR3 region comprises SEQ ID NO: 152; the light chain CDR1 region comprises SEQ ID NO: 160; the light chain CDR2 region comprises SEQ ID NO: 174; and the light chain CDR3 region comprises SEQ ID NO: 190 (e.g., 30G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 99; the heavy chain CDR2 region comprises SEQ ID NO: 126; the heavy chain CDR3 region comprises SEQ ID NO: 133; the light chain CDR1 region comprises SEQ ID NO: 162; the light chain CDR2 region comprises SEQ ID NO: 176; and the light chain CDR3 region comprises SEQ ID NO: 199 (e.g., 31G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO:100; the heavy chain CDR2 region comprises SEQ ID NO: 127; the heavy chain CDR3 region comprises SEQ ID NO: 153; the light chain CDR1 region comprises SEQ ID NO: 166; the light chain CDR2 region comprises SEQ ID NO: 179; and the light chain CDR3 region comprises SEQ ID NO: 200 (e.g., 32G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 101; the heavy chain CDR2 region comprises SEQ ID NO: 128; the heavy chain CDR3 region comprises SEQ ID NO: 154; the light chain CDR1 region comprises SEQ ID NO: 167; the light chain CDR2 region comprises SEQ ID NO: 180; and the light chain CDR3 region comprises SEQ ID NO: 201 (e.g., 33G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO:101; the heavy chain CDR2 region comprises SEQ ID NO: 128; the heavy chain CDR3 region comprises SEQ ID NO: 154; the light chain CDR1 region comprises SEQ ID NO: 168; the light chain CDR2 region comprises SEQ ID NO: 181; and the light chain CDR3 region comprises SEQ ID NO: 202 (e.g., 34G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 102; the heavy chain CDR2 region comprises SEQ ID NO: 129; the heavy chain CDR3 region comprises SEQ ID NO: 155; the light chain CDR1 region comprises SEQ ID NO: 169; the light chain CDR2 region comprises SEQ ID NO: 182; and the light chain CDR3 region comprises SEQ ID NO: 203 (e.g., 35G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 103; the heavy chain CDR2 region comprises SEQ ID NO: 130; the heavy chain CDR3 region comprises SEQ ID NO: 156; the light chain CDR1 region comprises SEQ ID NO: 170; the light chain CDR2 region comprises SEQ ID NO: 183; and the light chain CDR3 region comprises SEQ ID NO: 204 (e.g., 36G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 104; the heavy chain CDR2 region comprises SEQ ID NO: 131; the heavy chain CDR3 region comprises SEQ ID NO: 157; the light chain CDR1 region comprises SEQ ID NO: 171; the light chain CDR2 region comprises SEQ ID NO: 184; and the light chain CDR3 region comprises SEQ ID NO: 205 (e.g., 37G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 105; the heavy chain CDR2 region comprises SEQ ID NO: 132; the heavy chain CDR3 region comprises SEQ ID NO: 158; the light chain CDR1 region comprises SEQ ID NO: 172; the light chain CDR2 region comprises SEQ ID NO: 185; and the light chain CDR3 region comprises SEQ ID NO: 206 (e.g., 38G04).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 90; the heavy chain CDR2 region comprises SEQ ID NO: 248; the heavy chain CDR3 region comprises SEQ ID NO: 139; the light chain CDR1 region comprises SEQ ID NO: 160; the light chain CDR2 region comprises SEQ ID NO: 174; and the light chain CDR3 region comprises SEQ ID NO: 247 (e.g., 73G06, 74G06, 75G06, 76G06).

In certain embodiments, the anti-CD36 antibody comprises a light chain CDR1 region, a light chain CDR2 region, a light chain CDR3 region, a heavy chain CDR1 region, a heavy chain CDR2 region, and a heavy chain CDR3 region, wherein the heavy chain CDR1 region comprises SEQ ID NO: 90; the heavy chain CDR2 region comprises SEQ ID NO: 248; the heavy chain CDR3 region comprises SEQ ID NO: 139; the light chain CDR1 region comprises SEQ ID NO: 160; the light chain CDR2 region comprises SEQ ID NO: 246; and the light chain CDR3 region comprises SEQ ID NO: 247 (e.g., 77G06, 78G06, 79G06, and 80G06).

In certain embodiments, an antibody described herein binds to human CD36 and comprises the VH sequence of an antibody disclosed herein. In some embodiments, the anti-CD36 antibody comprises the VH sequence provided as one of SEQ ID NOs: 7, 13-44, 241, and 243. In certain embodiments, the antibody comprises a VH comprising at least 85%, at least 90%, at least 95%, at least 96%, at least 97%, at least 98%, or at least 99% identity to the VH sequence of one of SEQ ID NOs: 7, 13-44, 241, and 243. In certain embodiments, an antibody described herein binds to human CD36 and comprises the VL sequence of an antibody disclosed herein. In some embodiments, the anti-CD36 antibody comprises the VH sequence provided as one of SEQ ID NOs: 8, 45-78, 240, 242, 244, and 245. In certain embodiments, the antibody comprises a VL comprising at least 85%, at least 90%, at least 95%, at least 96%, at least 97%, at least 98%, or at least 99% identity to the VL sequence of one of SEQ ID NOs: 8, 45-78, 240, 242, 244, and 245.

In certain embodiments, the anti-CD36 antibody comprises both a VH and a VL disclosed herein. In certain embodiments, the anti-CD36 antibody comprises both a VH and a VL, wherein the VH comprises one of SEQ ID NOs: 7, 13-44, 241, and 243, and wherein the VL comprises one of SEQ ID NOs: 8, 45-78, 240, 242, 244, and 245. In certain embodiments, the anti-CD36 antibody comprises both a VH and a VL, wherein
the heavy chain variable region comprises SEQ ID NO: 13 and the light chain variable region comprises SEQ ID NO: 45;
the heavy chain variable region comprises SEQ ID NO: 14 and the light chain variable region comprises SEQ ID NO: 46;
the heavy chain variable region comprises SEQ ID NO: 15 and the light chain variable region comprises SEQ ID NO: 47;
the heavy chain variable region comprises SEQ ID NO: 16 and the light chain variable region comprises SEQ ID NO: 48;
the heavy chain variable region comprises SEQ ID NO: 17 and the light chain variable region comprises SEQ ID NO: 49;
the heavy chain variable region comprises SEQ ID NO: 18 and the light chain variable region comprises SEQ ID NO: 50;
the heavy chain variable region comprises SEQ ID NO: 19 and the light chain variable region comprises SEQ ID NO: 51;
the heavy chain variable region comprises SEQ ID NO: 20 and the light chain variable region comprises SEQ ID NO: 52;
the heavy chain variable region comprises SEQ ID NO: 21 and the light chain variable region comprises SEQ ID NO: 53;
the heavy chain variable region comprises SEQ ID NO: 22 and the light chain variable region comprises SEQ ID NO: 54;
the heavy chain variable region comprises SEQ ID NO: 23 and the light chain variable region comprises SEQ ID NO: 55;
the heavy chain variable region comprises SEQ ID NO: 24 and the light chain variable region comprises SEQ ID NO: 56;
the heavy chain variable region comprises SEQ ID NO: 25 and the light chain variable region comprises SEQ ID NO: 57;
the heavy chain variable region comprises SEQ ID NO: 26 and the light chain variable region comprises SEQ ID NO: 58;
the heavy chain variable region comprises SEQ ID NO: 26 and the light chain variable region comprises SEQ ID NO: 59;
the heavy chain variable region comprises SEQ ID NO: 27 and the light chain variable region comprises SEQ ID NO: 60;
the heavy chain variable region comprises SEQ ID NO: 28 and the light chain variable region comprises SEQ ID NO: 61;
the heavy chain variable region comprises SEQ ID NO: 29 and the light chain variable region comprises SEQ ID NO: 62;
the heavy chain variable region comprises SEQ ID NO: 30 and the light chain variable region comprises SEQ ID NO: 63;
the heavy chain variable region comprises SEQ ID NO: 31 and the light chain variable region comprises SEQ ID NO: 64;
the heavy chain variable region comprises SEQ ID NO: 32 and the light chain variable region comprises SEQ ID NO: 65;
the heavy chain variable region comprises SEQ ID NO: 33 and the light chain variable region comprises SEQ ID NO: 66;
the heavy chain variable region comprises SEQ ID NO: 34 and the light chain variable region comprises SEQ ID NO: 67;
the heavy chain variable region comprises SEQ ID NO: 35 and the light chain variable region comprises SEQ ID NO: 68;
the heavy chain variable region comprises SEQ ID NO: 36 and the light chain variable region comprises SEQ ID NO: 69;
the heavy chain variable region comprises SEQ ID NO: 37 and the light chain variable region comprises SEQ ID NO: 70;
the heavy chain variable region comprises SEQ ID NO: 38 and the light chain variable region comprises SEQ ID NO: 71;
the heavy chain variable region comprises SEQ ID NO: 39 and the light chain variable region comprises SEQ ID NO: 72;
the heavy chain variable region comprises SEQ ID NO: 40 and the light chain variable region comprises SEQ ID NO: 73;
the heavy chain variable region comprises SEQ ID NO: 40 and the light chain variable region comprises SEQ ID NO: 74;
the heavy chain variable region comprises SEQ ID NO: 41 and the light chain variable region comprises SEQ ID NO: 75;
the heavy chain variable region comprises SEQ ID NO: 42 and the light chain variable region comprises SEQ ID NO: 76;
the heavy chain variable region comprises SEQ ID NO: 43 and the light chain variable region comprises SEQ ID NO: 77;
the heavy chain variable region comprises SEQ ID NO: 44 and the light chain variable region comprises SEQ ID NO: 78;
the heavy chain variable region comprises SEQ ID NO: 241 and the light chain variable region comprises SEQ ID NO: 240;
the heavy chain variable region comprises SEQ ID NO: 243 and the light chain variable region comprises SEQ ID NO: 240;
the heavy chain variable region comprises SEQ ID NO: 241 and the light chain variable region comprises SEQ ID NO: 242;
the heavy chain variable region comprises SEQ ID NO: 243 and the light chain variable region comprises SEQ ID NO: 242;
the heavy chain variable region comprises SEQ ID NO: 241 and the light chain variable region comprises SEQ ID NO: 244;
the heavy chain variable region comprises SEQ ID NO: 243 and the light chain variable region comprises SEQ ID NO: 244;
the heavy chain variable region comprises SEQ ID NO: 241 and the light chain variable region comprises SEQ ID NO: 245; or
the heavy chain variable region comprises SEQ ID NO: 243 and the light chain variable region comprises SEQ ID NO: 245.

In certain embodiments, the anti-CD36 antibody is a bispecific antibody. The term “bispecific” means that the antibody in question is able to specifically bind to at least two distinct epitopes or antigens. Typically, a bispecific antibody comprises two antigen binding sites, each of which is specific for a different epitope or antigen. Accordingly, in some embodiments the bispecific anti-CD36 antibody also binds to a second epitope or antigen. In some embodiments, the bispecific anti-CD36 antibody comprises a first antigen-binding domain that specifically binds to CD36 and a second antigen-binding domain that specifically binds to a second antigen. In some embodiments, the bispecific anti-CD36 antibody comprises a first antigen-binding domain that specifically binds to CD36 and a second antigen-binding domain that specifically binds to an immune cell antigen (see e.g. Wang, et al. 2021). In some embodiments, the immune cell antigen is a T-cell receptor antigen. In some embodiments the immune cell antigen is selected from a group consisting of: PD-1, PD-L1, CTLA4, CD3, LAG3, OX40, CD28, CD33, B7H3, CD47, TIM3, ICOS, LGR5, 4-1BB, CD40, CD40-L and TIGIT. In some embodiments, the immune cell antigen is CD3. In some embodiments the immune cell antigen is PD-1. In some embodiments, the immune cell antigen is PD-L1. In some embodiments, the bispecific anti-CD36 antibody comprises a first antigen-binding domain that specifically binds to CD36 and a second antigen-binding domain that specifically binds to a tumor-specific antigen. In some embodiments, the tumor-specific antigen is selected from the group consisting of: HER2, HER3, EGFR, VEGF, IGF-1, IGF-2, ANG2, DLL1, IGF-1R, cMET, DLL4, FAP, DR5, IL15, IL15Ra, CD3, CEA, EpCAM, PSMA, PMEL, and GPC3. In some embodiments, the tumor-specific antigen is CD3. In some embodiments, the bispecific anti-CD36 antibody comprises one or more CDRs provides in SEQ ID NOs: 79-206 and 246-248. Embodiments of the invention include methods of using such bispecific antibodies to recruit T cells to tumors. In some embodiments of these methods, the recruited T cells lyse tumor cells while bypassing antigen presentation through the major histocompatibility complex. Exemplary methods for preparing and using bispecific antibodies can be found in WO 2016/141287 A1, which is incorporated herein by reference in its entirety.

In certain embodiments, the bispecific anti-CD36 antibody is a biparatopic antibody. The term “biparatopic” means that the antibody in question is able to specifically bind to at least two distinct epitopes of a target antigen. In some embodiments, the two distinct epitopes are unique and non-overlapping. Typically, a biparatopic antibody comprises two antigen-binding sites, each of which is specific for a different epitope on the same antigen. Accordingly, in some embodiments, the biparatropic anti-CD36 antibody binds to a first and second epitope on CD36. In some embodiments, the biparatopic anti-CD36 antibody comprises a first antigen-binding domain that specifically binds to a first epitope on CD36 and a second antigen-binding domain that specifically binds to a second epitope on CD36. In some embodiments, the biparatopic anti-CD36 antibody comprises a first antigen-binding domain, which comprises the antigen-binding domain of 1G04. In some embodiments, the biparatopic anti-CD36 antibody comprises a first antigen-binding domain, which comprises the antigen-binding domain of 1G04, and a second antigen-binding domain, which comprises an antigen-binding domain of an antibody selected from the group consisting of 10G04, 11G04, 19G04, 20G04, and 30G04. In some embodiments the biparatopic anti-CD36 antibody comprises a first antigen-binding domain, which comprises the antigen-binding domain of 1G04, and a second antigen-binding domain, which comprises an antigen-binding domain of 11G04. In some embodiments, the biparatopic anti-CD36 antibody comprises a first antigen-binding domain, which comprises the antigen-binding domain of 11G04. In some embodiments, the biparatopic anti-CD36 antibody comprises a first antigen-binding domain, which comprises the antigen-binding domain of 11G04, and a second antigen-binding domain, which comprises an antigen-binding domain of an antibody selected from the group consisting of 1G04, 10G04, 19G04, 20G04, and 30G04. In some embodiments the biparatopic anti-CD36 antibody comprises a first antigen-binding domain, which comprises the antigen-binding domain of 11G04, and a second antigen-binding domain, which comprises an antigen-binding domain of 10G04. In some embodiments, the biparatropic anti-CD36 antibody provides improved properties compared to an anti-CD36 antibody which recognizes only a first CD36 epitope. For example, the biparatopic anti-CD36 antibody may have an enhanced, additive or synergistic effect. In some embodiments, the biparatopic anti-CD36 antibody binds two epitopes on a single CD36 monomer. In other embodiments, the first binding-domain of the biparatopic anti-CD36 antibody binds one CD36 molecule and the second binding-domain of the biparatopic anti-CD36 antibody binds a second CD36 molecule. In some embodiments, the biparatopic anti-CD36 antibody comprises one or more CDRs provides in SEQ ID NOs: 79-206 and 246-248. Embodiments of the invention include methods of using such biparatopic antibodies to recruit T cells to tumors. In some embodiments of these methods, the recruited T cells lyse tumor cells while bypassing antigen presentation through the major histocompatibility complex. Exemplary methods for preparing and using biparatopic antibodies can be found in Wang, S. et al., EMBO Mol. Med. 13:e14291 (2021), which is incorporated herein by reference in its entirety.

In certain embodiments, the invention is directed to the use of an anti-CD36 antibody described in any of the above embodiments either alone or in combination with one or more additional therapeutic agents, e.g., an immunotherapy, for use in any method of treatment disclosed herein, particularly a method of treating cancer and/or cancer metastasis.

Amino acid sequences relating to anti-CD36 antibodies disclosed in this application are provided below in Table 3.

TABLE 3
Amino Acid Sequences

| SEQ ID NO | Name | Sequence |
|---|---|---|
| 1 | Human CD36 | MGCDRNCGLIAGAVIGAVLAVFGGILMPVGDLLIQKTIKKQ |
| | | VVLEEGTIAFKNWVKTGTEVYRQFWIFDVQNPQEVMMNSSN |
| | | IQVKQRGPYTYRVRFLAKENVTQDAEDNTVSFLQPNGAIFE |
| | | PSLSVGTEADNFTVLNLAVAAASHIYQNQFVQMILNSLINK |
| | | SKSSMFQVRTLRELLWGYRDPFLSLVPYPVTTTVGLFYPYN |
| | | NTADGVYKVFNGKDNISKVAIIDTYKGKRNLSYWESHCDMI |
| | | NGTDAASFPPFVEKSQVLQFFSSDICRSIYAVFESDVNLKG |
| | | IPVYRFVLPSKAFASPVENPDNYCFCTEKIISKNCTSYGVL |
| | | DISKCKEGRPVYISLPHFLYASPDVSEPIDGLNPNEEEHRT |
| | | YLDIEPITGFTLQFAKRLQVNLLVKPSEKIQVLKNLKRNYI |
| | | VPILWLNETGTIGDEKANMFRSQVTGKINLLGLIEMILLSV |
| | | GVVMFVAFMISYCACRSKTIK |
| 2 | Cynomolgus | MGCDRNCGLITGAVIGAVLAVFGGILMPVGDMLIQKTIKKE |
| | monkey/ | VVLEEGTIAFKNWVKTGTEIYRQFWIFDVQNPQEVMMNSSN |
| | Rhesus | IQVKQRGPYTYRVRFLAKENITQDPKDNTVSFLQPNGAIFE |
| | macaque CD36 | PSLSVGTEADNFTVLNLAVAAASHIYPNPFVQVVLNSLINK |
| | | SKSSMFQVRTLRELLWGYTDPFLSLVPYPVSTRVGMFYPYN |
| | | NTADGVYKVFNGKDSISKVAIIDTYKGKRNLSYWESYCDMI |
| | | NGTDAASFPPFVEKSQVLQFFSSDICRSIYAVFESDVNLKG |
| | | IPVYRFVLPSKAFASPVQNPDNHCFCTEKIISKNCTSYGVL |
| | | DISKCKEGKPVYISLPHFLYASPDVSETIDGLNPNEEEHRT |
| | | YLDIEPITGFTLQFAKRLQVNLLVKPSNKIQVLKRLKRNYI |
| | | VPILWLNETGTIGDEKAKMFRSQVTGKINLLGLIEMILLSV |
| | | GVVMFVAFMISYCACRSKTIK |
| 3 | Murine CD36 | MGCDRNCGLIAGAVIGAVLAVFGGILMPVGDMLIEKTIKRE |
| | | VVLEEGTTAFKNWVKTGTTVYRQFWIFDVQNPDDVAKNSSK |
| | | IKVKQRGPYTYRVRYLAKENITQDPEDHTVSFVQPNGAIFE |
| | | PSLSVGTEDDNFTVLNLAVAAAPHIYQNSFVQVVLNSLIKK |
| | | SKSSMFQTRSLKELLWGYKDPFLSLVPYPISTTVGVFYPYN |
| | | DTVDGVYKVFNGKDNISKVAIIESYKGKRNLSYWPSYCDMI |
| | | NGTDAASFPPFVEKSRTLRFFSSDICRSIYAVFGSEIDLKG |
| | | IPVYRFVLPANAFASPLQNPDNHCFCTEKVISNNCTSYGVL |
| | | DIGKCKEGKPVYISLPHFLHASPDVSEPIEGLHPNEDEHRT |
| | | YLDVEPITGFTLQFAKRLQVNILVKPARKIEALKNLKRPYI |
| | | VPILWLNETGTIGDEKAEMFKTQVTGKIKLLGMVEMALLGI |
| | | GVVMFVAFMISYCACKSKNGK |
| 4 | Rat CD36 | MGCDRNCGLITGAVIGAVLAVFGGILMPVGDLLIEKTIKRE |
| | | VVLEEGTIAFKNWVKTGTTVYRQFWIFDVQNPEEVAKNSSK |
| | | IKVKQRGPYTYRVRYLAKENITQDPKDSTVSFVQPNGAIFE |
| | | PSLSVGTENDNFTVLNLAVAAAPHIYTNSFVQGVLNSLIKK |
| | | SKSSMFQTRSLKELLWGYKDPFLSLVPYPISTTVGVFYPYN |
| | | NTVDGVYKVFNGKDNISKVAIIDTYKGKRNLSYWESYCDMI |
| | | NGTDAASFPPFVEKSQTLRFFSSDICRSIYAVFESEVNLKG |
| | | IPVYRFVLPANAFASPLQNPDNHCFCTEKVISNNCTSYGVL |
| | | DIGKCKEGKPVYISLPHFLHASPDVSEPIEGLNPNEDEHRT |
| | | YLDVEPITGFTLQFAKRLQVNILVKPARKIEALKNLKRPYI |
| | | VPILWLNETGTIGDEKAEMFRNQVTGKIKLLGLVEMVLLGV |
| | | GVVMFVAFMISYCACRSKNGK |
| 5 | ONA-0-v1 | QVQLKQSGADLVRPGASVKLSCKASGYTFTDYYINWVKQRP |
| | heavy chain | GQGLEWIARIYPGSGNTYYNEKFKGKATLTAEKSSSTAYMQ |
| | | LSSLTSEDSAVYFCARGIGGGFGMDYWGQGTSVTVSSESAR |
| | | NPTIYPLTLPPVLCSDPVIIGCLIHDYFPFGTMNVTWGKSG |
| | | KDITTVNFPPALASGGRYTMSSQLTLPAVECPEGESVKCSV |
| | | QHDSNPVQELDVNCSPTPPPPITIPSCQPSLSLQRPALEDL |
| | | LLGSDASITCTLNGLRNPEGAAFTWEPSTGKDAVQKKAAQN |
| | | SCGCYSVSSVLPGCAERWNSGASFKCTVTHPESGTLTGTIA |
| | | KVTVNTFPPQVHLLPPPSEELALNELLSLTCLVRAFNPKEV |
| | | LVRWLHGNEELSPESYLVFEPLKEPGEGATTYLVTSVLRVS |
| | | AETWKQGDQYSCMVGHEALPMNFTQKTIDRLSGKPTNVSVS |
| | | VIMSEGDGICY |
| 6 | ONA-0-v1 | SIVMTQTPKFLLVSAGDRITITCKASQSVSDDVAWYQQKPG |
| | light chain | QSPKLLIYYASNRYTGVPDRFTGSGYGTDFTFTISTVQAED |
| | | LAVYFCQQDYSSPLTFGAGTKLEIKRADAAPTVSIFPPSSE |
| | | QLTSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| 7 | ONA-0-v1 VH | QVQLKQSGADLVRPGASVKLSCKASGYTFTDYYINWVKQRP |
| | | GQGLEWIARIYPGSGNTYYNEKFKGKATLTAEKSSSTAYMQ |
| | | LSSLTSEDSAVYFCARGIGGGFGMDYWGQGTSVTVSS |
| 8 | ONA-0-v1 VL | SIVMTQTPKFLLVSAGDRITITCKASQSVSDDVAWYQQKPG |
| | | QSPKLLIYYASNRYTGVPDRFTGSGYGTDFTFTISTVQAED |
| | | LAVYFCQQDYSSPLTFGAGTKLEIK |
| 9 | 1G04 | QVQLKQSGADLVRPGASVKLSCKASGYTFTDYYINWVKQRP |
| | heavy chain | GQGLEWIARIYPGSGNTYYNEKFKGKATLTAEKSSSTAYMQ |
| | | LSSLTSEDSAVYFCARGIGGGFGMDYWGQGTSVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKKVEPKSCDKTHTCPPCPAPEAAGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALPAPIEKTISKAKGQPREPQVYTLPPSRDELTKNQVSLTC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| 10 | 1G04 | SIVMTQTPKFLLVSAGDRITITCKASQSVSDDVAWYQQKPG |
| | light chain | QSPKLLIYYASNRYTGVPDRFTGSGYGTDFTFTISTVQAED |
| | | LAVYFCQQDYSSPLTFGAGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 11 | 1G06 | QVQLKQSGADLVRPGASVKLSCKASGYTFTDYYINWVKQRP |
| | heavy chain | GQGLEWIARIYPGSGNTYYNEKFKGKATLTAEKSSSTAYMQ |
| | | LSSLTSEDSAVYFCARGIGGGFGMDYWGQGTSVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKKVEPKSCDKTHTCPPCPAPESTRGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALPAPIEKTISKAKGQPREPQVYTLPPSRDELTKNQVSLTC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| 12 | 1G06 | SIVMTQTPKFLLVSAGDRITITCKASQSVSDDVAWYQQKPG |
| | light chain | QSPKLLIYYASNRYTGVPDRFTGSGYGTDFTFTISTVQAED |
| | | LAVYFCQQDYSSPLTFGAGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 13 | 4G04 VH | QVQLQQPGSELRSPGSSVKLSCKDFDSEVFPIAYMTWVRQK |
| | sequence | PGHGFEWIGDILPSIGGTIYGEKFEDKATLDADTVSNTAYL |
| | | ELNSLTSEDSAIYYCARDYYGSSYGYFDVWGAGTAVTVSS |
| 14 | 5G04 VH | QVQLQQSGAELVMPGASVKMSCKASGYTFTDYWMHWVKQRP |
| | sequence | GQGLEWIGAIDTSDSYTSYNQKFKGKATLTVDESSSTAYMQ |
| | | LSSLTSEDSAVYYCARDYYGSSTAWFAYWGQGTLVTVSA |
| 15 | 6G04 VH | QVQLQQSGAELVMPGASVKMSCKASGYTFTDYWMHWVKQRP |
| | sequence | GQGLEWIGSIDTSDSYTTYSQKFKGKATLTVDESSSTAYME |
| | | LARLTSEDSAIYYCVRGEDYEGTWFAYWGQGTLVTVSA |
| 16 | 7G04 VH | QVQMKESGAELVRPGASVKLSCKALGYTFTDYEIQWVKQTP |
| | sequence | VHGLEWIGGIHPGSSGIVYNQKFKGKATLTADKSSSTAYME |
| | | LSSLTSEDSAVYYCTRGGGYDGAWFAYWGQGSLVTVSA |
| 17 | 9G04 VH | QVQLQQSGAELVRPGASVTLSCKASGYTFTDYEMHWVKQTP |
| | sequence | VHGLEWIGTVDPETGGTAFNQKFKDKASLTADKSSSTVYME |
| | | LRSLTSEDSAVYYCSRGYGNYGAWFAYWGQGTLVTVSA |
| 18 | 10G04 VH | DVQLQESGPELVKPGASVKISCKASGYTFTDYNMHWVKQSH |
| | sequence | GKSLEWIGYIYPYNGGTGYNQKFKSKATLTVDNSSSTAYME |
| | | LRSLTSEDSAVYYCARDGGYDVYFDYWGQGTTLTVSS |
| 19 | 11G04 VH | EVQLQQSGAELVRPGASVTLSCKASGYRFSDYEMQWVKQTP |
| | sequence | VHGLEWIGGIDPETGGIAYNQKFKVKATLTADKSSSTAFME |
| | | LRSLTSEDSAVYYCTRKLDFDYWGQGTTLTVSS |
| 20 | 12G04 VH | EVQLVESGGGLVQPGESLKLSCESNEYEFPSHDMSWVRKTP |
| | sequence | EKRLELVAAINSDGGYTYYPDTMERRFIISRDNTKKTLYLQ |
| | | MSSLRSEDTALYYCARHGYDYDEEGAWFAYWGQGTLVTVSA |
| 21 | 13G04 VH | QVQLQQSGAELVRPGASVKLSCKASGYTFTSYWMNWVKQRP |
| | sequence | GQGLEWIGMIDPSDNETHYNQMFKDKATLTVDKSSSTAYMQ |
| | | LSSLTSEDSAVYYCARSDYGNGYSFYLDVWGAGTTVTVSS |
| 22 | 14G04 VH | QVQLQQSGAELVRSGASVRLSCTASGFNIKDYYIHWVKQRP |
| | sequence | EQGLEWIGWIDPENGDTEYAPRFQDKATMTADTSSNTAYLQ |
| | | LSSLTSEDTAVYYCNGWLLSGNGMDYWGQGTSVTVSS |
| 23 | 15G04 VH | QVQLQQSGPELVKPGASVKISCKASGYTFSTSWMNWVKQRP |
| | sequence | GQGLEWIGRIYPGDGDTNYNGKFKGKATLTADTSSSTAYMQ |
| | | LSSLTSVDSAVYFCAREVYYGGYEDYGMDYWGQGTSVTVSS |
| 24 | 16G04 VH | EVQLQQSGPELVKPGASVKISCKTSGYTFTEYTMHWVKQSH |
| | sequence | GKSLEWIGGINPYNGGTAYNQKFKNKATLTVDISSNTAFME |
| | | FRSLTSEDSAVYYCASDYFYGDANPWFAYWGQGTLVTVSA |
| 25 | 17G04 VH | EVQLQQSGPELVKPGASVKISCKTSGYTFTEYTMHWVKQSH |
| | sequence | GKSLEWIGGIYPYNGGTAYNQKFKNKATLTVDISSNTAYMA |
| | | LRSLASDDSAVYYCASDYFYGDGYPWFTYWGQGTLVTVSA |
| 26 | 18G04 and | EVQLQQSGPELVKPGASVKISCETSGHTFTEYTMHWVKQSH |
| | 19G04 VH | GKSLEWIGGIYPNNGATKYNQKFKGKATLTVDKSSSTAYME |
| | sequence | LRSLTSEDSAVYYCARDRYDVWFAYWGQGTLVTVSA |
| 27 | 20G04 VH | QIQLVQSGPELVKPGASVKISCETSGHTFTEYTMHWVKQSH |
| | sequence | GKSLEWIGGIYPNNGATKYNQKFKGKATLTVDKSSSTAYME |
| | | LRSLTSEDSAVYYCARDRYDVWFAYWGQGTLVTVSA |
| 28 | 21G04 VH | QVQLQQSGAELVKPGASVKLSCKASGYTFTSYWMHWVKQRP |
| | sequence | GQGLEWIGEIDPSDSHTNYNQKFKGKATLTVDKSSSTAYMQ |
| | | LSSLTSEDSAVYYCAPIYYGLDNWGQGTTLTVSS |
| 29 | 22G04 VH | QVQLQQPGAELVKPGASVKLSCKASGYTFTSYWMHWVKQRP |
| | sequence | GQGLEWIGEIDPSDSHANYNQKFKGKATLTVDKSSSTAYMQ |
| | | LSSLTSEDSAVYYCAPIHYGLDNWGQGTTLTVSS |
| 30 | 23G04 VH | QVQLQQPGTELVKPGASVKLSCKASGYTFTSYWMHWVKQRP |
| | sequence | GQGLEWIGEIDPSDSHTNYNQKFKGKATLTVDKSSSTAYMQ |
| | | LSSLTSEDSAVYYCAPIYYGLDNWGQGTTLTVSS |
| 31 | 24G04 VH | QVQLQQSGAELVKPGASVKLSCKASGYTFTSYWMHWVKQRP |
| | sequence | GQGLEWIGEIDPSDSHANYNQKFKGKATLTVDKSSSTAYMQ |
| | | LSSLTSEDSAVYYCAPIYYGLDNWGQGTTLTVSS |
| 32 | 25G04 VH | EVQLQQSGAELVKPGASVKLSCKASGYTFTSYWMHWVKQRP |
| | sequence | GQGLEWIGEIDPSDSHANYNQKFKGKATLTVDKSSSTAYMQ |
| | | LSSLTSEDSAVYYCAPIYYGLDNWGQGTTLTVSS |
| 33 | 26G04 VH | QVQMKESGAELMKPGASVKISCKATGYIFSNYWIEWLKERP |
| | sequence | GHGLEWIGDFLPGSSSANYNEKFKGKATFTADTSSNTAYMQ |
| | | LSSLTSEDSAVYYCVPFTVEVTDAMDYWGQGTSVTVSS |
| 34 | 27G04 VH | EVQLQQSGAELLKPGASVKISCKTTGYTFSNYWIEWVKQRP |
| | sequence | GHGLEWIGEILPGSGSPKYNEKFKGKATLTADTSSNTAFMQ |
| | | LSSLTSEDSAVYYCAPFILENYFDYWGQGTTLTVSS |
| 35 | 28G04 VH | EVQLQQSGPELVKPGASVKISCKTSGYTFTEYTIHWVKQSH |
| | sequence | GKSLEWIGGIIPNNGGSSHKQNFKDKATLTVDKSSSTAYME |
| | | LRSLTSEDSAVYYCARAGDYAFDYWGQGTTLTVSS |
| 36 | 29G04 VH | QVQLQQSGTELVKPGASVKLSCKASGYTFTSYWMHWVKQRP |
| | sequence | GQGLEWIGEIDPSDSHTNYNQKFKGKATLTVDKSSSTAYMQ |
| | | LSSLTSEDSAVYYCAPIYYGLDNWGQGTTLTVSS |
| 37 | 30G04 VH | EVQLVESGGGVVQPGESLKLSCESNEYEFPSHDMSWVRKTP |
| | sequence | ERRLELVAAINSDGAITYYPDTMERRFIISRDNTKKTLYLQ |
| | | MSSLKSEDTAMYYCARHGQTGTWFAYWGQGTLVTVSA |
| 38 | 31G04 VH | QVQLQQSGSELRSPGSSVKLSCKDFDSEVFPIAYMSWVRQK |
| | sequence | PGHGFEWIGDILPSIGSTIYGEKFEDKATLDADTVSNTAYL |
| | | ELNSLTSEDSAIYYCARDYYGSSYGYFDVWGAGTTVTVPS |
| 39 | 32G04 VH | EEKLEESGGGLVQPGGSMKLSCVASGFTFSNYWMNWVRQSP |
| | sequence | EKGLEWVAEIRLKSNNYATHYAESVKGRFTISRDDSKSSVY |
| | | LQMNNLRAEDTGIYYCTPLLLRWGQGTTLTVSS |
| 40 | 33G04 and | AVTLDESGGGLQTPGGALSLVCKASGFDFSSYAMGWVRQAP |
| | 34G04 VH | GKGLEWVAGKAKDGGGTAYGSAVKGRATISRDNGQSTVRLQ |
| | sequence | LSNLRAEDTGVYYCAKSAYGDWFYGSSGPYADSIDAWGHGT |
| | | EVIVSS |
| 41 | 35G04 VH | AVTLDESGGGLQTPGGALSLVCKASGFTFSNYGMGWMRQAP |
| | sequence | GKGLEFVAKIYKDGGYTGYGAAVDGRATISRDDGQSTVRLQ |
| | | LNDLRAEDTATYFCAKAADSGYLYITDSIDAWGHGTEVIVS |
| | | S |
| 42 | 36G04 VH | AVTLDESGGGLQTPGGALSLVCKASGFIFSSYGMGWVRQAP |
| | sequence | GKGLEYIAAISNDGSKADYGAAVKGRATISRDNGQSTVRLQ |
| | | LNNLRAEDTATYYCAKSADTGYCSWSACIADSIDAWGHGTE |
| | | VIVSS |
| 43 | 37G04 VH | AVTLDESGGGLQTPGGALSLVCKASGFTFSDYDMLWVRQAP |
| | sequence | GKGLEFVAGINAASTYTDYGAAVKGRATISRDNGQSTVRLQ |
| | | VNNLRAEDTGTYYCAKSSYGGGWSDVGSIDAWGHGTEVIVS |
| | | S |
| 44 | 38G04 VH | AVTLDESGGGLQTPGRALSLVCKASGFTFSSYNMFWVRQAP |
| | sequence | GKGLEWVAGIDDDGSFTLYGAAVKGRATISRDNGQSTVRLQ |
| | | LNNLRAEDTGTYYCARDSGAVSIDAWGHGTEVIVSS |
| 45 | 4G04 VL | DIVLTQSQKFMSTSVGDRVSVTCKASQNVGTNVAWYQQKPG |
| | sequence | QSPKALIYSASYRYSGVPDRFTGSGSGTDFTLTISNVQSED |
| | | LAEYFCQQYNSYPYTFGGGTKLEIK |
| 46 | 5G04 VL | ETTVTQSPASLSMAIGEKVTIRCITSTDIDDDMNWYQQKPG |
| | sequence | EPPKLLISEGNTLRPGVPSRFSSSGYGTDFVFTIDNMLSED |
| | | VADYYCLQSDNLPFTFGSGTKLEIK |
| 47 | 6G04 VL | ETTVTQSPASLSVATGEKVTIRCITSTDIDDDMNWYQQKPG |
| | sequence | EPPKLLISEGNTLRPGVPSRFSSSGYGTDFVFTIENTLSED |
| | | VADYYCLQSDNMPLTFGAGTKLELK |
| 48 | 7G04 VL | ETTVTQSPASLSMAIGEKVTIRCITSTDIDDDMNWYQQKPG |
| | sequence | EPPKLLISEGNTLRPGVPSRFSSSGYGTDFVFTIENMLSED |
| | | VADYYCLQSDNLPFTFGSGTKLEIK |
| 49 | 9G04 VL | ETTVTQSPASLSVATGEKVTIRCITSTDIDDDMNWYQQKPG |
| | sequence | EPPKLLISEGNTLRPGVPSRFSSSGYGTDFVFTIENMLSED |
| | | VADYYCLQSDNLPFTFGSGTKLEIK |
| 50 | 10G04 VL | DVVMTQTPLSLPVSLGDQASISCRSSQSLVHSNGNTYLHWY |
| | sequence | LQKPGQSPKLLIYKVSNRFSGVPDRFSGSGSGTDFTLKISR |
| | | VEAEDLGVYYCFQGSHVPWTFGGGTKLEIK |
| 51 | 11G04 VL | ETTVTQSPASLSVATGEKVTIRCITSTDIDDDMNWYQQKPG |
| | sequence | EPPKLLISEGNTLRPGVPSRFSSSGYGTDFVFTIENMLSED |
| | | VADYYCLQSDNLPLTFGAGTKLELK |
| 52 | 12G04 VL | ETTVTQSPASLSMAIGEKVTIRCITSTDIDDDMNWYQQKPG |
| | sequence | EPPKLLISEGNTLRPGVPSRFSSTGYGTDFVFSIENMLSED |
| | | VADYYCLQSDNLPFTFGSGTKLEIK |
| 53 | 13G04 VL | DIVLTQSPASLAVSLGQRATISCRASESVDSYGNSFMHWYQ |
| | sequence | QKPGQPPKLLIYRASNLESGIPARFSGSGSRTDFTLTINPV |
| | | EADDVATYYCQQSNEDPWTFGGGTKLEIK |
| 54 | 14G04 VL | DIVMTQSHKFMSTSVGDRVSITCKASQDVGTAVAWYQQKPG |
| | sequence | QSPKLLIYWASTRHTGVPDRFTGSGSGTDFTLTISNVQSED |
| | | LADYFCQQYSSYPTFGGGTKLEIK |
| 55 | 15G04 VL | DVVMTQTPLSLPVSLGDQASISCRSSQSLLHTNVNTYLHWY |
| | sequence | LQKPGQSPKLLIYKVSNRFSGVPDRFSGSGSGTDFTLKISR |
| | | VEAEDLGVYFCSQSTHVPYTFGGGTKLEIK |
| 56 | 16G04 VL | ETTVTQSPASLSMAIGEKVTIRCITSTDIDDDMNWYQQKPG |
| | sequence | EPPKLLISEGNTLRPGVPSRFSSSGYGTDFVFTIENMLSED |
| | | VADYYCLQSDNLPLTFGAGTKLELK |
| 57 | 17G04 VL | ETTVTQSPASLSVATGEKVTIRCITSTDIDDDMNWYQQRPG |
| | sequence | EPPNLLISEGNTLRPGVPSRFSSSGYGTDFVFTIENTLSED |
| | | VADYYCLQSDNLPFTFGSGTRLEIK |
| 58 | 18G04 VL | ETTVTQSPASLSMAIGEKVTIRCITSTDIDDDMNWYQQKPG |
| | sequence | EPPKLLISEGNTLRPGVPSRFSSSGYGTDFVFTIENLLSED |
| | | VADYYCLQSDNLPLTFGGGTKLEIK |
| 59 | 19G04 VL | ETTVTQSPASLSMAIGEKVTIRCITSTDIDDDMNWYQQKPG |
| | sequence | EPPKLLISEGNTLRPGVPSRFSSSGYGTDFIFTIENMLSED |
| | | VADYYCLQSDNLPLTFGAGTKLELK |
| 60 | 20G04 VL | ETTVTQSPASLSVATGEKVTIRCITSTDIDDDMNWYQQKPG |
| | sequence | EPPKLLISEGNTLRPGVPSRFSSGGYGTDFVFTVENMLSED |
| | | VADYYCLQSDNLPLTFGAGTKLELK |
| 61 | 21G04 VL | ETTVTQSQKFMSTSVGDRVSVTCKASQNVGTNVAWYQQKPG |
| | sequence | QSPKALIYSASYRYSGVPDRFTGSGSGTDFTLTISNVQSED |
| | | LAEYFCQQYNSYPFTFGSGTKLEIK |
| 62 | 22G04 VL | DIVLTQSQKFMSTSVGDRVSVTCKASQNVGTNVAWYQQKPG |
| | sequence | QSPKALIYSASYRYSGVPDRFTGSGSGTDFTLTISNVQSED |
| | | LAEYFCQQYNSYPLTFGAGTKLELK |
| 63 | 23G04 VL | DIVLTQSQKFMSTSVGDRVSVTCKASQNVGTNVAWYQQKPG |
| | sequence | QSPKALIYSASYRYSGVPDRFTGSGSGTDFTLTISNVQSED |
| | | LAEYFCQQYNSYPYTFGGGTKLEIK |
| 64 | 24G04 VL | DIVMTQSQKFLSTSVGDRVSVTCKASQNVGTNVAWYQQKPG |
| | sequence | QSPKVLIYSASYRYSGVPDRFTGSGSGTDFTLTVSNVQSED |
| | | LAEYFCQQYNNYPLTFGAGTKLELK |
| 65 | 25G04 VL | DIVLTQSQKFMSTSVGDRVSVTCKASQNVGTNVAWYQQKPG |
| | sequence | QSPKALIYSASYRYSGVPDRFTGSGSGTDFTLTISNVQPED |
| | | LAEYFCQQYNSYPLTFGGGTKLEIK |
| 66 | 26G04 VL | DIVLTQSHKFMSTSVGDRVSITCKASQDVSTAVAWYQQKPG |
| | sequence | QSPKLLIYSASYRYTGVPDRFTGSGSGTDFTFTISSVQAED |
| | | LAVYYCQQHYSTPRTFGGGTKLEIK |
| 67 | 27G04 VL | DIQMTQSHKFMSTSVGDRVSITCKASQDVSTAVAWYQQKPG |
| | sequence | QSPKLLIYSASYRYTGVPDRFTGSGSGTDFTFTISSVQAED |
| | | LAVYYCQQHYSTPRTFGGGTKLEIK |
| 68 | 28G04 VL | ETTVTQSPASLSMAIGEKVTIRCITSTDIDDDMNWYQQKPG |
| | sequence | EPPKLLISEGNTLRPGVPSRFSSSGYGTDFVFTIENMLSED |
| | | VADYYCLQSGTLPFTFGSGTKLEIK |
| 69 | 29G04 VL | DIVLTQSQKFMSTSVGDRVSVTCKASQNVGTNVAWYQQKPG |
| | sequence | QSPKALIYSASYRYSGVPDRFTGSGSGTDFTLTISNVQSED |
| | | LAEYFCQQYNSYPLTFGAGTKLELK |
| 70 | 30G04 VL | ETTVTQSPASLSMAIGEKVTIRCITSTDIDDDMNWYQQKPG |
| | sequence | EPPKLLISEGNTLRPGVPSRFSSSGYGTDFVFTIENMLSED |
| | | VADYYCLQSDNLPLTFGAGTKLELK |
| 71 | 31G04 VL | DIVLTQSPASLAVSLGQRATISCRASESVDSYGNSFMHWYQ |
| | sequence | QKPGQPPKLLIYRASNLESGIPARFSGSGSRTDFTLTINPV |
| | | EADDVATYYCQQSNEDPYTFGGGTKLEIK |
| 72 | 32G04 VL | ENVLTQSPAIMSASPGEKVTMTCRASSSLSSTYLHWYQQKS |
| | sequence | GASPKLWIYSTSNLASGVPARFSGSGSGTSYSLTISSVEAE |
| | | DAATYYCQQYSGYPLTFGSGTKLEIK |
| 73 | 33G04 VL | ALTQPSSVSASPGETVKITCSGSSDSWYGWYQQKSPGSALV |
| | sequence | TVIYDSTNRPSSIPSRFSGSTSGSTGTLTITGVRADDEAVY |
| | | FCGSFDSSTDSTAFGAGTTLTVL |
| 74 | 34G04 VL | ALTQPSSVSANPGETVKITCSGGDNYAGSYYYGWYQQKSPG |
| | sequence | SAPVTVIYDDTNRPADIPSRFSGSTSGSTNTLTITGVRAED |
| | | EAVYFCGGWDSINDRNIFGAGTTLTVL |
| 75 | 35G04 VL | ALTQPSSVSANPGETVKITCSGGSGSYGWFQQKSPGNAPVT |
| | sequence | VIYHNDQRPSDIPSRFSGSKSGSTATLTITEVQADDEAVYV |
| | | CGGYDNDNDAGIFGAGTTLTVL |
| 76 | 36G04 VL | ALTQPSSVSAIPGETVKITCSGGGSDYGWYQQKSPGSAPVT |
| | sequence | VIYWSDNRPSNIPSRFSGSESGSTATLTITGVRAEDEAVYF |
| | | CGGYDSSGSGMFGAGTTLTVL |
| 77 | 37G04 VL | ALTQPSSVSANPGETVKITCSGDRSYYGYGWYQQKAPGSAP |
| | sequence | VTVIYWDDKRPSDIPSRFSGSTSGSTATLTITGVQAEDEAV |
| | | YFCGNMDSSYVGMFGAGTTLTVL |
| 78 | 38G04 VL | ALTQPSSVSANPGETVKITCSGGDSDYGWYQQKSPGSAPVT |
| | sequence | VIYQNDKRPSDIPSRFSGSASGSTATFTITGVQAEDEAVYY |
| | | CGGWDGSYDGAVFGAGTTLTVL |
| 79 | ONA-0-v1 | DYYIN |
| | HDCR1 | |
| 80 | ONA-0-v1 | RIYPGSGNTYYNEKFKG |
| | HDCR2 | |
| 81 | ONA-0-v1 | GIGGGFGMDY |
| | HDCR3 | |
| 82 | ONA-0-v1 | KASQSVSDDVA |
| | LDCR1 | |
| 83 | ONA-0-v1 | YASNRYT |
| | LDCR2 | |
| 84 | ONA-0-v1 | QQDYSSPLT |
| | LDCR3 | |
| 85 | HCDR1 | PIAYMT |
| 86 | HCDR1 | DYWMH |
| 87 | HCDR1 | DYEIQ |
| 88 | HCDR1 | DYEMH |
| 89 | HCDR1 | DYNMH |
| 90 | HCDR1 | DYEMQ |
| 91 | HCDR1 | SHDMS |
| 92 | HCDR1 | SYWMN |
| 93 | HCDR1 | DYYIH |
| 94 | HCDR1 | TSWMN |
| 95 | HCDR1 | EYTMH |
| 96 | HCDR1 | SYWMH |
| 97 | HCDR1 | NYWIE |
| 98 | HCDR1 | EYTIH |
| 99 | HCDR1 | PIAYMS |
| 100 | HCDR1 | NYWMN |
| 101 | HCDR1 | SYAMG |
| 102 | HCDR1 | NYGMG |
| 103 | HCDR1 | SYGMG |
| 104 | HCDR1 | DYDML |
| 105 | HCDR1 | SYNMF |
| 106 | HCDR2 | DILPSIGGTIYGEKFED |
| 107 | HCDR2 | AIDTSDSYTSYNQKFKG |
| 108 | HCDR2 | SIDTSDSYTTYSQKFKG |
| 109 | HCDR2 | GIHPGSSGIVYNQKFKG |
| 110 | HCDR2 | TVDPETGGTAFNQKFKD |
| 111 | HCDR2 | YIYPYNGGTGYNQKFKS |
| 112 | HCDR2 | GIDPETGGIAYNQKFKV |
| 113 | HCDR2 | AINSDGGYTYYPDTMER |
| 114 | HCDR2 | MIDPSDNETHYNQMFKD |
| 115 | HCDR2 | WIDPENGDTEYAPRFQD |
| 116 | HCDR2 | RIYPGDGDTNYNGKFKG |
| 117 | HCDR2 | GINPYNGGTAYNQKFKN |
| 118 | HCDR2 | GIYPYNGGTAYNQKFKN |
| 119 | HCDR2 | GIYPNNGATKYNQKFKG |
| 120 | HCDR2 | EIDPSDSHTNYNQKFKG |
| 121 | HCDR2 | EIDPSDSHANYNQKFKG |
| 122 | HCDR2 | DFLPGSSSANYNEKFKG |
| 123 | HCDR2 | EILPGSGSPKYNEKFKG |
| 124 | HCDR2 | GIIPNNGGSSHKQNFKD |
| 125 | HCDR2 | AINSDGAITYYPDTMER |
| 126 | HCDR2 | DILPSIGSTIYGEKFED |
| 127 | HCDR2 | EIRLKSNNYATHYAESVKG |
| 128 | HCDR2 | GKAKDGGGTAYGSAVKG |
| 129 | HCDR2 | KIYKDGGYTGYGAAVDG |
| 130 | HCDR2 | AISNDGSKADYGAAVKG |
| 131 | HCDR2 | GINAASTYTDYGAAVKG |
| 132 | HCDR2 | GIDDDGSFTLYGAAVKG |
| 133 | HCDR3 | DYYGSSYGYFDV |
| 134 | HCDR3 | DYYGSSTAWFAY |
| 135 | HCDR3 | GEDYEGTWFAY |
| 136 | HCDR3 | GGGYDGAWFAY |
| 137 | HCDR3 | GYGNYGAWFAY |
| 138 | HCDR3 | DGGYDVYFDY |
| 139 | HCDR3 | KLDFDY |
| 140 | HCDR3 | HGYDYDEEGAWFAY |
| 141 | HCDR3 | SDYGNGYSFYLDV |
| 142 | HCDR3 | WLLSGNGMDY |
| 143 | HCDR3 | EVYYGGYEDYGMDY |
| 144 | HCDR3 | DYFYGDANPWFAY |
| 145 | HCDR3 | DYFYGDGYPWFTY |
| 146 | HCDR3 | DRYDVWFAY |
| 147 | HCDR3 | IYYGLDN |
| 148 | HCDR3 | IHYGLDN |
| 149 | HCDR3 | FTVEVTDAMDY |
| 150 | HCDR3 | FILENYFDY |
| 151 | HCDR3 | AGDYAFDY |
| 152 | HCDR3 | HGQTGTWFAY |
| 153 | HCDR3 | LLLR |
| 154 | HCDR3 | SAYGDWFYGSSGPYADSIDA |
| 155 | HCDR3 | AADSGYLYITDSIDA |
| 156 | HCDR3 | SADTGYCSWSACIADSIDA |
| 157 | HCDR3 | SSYGGGWSDVGSIDA |
| 158 | HCDR3 | DSGAVSIDA |
| 159 | LCDR1 | KASQNVGTNVA |
| 160 | LCDR1 | ITSTDIDDDMN |
| 161 | LCDR1 | RSSQSLVHSNGNTYLH |
| 162 | LCDR1 | RASESVDSYGNSFMH |
| 163 | LCDR1 | KASQDVGTAVA |
| 164 | LCDR1 | RSSQSLLHTNVNTYLH |
| 165 | LCDR1 | KASQDVSTAVA |
| 166 | LCDR1 | RASSSLSSTYLH |
| 167 | LCDR1 | SGSSDSWYG |
| 168 | LCDR1 | SGGDNYAGSYYYG |
| 169 | LCDR1 | SGGSGSYG |
| 170 | LCDR1 | SGGGSDYG |
| 171 | LCDR1 | SGDRSYYGYG |
| 172 | LCDR1 | SGGDSDYG |
| 173 | LCDR2 | SASYRYS |
| 174 | LCDR2 | EGNTLRP |
| 175 | LCDR2 | KVSNRFS |
| 176 | LCDR2 | RASNLES |
| 177 | LCDR2 | WASTRHT |
| 178 | LCDR2 | SASYRYT |
| 179 | LCDR2 | STSNLAS |
| 180 | LCDR2 | DSTNRPS |
| 181 | LCDR2 | DDTNRPA |
| 182 | LCDR2 | HNDQRPS |
| 183 | LCDR2 | WSDNRPS |
| 184 | LCDR2 | WDDKRPS |
| 185 | LCDR2 | QNDKRPS |
| 186 | LCDR3 | QQYNSYPYT |
| 187 | LCDR3 | LQSDNLPFT |
| 188 | LCDR3 | LQSDNMPLT |
| 189 | LCDR3 | FQGSHVPWT |
| 190 | LCDR3 | LQSDNLPLT |
| 191 | LCDR3 | QQSNEDPWT |
| 192 | LCDR3 | QQYSSYPT |
| 193 | LCDR3 | SQSTHVPYT |
| 194 | LCDR3 | QQYNSYPFT |
| 195 | LCDR3 | QQYNSYPLT |
| 196 | LCDR3 | QQYNNYPLT |
| 197 | LCDR3 | QQHYSTPRT |
| 198 | LCDR3 | LQSGTLPFT |
| 199 | LCDR3 | QQSNEDPYT |
| 200 | LCDR3 | QQYSGYPLT |
| 201 | LCDR3 | GSFDSSTDSTA |
| 202 | LCDR3 | GGWDSINDRNI |
| 203 | LCDR3 | GGYDNDNDAGI |
| 204 | LCDR3 | GGYDSSGSGM |
| 205 | LCDR3 | GNMDSSYVGM |
| 206 | LCDR3 | GGWDGSYDGAV |
| 240 | 11G06 VL 1 | ETTVTQSPAFMSATTGDKVTISCITSTDIDDDMNWYQQKPG |
| | | EPPKLLISEGNTLRPGVPSRFSSSGYGTDFTFTINNIESED |
| | | AAYYYCLQSDNLPLTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 241 | 11G06 VH 1 | EVQLVQSGAEVKKPGSSVKVSCKASGYRFSDYEMQWVRQAP |
| | | GQGLEWMGGIDPETGGIAYAQKFQGRVTLTADKSTSTAYME |
| | | LSSLRSEDTAVYYCTRKLDFDYWGQGTLVTVSSASTKGPSV |
| | | FPLAPSSKSTSGGTAALGCLVKDYFPEPVTVSWNSGALTSG |
| | | VHTFPAVLQSSGLYSLSSVVTVPSSSLGTQTYICNVNHKPS |
| | | NTKVDKKVEPKSCDKTHTCPPCPAPESTRGPSVFLFPPKPK |
| | | DTLYITREPEVTCVVVDVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKG |
| | | FYPSDIAVEWESNGQPENNYKTTPPVLDSDGSFFLYSKLTV |
| | | DKSRWQQGNVFSCSVMHEALHNHYTQKSLSLSPGK |
| 242 | 11G06 VL 2 | ETTVTQSPAFMSATPGDKVTISCITSTDIDDDMNWYQQKPG |
| | | EPPKLLISEGNTLRPGVPSRFSSSGYGTDFTFTINNIESED |
| | | AAYYYCLQSDNLPLTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 243 | 11G06 VH 2 | QVQLVQSGAEVKKPGSSVKVSCKASGYRFSDYEMQWVRQAP |
| | | GQGLEWMGGIDPETGGIAYAQKFQGRVTLTADKSTSTAYME |
| | | LSSLRSEDTAVYYCTRKLDFDYWGQGTLVTVSSASTKGPSV |
| | | FPLAPSSKSTSGGTAALGCLVKDYFPEPVTVSWNSGALTSG |
| | | VHTFPAVLQSSGLYSLSSVVTVPSSSLGTQTYICNVNHKPS |
| | | NTKVDKKVEPKSCDKTHTCPPCPAPESTRGPSVFLFPPKPK |
| | | DTLYITREPEVTCVVVDVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKG |
| | | FYPSDIAVEWESNGQPENNYKTTPPVLDSDGSFFLYSKLTV |
| | | DKSRWQQGNVFSCSVMHEALHNHYTQKSLSLSPGK |
| 244 | 11G06 VL 3 | ETTVTQSPAFMSATTGDKVTISCITSTDIDDDMNWYQQKPG |
| | | EPPKLLISEGSTLRPGVPSRFSSSGYGTDFTFTINNIESED |
| | | AAYYYCLQSDNLPLTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 245 | 11G06 VL 4 | ETTVTQSPAFMSATPGDKVTISCITSTDIDDDMNWYQQKPG |
| | | EPPKLLISEGSTLRPGVPSRFSSSGYGTDFTFTINNIESED |
| | | AAYYYCLQSDNLPLTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 246 | 11G06 VL 3-4 | EGSTLRP |
| | CDR2 | |
| 247 | 11G06 VL 1-4 | LQSGNSQES |
| | CDR3 | |
| 248 | 11G06 VH 1-2 | GIDPETGGIAYAQKFQG |
| | CDR2 | |

In another aspect, provided herein are antibodies that bind the same epitope of CD36 as an antibody described herein (e.g., to an epitope in human CD36, as provided in SEQ ID NO: 1). In some embodiments, the amino acids comprising an epitope can be identified by alanine scanning mutagenesis mapping. In some embodiments, the amino acids comprising an epitope can be identified by hydrogen-deuterium exchange mass spectrometry.

In some embodiments, the antibody binds to an epitope in human CD36 comprising at least one amino acid, preferably comprising or consisting of all the amino acids, selected from the group consisting of 145A, 146S, 147H, 148I, 149Y, 150Q, 151N, 152Q, 153F, 154V, 155Q, 156M, 1571, 158L, 159N, 160S, 185P, 186F, 187L, 188S, 189L, 190V, 191P, 192Y, 193P, 194V, 195T, 196T, 197T, 198V, 199G, 398K, 399I, 400Q, 401V, 402L, 403K, 404N, 405L, 406K, 407R, 408N, 409Y, 4101, 411V, 412P, 4131, and 414L (e.g., 1G04). In some embodiments, the antibody binds to an epitope in human CD36 comprising at least one amino acid selected from the group consisting of 149Y, 150Q, 151N, 152Q, 153F, 154V, 155Q, 156M, 188S, 189L, 190V, 191P, 192Y, 193P, 194V, 195T, 196T, 400Q, 401V, 402L and 403K. In some embodiments, the antibody binds to an epitope comprising or consisting of 149Y, 150Q, 151N, 152Q, 153F, 154V, 155Q and 156M. In some embodiments, the antibody binds to an epitope comprising or consisting of 188S, 189L, 190V, 191P, 192Y, 193P, 194V, 195T, and 196T. In some embodiments, the antibody binds to an epitope comprising or consisting of 400Q, 401V, 402L, and 403K.

In some embodiments, the antibody that binds to human CD36 to an epitope as defined under the paragraph above is selected from the group consisting of: 04G04, 07G04, 09G04, 14G04, 31G04, 13G04, 21G04, 22G04, 23G04, 24G04, 25G04, 29G04, 15G04, 27G04, 26G04, 28G04, 18G04, 16G04, 17G04, 05G04, 06G04, 12G04, 30G04, 32G04, 33G04, 34G04, 35G04, 36G04, 37G04 and 38G04; or antibodies comprising the same six CDRs or the same VH and VL sequences.

In some embodiments, the anti-CD36 antibody of the invention inhibits the binding of 01G04 to the 01G04 epitope on CD36. In one embodiment, the antibody that inhibits the binding of 01G04 to its epitope on CD36 is selected from the group consisting of: 04G04, 07G04, 09G04, 14G04, 31G04, 13G04, 21G04, 22G04, 23G04, 24G04, 25G04, 29G04, 15G04, 27G04, 26G04, 28G04, 18G04, 16G04, 17G04, 05G04, 06G04, 12G04, 30G04, 32G04, 33G04, 34G04, 35G04, 36G04, 37G04 and 38G04; or antibodies comprising the same six CDRs or the same VH and VL sequences.

In some embodiments, the anti-CD36 antibody of the invention inhibits the binding of FA6-152 to its epitope on CD36. In one embodiment, the antibody that inhibits the binding of FA6-152 to its epitope on CD36 is selected from the group consisting of: 04G04, 31G04, 13G04, 24G04, 25G04, 29G04, 15G04, 27G04, 26G04 and 06G04; or antibodies comprising the same six CDRs or the same VH and VL sequences.

In some embodiments, the anti-CD36 antibody of the invention inhibits the binding of 11G04 to its epitope on CD36. In one embodiment, the antibody that inhibits the binding of 11G04 to its epitope on CD36 is selected from the group consisting of: 18G04, 19G04, 20G04, 16G04, 30G04, 33G04, 34G04, 35G04, 36G04, 37G04 and 38G04; or antibodies comprising the same six CDRs or the same VH and VL sequences.

In other embodiments, the antibody binds to an epitope in human CD36 comprising at least one amino acid, preferably comprising or consisting of all the amino acids, selected from the group consisting of 280E, 281S, 282D, 283V, 284N, 285L, 286K, 287G, 2881, 289P, 290V, 291Y, 292R, 293F, 294V, 295L, 296P, 297S, 298K, 341I, 342S, 343L, 344P, 345H, 346F, 347L, 348Y, 349A, 350S 351P 352D, 353V, 354S, 355E, 356P, 357I, 358D, 359G, 360L, 361N 362P, 363N, 364E, 365E (e.g., 11G04). In some embodiments, the antibody binds to an epitope in human CD36 comprising at least one amino acid, preferably comprising or consisting of all the amino acids, selected from the group consisting of 286K, 287G, 2881, 289P, 290V, 291Y, 292R, 341I, 342S, 343L, 344P, 345H, 346F, 347L, 348Y, 349A, 350S. In some embodiments, the antibody binds to an epitope in human CD36 comprising at least one amino acid selected from the group consisting of 286K, 287G, 2881, 289P, 290V, 291Y, and 292R. In some embodiments, the antibody binds to an epitope in human CD36 comprising at least one amino acid selected from the group consisting of 341I, 342S, 343L, 344P, 345H, 346F, 347L, 348Y, 349A and 350S.

In some embodiments, the antibody that binds to human CD36 to an epitope as defined under the paragraph above is selected from the group consisting of: 18G04, 19G04, 20G04, 16G04, 30G04, 33G04, 34G04, 35G04, 36G04, 37G04 and 38G04; or antibodies comprising the same six CDRs or the same VH and VL sequences.

Competition binding assays can be used to determine whether two antibodies bind to overlapping epitopes. Competitive binding can be determined in an assay in which the immunoglobulin under test inhibits specific binding of a reference antibody to a common antigen, such as CD36. Numerous types of competitive binding assays are known, for example: epitope binning in the AlphaScreen® competition assay (see, e.g., Bembenek M E et al., Analytical Bioch. 408(2):321-327); competition FACS; solid phase direct or indirect radioimmunoassay (RIA); solid phase direct or indirect enzyme immunoassay (EIA); sandwich competition assay (see Stahli C et al., (1983)Methods Enzymol 9: 242-253); solid phase direct biotin-avidin EIA (see Kirkland T N et al., (1986) J Immunol 137: 3614-9); solid phase direct labeled assay, solid phase direct labeled sandwich assay (see Harlow E & Lane D, (1988) Antibodies: A Laboratory Manual, Cold Spring Harbor Press); solid phase direct label RIA using I-125 label (see Morel G A et al., (1988) Mol Immunol 25(1): 7-15); solid phase direct biotin-avidin EIA (Cheung R C et al., (1990) Virology 176: 546-52); and direct labeled RIA (Moldenhauer G et al., (1990) Scand J Immunol 32: 77-82). Typically, such an assay involves the use of purified antigen (e.g., CD36 such as human CD36) bound to a solid surface or cells bearing either of these, an unlabeled test immunoglobulin and a labeled reference immunoglobulin. Competitive inhibition can be measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70%, 70-75% or more. A competition binding assay can be configured in a large number of different formats using either labeled antigen or labeled antibody. In a common version of this assay, the antigen is immobilized on a 96-well plate. The ability of unlabeled antibodies to block the binding of labeled antibodies to the antigen is then measured using radioactive or enzyme labels. For further details see, for example, Wagener C et al., (1983) J Immunol 130: 2308-2315; Wagener C et al., (1984) J Immunol Methods 68: 269-274; Kuroki M et al., (1990) Cancer Res 50: 4872-4879; Kuroki M et al., (1992) Immunol Invest 21: 523-538; Kuroki M et al., (1992) Hybridoma 11: 391-407 and Antibodies: A Laboratory Manual, Ed Harlow E & Lane D editors supra, pp. 386-389.

In one embodiment, a competition assay is performed using surface plasmon resonance (BIAcore®), e.g., by an ‘in tandem approach’ such as that described by Abdiche Y N et al., (2009) Analytical Biochem 386: 172-180, whereby CD36 antigen is immobilized on the chip surface, for example, a CM5 sensor chip and the anti-CD36 antibodies are then run over the chip. To determine if an antibody competes with an anti-CD36 antibody described herein, the anti-CD36 antibody is first run over the chip surface to achieve saturation and then the potential, competing antibody is added. Binding of the competing antibody can then be determined and quantified relative to a non-competing control.

In one embodiment, a competition assay is performed using surface plasmon resonance (BIAcore®) to determine whether anti-CD36 antibodies can bind to CD36 at the same time as other molecules known to bind to CD36 (e.g., TSP-1). In some embodiments of such an assay, the anti-CD36 antibody is first run over the chip surface to achieve saturation and then the potential, competing molecule (e.g., TSP-1) is added. Binding of the competing molecule can then be determined by examining the resultant change in response units, and comparing to a non-competing control.

In one embodiment, Fortebio Octet competition binding is used to determine that a CD36 antibody competitively inhibits the binding of another CD36 antibody to CD36.

In another aspect, provided herein are antibodies that competitively inhibit (e.g., in a dose dependent manner) an antibody described herein from binding to CD36 (e.g., human CD36), as determined using assays known to one of skill in the art or described herein (e.g., ELISA competitive assays, or suspension array, or surface plasmon resonance assay).

It is preferred that the anti-CD36 antibody modulates the activity of CD36, antagonizing or blocking it. The antibody that blocks or inhibits CD36 activity can be a full length antibody. It is also possible to use analogues or fragments of antibodies, such as single chain antibodies, single chain variable domain fragments (scFv), F(ab′)₂ fragments (which can be obtained by pepsin digestion of an antibody molecule), or Fab fragments (which can be obtained by reducing the disulphide bridges of the F(ab′)₂ fragments. Humanized antibodies can be used when the subject is a human being.

As CD36 has several known functions, the antibody can be selected so that it inhibits all known functions of CD36, including its interaction with thrombospondin, collagens and fatty acids, or so that it inhibits only specific functions of CD36 (e.g., blocking only fatty acid and oxidized-LDL uptake). Therefore, in some embodiments, the anti-CD36 antibody blocks the CD36-mediated uptake of fatty acids and/or oxidized-LDL. In some embodiments, the anti-CD36 antibody blocks the CD36-mediated uptake of fatty acids and/or oxLDL while blocking less than 50% of CD36's binding to TSP-1, as measured by surface plasmon resonance. And in some embodiments, the anti-CD36 antibody blocks the CD36-mediated uptake of fatty acids and/or oxLDL while blocking less than 25% of CD36's binding to TSP-1, as measured by surface plasmon resonance. In some embodiments, the anti-CD36 antibody blocks the CD36-mediated uptake of fatty acids and/or oxidized-LDL by at least about 10%, at least about 15%, at least about 16%, at least about 17%, at least about 18%, at least about 19%, at least about 20%, at least about 25%, at least about 30%, at least about 35%, at least about 40%, at least about 45%, at least about 50%, at least about 55%, at least about 60%, at least about 65%, at least about 70%, or at least about 75%, relative to untreated controls. In some embodiments, the anti-CD36 antibody blocks the CD36-mediated uptake of fatty acids and/or oxidized-LDL by at least about 17%.

When the subject to be treated is a human being, any known anti-CD36 antibody can be used or the antibody can be prepared for being administered to human beings. For antibodies that have been generated in a non-human immune system (as those used in the assays of the present application), such as in mice, humanization can be necessary to enable their administration to human beings, in order to avoid adverse reactions. Humanized antibodies are antibodies, usually monoclonal antibodies, initially generated in a non-human species and whose protein sequences have been modified to increase their similarity to antibody variants produced naturally in humans, so that minimal sequence derived from non-human immunoglobulins remain. Even after humanization, the amino acid sequence of humanized antibodies is partially distinct from antibodies occurring naturally in human beings. Several processes are known for those skilled in the art for antibody humanization, as it has been reviewed, for instance, by Almagro and Fransson (2008), including: humanizing through production of a mouse-human (mouse Fab spliced to human Fc) chimera, which chimera might be further humanized by selective alteration of the amino acid sequence of the Fab portion; insertion of one or more CDR segments of the “donor” (non-human antibody) by replacing the corresponding segments of a human antibody, which can be done using recombinant DNA techniques to create constructs capable of expression in mammalian cell culture, or even avoiding the use of non-human mammals by creating antibody gene libraries usually derived from human RNA isolated from peripheral blood and displayed by micro-organisms or viruses (as in phage display) or even cell free extracts (as in ribosome display), selection of the appropriate intermediate product (usually, antibody fragments such as Fab or scFv) and obtaining full antibodies for instance, again, recombinant DNA techniques. Several patent documents have been dedicated to humanization methods like, for instance U.S. Pat. No. 6,054,297, assigned to Genentech; U.S. Pat. Nos. 5,225,539 and 4,816,397 are also useful references, and are incorporated herein by reference in their entirety.

The method for obtaining monoclonal antibodies is well known for those skilled in the art. In general, antibodies against CD36 receptor can be raised according to known methods, such as those mentioned in classic laboratory manuals as “Antibodies: A Laboratory Manual, Second edition”, edited by E. A. Greenfield in 2014, by administering CD36 whole protein or a fragment or epitope thereof to a host animal which is a different from the mammal where a therapeutic effect is sought. Monoclonal antibodies in particular can be prepared and isolated by any technique that provides for the production of antibody molecules by continuous cell lines in culture, such as the hybridoma technique originally described by Kohler and Milstein (1975), the human B-cell hybridoma technique (Cote et al., 1983), or the EBV-hybridoma technique (Cole et al., 1985). Other methods for the preparation of clonal cell lines and of monoclonal antibodies and antigen-binding fragments thereof expressed thereby are well known in the art (see, for example, Chapter 11 in: Short Protocols in Molecular Biology, (2002) 5th Ed., Ausubel F M et al., supra). Alternatively, as commented above, Fab and/or scFv expression libraries can be constructed to allow rapid identification of fragments having the desired specificity to the CD36 receptor. Examples of phage display methods that can be used to make the antibodies or fragments described herein include those disclosed in Brinkman U et al., (1995) J Immunol Methods 182: 41-50; Ames R S et al., (1995) J Immunol Methods 184: 177-186; Kettleborough C A et al., (1994) Eur J Immunol 24: 952-958; Persic L et al., (1997) Gene 187: 9-18; Burton D R & Barbas C F (1994) Advan Immunol 57: 191-280; PCT Application No. PCT/GB91/001134; International Publication Nos. WO 90/02809, WO 91/10737, WO 92/01047, WO 92/18619, WO 93/11236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108.

For the design of antibodies with a particular specificity, it is advantageous to resource to annotated NCBI Reference Sequence (NC_000007.14, Homo sapiens annotation release: 107, which is the current release on 29 Sep. 2015) or UniProtKB P16671, in order to choose as immunogen, if wished, a particular domain or region of the antibody to be targeted or mutated before generating the antibodies.

For achieving a therapeutic effect, the anti-CD36 antibody, which is a blocker of activity of CD36, will be administered preferably in therapeutically effective amounts. The precise determination of what would be considered an effective dose may be based on factors individual to each patient, including their size, age, cancer stage, and nature of the blocker (e.g. expression construct, antisense oligonucleotide, antibody or fragment thereof, etc.). Therefore, dosages can be readily ascertained by those of ordinary skill in the art from this disclosure and the knowledge in the art. Multiple doses can be also administered to the subject over a particular treatment period, for instance, daily, weekly, monthly, every two months, every three months, or every six months. Therapeutically effective plasma levels may also be achieved by administering multiple doses each day. In certain dose schedules, the subject receives an initial dose at a first time point that is higher than one or more subsequent or maintenance doses. For repeated administrations over several days or longer, depending on the condition, the treatment would generally be sustained until a desired effect occurs. The progress of this therapy is easily monitored by conventional techniques and assays.

Depending on the type and severity of the disease, about 1 g/kg to 40 mg/kg (e.g. 0.1 mg/kg-10 mg/kg) of the anti-CD36 antibody can be an initial candidate dosage for administration to the patient. The dosage may be administered for example, by one or more separate administrations, or by continuous infusion. A daily dosage might range from about 1 μg/kg to 100 mg/kg or more. One exemplary dosage of the anti-CD36 antibody would be in the range from about 0.005 mg/kg to about 10 mg/kg. In other examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein. In examples of a derivable range from the numbers listed herein, a range of about 5 mg/kg body weight to about 100 mg/kg body weight, about 5 μg/kg body weight to about 500 mg/kg body weight etc., can be administered, based on the numbers described above. Thus, one or more doses of about 0.5 mg/kg, 2.0 mg/kg, 5.0 mg/kg or 10 mg/kg (or any combination thereof) may be administered to the patient.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data. Dosage amount and interval may be adjusted individually to provide plasma levels of the anti-CD36 antibody which are sufficient to maintain therapeutic effect. Levels in plasma may be measured, for example, by HPLC.

An anti-CD36 antibody can be fused or conjugated (e.g., covalently or noncovalently linked) to a detectable label or substance. Examples of detectable labels or substances include enzyme labels, such as, glucose oxidase; radioisotopes, such as iodine (125I, 121I), carbon (14C), sulfur (35S), tritium (3H), indium (121In), and technetium (99Tc); luminescent labels, such as luminol; and fluorescent labels, such as fluorescein and rhodamine, and biotin. Such labeled antibodies can be used to detect CD36 (e.g., human CD36) protein.

Antibodies with reduced fucose content have been reported to have an increased affinity for Fc receptors, such as, e.g., FcγRIIIA. Accordingly, in certain embodiments, an antibody described herein has reduced fucose content or lacks fucose (i.e., is “afucosylated”). Such antibodies can be produced using techniques known to one skilled in the art. For example, they can be expressed in cells deficient or lacking the ability to fucosylate. In a specific example, cell lines with a knockout of both alleles of α1,6-fucosyltransferase can be used to produce antibodies with reduced fucose content. The Potelligent® system (Lonza) is an example of such a system that can be used to produce antibodies with reduced fucose content. Alternatively, antibodies with reduced fucose content or no fucose content can be produced by, e.g.: (i) culturing cells under conditions which prevent or reduce fucosylation; (ii) posttranslational removal of fucose (e.g., with a fucosidase enzyme); (iii) post-translational addition of the desired carbohydrate, e.g., after recombinant expression of a non-glycosylated glycoprotein; or (iv) purification of the glycoprotein so as to select for antibodies which are not fucosylated. See, e.g., Longmore GD & Schachter H (1982) Carbohydr Res 100: 365-92 and Imai-Nishiya H et al., (2007) BMC Biotechnol. 7: 84 for methods for producing antibodies thereof with no fucose content or reduced fucose content.

In some embodiments, the CD36 antibody has enhanced ADCC activity in vitro compared to fucosylated CD36 antibodies having the same amino acid sequence. In some embodiments, the afucosylated CD36 antibodies cause specific lysis that is at least 10, at least 15, at least 20, at least 25, at least 30, at least 35, at least 40, at least 45, at least 50, at least 60, at least 65, at least 70, or at least 75 percentage points greater than specific lysis with fucosylated CD36 antibodies.

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions. In some embodiments, the Fc domain comprises one or more amino acid substitution that reduces binding to an Fc receptor, in particular towards Fcγ receptor. In some embodiments, the Fc domain is of human IgG1 subclass with the amino acid mutations L234A, L235A and/or P329G (numbering according to Kabat EU index. In some embodiments, the Fc domain is of human IgG1 subclass with the amino acid mutations L234G, L235S, and G236R. In some embodiments, the Fc domain is of human IgG1 subclass with the amino acid mutations L234S, L235T, and G236R. In some embodiments, the Fc domain is of human IgG1 subclass with the amino acid mutations L234S, L235V, and G236R. In some embodiments, the Fc domain is of human IgG1 subclass with the amino acid mutations L234T, L235Q, and G236R. In some embodiments, the Fc domain is of human IgG1 subclass with the amino acid mutations L234T, L235T, and G236R. In some embodiments, the heavy chain constant region comprises an IgG constant region containing the L234A, L235S, and G236R mutations. In some embodiments, the heavy chain constant region comprises an IgG constant region containing the L234Q, L235S, and G236R mutations. In some embodiments, the heavy chain constant region comprises an IgG constant region containing the L234S, L235G, and G236R mutations. In some embodiments, the heavy chain constant region comprises an IgG constant region containing the L234T, L235S, and G236R mutations. In some embodiments, the heavy chain constant region comprises an IgG constant region containing the L234Q, L235S, and G236R mutations. In some embodiments, the Fc domain is of human IgG1 subclass with the amino acid mutations L234A and L235A.

The Fc domain confers favorable pharmacokinetic properties to the antibodies of the invention, including a long serum half-life which contributes to good accumulation in the target tissue and a favorable tissue-blood distribution ratio. At the same time it may, however, lead to undesirable targeting of the antibodies of the invention to cells expressing Fc receptors rather than to the preferred antigen-bearing cells. Accordingly, in particular embodiments the Fc domain of the antibodies of the invention exhibits reduced binding affinity to an Fc receptor and/or reduced effector function, as compared to a native IgG Fc domain, in particular an IgG1 Fc domain or an IgG4 Fc domain. More particularly, the Fc domain is an IgG1 Fc domain.

In a particular aspect, the Fc domain is engineered to have reduced binding affinity to an Fc receptor and/or reduced effector function, as compared to a non-engineered Fc domain. In one such embodiment the Fc domain exhibits less than 50%, preferably less than 20%, more preferably less than 10% and most preferably less than 5% of the binding affinity to an Fc receptor, as compared to a native IgG1 Fc domain, and/or less than 50%, preferably less than 20%, more preferably less than 10% and most preferably less than 5% of the effector function, as compared to a native IgG1 Fc domain. In one embodiment, the Fc domain does not substantially bind to an Fc receptor and/or induce effector function. In a particular embodiment the Fc receptor is an Fcγ receptor. In one embodiment, the Fc receptor is a human Fc receptor. In one embodiment, the Fc receptor is an activating Fc receptor. In a specific embodiment, the Fc receptor is an activating human Fcγ receptor, more specifically human FcγRIIIa, FcγRI or FcγRIIa, most specifically human FcγRIIIa. In one embodiment, the Fc receptor is an inhibitory Fc receptor. In a specific embodiment, the Fc receptor is an inhibitory human Fcγ receptor, more specifically human FcγRIIB. In one embodiment the effector function is one or more of CDC, ADCC, ADCP, and cytokine secretion. In a particular embodiment, the effector function is ADCC. In one embodiment, the Fc domain exhibits substantially similar binding affinity to neonatal Fc receptor (FcRn), as compared to a native IgG1 Fc domain. Substantially similar binding to FcRn is achieved when the Fc domain exhibits greater than about 70%, particularly greater than about 80%, more particularly greater than about 90% of the binding affinity of a native IgG1 Fc domain to FcRn. In some embodiments, binding affinity to a complement component, specifically binding affinity to C1q, is also reduced. In one aspect, binding affinity to neonatal Fc receptor (FcRn) is not reduced.

In certain embodiments the Fc domain of the antibody of the invention is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). Certain antibody variants with improved or diminished binding to FcRs are described. (e.g. U.S. Pat. No. 6,737,056; WO 2004/056312, and Shields, R. L. et al., J. Biol. Chem. 276 (2001) 6591-6604).

In one aspect of the invention, the Fc domain comprises an amino acid substitution at one or more of positions E233, L234, L235, G236, N297, P331 and P329. In some aspects, the Fc domain comprises at least one amino acid substitution selected from the group consisting of L234A, L234G, L234Q, L234S, L234T, L235S, L235G, L235T, L235V, L235Q, and G236R. In some aspects, the Fc domain comprises the amino acid substitutions L234A and L235A (“LALA”). In one such embodiment, the Fc domain is an IgG1 Fc domain, particularly a human IgG1 Fc domain. In one aspect, the Fc domain comprises an amino acid substitution at position P329. In a more specific aspect, the amino acid substitution is P329A or P329G, particularly P329G. In one embodiment the Fc domain comprises an amino acid substitution at position P329 and a further amino acid substitution selected from the group consisting of E233P, L234A, L234G, L324Q, L234S, L234T, L235A, L235E, L235S, L235G, L235T, L235V, L235Q, G236R, N297A, N297D or P331S. In more particular embodiments the Fc domain comprises the amino acid mutations L234A, L235A and P329G (“P329G LALA”). The “P329G LALA” combination of amino acid substitutions almost completely abolishes Fcγ receptor binding of a human IgG1 Fc domain, as described in PCT Patent Application No. WO 2012/130831 A1. Said document also describes methods of preparing such mutant Fc domains and methods for determining its properties such as Fc receptor binding or effector functions. Such an antibody is an IgG1 with mutations L234A and L235A or with mutations L234A, L235A and P329G (numbering according to EU index of Kabat et al, Kabat et al., Sequences of Proteins of Immunological Interest, 5th Ed. Public Health Service, National Institutes of Health, Bethesda, Md., 1991).

In some aspects of the invention, the heavy chain constant region comprises an IgG constant region containing mutations at amino acid positions L234, L235, and/or G236. Sets of mutations that can be particularly beneficial for use with anti-CD36 antibodies include embodiments in which the heavy chain constant region comprises an IgG constant region containing a set of mutations selected from the group consisting of L234A, L235S, and G236R; L234G, L235S, and G236R; L234Q, L235S, and G236R; L234S, L235G, and G236R; L234S, L235T, and G236R; L234S, L235V, and G236R; L234T, L235Q, and G236R; L234T, L235S, and G236R; L234T, L235T, and G236R; L234Q, L235S, and G236R; L234A and L235A; L234A, L235A, and P329G; G236R and L328R; L234A and G237A; L234A, L235A, and G237A; L234A and L235E; L235V, F243L, R292P, Y300L, and P396L; D265A and P329A; L234A, L235A, and K322A; L234F, L235E, and P331S; L234F, L235Q, and K322Q; L234A, L235A, G237A, P238S, H268A, A330S, and P331S; E233P, L234V, L235A, G236A, A327G, A330S, and P331S; L235A and G236R; L235S and G236R; G236R; L234Q and L235S; L235G and G236R; L234Q, L235S. and A236R; L234Q and L235S; L234Q, L235S, and G236R; L234Q, L235S, and G236R; L234Q, L235S, and G236R; L234Q, L235S, and G236R; L234Q, L235S, G236R, M252Y, S254T, and T256E; and L234Q, L235S, G236R, T250Q, and M428L. In some embodiments, the heavy chain constant region comprises an IgG constant region containing the L234G, L235S, and G236R mutations. In some embodiments, the heavy chain constant region comprises an IgG constant region containing the L234S, L235T, and G236R mutations. In some embodiments, the heavy chain constant region comprises an IgG constant region containing the L234S, L235V, and G236R mutations. In some embodiments, the heavy chain constant region comprises an IgG constant region containing the L234T, L235Q, and G236R mutations. In some embodiments, the heavy chain constant region comprises an IgG constant region containing the L234T, L235T, and G236R mutations. In some embodiments, the heavy chain constant region comprises an IgG constant region containing the L234A, L235S, and G236R mutations. In some embodiments, the heavy chain constant region comprises an IgG constant region containing the L234Q, L235S, and G236R mutations. In some embodiments, the heavy chain constant region comprises an IgG constant region containing the L234S, L235G, and G236R mutations. In some embodiments, the heavy chain constant region comprises an IgG constant region containing the L234T, L235S, and G236R mutations. In some embodiments, the heavy chain constant region comprises an IgG constant region containing the L234Q, L235S, and G236R mutations. In some embodiments, the heavy chain constant region comprises an IgG constant region containing the L234A and L235A mutations. In some embodiments, the heavy chain constant region comprises an IgG constant region containing the L234A, L235A, and P329G mutations.

In some embodiments, the Fc region which is altered to create a variant Fc region, may be selected from: lgG1, lgG2, lgG3 or lgG4. (See, for example, WO 2021/234402, the entire contents of which is incorporated by reference in its entirety.) In some embodiments, the Fc region is lgG1. In humans, the wild-type residues corresponding to L234, L235 and G236 (EU numbering) in lgG1 are: V234, A235, D236 in lgG2; L234, L235 and G236 in lgG3 and F234, L235 and G236 in lgG4. lgG2 has a deletion at position 236 which is responsible for reduced binding to FcRn and reduced transplacental transport. In some embodiments, an Arg residue inserted at 236 restores the binding of lgG2 to FcRn to be more similar to that of lgG1 and thus can increase the half-life of lgG2 in the circulation. In other embodiments, retention of the deletion at 236 preserves the reduced binding of lgG2 to FcRn.

In some embodiments a protein is provided which comprises a human lgG1 variant Fc region comprising a set of amino acid substitutions selected from: L234A/L235A/G236R, L234A/L235S/G236 R, L234A/L235T/G236R, L234D/L235H/G236R, L234D/L235K/G236R, L234D/L235Q/G236R, L234D/L235S/G236R, L234D/L235T/G236R, L234E/L235D/G236R, L234E/L235H/G236R, L234E/L235I/G236R, L234E/L235V/G236R, L234G/L235H/G236R, L234G/L235Q/G236R, L234G/L235S/G236R, L234H/L235I/G236R, L234H/L235S/G236R, L234K/L235Q/G236R, L234K/L235R/G236R, L234K/L235S/G236R, L234K/L235T/G236R, L234K/L235V/G236R, L234Q/L235A/G236R, L234Q/L235D/G236R, L234Q/L235H/G236R, L234Q/L235Q/G236 R, L234Q/L235R/G236R, L234Q/L235S/G236R, L234Q/L235T/G236R, L234Q/L235V/G236R, L234R/L235D/G236R, L234R/L235E/G236R, L234R/L235H/G236R, L234R/L235I/G236R, L234R/L235K/G236R, L234R/G236R, L234R/L235Q/G236R,
L234 R/L235 R/G236 R, L234R/L235T/G236R, L234S/L235D/G236R, L234S/L235E/G236R, L234S/L235G/G236R, L234S/L235H/G236R, L234S/L235I/G236R, L234S/G236R, L234S/L235R/G236R, L234S/L235T/G236R, L234S/L235V/G236R, L234T/L235A/G236R, L234T/L235I/G236R, L234T/L235K/G236R, L234T/L235Q/G236R, L234T/L235R/G236R, L234T/L235S/G236R, L234T/L235T/G236R, L234T/L235V/G236R.

In some embodiments a protein is provided which comprises a human igG1 variant Fc region comprising a set of amino acid substitutions selected from: L234A/L235A/G236R,
L234A/L235S/G236 R, L234A/L235T/G236R, L234D/L235H/G236R, L234D/L235K/G236R,
L234D/L235Q/G236R, L234D/L235S/G236R, L234D/L235T/G236R, L234E/L235D/G236R,
L234E/L235H/G236R, L234E/L235I/G236R, L234E/L235V/G236R, L234G/L235H/G236R,
L234G/L235Q/G236R, L234G/L235S/G236R, L234H/L235I/G236R, L234H/L235S/G236R,
L234K/L235Q/G236R, L234K/L235R/G236R, L234K/L235S/G236R, L234K/L235T/G236R,
L234K/L235V/G236R, L234Q/L235A/G236R, L234Q/L235D/G236R, L234Q/L235H/G236R,
L234Q/L235Q/G236 R, L234Q/L235R/G236R, L234Q/L235S/G236R, L234Q/L235T/G236R,
L234Q/L235V/G236R, L234R/L235D/G236R, L234R/L235E/G236R, L234R/L235H/G236R, L234R/L235I/G236R, L234R/L235K/G236R, L234R/G236R, L234R/L235Q/G236R, L234R/L235R/G236R, L234R/L235T/G236R, L234S/L235D/G236R, L234S/L235E/G236R, L234S/L235G/G236R, L234S/L235H/G236R, L234S/L235I/G236R, L234S/G236R, L234S/L235R/G236R, L234S/L235T/G236R, L234S/L235V/G236R, L234T/L235A/G236R, L234T/L235I/G236R, L234T/L235K/G236R, L234T/L235Q/G236R, L234T/L235R/G236R, L234T/L235S/G236R, L234T/L235T/G236R, L234T/L235V/G236R, L235T/G236R.

In some embodiments a protein is provided which comprises a human igG1 variant Fc region comprising a set of amino acid substitutions selected from: L234A/L235A/G236R, L234A/L235S/G236 R, L234A/L235T/G236R, L234D/L235H/G236R, L234D/L235K/G236R, L234D/L235Q/G236R, L234D/L235S/G236R, L234D/L235T/G236R, L234E/L235D/G236R, L234E/L235H/G236R, L234E/L235I/G236R, L234E/L235V/G236R, L234G/L235Q/G236R, L234G/L235S/G236R, L234Q/L235A/G236R, L234Q/L235D/G236R, L234Q/L235Q/G236R, L234Q/L235S/G236R, L234Q/L235T/G236R, L234Q/L235V/G236R, L234R/L235D/G236R, L234R/L235E/G236R, L234S/L235D/G236R, L234S/L235E/G236R, L234S/L235G/G236R, L234S/L235I/G236R, L234S/G236R, L234S/L235T/G236R, L234S/L235V/G236R, L234T/L235A/G236R, L234T/L235I/G236R, L234T/L235Q/G236R, L234T/L235S/G236R, L234T/L235T/G236R, L234T/L235V/G236R, L235T/G236R.

In some embodiments a protein is provided which comprises a human igG1 variant Fc region comprising a set of amino acid substitutions selected from: L234A/L235A/G236R, L234A/L235S/G236R, L234D/L235K/G236R, L234D/L235S/G236R, L234D/L235T/G236R, L234G/L235S/G236R, L234H/L235S/G236R, L234K/L235Q/G236R, L234K/L235R/G236R, L234K/L235S/G236R, L234K/L235T/G236R, L234K/L235V/G236R, L234Q/L235A/G236R, L234Q/L235D/G236R, L234Q/L235R/G236R, L234Q/L235S/G236R, L234Q/L235T/G236R, L234Q/L235V/G236R, L234R/L235D/G236R, L234R/L235E/G236R, L234R/L235H/G236R, L234R/L235I/G236R, L234R/L235K/G236R, L234R/L235Q/G236R, L234R/L235R/G236R, L234S/L235G/G236R, L234S/L235H/G236R, L234S/L235I/G236R, L234S/L235R/G236R, L234S/L235T/G236R, L234S/L235V/G236R, L234T/L235K/G236R, L234T/L235Q/G236R, L234T/L235R/G236R, L234T/L235S/G236R, L234T/L235T/G236R, L234T/L235V/G236R.

In some embodiments a protein is provided which comprises a human lgG2 variant Fc region comprising a set of amino acid substitutions selected from: V234A/A236R, V234A/A235S/A236R,
V234A/A235T/A236R, V234D/A235H/A236R, V234D/A235K/A236R, V234D/A235Q/A236R,
V234D/A235S/A236R, V234D/A235T/A236R, V234E/A235D/A236R, V234E/A235H/A236R,
V234E/A235I/A236R, V234E/A235V/A236R, V234G/A235H/A236R, V234G/A235Q/A236R,
V234G/A235S/A236R, V234H/A235I/A236R, V234H/A235S/A236R, V234K/A235Q/A236R, V234K/A235R/A236R, V234K/A235S/A236R, V234K/A235T/A236R, V234K/A235V/A236R, V234Q/A235A/A236R, V234Q/A235D/A236R, V234Q/A235H/A236R, V234Q/A235Q/A236R, V234Q/A235R/A236R, V234Q/A235S/A236R, V234Q/A235T/A236R, V234Q/A235V/A236R, V234R/A235D/A236R, V234R/A235E/A236R, V234R/A235H/A236R, V234R/A235I/A236R, V234R/A235K/A236R, V234R/A235L/A236R, V234R/A235Q/A236R, V234R/A235R/A236R, V234R/A235T/A236R, V234S/A235D/A236R, V234S/A235E/A236R, V234S/A235G/A236R, V234S/A235H/A236R, V234S/A235I/A236R, V234S/A235L/A236R, V234S/A235R/A236R, V234S/A235T/A236R, V234S/A235V/A236R, V234T/A236R, V234T/A235I/A236R, V234T/A235K/A236R, V234T/A235Q/A236R, V234T/A235R/A236R, V234T/A235S/A236R, V234T/A235T/A236R, V234T/A235V/A236R.

In some embodiments a protein is provided which comprises a human lgG2 variant Fc region comprising a set of amino acid substitutions selected from: V234A/A236R, V234A/A235S/A236R, V234A/A235T/A236R, V234D/A235H/A236R, V234D/A235K/A236R, V234D/A235Q/A236R, V234D/A235S/A236R, V234D/A235T/A236R, V234E/A235D/A236R, V234E/A235H/A236R, V234E/A235I/A236R, V234E/A235V/A236R, V234G/A235H/A236R, V234G/A235Q/A236R, V234G/A235S/A236R, V234H/A235I/A236R, V234H/A235S/A236R, V234K/A235Q/A236R, V234K/A235R/A236R, V234K/A235S/A236R, V234K/A235T/A236R, V234K/A235V/A236R, V234Q/A235A/A236R, V234Q/A235D/A236R, V234Q/A235H/A236R, V234Q/A235Q/A236R, V234Q/A235R/A236R, V234Q/A235S/A236R, V234Q/A235T/A236R, V234Q/A235V/A236R, V234R/A235D/A236R, V234R/A235E/A236R, V234R/A235H/A236R, V234R/A235I/A236R, V234R/A235K/A236R, V234R/A235L/A236R, V234R/A235Q/A236R, V234R/A235R/A236R, V234R/A235T/A236R, V234S/A235D/A236R, V234S/A235E/A236R, V234S/A235G/A236R, V234S/A235H/A236R, V234S/A235I/A236R, V234S/A235L/A236R, V234S/A235R/A236R, V234S/A235T/A236R, V234S/A235V/A236R, V234T/A236R, V234T/A235I/A236R,
V234T/A235K/D236 R, V234T/A235Q/A236R, V234T/A235R/A236R, V234T/A235S/A236R, V234T/A235T/A236R, V234T/A235V/A236R, A235T/A236R.

In some embodiments a protein is provided which comprises a human lgG2 variant Fc region comprising a set of amino acid substitutions selected from: V234A/A236R, V234A/A235S/A236R, V234A/A235T/A236R, V234D/A235H/A236R, V234D/A235K/A236R, V234D/A235Q/A236R, V234D/A235S/A236R, V234D/A235T/A236R, V234E/A235D/A236R, V234E/A235H/A236R, V234E/A235I/A236R, V234E/A235V/A236R, V234G/A235Q/A236R, V234G/A235S/A236R, V234Q/A235A/A236R, V234Q/A235D/A236R, V234Q/A235Q/A236R, V234Q/A235S/A236R, V234Q/A235T/A236R, V234Q/A235V/A236R, V234R/A235D/A236R, V234R/A235E/A236R, V234S/A235D/A236R, V234S/A235E/A236R, V234S/A235G/A236R, V234S/A235I/A236R, V234S/A235L/A236R, V234S/A235T/A236R, V234S/A235V/A236R, V234T/A236R, V234T/A235I/A236R, V234T/A235Q/A236R, V234T/A235S/A236R, V234T/A235T/A236R, V234T/A235V/A236R, A235T/A236R.

In some embodiments a protein is provided which comprises a human lgG2 variant Fc region comprising a set of amino acid substitutions selected from: V234A/A235S, V234A/A235T, V234D/A235H, V234D/A235K, V234D/A235Q, V234D/A235S, V234D/A235T, V234E/A235D, V234E/A235H, V234E/A235I, V234E/A235V, V234G/A235H, V234G/A235Q, V234G/A235S, V234H/A235I, V234H/A235S, V234K/A235Q, V234K/A235R, V234K/A235S, V234K/A235T, V234K/A235V, V234Q/A235A, V234Q/A235D, V234Q/A235H, V234Q/A235Q, V234Q/A235R, V234Q/A235S, V234Q/A235T, V234Q/A235V, V234R/A235D, V234R/A235E, V234R/A235H, V234R/A235I, V234R/A235K, V234R/A235L, V234R/A235Q, V234R/A235R, V234R/A235T, V234S/A235D, V234S/A235E, V234S/A235G, V234S/A235H, V234S/A235I, V234S/A235L, V234S/A235R, V234S/A235T, V234S/A235V, V234T/A235I, V234T/A235K, V234T/A235Q, V234T/A235R, V234T/A235S, V234T/A235T, V234T/A235V.

In some embodiments a protein is provided which comprises a human lgG2 variant Fc region comprising a set of amino acid substitutions selected from: V234A/A235S, V234A/A235T, V234D/A235H, V234D/A235K, V234D/A235Q, V234D/A235S, V234D/A235T, V234E/A235H, V234E/A235I, V234E/A235V, V234G/A235H, V234G/A235Q, V234G/A235S, V234H/A235I, V234H/A235S, V234K/A235Q, V234K/A235S, V234K/A235T, V234K/A235V, V234Q/A235A, V234Q/A235D, V234Q/A235H, V234Q/A235Q, V234Q/A235R, V234Q/A235S, V234Q/A235T, V234Q/A235V, V234R/A235D, V234R/A235E, V234R/A235I, V234R/A235L, V234R/A235Q, V234R/A235T, V234S/A235D, V234S/A235E, V234S/A235G, V234S/A235H, V234S/A235I, V234S/A235L, V234S/A235R, V234S/A235T, V234S/A235V, V234T/A235I, V234T/A235K, V234T/A235Q, V234T/A235R, V234T/A235S, V234T/A235T, V234T/A235V.

In some embodiments a protein is provided which comprises a human lgG2 variant Fc region comprising a set of amino acid substitutions selected from: V234A/A235S, V234A/A235T, V234D/A235H, V234D/A235K, V234D/A235Q, V234D/A235S, V234D/A235T, V234E/A235D, V234E/A235H, V234E/A235I, V234E/A235V, V234G/A235Q, V234G/A235S, V234H/A235I, V234H/A235S, V234K/A235Q, V234K/A235R, V234K/A235S, V234K/A235T, V234K/A235V, V234Q/A235Q, V234Q/A235R, V234Q/A235S, V234Q/A235T, V234R/A235D, V234R/A235E, V234R/A235H, V234R/A235I, V234R/A235K, V234R/A235L, V234R/A235Q, V234R/A235R, V234R/A235T, V234S/A235D, V234S/A235E, V234S/A235G, V234S/A235H, V234S/A235I, V234S/A235L, V234S/A235R, V234S/A235T, V234S/A235V, V234T/A235I, V234T/A235K, V234T/A235Q, V234T/A235R, V234T/A235S, V234T/A235T.

In some embodiments a protein is provided which comprises a human lgG2 variant Fc region comprising a set of amino acid substitutions selected from: V234D/A235K, V234D/A235Q, V234D/A235S, V234D/A235T, V234E/A235I, V234K/A235Q, V234K/A235R, V234K/A235S, V234K/A235T, V234R/A235D, V234R/A235E, V234R/A235I, V234R/A235K, V234R/A235L, V234R/A235Q, V234R/A235R, V234R/A235T, V234S/A235I, V234S/A235L, V234S/A235R, V234S/A235T.

In some embodiments a protein is provided which comprises a human lgG4 variant Fc region comprising a set of amino acid substitutions selected from: F234A/L235A/G236R, F234A/L235S/G236R, F234A/L235T/G236R, F234D/L235H/G236R, F234D/L235K/G236R, F234D/L235Q/G236R, F234D/L235S/G236R, F234D/L235T/G236R, F234E/L235D/G236R, F234E/L235H/G236R, F234E/L235I/G236R, F234E/L235V/G236R, F234G/L235H/G236R, F234G/L235Q/G236R, F234G/L235S/G236R, F234H/L235I/G236R, F234H/L235S/G236R, F234K/L235Q/G236R, F234K/L235R/G236R, F234K/L235S/G236R, F234K/L235T/G236R, F234K/L235V/G236R, F234Q/L235A/G236R, F234Q/L235D/G236R, F234Q/L235H/G236R, F234Q/L235Q/G236R, F234Q/L235R/G236R, F234Q/L235S/G236R, F234Q/L235T/G236R, F234Q/L235V/G236R, F234R/L235D/G236R, F234R/L235E/G236R, F234R/L235H/G236R, F234R/L235I/G236R, F234R/L235K/G236R, F234R/G236R, F234R/L235Q/G236R, F234R/L235R/G236R, F234R/L235T/G236R, F234S/L235D/G236R, F234S/L235E/G236R, F234S/L235G/G236R, F234S/L235H/G236R, F234S/L235I/G236R, F234S/G236R, F234S/L235R/G236R, F234S/L235T/G236R, F234S/L235V/G236R, F234T/L235A/G236R, F234T/L235I/G236R, F234T/L235K/G236R, F234T/L235Q/G236R, F234T/L235R/G236R, F234T/L235S/G236R, F234T/L235T/G236R, F234T/L235V/G236R.

In some embodiments a protein is provided which comprises a human lgG4 variant Fc region comprising a set of amino acid substitutions selected from: F234A/L235A/G236R,
F234A/L235S/G236R, F234A/L235T/G236R, F234D/L235H/G236R, F234D/L235K/G236R,
F234D/L235Q/G236R, F234D/L235S/G236R, F234D/L235T/G236R, F234E/L235D/G236R,
F234E/L235H/G236R, F234E/L235I/G236R, F234E/L235V/G236R, F234G/L235H/G236R,
F234G/L235Q/G236R, F234G/L235S/G236R, F234H/L235I/G236R, F234H/L235S/G236R,
F234K/L235Q/G236R, F234K/L235R/G236R, F234K/L235S/G236R, F234K/L235T/G236R,
F234K/L235V/G236R, F234Q/L235A/G236R, F234Q/L235D/G236R, F234Q/L235H/G236R,
F234Q/L235Q/G236R, F234Q/L235R/G236R, F234Q/L235S/G236R, F234Q/L235T/G236R,
F234Q/L235V/G236R, F234R/L235D/G236R, F234R/L235E/G236R, F234R/L235H/G236R, F234R/L235I/G236R, F234R/L235K/G236R, F234R/G236R, F234R/L235Q/G236R, F234R/L235R/G236R, F234R/L235T/G236R, F234S/L235D/G236R, F234S/L235E/G236R, F234S/L235G/G236R, F234S/L235H/G236R, F234S/L235I/G236R, F234S/G236R, F234S/L235R/G236R, F234S/L235T/G236R, F234S/L235V/G236R, F234T/L235A/G236R, F234T/L235I/G236R, F234T/L235K/G236R, F234T/L235Q/G236R, F234T/L235R/G236R, F234T/L235S/G236R, F234T/L235T/G236R, F234T/L235V/G236R, L235T/G236R.

In some embodiments a protein is provided which comprises a human lgG4 variant Fc region comprising a set of amino acid substitutions selected from: F234A/L235A/G236R, F234A/L235S/G236R, F234A/L235T/G236R, F234D/L235H/G236R, F234D/L235K/G236R, F234D/L235Q/G236R, F234D/L235S/G236R, F234D/L235T/G236R, F234E/L235D/G236R, F234E/L235H/G236R, F234E/L235I/G236R, F234E/L235V/G236R, F234G/L235Q/G236R, F234G/L235S/G236R, F234Q/L235A/G236R, F234Q/L235D/G236R, F234Q/L235Q/G236R, F234Q/L235S/G236R, F234Q/L235T/G236R, F234Q/L235V/G236R, F234R/L235D/G236R, F234R/L235E/G236R, F234S/L235D/G236R, F234S/L235E/G236R, F234S/L235G/G236R, F234S/L235I/G236R, F234S/G236R, F234S/L235T/G236R, F234S/L235V/G236R, F234T/L235A/G236R, F234T/L235I/G236R, F234T/L235Q/G236R, F234T/L235S/G236R, F234T/L235T/G236R, F234T/L235V/G236R, L235T/G236R.

In one aspect, the antibody of the invention comprises (all positions according to EU index of Kabat) (i) a homodimeric Fc-region of the human IgG1 subclass optionally with the mutations P329G, L234A and L235A, or (ii) a homodimeric Fc-region of the human IgG4 subclass optionally with the mutations P329G, S228P and L235E, or (iii) a homodimeric Fc-region of the human IgG1 subclass optionally with the mutations P329G, L234A, L235A, I253A, H310A, and H435A, or optionally with the mutations P329G, L234A, L235A, H310A, H433A, and Y436A, or (iv) a heterodimeric Fc-region wherein one Fc-region polypeptide comprises the mutation T366W, and the other Fc-region polypeptide comprises the mutations T366S, L368A and Y407V, or wherein one Fc-region polypeptide comprises the mutations T366W and Y349C, and the other Fc-region polypeptide comprises the mutations T366S, L368A, Y407V, and S354C, or wherein one Fc-region polypeptide comprises the mutations T366W and S354C, and the other Fc-region polypeptide comprises the mutations T366S, L368A, Y407V and Y349C, or (v) a heterodimeric Fc-region of the human IgG1 subclass wherein both Fc-region polypeptides comprise the mutations P329G, L234A and L235A and one Fc-region polypeptide comprises the mutation T366W, and the other Fc-region polypeptide comprises the mutations T366S, L368A and Y407V, or wherein one Fc-region polypeptide comprises the mutations T366W and Y349C, and the other Fc-region polypeptide comprises the mutations T366S, L368A, Y407V, and S354C, or wherein one Fc-region polypeptide comprises the mutations T366W and S354C, and the other Fc-region polypeptide comprises the mutations T366S, L368A, Y407V and Y349C.

In one aspect, the Fc domain is an IgG4 Fc domain. In a more specific embodiment, the Fc domain is an IgG4 Fc domain comprising an amino acid substitution at position S228 (Kabat numbering), particularly the amino acid substitution S228P. In a more specific embodiment, the Fc domain is an IgG4 Fc domain comprising amino acid substitutions S228P and L235E. In a more specific embodiment, the Fc domain is an IgG4 Fc domain comprising amino acid substitutions L235E and S228P and P329G. This amino acid substitution reduces in vivo Fab arm exchange of IgG4 antibodies (see Stubenrauch et al., Drug Metabolism and Disposition 38, 84-91 (2010)). Thus, in one aspect, provided is an antibody, comprising (all positions according to EU index of Kabat) a heterodimeric Fc-region of the human IgG4 subclass wherein both Fc-region polypeptides comprise the mutations S228P and L235E or P329G, S228P and L235E and one Fc-region polypeptide comprises the mutation T366W, and the other Fc-region polypeptide comprises the mutations T366S, L368A and Y407V, or wherein one Fc-region polypeptide comprises the mutations T366W and Y349C, and the other Fc-region polypeptide comprises the mutations T366S, L368A, Y407V, and S354C, or wherein one Fc-region polypeptide comprises the mutations T366W and S354C, and the other Fc-region polypeptide comprises the mutations T366S, L368A, Y407V and Y349C.

Antibodies with increased half-lives and improved binding to the neonatal Fc receptor (FcRn), which is responsible for the transfer of maternal IgGs to the fetus (Guyer, R. L. et al., J. Immunol. 117 (1976) 587-593, and Kim, J. K. et al., J. Immunol. 24 (1994) 2429-2434), are described in US 2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also, Duncan, A. R. and Winter, G., Nature 322 (1988) 738-740; U.S. Pat. Nos. 5,648,260; 5,624,821; and WO 94/29351 concerning other examples of Fc region variants.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating antibodies comprising an Fc domain for Fc receptors may be evaluated using cell lines known to express particular Fc receptors, such as human NK cells expressing FcTIIIa receptor. Effector function of an Fc domain, or antibodies of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al., Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, a reporter-based assay that uses an engineered Jurkat stable cell line as the source of effector cells may be employed to measure ADCC, CDC and ADCP (Promega, Madison, Wis.). Alternatively, or additionally, ADCC activity of the molecule of interest may be assessed in vivo, e.g. in an animal model such as that disclosed in Clynes et al., Proc Natl Acad Sci USA 95, 652-656 (1998).

### Nucleotides Encoding Anti-CD36 Antibodies

In certain aspects, provided herein are polynucleotides comprising a nucleotide sequence encoding an antibody described herein or a domain thereof (e.g., a variable light chain region and/or variable heavy chain region) that immunospecifically binds to a CD36 (e.g., human CD36) antigen, and vectors, e.g., vectors comprising such polynucleotides for recombinant expression in host cells (e.g., E. coli and mammalian cells).

In particular aspects, provided herein are polynucleotides comprising nucleotide sequences encoding antibodies that immunospecifically bind to a CD36 polypeptide (e.g., human CD36) and comprise an amino acid sequence as described herein, as well as antibodies that compete with such antibodies for binding to a CD36 polypeptide (e.g., in a dose-dependent manner), or which bind to the same epitope as that of such antibodies.

In certain aspects, provided herein are polynucleotides comprising a nucleotide sequence encoding the light chain or heavy chain of an antibody described herein. The polynucleotides can comprise nucleotide sequences encoding a heavy chain comprising the VHs or CDRs of antibodies described herein. The polynucleotides can comprise nucleotide sequences encoding a light chain comprising the VLs or CDRs of antibodies described herein.

In particular embodiments, provided herein are polynucleotides comprising a nucleotide sequence encoding an anti-CD36 antibody comprising three VH chain CDRs, e.g., containing VH CDR1, VH CDR2, VH CDR3 of any one of antibodies described herein. In specific embodiments, provided herein are polynucleotides comprising three VL chain CDRs, e.g., containing VL CDR1, VL CDR2, and VL CDR3 of any one of antibodies described herein. In specific embodiments, provided herein are polynucleotides comprising a nucleotide sequence encoding an anti-CD36 antibody comprising three VH chain CDRs, e.g., containing VH CDR1, VH CDR2, and VH CDR3 of any one of antibodies described herein and three VL chain CDRs, e.g., containing VL CDR1, VL CDR2, and VL CDR3 of any one of antibodies described herein.

In particular embodiments, provided herein are polynucleotides comprising a nucleotide sequence encoding an anti-CD36 antibody or a fragment thereof comprising a VH domain, e.g., containing FR1-CDR1-FR2-CDR2-FR3-CDR3-FR4, comprising an amino acid sequence described herein. In specific embodiments, provided herein are polynucleotides comprising a nucleotide sequence encoding an anti-CD36 antibody or a fragment thereof comprising a VL domain, e.g., containing FR1-CDR1-FR2-CDR2-FR3-CDR3-FR4, comprising an amino acid sequence described herein.

Also provided herein are polynucleotides encoding an anti-CD36 antibody described herein or a domain thereof that are optimized, e.g., by codon/RNA optimization, replacement with heterologous signal sequences, and elimination of mRNA instability elements. Methods to generate optimized nucleic acids encoding an anti-CD36 antibody or a domain thereof (e.g., heavy chain, light chain, VH domain, or VL domain) for recombinant expression by introducing codon changes (e.g., a codon change that encodes the same amino acid due to the degeneracy of the genetic code) and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly. The contents of each of these patents are incorporate herein by reference in their entireties.

In some embodiments, provided herein are polynucleotides encoding any of the antibodies or antibody fragments described in this application. Exemplary nucleotide sequences are provided below in Table 4.

TABLE 4
Nucleotide Sequences

| SEQ | | |
|---|---|---|
| ID NO | Name | Sequence |
| 207 | ONA-0 heavy | CAAGTGCAGCTGAAGCAGTCCGGAGCTGATCTGGTGAGACC |
| | chain | CGGAGCCAGCGTGAAGCTGAGCTGCAAGGCCAGCGGCTACA |
| | | CCTTCACCGACTACTACATCAACTGGGTGAAGCAGAGGCCC |
| | | GGCCAAGGACTGGAGTGGATCGCTAGAATCTACCCCGGCTC |
| | | CGGCAATACATACTACAACGAGAAGTTCAAAGGCAAGGCCA |
| | | CACTGACCGCCGAGAAGAGCAGCAGCACCGCCTACATGCAG |
| | | CTGAGCTCTCTGACCTCCGAGGACAGCGCCGTGTACTTTTG |
| | | CGCCAGAGGCATCGGAGGCGGATTCGGCATGGATTACTGGG |
| | | GCCAAGGCACCTCCGTGACCGTCTCGAGCGAATCGGCCAGA |
| | | AACCCCACTATCTACCCTCTGACCCTGCCTCCTGTCCTGTG |
| | | TTCCGACCCCGTGATCATCGGATGCCTGATCCACGACTACT |
| | | TCCCTTTCGGCACCATGAACGTGACCTGGGGGAAGTCGGGA |
| | | AAGGACATTACTACCGTGAACTTCCCACCGGCCCTGGCGTC |
| | | GGGGGGTCGCTACACCATGTCCAGCCAGCTTACTCTGCCCG |
| | | CTGTGGAGTGCCCCGAAGGAGAGTCAGTGAAGTGCTCCGTG |
| | | CAACATGACTCCAACCCGGTCCAGGAATTGGACGTCAATTG |
| | | CTCCCCGACTCCGCCTCCGCCTATCACGATCCCAAGCTGCC |
| | | AGCCCTCCCTGAGCCTCCAGCGGCCAGCCCTGGAGGATCTT |
| | | CTGCTGGGCTCCGACGCCTCCATTACATGCACTCTGAACGG |
| | | CCTGAGAAACCCGGAAGGGGCGGCCTTTACTTGGGAGCCCT |
| | | CCACCGGGAAGGATGCGGTCCAGAAGAAGGCAGCCCAAAAT |
| | | TCCTGCGGATGCTACTCAGTGTCTAGCGTGCTGCCTGGTTG |
| | | TGCCGAACGGTGGAACTCCGGAGCGTCATTCAAGTGTACCG |
| | | TGACCCACCCTGAGTCCGGAACTCTGACCGGCACCATCGCC |
| | | AAGGTCACCGTGAACACCTTTCCGCCACAAGTGCACCTCCT |
| | | GCCGCCGCCGTCGGAGGAACTCGCTCTGAACGAGTTGCTCT |
| | | CGCTGACTTGTCTCGTGCGCGCCTTCAACCCTAAGGAGGTG |
| | | CTCGTGCGCTGGCTGCATGGCAACGAAGAACTGTCCCCCGA |
| | | ATCGTACCTGGTGTTCGAACCGCTGAAAGAGCCCGGAGAGG |
| | | GTGCAACCACCTACCTTGTGACGAGCGTGCTCCGGGTGTCC |
| | | GCCGAAACCTGGAAGCAGGGCGACCAGTACAGCTGCATGGT |
| | | CGGCCACGAGGCCCTCCCCATGAACTTCACTCAGAAAACCA |
| | | TTGATAGGTTGTCCGGAAAGCCCACCAACGTGTCAGTGTCC |
| | | GTGATTATGAGCGAAGGAGATGGAATCTGCTAT |
| 208 | ONA-0-v1 | TCCATCGTGATGACCCAGACCCCCAAGTTTCTGCTGGTGTC |
| | light chain | CGCCGGAGACAGAATCACCATCACATGCAAGGCCAGCCAGA |
| | | GCGTGAGCGATGACGTGGCTTGGTACCAGCAGAAGCCCGGC |
| | | CAGAGCCCTAAGCTGCTGATCTACTACGCCAGCAATAGATA |
| | | CACCGGAGTGCCCGATAGATTCACCGGCAGCGGCTACGGCA |
| | | CCGACTTCACCTTCACAATCTCCACCGTGCAAGCCGAGGAT |
| | | CTGGCCGTGTACTTCTGTCAGCAAGACTACTCCAGCCCTCT |
| | | GACCTTCGGAGCCGGCACCAAGCTCGAGATCAAGCGCGCAG |
| | | ATGCTGCTCCTACCGTGAGCATCTTCCCGCCGTCCAGCGAA |
| | | CAACTCACTAGCGGAGGCGCGTCAGTGGTCTGCTTCCTTAA |
| | | CAATTTCTACCCTAAGGACATCAACGTCAAGTGGAAGATTG |
| | | ACGGATCGGAACGCCAGAACGGAGTGCTGAACTCATGGACT |
| | | GATCAGGATTCCAAAGACTCGACTTACTCCATGTCCAGCAC |
| | | CCTGACCCTGACCAAAGACGAGTACGAAAGGCACAACTCGT |
| | | ACACGTGCGAAGCCACCCACAAGACTTCCACCTCGCCCATC |
| | | GTGAAGTCCTTCAATCGCAATGAGTGC |
| 209 | ONA-0-v1 VH | CAAGTGCAGCTGAAGCAGTCCGGAGCTGATCTGGTGAGACC |
| | | CGGAGCCAGCGTGAAGCTGAGCTGCAAGGCCAGCGGCTACA |
| | | CCTTCACCGACTACTACATCAACTGGGTGAAGCAGAGGCCC |
| | | GGCCAAGGACTGGAGTGGATCGCTAGAATCTACCCCGGCTC |
| | | CGGCAATACATACTACAACGAGAAGTTCAAAGGCAAGGCCA |
| | | CACTGACCGCCGAGAAGAGCAGCAGCACCGCCTACATGCAG |
| | | CTGAGCTCTCTGACCTCCGAGGACAGCGCCGTGTACTTTTG |
| | | CGCCAGAGGCATCGGAGGCGGATTCGGCATGGATTACTGGG |
| | | GCCAAGGCACCTCCGTGACCGTCTCGAGC |
| 210 | ONA-0-v1 VL | TCCATCGTGATGACCCAGACCCCCAAGTTTCTGCTGGTGTC |
| | | CGCCGGAGACAGAATCACCATCACATGCAAGGCCAGCCAGA |
| | | GCGTGAGCGATGACGTGGCTTGGTACCAGCAGAAGCCCGGC |
| | | CAGAGCCCTAAGCTGCTGATCTACTACGCCAGCAATAGATA |
| | | CACCGGAGTGCCCGATAGATTCACCGGCAGCGGCTACGGCA |
| | | CCGACTTCACCTTCACAATCTCCACCGTGCAAGCCGAGGAT |
| | | CTGGCCGTGTACTTCTGTCAGCAAGACTACTCCAGCCCTCT |
| | | GACCTTCGGAGCCGGCACCAAGCTCGAGATCAAG |
| 211 | 1G04 heavy | CAAGTGCAGCTGAAGCAGTCCGGAGCTGATCTGGTGAGACC |
| | chain | CGGAGCCAGCGTGAAGCTGAGCTGCAAGGCCAGCGGCTACA |
| | | CCTTCACCGACTACTACATCAACTGGGTGAAGCAGAGGCCC |
| | | GGCCAAGGACTGGAGTGGATCGCTAGAATCTACCCCGGCTC |
| | | CGGCAATACATACTACAACGAGAAGTTCAAAGGCAAGGCCA |
| | | CACTGACCGCCGAGAAGAGCAGCAGCACCGCCTACATGCAG |
| | | CTGAGCTCTCTGACCTCCGAGGACAGCGCCGTGTACTTTTG |
| | | CGCCAGAGGCATCGGAGGCGGATTCGGCATGGATTACTGGG |
| | | GCCAAGGCACCTCCGTGACCGTCTCGAGCGCCAGCACCAAA |
| | | GGTCCATCCGTGTTTCCGCTCGCCCCGTCCTCAAAGTCGAC |
| | | CTCCGGAGGCACTGCCGCCCTGGGCTGCCTTGTCAAGGACT |
| | | ATTTCCCCGAACCTGTCACGGTGTCCTGGAACAGCGGCGCT |
| | | CTGACTTCCGGAGTGCACACCTTCCCCGCCGTCCTGCAATC |
| | | CAGCGGCCTGTACTCACTGTCATCCGTTGTGACTGTCCCGT |
| | | CGTCCAGCCTGGGAACCCAAACCTACATTTGCAACGTGAAT |
| | | CACAAACCATCGAATACCAAGGTCGATAAGAAAGTCGAGCC |
| | | GAAGTCATGCGACAAGACTCACACCTGTCCGCCTTGCCCGG |
| | | CGCCAGAAGCGGCCGGCGGCCCTTCGGTGTTTTTGTTTCCG |
| | | CCGAAGCCGAAGGACACTCTGATGATCTCACGCACTCCAGA |
| | | GGTGACTTGCGTGGTGGTCGATGTTTCGCACGAGGACCCGG |
| | | AAGTGAAATTCAACTGGTATGTCGACGGGGTGGAAGTGCAT |
| | | AATGCCAAGACGAAGCCGAGGGAGGAACAGTACAACTCCAC |
| | | CTACAGAGTGGTTTCAGTCCTTACCGTCCTCCATCAAGATT |
| | | GGCTGAACGGAAAGGAGTACAAATGTAAGGTGTCGAACAAA |
| | | GCGTTGCCGGCCCCTATCGAAAAGACTATCAGCAAGGCCAA |
| | | AGGACAGCCGCGGGAGCCGCAAGTGTACACCCTCCCGCCTT |
| | | CGCGGGACGAGCTGACCAAGAATCAGGTGTCCCTTACTTGC |
| | | CTGGTGAAGGGATTCTACCCCTCGGATATCGCAGTCGAATG |
| | | GGAATCGAATGGACAGCCAGAAAACAACTACAAGACCACTC |
| | | CCCCGGTGCTCGACTCCGACGGTTCCTTCTTCCTGTACTCG |
| | | AAGCTGACCGTGGACAAATCACGCTGGCAGCAGGGAAACGT |
| | | GTTTAGCTGCAGCGTGATGCATGAGGCGCTGCATAATCACT |
| | | ACACCCAGAAGTCACTCTCGCTCAGCCCAGGGAAG |
| 212 | 1G04 light | TCCATCGTGATGACCCAGACCCCCAAGTTTCTGCTGGTGTC |
| | chain | CGCCGGAGACAGAATCACCATCACATGCAAGGCCAGCCAGA |
| | | GCGTGAGCGATGACGTGGCTTGGTACCAGCAGAAGCCCGGC |
| | | CAGAGCCCTAAGCTGCTGATCTACTACGCCAGCAATAGATA |
| | | CACCGGAGTGCCCGATAGATTCACCGGCAGCGGCTACGGCA |
| | | CCGACTTCACCTTCACAATCTCCACCGTGCAAGCCGAGGAT |
| | | CTGGCCGTGTACTTCTGTCAGCAAGACTACTCCAGCCCTCT |
| | | GACCTTCGGAGCCGGCACCAAGCTCGAGATCAAGAGAACTG |
| | | TGGCCGCGCCGTCAGTGTTTATCTTCCCTCCATCGGATGAA |
| | | CAGCTTAAGTCCGGCACGGCGTCTGTGGTCTGCCTGCTCAA |
| | | TAACTTTTACCCTAGGGAAGCTAAAGTCCAATGGAAAGTGG |
| | | ATAACGCCCTGCAGTCAGGAAACAGCCAGGAATCGGTTACC |
| | | GAACAGGACAGCAAGGACAGCACTTACTCCTTGTCGTCGAC |
| | | TCTTACTCTGAGCAAGGCCGATTACGAGAAGCACAAGGTCT |
| | | ACGCCTGCGAGGTCACCCATCAGGGACTCTCGTCCCCGGTG |
| | | ACCAAATCCTTCAATAGAGGCGAATGC |
| 226 | 6G04 VH | CAAGTGCAGCTGCAGCAGAGCGGCGCTGAACTGGTCATGCC |
| | | CGGCGCTAGCGTGAAGATGAGCTGCAAGGCCAGCGGCTACA |
| | | CTTTCACTGACTACTGGATGCACTGGGTGAAGCAGAGGCCC |
| | | GGCCAAGGACTGGAGTGGATCGGCAGCATCGACACTTCCGA |
| | | CAGCTACACAACTTACTCCCAGAAATTCAAGGGCAAGGCCA |
| | | CACTGACAGTGGACGAGAGCAGCAGCACAGCCTACATGGAG |
| | | CTGGCTAGGCTGACTAGCGAGGATAGCGCCATCTACTACTG |
| | | TGTGAGGGGCGAGGATTACGAGGGAACATGGTTCGCCTACT |
| | | GGGGCCAAGGCACACTGGTGACTGTGAGCGCC |
| 227 | 6G04 VL | GAGACTACAGTGACTCAGAGCCCAGCCTCTCTGAGCGTCGC |
| | | TACTGGCGAGAAGGTCACAATTAGGTGCATCACTAGCACAG |
| | | ACATCGACGACGACATGAACTGGTATCAGCAGAAGCCCGGC |
| | | GAGCCTCCTAAGCTGCTGATCTCCGAGGGCAACACACTGAG |
| | | GCCCGGCGTGCCTTCTAGGTTCAGCAGCAGCGGCTACGGCA |
| | | CAGACTTCGTCTTCACTATCGAGAACACTCTGAGCGAGGAC |
| | | GTGGCCGATTACTACTGTCTGCAGAGCGACAACATGCCTCT |
| | | GACTTTCGGCGCCGGCACAAAGCTGGAGCTGAAA |
| 228 | 7G04 VH | CAAGTGCAGATGAAGGAAAGCGGCGCTGAGCTGGTGAGACC |
| | | CGGCGCTAGCGTGAAGCTGAGCTGTAAGGCTCTGGGCTACA |
| | | CATTCACAGACTACGAGATCCAGTGGGTGAAGCAGACACCA |
| | | GTGCACGGACTGGAATGGATCGGAGGCATCCACCCCGGCAG |
| | | CTCCGGCATCGTGTACAACCAGAAGTTCAAGGGCAAGGCCA |
| | | CTCTGACTGCTGACAAGTCCAGCAGCACTGCCTACATGGAG |
| | | CTGAGCTCTCTGACTAGCGAGGATAGCGCCGTGTACTACTG |
| | | TACAAGGGGCGGCGGCTACGATGGAGCTTGGTTCGCCTATT |
| | | GGGGACAAGGCTCTCTGGTCACTGTGAGCGCC |
| 229 | 7G04 VL | GAGACAACTGTGACTCAGAGCCCAGCCTCTCTGTCCATGGC |
| | | TATCGGCGAGAAGGTGACAATTAGGTGCATCACTAGCACAG |
| | | ACATCGACGACGACATGAACTGGTATCAGCAAAAGCCCGGC |
| | | GAGCCTCCTAAGCTGCTGATCAGCGAGGGCAATACACTGAG |
| | | GCCCGGCGTGCCTTCTAGGTTCAGCAGCAGCGGCTACGGCA |
| | | CAGACTTCGTGTTCACTATCGAGAACATGCTGAGCGAGGAC |
| | | GTGGCTGATTACTACTGTCTGCAGAGCGACAATCTGCCTTT |
| | | CACATTCGGCAGCGGCACAAAGCTGGAGATCAAG |
| 230 | 11G04 VH | GAGGTGCAGCTCCAGCAGAGCGGCGCCGAACTGGTGAGACCCGGCG |
| | | CCAGCGTGACACTGAGCTGTAAGGCCAGCGGCTATAGGTTCTCCGATT |
| | | ACGAGATGCAGTGGGTGAAGCAGACTCCAGTCCACGGACTGGAATG |
| | | GATCGGCGGCATCGATCCAGAGACTGGCGGCATCGCCTACAACCAGA |
| | | AGTTCAAGGTCAAGGCCACTCTCACTGCCGACAAGAGCTCCTCCACAG |
| | | CCTTCATGGAGCTGAGGTCTCTGACAAGCGAGGATAGCGCCGTCTACT |
| | | ACTGCACTAGGAAGCTGGACTTCGACTACTGGGGCCAAGGCACTACA |
| | | CTGACTGTGAGCAGC |
| 231 | 11G04 VL | GAGACAACTGTGACACAGAGCCCAGCCTCTCTGAGCGTCGCTACTGG |
| | | CGAGAAGGTGACTATTAGGTGCATCACAAGCACAGACATCGACGACG |
| | | ACATGAACTGGTATCAGCAGAAGCCCGGCGAGCCTCCTAAGCTGCTG |
| | | ATCAGCGAGGGAAACACTCTGAGGCCCGGCGTGCCATCTAGGTTCAG |
| | | CAGCAGCGGCTACGGCACTGACTTCGTGTTCACAATCGAGAACATGCT |
| | | GAGCGAGGACGTGGCCGACTACTACTGTCTGCAGAGCGACAATCTGC |
| | | CTCTGACTTTCGGCGCCGGCACAAAGCTGGAGCTGAAG |
| 232 | 13G04 VH | CAAGTGCAGCTGCAGCAGAGCGGCGCTGAACTGGTGAGACCCGGCG |
| | | CCAGCGTCAAGCTGAGCTGCAAGGCCTCCGGCTACACTTTCACAAGCT |
| | | ACTGGATGAACTGGGTGAAGCAGAGACCCGGCCAAGGACTGGAGTG |
| | | GATCGGCATGATCGACCCTAGCGACAACGAGACACACTACAACCAGA |
| | | TGTTCAAGGACAAGGCCACACTGACTGTCGACAAGAGCTCCTCCACAG |
| | | CCTACATGCAGCTGAGCTCTCTGACTAGCGAGGATAGCGCCGTGTACT |
| | | ACTGCGCTAGGAGCGATTACGGCAACGGCTATAGCTTCTATCTGGACG |
| | | TCTGGGGCGCTGGCACAACTGTGACTGTGAGCAGC |
| 233 | 13G04 VL | GATATCGTGCTGACACAGAGCCCAGCCTCTCTGGCTGTGTCTCTGGGA |
| | | CAGAGAGCCACAATCAGCTGTAGGGCCAGCGAGAGCGTGGATAGCT |
| | | ACGGCAACTCCTTCATGCACTGGTATCAGCAGAAGCCCGGCCAGCCTC |
| | | CTAAGCTGCTGATCTATAGGGCCAGCAATCTGGAGAGCGGCATCCCA |
| | | GCTAGGTTTAGCGGCTCCGGCTCTAGGACTGACTTCACTCTGACTATC |
| | | AACCCAGTGGAGGCCGACGATGTGGCCACTTACTACTGCCAGCAGAG |
| | | CAACGAGGATCCTTGGACATTCGGCGGCGGCACTAAGCTGGAGATCA |
| | | AG |
| 234 | 14G04 VH | CAAGTGCAGCTGCAGCAGAGCGGCGCCGAACTGGTGAGAAGCGGAG |
| | | CTAGCGTGAGGCTGAGCTGTACAGCCTCCGGCTTCAACATCAAGGACT |
| | | ACTACATCCACTGGGTGAAGCAGAGGCCAGAGCAAGGACTGGAGTG |
| | | GATCGGCTGGATCGATCCAGAGAATGGCGACACAGAGTACGCTCCAA |
| | | GGTTCCAAGATAAGGCCACTATGACTGCCGACACTAGCAGCAACACA |
| | | GCCTATCTGCAGCTGAGCTCTCTGACAAGCGAGGACACAGCTGTGTAC |
| | | TACTGCAATGGCTGGCTGCTGAGCGGCAATGGCATGGATTACTGGGG |
| | | CCAAGGCACATCCGTGACAGTGAGCTCC |
| 235 | 14G04 VL | GACATCGTGATGACACAGAGCCACAAGTTCATGAGCACAAGCGTGGG |
| | | CGATAGGGTGAGCATCACTTGCAAGGCTAGCCAAGATGTGGGCACAG |
| | | CTGTGGCTTGGTATCAGCAGAAGCCCGGCCAGAGCCCAAAGCTGCTG |
| | | ATCTACTGGGCCAGCACTAGGCACACTGGCGTGCCAGATAGGTTCACT |
| | | GGCAGCGGCAGCGGCACAGACTTCACTCTGACTATCAGCAACGTGCA |
| | | GAGCGAGGATCTGGCCGATTACTTCTGCCAGCAGTACTCCAGCTACCC |
| | | TACATTCGGCGGCGGCACAAAGCTGGAGATCAAG |
| 236 | 28G04 VH | GAGGTGCAGCTGCAGCAGAGCGGACCAGAGCTGGTGAAGCCCGGCG |
| | | CCAGCGTCAAGATCAGCTGCAAGACTAGCGGCTACACTTTCACTGAGT |
| | | ACACTATCCACTGGGTGAAGCAGAGCCACGGCAAGTCTCTGGAGTGG |
| | | ATCGGCGGCATCATCCCTAACAATGGCGGCAGCAGCCACAAGCAGAA |
| | | CTTCAAGGACAAGGCCACTCTGACAGTGGATAAGAGCAGCAGCACAG |
| | | CCTACATGGAGCTGAGGTCTCTGACAAGCGAGGATTCCGCCGTCTACT |
| | | ACTGTGCTAGGGCCGGCGACTACGCCTTCGATTACTGGGGCCAAGGC |
| | | ACTACACTGACAGTCAGCAGC |
| 237 | 28G04 VL | GAGACAACTGTGACACAGAGCCCAGCTTCTCTGAGCATGGCCATCGG |
| | | CGAGAAGGTCACAATTAGGTGCATCACTTCCACAGACATCGACGACG |
| | | ACATGAACTGGTATCAGCAGAAGCCCGGCGAGCCTCCTAAGCTGCTG |
| | | ATCAGCGAGGGAAACACTCTGAGGCCCGGCGTGCCATCTAGGTTCAG |
| | | CAGCAGCGGCTACGGCACAGATTTCGTGTTCACTATCGAGAACATGCT |
| | | GAGCGAGGACGTGGCCGACTACTACTGTCTGCAGAGCGGAACTCTGC |
| | | CATTCACTTTCGGCTCCGGCACTAAGCTGGAGATCAAG |
| 249 | 11G06 VL | AAGCTTGCCGCCACCATGTCTGTGCCTACACAGGTTCTGGGACTGCTG |
| | | CTGCTGTGGCTGACCGACGCTAGATGCGAGACAACCGTGACTCAGTC |
| | | CCCTGCCTCTCTGTCTGTGGCTACCGGCGAGAAAGTGACCATCCGGTG |
| | | CATCACCTCCACCGACATCGACGACGACATGAACTGGTATCAGCAGAA |
| | | GCCCGGCGAGCCTCCTAAGCTGCTGATCTCTGAGGGCAACACACTGA |
| | | GGCCTGGCGTGCCCTCCAGATTCTCCTCTTCTGGCTACGGCACCGACTT |
| | | CGTGTTCACCATCGAGAACATGCTGTCCGAGGACGTGGCCGACTACTA |
| | | CTGCCTGCAGTCTGACAACCTGCCTCTGACCTTTGGCGCTGGCACCAA |
| | | GCTGGAACTGAAGAGAACAGTGGCCGCTCCTTCCGTGTTCATCTTCCC |
| | | ACCTTCCGACGAGCAGCTGAAGTCTGGCACAGCCTCTGTCGTGTGCCT |
| | | GCTGAACAACTTCTACCCTCGGGAAGCCAAGGTGCAGTGGAAGGTGG |
| | | ACAACGCTCTGCAGTCCGGCAACTCCCAAGAGTCTGTGACCGAGCAG |
| | | GACTCCAAGGACAGCACCTACAGCCTGTCCTCCACACTGACCCTGTCC |
| | | AAGGCCGATTACGAGAAGCACAAGGTGTACGCCTGCGAAGTGACCCA |
| | | TCAGGGCCTGTCTAGCCCTGTGACCAAGTCTTTCAACCGGGGCGAGTG |
| | | CTGATGAATTC |
| 250 | 11G06 VH | AAGCTTGCCGCCACCATGGAATGGTCCTGGGTGTTCCTGTTCTTCCTGT |
| | | CCGTGACCACCGGCGTGCACTCTGAAGTTCAGTTGCAGCAGTCTGGCG |
| | | CCGAACTCGTTAGACCTGGCGCTTCTGTGACCCTGTCCTGCAAGGCTT |
| | | CCGGCTACCGGTTCTCCGACTACGAGATGCAGTGGGTCAAGCAGACC |
| | | CCTGTGCATGGCCTGGAATGGATCGGCGGCATCGATCCTGAGACAGG |
| | | CGGAATCGCCTACAACCAGAAGTTCAAAGTCAAGGCTACCCTGACCGC |
| | | CGACAAGTCCTCTTCCACCGCCTTCATGGAACTGCGGTCCCTGACCTCT |
| | | GAGGACTCCGCCGTGTACTACTGCACCCGGAAGCTGGACTTCGACTAT |
| | | TGGGGCCAGGGCACCACACTGACCGTGTCCTCTGCTTCTACCAAGGGA |
| | | CCCAGCGTGTTCCCTCTGGCTCCTTCCAGCAAGTCTACCTCTGGCGGA |
| | | ACAGCTGCTCTGGGCTGCCTGGTCAAGGACTACTTTCCTGAGCCTGTG |
| | | ACAGTGTCCTGGAACTCTGGCGCTCTGACATCTGGCGTGCACACCTTT |
| | | CCAGCTGTGCTGCAGTCCTCCGGCCTGTACTCTCTGTCCTCTGTCGTGA |
| | | CCGTGCCTTCCAGCTCTCTGGGAACCCAGACCTACATCTGCAATGTGA |
| | | ACCACAAGCCTTCCAACACCAAGGTGGACAAGAAGGTGGAACCCAAG |
| | | TCCTGCGACAAGACCCACACCTGTCCTCCATGTCCTGCTCCAGAGTCCA |
| | | CCAGAGGACCCTCCGTGTTTCTGTTCCCTCCAAAGCCTAAGGACACCCT |
| | | GTACATCACCCGCGAGCCTGAAGTGACCTGCGTGGTGGTGGATGTGT |
| | | CTCACGAGGACCCCGAAGTGAAGTTCAATTGGTACGTGGACGGCGTG |
| | | GAAGTGCACAACGCCAAGACCAAGCCTAGAGAGGAACAGTACAACTC |
| | | CACCTACAGAGTGGTGTCCGTGCTGACCGTGCTGCACCAGGATTGGCT |
| | | GAACGGCAAAGAGTACAAGTGCAAGGTGTCCAACAAGGCCCTGCCTG |
| | | CTCCTATCGAAAAGACCATCTCCAAGGCCAAGGGCCAGCCTAGGGAA |
| | | CCCCAGGTTTACACCTTGCCTCCATCTCGGGACGAGCTGACCAAGAAC |
| | | CAGGTGTCCCTGACATGCCTCGTGAAGGGCTTCTACCCCTCCGATATC |
| | | GCCGTGGAATGGGAGTCTAATGGCCAGCCTGAGAACAACTACAAGAC |
| | | AACCCCTCCTGTGCTGGACTCCGACGGCTCATTCTTTCTGTACTCCAAG |
| | | CTGACAGTGGACAAGTCCAGATGGCAGCAGGGCAACGTGTTCTCCTG |
| | | CAGCGTGATGCACGAGGCCCTGCACAATCACTATACCCAGAAGTCCCT |
| | | GTCTCTGTCCCCTGGCAAGTGATGAATTC |
| 253 | 11G06 VL_v1 | AAGCTTGCCGCCACCATGTCTGTGCCTACACAGGTTCTGGGACTGCTG |
| | | CTGCTGTGGCTGACCGACGCTAGATGCGAGACAACCGTGACTCAGTC |
| | | CCCTGCCTTCATGTCTGCTACCACCGGCGACAAAGTGACCATCTCCTGC |
| | | ATCACCTCCACCGACATCGACGACGACATGAACTGGTATCAGCAGAA |
| | | GCCCGGCGAGCCTCCTAAGCTGCTGATCTCTGAGGGCAACACACTGA |
| | | GGCCTGGCGTGCCCTCCAGATTCTCCTCTTCTGGCTACGGCACCGACTT |
| | | CACCTTCACCATCAACAACATCGAGTCCGAGGACGCCGCCTACTACTA |
| | | CTGCCTGCAGTCTGACAACCTGCCTCTGACCTTTGGCCAGGGCACCAA |
| | | GCTGGAAATCAAGCGGACAGTGGCCGCTCCTTCCGTGTTCATCTTCCC |
| | | ACCTTCCGACGAGCAGCTGAAGTCTGGCACAGCCTCTGTCGTGTGCCT |
| | | GCTGAACAACTTCTACCCTCGGGAAGCCAAGGTGCAGTGGAAGGTGG |
| | | ACAACGCTCTGCAGTCCGGCAACTCCCAAGAGTCTGTGACCGAGCAG |
| | | GACTCCAAGGACAGCACCTACAGCCTGTCCTCCACACTGACCCTGTCC |
| | | AAGGCCGACTACGAGAAGCACAAGGTGTACGCCTGCGAAGTGACCCA |
| | | TCAGGGCCTGTCTAGCCCTGTGACCAAGTCTTTCAACCGGGGCGAGTG |
| | | CTGATGAATTC |
| 254 | 11G06 VL v2 | AAGCTTGCCGCCACCATGTCTGTGCCTACACAGGTTCTGGGACTGCTG |
| | | CTGCTGTGGCTGACCGACGCTAGATGCGAGACAACCGTGACTCAGTC |
| | | CCCTGCCTTCATGTCTGCTACCCCTGGCGACAAAGTGACCATCTCCTGC |
| | | ATCACCTCCACCGACATCGACGACGACATGAACTGGTATCAGCAGAA |
| | | GCCCGGCGAGCCTCCTAAGCTGCTGATCTCTGAGGGCAACACACTGA |
| | | GGCCTGGCGTGCCCTCCAGATTCTCCTCTTCTGGCTACGGCACCGACTT |
| | | CACCTTCACCATCAACAACATCGAGTCCGAGGACGCCGCCTACTACTA |
| | | CTGCCTGCAGTCTGACAACCTGCCTCTGACCTTTGGCCAGGGCACCAA |
| | | GCTGGAAATCAAGCGGACAGTGGCCGCTCCTTCCGTGTTCATCTTCCC |
| | | ACCTTCCGACGAGCAGCTGAAGTCTGGCACAGCCTCTGTCGTGTGCCT |
| | | GCTGAACAACTTCTACCCTCGGGAAGCCAAGGTGCAGTGGAAGGTGG |
| | | ACAACGCTCTGCAGTCCGGCAACTCCCAAGAGTCTGTGACCGAGCAG |
| | | GACTCCAAGGACAGCACCTACAGCCTGTCCTCCACACTGACCCTGTCC |
| | | AAGGCCGACTACGAGAAGCACAAGGTGTACGCCTGCGAAGTGACCCA |
| | | TCAGGGCCTGTCTAGCCCTGTGACCAAGTCTTTCAACCGGGGCGAGTG |
| | | CTGATGAATTC |
| 255 | 11G06 VL_v3 | AAGCTTGCCGCCACCATGTCTGTGCCTACACAGGTTCTGGGACTGCTG |
| | | CTGCTGTGGCTGACCGACGCTAGATGCGAGACAACCGTGACTCAGTC |
| | | CCCTGCCTTCATGTCTGCTACCACCGGCGACAAAGTGACCATCTCCTGC |
| | | ATCACCTCCACCGACATCGACGACGACATGAACTGGTATCAGCAGAA |
| | | GCCCGGCGAGCCTCCTAAGCTGCTGATCTCTGAGGGCTCTACACTGAG |
| | | GCCTGGCGTGCCCTCCAGATTCTCCTCTTCTGGCTACGGCACCGACTTC |
| | | ACCTTCACCATCAACAACATCGAGTCCGAGGACGCCGCCTACTACTAC |
| | | TGCCTGCAGTCTGACAACCTGCCTCTGACCTTTGGCCAGGGCACCAAG |
| | | CTGGAAATCAAGCGGACAGTGGCCGCTCCTTCCGTGTTCATCTTCCCA |
| | | CCTTCCGACGAGCAGCTGAAGTCTGGCACAGCCTCTGTCGTGTGCCTG |
| | | CTGAACAACTTCTACCCTCGGGAAGCCAAGGTGCAGTGGAAGGTGGA |
| | | CAACGCTCTGCAGTCCGGCAACTCCCAAGAGTCTGTGACCGAGCAGG |
| | | ACTCCAAGGACAGCACCTACAGCCTGTCCTCCACACTGACCCTGTCCA |
| | | AGGCCGACTACGAGAAGCACAAGGTGTACGCCTGCGAAGTGACCCAT |
| | | CAGGGCCTGTCTAGCCCTGTGACCAAGTCTTTCAACCGGGGCGAGTGC |
| | | TGATGAATTC |
| 256 | 11G06 VL_v4 | AAGCTTGCCGCCACCATGTCTGTGCCTACACAGGTTCTGGGACTGCTG |
| | | CTGCTGTGGCTGACCGACGCTAGATGCGAGACAACCGTGACTCAGTC |
| | | CCCTGCCTTCATGTCTGCTACCCCTGGCGACAAAGTGACCATCTCCTGC |
| | | ATCACCTCCACCGACATCGACGACGACATGAACTGGTATCAGCAGAA |
| | | GCCCGGCGAGCCTCCTAAGCTGCTGATCTCTGAGGGCTCTACACTGAG |
| | | GCCTGGCGTGCCCTCCAGATTCTCCTCTTCTGGCTACGGCACCGACTTC |
| | | ACCTTCACCATCAACAACATCGAGTCCGAGGACGCCGCCTACTACTAC |
| | | TGCCTGCAGTCTGACAACCTGCCTCTGACCTTTGGCCAGGGCACCAAG |
| | | CTGGAAATCAAGCGGACAGTGGCCGCTCCTTCCGTGTTCATCTTCCCA |
| | | CCTTCCGACGAGCAGCTGAAGTCTGGCACAGCCTCTGTCGTGTGCCTG |
| | | CTGAACAACTTCTACCCTCGGGAAGCCAAGGTGCAGTGGAAGGTGGA |
| | | CAACGCTCTGCAGTCCGGCAACTCCCAAGAGTCTGTGACCGAGCAGG |
| | | ACTCCAAGGACAGCACCTACAGCCTGTCCTCCACACTGACCCTGTCCA |
| | | AGGCCGACTACGAGAAGCACAAGGTGTACGCCTGCGAAGTGACCCAT |
| | | CAGGGCCTGTCTAGCCCTGTGACCAAGTCTTTCAACCGGGGCGAGTGC |
| | | TGATGAATTC |
| 257 | 11G06 VH_v1 | AAGCTTGCCGCCACCATGGAATGGTCCTGGGTGTTCCTGTTCTTCCTGT |
| | | CCGTGACCACCGGCGTGCACTCTGAAGTTCAGCTGGTTCAGTCTGGCG |
| | | CCGAAGTGAAGAAACCTGGCTCCTCCGTGAAGGTGTCCTGCAAGGCT |
| | | TCCGGCTACCGGTTCTCCGACTACGAGATGCAGTGGGTCCGACAGGCT |
| | | CCTGGACAAGGCTTGGAATGGATGGGCGGCATCGATCCTGAGACAGG |
| | | CGGAATCGCTTACGCCCAGAAATTCCAGGGCAGAGTGACCCTGACCG |
| | | CCGACAAGTCTACCTCCACCGCCTACATGGAACTGTCCAGCCTGAGAT |
| | | CTGAGGACACCGCCGTGTACTACTGCACCCGGAAGCTGGACTTCGACT |
| | | ATTGGGGCCAGGGCACCCTGGTCACAGTGTCCTCTGCTTCTACCAAGG |
| | | GACCCAGCGTGTTCCCTCTGGCTCCTAGCTCTAAGTCCACCTCTGGCG |
| | | GAACAGCTGCTCTGGGCTGTCTGGTCAAGGACTACTTCCCTGAGCCTG |
| | | TGACCGTGTCCTGGAACTCTGGCGCTCTGACATCTGGCGTGCACACCT |
| | | TTCCAGCTGTGCTGCAGTCCTCCGGCCTGTACTCTCTGTCCTCTGTCGT |
| | | GACCGTGCCTTCCAGCTCTCTGGGAACCCAGACCTACATCTGCAATGT |
| | | GAACCACAAGCCTTCCAACACCAAGGTGGACAAGAAGGTGGAACCCA |
| | | AGTCCTGCGACAAGACCCACACCTGTCCTCCATGTCCTGCTCCAGAGT |
| | | CCACCAGAGGACCCTCCGTGTTTCTGTTCCCTCCAAAGCCTAAGGACA |
| | | CCCTGTACATCACCCGCGAGCCTGAAGTGACCTGCGTGGTGGTGGAT |
| | | GTGTCTCACGAGGACCCAGAAGTGAAGTTCAATTGGTACGTGGACGG |
| | | CGTGGAAGTGCACAACGCCAAGACCAAGCCTAGAGAGGAACAGTACA |
| | | ACTCCACCTACAGAGTGGTGTCCGTGCTGACCGTGCTGCACCAGGATT |
| | | GGCTGAACGGCAAAGAGTACAAGTGCAAGGTGTCCAACAAGGCCCTG |
| | | CCTGCTCCTATCGAAAAGACCATCTCCAAGGCCAAGGGCCAGCCTAGG |
| | | GAACCCCAGGTTTACACCTTGCCTCCATCTCGGGACGAGCTGACCAAG |
| | | AACCAGGTGTCCCTGACCTGTCTCGTGAAGGGCTTCTACCCCTCCGAT |
| | | ATCGCCGTGGAATGGGAGTCTAATGGCCAGCCAGAGAACAACTACAA |
| | | GACAACCCCTCCTGTGCTGGACTCCGACGGCTCATTCTTTCTGTACTCC |
| | | AAGCTGACAGTGGACAAGTCCAGATGGCAGCAGGGCAACGTGTTCTC |
| | | CTGCAGCGTGATGCACGAGGCCCTGCACAATCACTACACACAGAAGT |
| | | CCCTGTCTCTGTCCCCTGGCAAGTGATGAATTC |
| 258 | 11G06 VH v2 | AAGCTTGCCGCCACCATGGAATGGTCCTGGGTGTTCCTGTTCTTCCTGT |
| | | CCGTGACCACCGGCGTGCACTCTCAGGTTCAGTTGGTTCAGTCTGGCG |
| | | CCGAAGTGAAGAAACCCGGCTCCTCTGTGAAGGTGTCCTGCAAGGCT |
| | | TCTGGCTACCGGTTCTCCGACTACGAGATGCAGTGGGTCCGACAGGCT |
| | | CCTGGACAAGGCTTGGAATGGATGGGCGGCATCGATCCTGAGACAGG |
| | | CGGAATCGCTTACGCCCAGAAATTCCAGGGCAGAGTGACCCTGACCG |
| | | CCGACAAGTCTACCTCCACCGCCTACATGGAACTGTCCAGCCTGAGAT |
| | | CTGAGGACACCGCCGTGTACTACTGCACCCGGAAGCTGGACTTCGACT |
| | | ATTGGGGCCAGGGCACCCTGGTCACAGTGTCCTCTGCTTCTACCAAGG |
| | | GACCCAGCGTGTTCCCTCTGGCTCCTAGCTCTAAGTCCACCTCTGGCG |
| | | GAACAGCTGCTCTGGGCTGTCTGGTCAAGGACTACTTCCCTGAGCCTG |
| | | TGACCGTGTCCTGGAACTCTGGCGCTCTGACATCTGGCGTGCACACCT |
| | | TTCCAGCTGTGCTGCAGTCCTCCGGCCTGTACTCTCTGTCCTCTGTCGT |
| | | GACCGTGCCTTCCAGCTCTCTGGGAACCCAGACCTACATCTGCAATGT |
| | | GAACCACAAGCCTTCCAACACCAAGGTGGACAAGAAGGTGGAACCCA |
| | | AGTCCTGCGACAAGACCCACACCTGTCCTCCATGTCCTGCTCCAGAGT |
| | | CCACCAGAGGACCCTCCGTGTTTCTGTTCCCTCCAAAGCCTAAGGACA |
| | | CCCTGTACATCACCCGCGAGCCTGAAGTGACCTGCGTGGTGGTGGAT |
| | | GTGTCTCACGAGGACCCAGAAGTGAAGTTCAATTGGTACGTGGACGG |
| | | CGTGGAAGTGCACAACGCCAAGACCAAGCCTAGAGAGGAACAGTACA |
| | | ACTCCACCTACAGAGTGGTGTCCGTGCTGACCGTGCTGCACCAGGATT |
| | | GGCTGAACGGCAAAGAGTACAAGTGCAAGGTGTCCAACAAGGCCCTG |
| | | CCTGCTCCTATCGAAAAGACCATCTCCAAGGCCAAGGGCCAGCCTAGG |
| | | GAACCCCAGGTTTACACCTTGCCTCCATCTCGGGACGAGCTGACCAAG |
| | | AACCAGGTGTCCCTGACCTGTCTCGTGAAGGGCTTCTACCCCTCCGAT |
| | | ATCGCCGTGGAATGGGAGTCTAATGGCCAGCCAGAGAACAACTACAA |
| | | GACAACCCCTCCTGTGCTGGACTCCGACGGCTCATTCTTTCTGTACTCC |
| | | AAGCTGACAGTGGACAAGTCCAGATGGCAGCAGGGCAACGTGTTCTC |
| | | CTGCAGCGTGATGCACGAGGCCCTGCACAATCACTACACACAGAAGT |
| | | CCCTGTCTCTGTCCCCTGGCAAGTGATGAATTC |

A polynucleotide encoding an antibody described herein or a domain thereof can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light chain region and/or the variable heavy chain region of an antibody. The amplified nucleic acids can be cloned into vectors for expression in host cells and for further cloning, for example, to generate chimeric and humanized antibodies.

Polynucleotides provided herein can be, e.g., in the form of RNA or in the form of DNA. DNA includes cDNA, genomic DNA, and synthetic DNA, and DNA can be double-stranded or single-stranded. If single stranded, DNA can be the coding strand or non-coding (anti-sense) strand. In certain embodiments, the polynucleotide is a cDNA or a DNA lacking one more endogenous introns. In certain embodiments, a polynucleotide is a non-naturally occurring polynucleotide. In certain embodiments, a polynucleotide is recombinantly produced. In certain embodiments, the polynucleotides are isolated. In certain embodiments, the polynucleotides are substantially pure. In certain embodiments, a polynucleotide is purified from natural components.

### Antibody Production

Antibodies that immunospecifically bind to CD36 (e.g., human CD36) can be produced by any method known in the art for the synthesis of full length antibodies or antigen-binding fragments thereof, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In a certain aspect, provided herein is a method of making an antibody that immunospecifically binds to CD36 (e.g., human CD36) comprising culturing a cell or host cell described herein. In a certain aspect, provided herein is a method of making an antibody which immunospecifically binds to CD36 (e.g., human CD36) comprising expressing (e.g., recombinantly expressing) the antibody using a cell or host cell described herein (e.g., a cell or a host cell comprising polynucleotides encoding an antibody described herein). In a particular embodiment, the cell is an isolated cell. In a particular embodiment, the exogenous polynucleotides have been introduced into the cell. In a particular embodiment, the method further comprises the step of purifying the antibody obtained from the cell or host cell.

### Pharmaceutical Compositions

Provided herein are compositions comprising an anti-CD36 antibody described herein having the desired degree of purity in a physiologically acceptable carrier, excipient or stabilizer (Remington's Pharmaceutical Sciences (1990) Mack Publishing Co., Easton, PA). Acceptable carriers, excipients, or stabilizers are nontoxic to recipients at the dosages and concentrations employed.

In various embodiments, compositions comprising an anti-CD36 antibody are provided in formulations with a pharmaceutically acceptable carrier (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)).

Pharmaceutical compositions described herein can be useful in blocking CD36 activity. Pharmaceutical compositions described herein can be useful in treating a condition such as cancer. Examples of cancer that can be treated in accordance with the methods described herein include, but are not limited to, solid cancers and metastases thereof. In some embodiments, the pharmaceutical compositions described herein can be useful in treating an oral squamous cell carcinoma, head and neck cancer, esophageal cancer, gastric cancer, ovarian cancer, cervical cancer, lung cancer, breast cancer, colon cancer, renal cancer, prostate cancer, sarcoma, e.g., liposarcoma, melanoma, leukemia, or lymphoma. In some embodiments, the pharmaceutical compositions described herein can be useful in treating metastatic oral squamous cell carcinoma, metastatic head and neck cancer, metastatic esophageal cancer, metastatic gastric cancer, metastatic ovarian cancer, metastatic cervical cancer, metastatic lung cancer, metastatic breast cancer, metastatic colon cancer, metastatic renal cancer, metastatic prostate cancer, metastatic sarcoma, e.g., liposarcoma, metastatic melanoma, metastatic leukemia, or metastatic lymphoma. In some embodiments, the pharmaceutical compositions described herein can be useful in treating both the primary tumor and metastases developed from an oral squamous cell carcinoma, head and neck cancer, esophageal cancer, gastric cancer, ovarian cancer, cervical cancer, lung cancer, breast cancer, colon cancer, renal cancer, prostate cancer, sarcoma, e.g., liposarcoma, melanoma, leukemia, or lymphoma.

The pharmaceutical compositions described herein are in one embodiment for use as a medicament. The pharmaceutical compositions described herein are in one embodiment for use as a diagnostic, e.g., to detect the presence of CD36 in a sample obtained from a patient (e.g., a human patient).

The compositions to be used for in vivo administration can be sterile. This is readily accomplished by filtration through, e.g., sterile filtration membranes.

In some embodiments, the pharmaceutical compositions comprise an isolated antibody. In some embodiments, the pharmaceutical compositions are substantially free of other antibodies.

In some embodiments, pharmaceutical compositions are provided, wherein the pharmaceutical composition comprises anti-CD36 antibodies described herein and a pharmaceutically acceptable carrier. In some embodiments, pharmaceutical compositions are provided, wherein the pharmaceutical composition comprises afucosylated anti-CD36 antibodies described herein and a pharmaceutically acceptable carrier.

Antibodies with Fc regions having reduced fucose content in glycan moieties may exhibit higher ADCC activity compared to a fully fucosylated antibody because of an increased affinity for Fc receptors, such as, e.g., FcγRIIIA (Niwa R et al., Clinical Cancer Research 11(6):2327-36 (2005)). In some embodiments, the CD36 antibody has enhanced ADCC activity in vitro compared to fucosylated CD36 antibodies having the same amino acid sequence. In specific embodiments, such pharmaceutical composition comprises afucosylated anti-CD36 antibodies, wherein at least 50% of the antibodies in the composition are afucosylated. In specific embodiments, such pharmaceutical composition comprises afucosylated anti-CD36 antibodies, wherein at least 60% of the antibodies in the composition are afucosylated. In specific embodiments, such pharmaceutical composition comprises afucosylated anti-CD36 antibodies, wherein at least 70% of the antibodies in the composition are afucosylated. In specific embodiments, such pharmaceutical composition comprises afucosylated anti-CD36 antibodies, wherein at least 80% of the antibodies in the composition are afucosylated. In specific embodiments, such pharmaceutical composition comprises afucosylated anti-CD36 antibodies, wherein at least 85% of the antibodies in the composition are afucosylated. In specific embodiments, such pharmaceutical composition comprises afucosylated anti-CD36 antibodies, wherein at least 90% of the antibodies in the composition are afucosylated. In specific embodiments, such pharmaceutical composition comprises afucosylated anti-CD36 antibodies, wherein at least 95% of the antibodies in the composition are afucosylated. In specific embodiments, such pharmaceutical composition comprises afucosylated anti-CD36 antibodies, wherein at least 96% of the antibodies in the composition are afucosylated. In specific embodiments, such pharmaceutical composition comprises afucosylated anti-CD36 antibodies, wherein at least 97% of the antibodies in the composition are afucosylated. In specific embodiments, such pharmaceutical composition comprises afucosylated anti-CD36 antibodies, wherein at least 98% of the antibodies in the composition are afucosylated. In specific embodiments, such pharmaceutical composition comprises afucosylated anti-CD36 antibodies, wherein at least 99% of the antibodies in the composition are afucosylated. In specific embodiments, such pharmaceutical composition comprises afucosylated anti-CD36 antibodies wherein fucose is undetectable in the composition.

### Methods of the Disclosure

In some embodiments, the present invention provides methods of treating cancer in a mammal using a combination of an anti-CD36 antibody and a second therapy. In some embodiments, the cancer is selected from the group consisting of oral squamous cell carcinoma, head and neck cancer, esophageal cancer, gastric cancer, ovarian cancer, cervical cancer, lung cancer, breast cancer, colon cancer, renal cancer, prostate cancer, sarcoma, e.g., liposarcoma, melanoma, leukemia, and lymphoma. In embodiments, the cancer is oral squamous cell carcinoma. In some embodiments, the cancer is ovarian cancer. In other embodiments, the cancer is melanoma. In a further embodiment, the cancer is any cancer disclosed herein. In some embodiments, the cancer is metastatic cancer. In some embodiments, the cancer is both a primary tumor and metastatic cancer. In some embodiments, the mammal is a human.

In some embodiments, the anti-CD36 antibody is a full length antibody, a single chain antibody, or a scFv, Fab or F(ab′)₂ fragment. In one embodiment, the anti-CD36 antibody is a full length antibody. In an embodiment, the anti-CD36 antibody is a humanized antibody. In certain embodiments, the anti-CD36 antibody is an antibody disclosed herein. In certain embodiments, the anti-CD36 antibody is a commercial anti-CD36 antibody, such as the antibody JC63.1.

In some embodiments, the second therapy is an immunotherapy. In one embodiment, the immunotherapy is a PD-1 inhibitor. In an embodiment, the PD-1 inhibitor is an anti-PD-1 antibody. In one embodiment, the anti-PD-1 antibody is pembrolizumab (KEYTRUDA; MK-3475), pidilizumab (CT-011), or nivolumab (OPDIVO; BMS-936558). In an embodiment, the immunotherapy is a PD-L1 inhibitor. In one embodiment, PD-L1 inhibitor is an anti-PD-L1 antibody. In an embodiment, the anti-PD-L1 antibody is atezolizumab (Tecentriq or RG7446), durvalumab (Imfinzi or MEDI4736), avelumab (Bavencio) or BMS-936559 In one embodiment, the immunotherapy is a CTLA-4 inhibitor. In an embodiment, the CTLA-4 inhibitor is an anti-CTLA-4 antibody. In one embodiment, the anti-CTLA-4 antibody is ipilimumab or an antigen-binding fragment thereof.

In one embodiment, the second therapy is a chemotherapeutic agent. In an embodiment, the chemotherapeutic agent is cisplatin. In certain embodiments, the chemotherapeutic agent comprises one of the anti-cancer drugs or anti-cancer drug combinations listed in Table 5.

TABLE 5
Chemotherapeutic Agents

| | | Abraxane (Paclitaxel | |
|---|---|---|---|
| | | Albumin-stabilized | |
| | Abiraterone | Nanoparticle | |
| Abemaciclib | Acetate | Formulation) | ABVD |
| ABVE | ABVE-PC | AC | Acalabrutinib |
| AC-T | Actemra | Adcetris (Brentuximab | ADE |
| | (Tocilizumab) | Vedotin) | |
| Ado-Trastuzumab | Adriamycin | Afatinib Dimaleate | Afinitor |
| Emtansine | (Doxorubicin | | (Everolimus) |
| | Hydrochloride) | | |
| Akynzeo | Aldara | Aldesleukin | Alecensa |
| (Netupitant and | (Imiquimod) | | (Alectinib) |
| Palonosetron | | | |
| Hydrochloride) | | | |
| Alectinib | Alemtuzumab | Alimta (Pemetrexed | Aliqopa |
| | | Disodium) | (Copanlisib |
| | | | Hydrochloride) |
| Alkeran for | Alkeran Tablets | Aloxi (Palonosetron | Alunbrig |
| Injection | (Melphalan) | Hydrochloride) | (Brigatinib) |
| (Melphalan | | | |
| Hydrochloride) | | | |
| Ameluz | Amifostine | Aminolevulinic Acid | Anastrozole |
| (Aminolevulinic | | | |
| Acid) | | | |
| Apalutamide | Aprepitant | Aranesp (Darbepoetin | Aredia |
| | | Alfa) | (Pamidronate |
| | | | Disodium) |
| Arimidex | Aromasin | Arranon (Nelarabine) | Arsenic Trioxide |
| (Anastrozole) | (Exemestane) | | |
| Arzerra | Asparaginase | Atezolizumab | Avastin |
| (Ofatumumab) | Erwinia | | (Bevacizumab) |
| | chrysanthemi | | |
| Avelumab | Axicabtagene | Axitinib | Azacitidine |
| | Ciloleucel | | |
| Azedra | Bavencio | BEACOPP | Beleodaq |
| (Iobenguane I 131) | (Avelumab) | | (Belinostat) |
| Belinostat | Bendamustine | Bendeka (Bendamustine | BEP |
| | Hydrochloride | Hydrochloride) | |
| Besponsa | Bevacizumab | Bexarotene | Bicalutamide |
| (Inotuzumab | | | |
| Ozogamicin) | | | |
| BiCNU | Binimetinib | Bleomycin | Blinatumomab |
| (Carmustine) | | | |
| Blincyto | Bortezomib | Bosulif (Bosutinib) | Bosutinib |
| (Blinatumomab) | | | |
| Braftovi | Brentuximab | Brigatinib | BuMel |
| (Encorafenib) | Vedotin | | |
| Busulfan | Busulfex | Cabazitaxel | Cabometyx |
| | (Busulfan) | | (Cabozantinib-S- |
| | | | Malate) |
| Cabozantinib-S- | CAF | Calquence | Campath |
| Malate | | (Acalabrutinib) | (Alemtuzumab) |
| Camptosar | Capecitabine | CAPOX | Carac |
| (Irinotecan | | | (Fluorouracil-- |
| Hydrochloride) | | | Topical) |
| Carboplatin | CARBOPLATIN- | Carfilzomib | Carmustine |
| | TAXOL | | |
| Carmustine | Casodex | CEM | Cemiplimab-rwlc |
| Implant | (Bicalutamide) | | |
| Ceritinib | Cerubidine | Cervarix (Recombinant | Cetuximab |
| | (Daunorubicin | HPV Bivalent Vaccine) | |
| | Hydrochloride) | | |
| CEV | Chlorambucil | CHLORAMBUCIL- | CHOP |
| | | PREDNISONE | |
| Cisplatin | Cladribine | Clofarabine | Clolar |
| | | | (Clofarabine) |
| CMF | Cobimetinib | Cometriq (Cabozantinib- | Copanlisib |
| | | S-Malate) | Hydrochloride |
| COPDAC | Copiktra | COPP | COPP-ABV |
| | (Duvelisib) | | |
| Cosmegen | Cotellic | Crizotinib | CVP |
| (Dactinomycin) | (Cobimetinib) | | |
| Cyclophosphamide | Cyramza | Cytarabine | Cytarabine |
| | (Ramucirumab) | | Liposome |
| Cytosar-U | Dabrafenib | Dacarbazine | Dacogen |
| (Cytarabine) | | | (Decitabine) |
| Dacomitinib | Dactinomycin | Daratumumab | Darbepoetin Alfa |
| Darzalex | Dasatinib | Daunorubicin | Daunorubicin |
| (Daratumumab) | | Hydrochloride | Hydrochloride |
| | | | and Cytarabine |
| | | | Liposome |
| Decitabine | Defibrotide | Defitelio (Defibrotide | Degarelix |
| | Sodium | Sodium) | |
| Denileukin | Denosumab | DepoCyt (Cytarabine | Dexamethasone |
| Diftitox | | Liposome) | |
| Dexrazoxane | Dinutuximab | Docetaxel | Doxil |
| Hydrochloride | | | (Doxorubicin |
| | | | Hydrochloride |
| | | | Liposome) |
| Doxorubicin | Doxorubicin | Dox-SL (Doxorubicin | Durvalumab |
| Hydrochloride | Hydrochloride | Hydrochloride | |
| | Liposome | Liposome) | |
| Duvelisib | Efudex | Eligard (Leuprolide | Elitek |
| | (Fluorouracil-- | Acetate) | (Rasburicase) |
| | Topical) | | |
| Ellence | Elotuzumab | Eloxatin (Oxaliplatin) | Eltrombopag |
| (Epirubicin | | | Olamine |
| Hydrochloride) | | | |
| Emend | Empliciti | Enasidenib Mesylate | Encorafenib |
| (Aprepitant) | (Elotuzumab) | | |
| Enzalutamide | Epirubicin | EPOCH | Epoetin Alfa |
| | Hydrochloride | | |
| Epogen (Epoetin | Erbitux | Eribulin Mesylate | Erivedge |
| Alfa) | (Cetuximab) | | (Vismodegib) |
| Erleada | Erlotinib | Erwinaze (Asparaginase | Ethyol |
| (Apalutamide) | Hydrochloride | Erwinia chrysanthemi) | (Amifostine) |
| Etopophos | Etoposide | Etoposide Phosphate | Evacet |
| (Etoposide | | | (Doxorubicin |
| Phosphate) | | | Hydrochloride |
| | | | Liposome) |
| Everolimus | Evista (Raloxifene | Evomela (Melphalan | Exemestane |
| | Hydrochloride) | Hydrochloride) | |
| 5-FU (Fluorouracil | 5-FU | Fareston (Toremifene) | Farydak |
| Injection) | (Fluorouracil-- | | (Panobinostat) |
| | Topical) | | |
| Faslodex | FEC | Femara (Letrozole) | Filgrastim |
| (Fulvestrant) | | | |
| Firmagon | Fludarabine | Fluoroplex (Fluorouracil-- | Fluorouracil |
| (Degarelix) | Phosphate | Topical) | Injection |
| Fluorouracil-- | Flutamide | FOLFIRI | FOLFIRI- |
| Topical | | | BEVACIZUMAB |
| FOLFIRI- | FOLFIRINOX | FOLFOX | Folotyn |
| CETUXIMAB | | | (Pralatrexate) |
| Fostamatinib | FU-LV | Fulvestrant | Fusilev |
| Disodium | | | (Leucovorin |
| | | | Calcium) |
| Gardasil | Gardasil 9 | Gazyva (Obinutuzumab) | Gefitinib |
| (Recombinant | (Recombinant | | |
| HPV Quadrivalent | HPV Nonavalent | | |
| Vaccine) | Vaccine) | | |
| Gemcitabine | GEMCITABINE- | GEMCITABINE- | Gemtuzumab |
| Hydrochloride | CISPLATIN | OXALIPLATIN | Ozogamicin |
| Gemzar | Gilotrif (Afatinib | Gleevec (Imatinib | Gliadel Wafer |
| (Gemcitabine | Dimaleate) | Mesylate) | (Carmustine |
| Hydrochloride) | | | Implant) |
| Glucarpidase | Goserelin Acetate | Granisetron | Granisetron |
| | | | Hydrochloride |
| Granix | Halaven (Eribulin | Hemangeol (Propranolol | Herceptin |
| (Filgrastim) | Mesylate) | Hydrochloride) | (Trastuzumab) |
| HPV Bivalent | HPV Nonavalent | HPV Quadrivalent | Hycamtin |
| Vaccine, | Vaccine, | Vaccine, Recombinant | (Topotecan |
| Recombinant | Recombinant | | Hydrochloride) |
| Hydrea | Hydroxyurea | Hyper-CVAD | Ibrance |
| (Hydroxyurea) | | | (Palbociclib) |
| Ibritumomab | Ibrutinib | ICE | Iclusig (Ponatinib |
| Tiuxetan | | | Hydrochloride) |
| Idarubicin | Idelalisib | Idhifa (Enasidenib | Ifex (Ifosfamide) |
| Hydrochloride | | Mesylate) | |
| Ifosfamide | IL-2 | Imatinib Mesylate | Imbruvica |
| | (Aldesleukin) | | (Ibrutinib) |
| Imfinzi | Imiquimod | Imlygic (Talimogene | Inlyta (Axitinib) |
| (Durvalumab) | | Laherparepvec) | |
| Inotuzumab | Interferon Alfa- | Interleukin-2 | Intron A |
| Ozogamicin | 2b, Recombinant | (Aldesleukin) | (Recombinant |
| | | | Interferon Alfa- |
| | | | 2b) |
| Iobenguane I 131 | Ipilimumab | Iressa (Gefitinib) | Irinotecan |
| | | | Hydrochloride |
| Irinotecan | Istodax | Ivosidenib | Ixabepilone |
| Hydrochloride | (Romidepsin) | | |
| Liposome | | | |
| Ixazomib Citrate | Ixempra | Jakafi (Ruxolitinib | JEB |
| | (Ixabepilone) | Phosphate) | |
| Jevtana | Kadcyla (Ado- | Kepivance (Palifermin) | Keytruda |
| (Cabazitaxel) | Trastuzumab | | (Pembrolizumab) |
| | Emtansine) | | |
| Kisqali | Kymriah | Kyprolis (Carfilzomib) | Lanreotide |
| (Ribociclib) | (Tisagenlecleucel) | | Acetate |
| Lapatinib | Larotrectinib | Lartruvo (Olaratumab) | Lenalidomide |
| Ditosylate | Sulfate | | |
| Lenvatinib | Lenvima | Letrozole | Leucovorin |
| Mesylate | (Lenvatinib | | Calcium |
| | Mesylate) | | |
| Leukeran | Leuprolide | Levulan | Libtayo |
| (Chlorambucil) | Acetate | Kerastik (Aminolevulinic | (Cemiplimab- |
| | | Acid) | rwlc) |
| LipoDox | Lomustine | Lonsurf (Trifluridine and | Lorbrena |
| (Doxorubicin | | Tipiracil Hydrochloride) | (Lorlatinib) |
| Hydrochloride | | | |
| Liposome) | | | |
| Lorlatinib | Lumoxiti | Lupron (Leuprolide | Lupron Depot |
| | (Moxetumomab | Acetate) | (Leuprolide |
| | Pasudotox-tdfk) | | Acetate) |
| Lutathera | Lutetium (Lu 177- | Lynparza (Olaparib) | Marqibo |
| (Lutetium Lu 177- | Dotatate) | | (Vincristine |
| Dotatate) | | | Sulfate |
| | | | Liposome) |
| Matulane | Mechlorethamine | Megestrol Acetate | Mekinist |
| (Procarbazine | Hydrochloride | | (Trametinib) |
| Hydrochloride) | | | |
| Mektovi | Melphalan | Melphalan | Mercaptopurine |
| (Binimetinib) | | Hydrochloride | |
| Mesna | Mesnex (Mesna) | Methotrexate | Methylnaltrexone |
| | | | Bromide |
| Midostaurin | Mitomycin C | Mitoxantrone | Mogamulizumab- |
| | | Hydrochloride | kpkc |
| Moxetumomab | Mozobil | Mustargen | MVAC |
| Pasudotox-tdfk | (Plerixafor) | (Mechlorethamine | |
| | | Hydrochloride) | |
| Myleran | Mylotarg | Nanoparticle Paclitaxel | Navelbine |
| (Busulfan) | (Gemtuzumab | (Paclitaxel Albumin- | (Vinorelbine |
| | Ozogamicin) | stabilized Nanoparticle | Tartrate) |
| | | Formulation) | |
| Necitumumab | Nelarabine | Neratinib Maleate | Nerlynx |
| | | | (Neratinib |
| | | | Maleate) |
| Netupitant and | Neulasta | Neupogen (Filgrastim) | Nexavar |
| Palonosetron | (Pegfilgrastim) | | (Sorafenib |
| Hydrochloride | | | Tosylate) |
| Nilandron | Nilotinib | Nilutamide | Ninlaro |
| (Nilutamide) | | | (Ixazomib |
| | | | Citrate) |
| Niraparib Tosylate | Nivolumab | Nplate (Romiplostim) | Obinutuzumab |
| Monohydrate | | | |
| Odomzo | OEPA | Ofatumumab | OFF |
| (Sonidegib) | | | |
| Olaparib | Olaratumab | Omacetaxine | Oncaspar |
| | | Mepesuccinate | (Pegaspargase) |
| Ondansetron | Onivyde | Ontak (Denileukin | Opdivo |
| Hydrochloride | (Irinotecan | Diftitox) | (Nivolumab) |
| | Hydrochloride | | |
| | Liposome) | | |
| OPPA | Osimertinib | Oxaliplatin | Paclitaxel |
| Paclitaxel | PAD | Palbociclib | Palifermin |
| Albumin-stabilized | | | |
| Nanoparticle | | | |
| Formulation | | | |
| Palonosetron | Palonosetron | Pamidronate Disodium | Panitumumab |
| Hydrochloride | Hydrochloride | | |
| | and Netupitant | | |
| Panobinostat | Pazopanib | PCV | PEB |
| | Hydrochloride | | |
| Pegaspargase | Pegfilgrastim | Peginterferon Alfa-2b | PEG-Intron |
| | | | (Peginterferon |
| | | | Alfa-2b) |
| Pembrolizumab | Pemetrexed | Perjeta (Pertuzumab) | Pertuzumab |
| | Disodium | | |
| Plerixafor | Pomalidomide | Pomalyst | Ponatinib |
| | | (Pomalidomide) | Hydrochloride |
| Portrazza | Poteligeo | Pralatrexate | Prednisone |
| (Necitumumab) | (Mogamulizumab- | | |
| | kpkc) | | |
| Procarbazine | Procrit (Epoetin | Proleukin (Aldesleukin) | Prolia |
| Hydrochloride | Alfa) | | (Denosumab) |
| Promacta | Propranolol | Provenge (Sipuleucel-T) | Purinethol |
| (Eltrombopag | Hydrochloride | | (Mercaptopurine) |
| Olamine) | | | |
| Purixan | Radium 223 | Raloxifene | Ramucirumab |
| (Mercaptopurine) | Dichloride | Hydrochloride | |
| Rasburicase | R-CHOP | R-CVP | Recombinant |
| | | | Human |
| | | | Papillomavirus |
| | | | (HPV) Bivalent |
| | | | Vaccine |
| Recombinant | Recombinant | Recombinant Interferon | Regorafenib |
| Human | Human | Alfa-2b | |
| Papillomavirus | Papillomavirus | | |
| (HPV) Nonavalent | (HPV) | | |
| Vaccine | Quadrivalent | | |
| | Vaccine | | |
| Relistor | R-EPOCH | Retacrit (Epoetin Alfa) | Revlimid |
| (Methylnaltrexone | | | (Lenalidomide) |
| Bromide) | | | |
| Rheumatrex | Ribociclib | R-ICE | Rituxan |
| (Methotrexate) | | | (Rituximab) |
| Rituxan Hycela | Rituximab | Rituximab and | Rolapitant |
| (Rituximab and | | Hyaluronidase Human | Hydrochloride |
| Hyaluronidase | | | |
| Human) | | | |
| Romidepsin | Romiplostim | Rubidomycin | Rubraca |
| | | (Daunorubicin | (Rucaparib |
| | | Hydrochloride) | Camsylate) |
| Rucaparib | Ruxolitinib | Rydapt (Midostaurin) | Sancuso |
| Camsylate | Phosphate | | (Granisetron) |
| Sclerosol | Siltuximab | Sipuleucel-T | Somatuline Depot |
| Intrapleural | | | (Lanreotide |
| Aerosol (Talc) | | | Acetate) |
| Sonidegib | Sorafenib | Sprycel (Dasatinib) | STANFORD V |
| | Tosylate | | |
| Sterile Talc | Steritalc (Talc) | Stivarga (Regorafenib) | Sunitinib Malate |
| Powder (Talc) | | | |
| Sustol | Sutent (Sunitinib | Sylatron (Peginterferon | Sylvant |
| (Granisetron) | Malate) | Alfa-2b) | (Siltuximab) |
| Synribo | Tabloid | TAC | Tafinlar |
| (Omacetaxine | (Thioguanine) | | (Dabrafenib) |
| Mepesuccinate) | | | |
| Tagrisso | Talc | Talimogene | Tamoxifen |
| (Osimertinib) | | Laherparepvec | Citrate |
| Tarabine PFS | Tarceva (Erlotinib | Targretin (Bexarotene) | Tasigna |
| (Cytarabine) | Hydrochloride) | | (Nilotinib) |
| Tavalisse | Taxol (Paclitaxel) | Taxotere (Docetaxel) | Tecentriq |
| (Fostamatinib | | | (Atezolizumab) |
| Disodium) | | | |
| Temodar | Temozolomide | Temsirolimus | Thalidomide |
| (Temozolomide) | | | |
| Thalomid | Thioguanine | Thiotepa | Tibsovo |
| (Thalidomide) | | | (Ivosidenib) |
| Tisagenlecleucel | Tocilizumab | Tolak (Fluorouracil-- | Topotecan |
| | | Topical) | Hydrochloride |
| Toremifene | Torisel | Totect (Dexrazoxane | TPF |
| | (Temsirolimus) | Hydrochloride) | |
| Trabectedin | Trametinib | Trastuzumab | Treanda |
| | | | (Bendamustine |
| | | | Hydrochloride) |
| Trexall | Trifluridine and | Trisenox (Arsenic | Tykerb (Lapatinib |
| (Methotrexate) | Tipiracil | Trioxide) | Ditosylate) |
| | Hydrochloride | | |
| Unituxin | Uridine Triacetate | VAC | Valrubicin |
| (Dinutuximab) | | | |
| Valstar | Vandetanib | VAMP | Varubi |
| (Valrubicin) | | | (Rolapitant |
| | | | Hydrochloride) |
| Vectibix | VeIP | Velcade (Bortezomib) | Vemurafenib |
| (Panitumumab) | | | |
| Venclexta | Venetoclax | Verzenio (Abemaciclib) | Vidaza |
| (Venetoclax) | | | (Azacitidine) |
| Vinblastine Sulfate | Vincristine | Vincristine Sulfate | Vinorelbine |
| | Sulfate | Liposome | Tartrate |
| VIP | Vismodegib | Vistogard (Uridine | Vitrakvi |
| | | Triacetate) | (Larotrectinib |
| | | | Sulfate) |
| Vizimpro | Voraxaze | Vorinostat | Votrient |
| (Dacomitinib) | (Glucarpidase) | | (Pazopanib |
| | | | Hydrochloride) |
| Vyxeos | Xalkori | Xeloda (Capecitabine) | XELIRI |
| (Daunorubicin | (Crizotinib) | | |
| Hydrochloride and | | | |
| Cytarabine | | | |
| Liposome) | | | |
| XELOX | Xgeva | Xofigo (Radium 223 | Xtandi |
| | (Denosumab) | Dichloride) | (Enzalutamide) |
| Yervoy | Yescarta | Yondelis (Trabectedin) | Zaltrap (Ziv- |
| (Ipilimumab) | (Axicabtagene | | Aflibercept) |
| | Ciloleucel) | | |
| Zarxio (Filgrastim) | Zejula (Niraparib | Zelboraf (Vemurafenib) | Zevalin |
| | Tosylate | | (Ibritumomab |
| | Monohydrate) | | Tiuxetan) |
| Zinecard | Ziv-Aflibercept | Zofran (Ondansetron | Zoladex |
| (Dexrazoxane | | Hydrochloride) | (Goserelin |
| Hydrochloride) | | | Acetate) |
| Zoledronic Acid | Zolinza | Zometa (Zoledronic | Zydelig |
| | (Vorinostat) | Acid) | (Idelalisib) |
| Zykadia | Zytiga | | |
| (Ceritinib) | (Abiraterone | | |
| | Acetate) | | |

In some embodiments, the present invention provides methods of treating cancer in a mammal using a combination of an anti-CD36 antibody and an anti-PD-1 antibody. In some embodiments, the cancer is selected from the group consisting of oral squamous cell carcinoma, head and neck cancer, esophageal cancer, gastric cancer, ovarian cancer, cervical cancer, lung cancer, breast cancer, colon cancer, renal cancer, prostate cancer, sarcoma, e.g., liposarcoma, melanoma, leukemia, and lymphoma. In some embodiments, the cancer is oral squamous cell carcinoma. In some embodiments, the cancer is ovarian cancer. In other embodiments, the cancer is melanoma. In a further embodiment, the cancer is any other cancer disclosed herein. In one embodiment, the cancer is metastatic cancer. In some embodiments, the cancer is both a primary tumor and a metastatic cancer. In some embodiments, the anti-CD36 antibody is a full length antibody, a single chain antibody, or a scFv, Fab or F(ab′)₂ fragment. In one embodiment, the anti-CD36 antibody is a full length antibody. In an embodiment, the anti-CD36 antibody is a humanized antibody. In certain embodiments, the anti-CD36 antibody is an antibody disclosed herein. In certain embodiments, the anti-CD36 antibody is a commercial anti-CD36 antibody such as the antibody JC63.1. In one embodiment, the anti-PD-1 antibody is pembrolizumab (KEYTRUDA; MK-3475), pidilizumab (CT-011), or nivolumab (OPDIVO; BMS-936558).

Examples of cancers and/or malignant tumors that may be treated using the methods of the invention, include liver cancer, hepatocellular carcinoma (HCC), bone cancer, pancreatic cancer, skin cancer, oral cancer, cancer of the head or neck, breast cancer, lung cancer, small cell lung cancer, NSCLC, cutaneous or intraocular malignant melanoma, Merkel cell carcinoma (MCC), cutaneous squamous cell carcinoma (cSCC), renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, squamous cell carcinoma of the head and neck (SCCHN), non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue (e.g., liposarcoma), cancer of the urethra, cancer of the penis, solid tumors of childhood, lymphocytic lymphoma, cancer of the bladder, urothelial carcinoma, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, and any combinations of said cancers. The present invention is also applicable to treatment of metastatic cancers. In embodiments, the cancer is oral squamous cell carcinoma. In some embodiments, the cancer is ovarian cancer. In other embodiments, the cancer is melanoma.

In particular embodiments, the methods disclosed herein reduce the size of a primary tumor within a treated patient. Methods that may be used to measure the size of a primary tumor include physical measurement (e.g., of diameter, weight, or number of cells), IVIS imaging, and H&E staining as part of immunohistochemical analysis. In some embodiments, the methods reduce the size of the primary tumor by at least about 10%, at least about 20%, at least about 30%, at least about 40%, at least about 50%, at least about 60%, at least about 70%, at least about 80%, or at least about 90% in size.

In particular embodiments, the methods disclosed herein reduce the size of at least one metastatic tumor within a treated patient. Methods that may be used to measure the size of a metastatic tumor include physical measurement (e.g., of diameter, weight, or number of cells), IVIS imaging, and H&E staining as part of immunohistochemical analysis. In some embodiments, the methods reduce the size of the one or more metastatic tumors by at least about 10%, at least about 20%, at least about 30%, at least about 40%, at least about 50%, at least about 60%, at least about 70%, at least about 80%, or at least about 90% in size.

In particular embodiments, the methods disclosed herein prevent the growth in size of a primary tumor within treated patients, relative to patients administered a control treatment. Methods that may be used to measure the size of a primary tumor include physical measurement (e.g., of diameter, weight, or number of cells), IVIS imaging, and H&E staining as part of immunohistochemical analysis. In some embodiments, the methods prevent the growth in size of the primary tumor by at least about 10%, at least about 20%, at least about 30%, at least about 40%, at least about 50%, at least about 60%, at least about 70%, at least about 80%, or at least about 90%, relative to tumors in patients administered a control treatment.

In particular embodiments, the methods disclosed herein prevent the growth in size of one or more metastatic tumors within treated patients, relative to patients administered a control treatment. Methods that may be used to measure the size of a metastatic tumor include physical measurement (e.g., of diameter, weight, or number of cells), IVIS imaging, and H&E staining as part of immunohistochemical analysis. In some embodiments, the methods prevent the growth in size of the one or more metastatic tumors by at least about 10%, at least about 20%, at least about 30%, at least about 40%, at least about 50%, at least about 60%, at least about 70%, at least about 80%, or at least about 90%, relative to metastatic tumors in patients administered a control treatment. In some embodiments, the methods increase the percentage of metastatic tumors that are limited in size to only a few cells, relative to the percentage of metastatic tumors that are limited in size to only a few cells in patients administered a control treatment. In some embodiments, the methods increase the percentage of metastatic tumors that are limited in size to only a few cells by at least about 10%, at least about 20%, at least about 30%, at least about 40%, at least about 50%, at least about 60%, at least about 70%, at least about 80%, or at least about 90%, relative to the percentage of metastatic tumors that are limited in size to only a few cells in patients administered a control treatment. In some embodiments, the methods reduce the percentage of metastatic tumors that are large (i.e., diameter>5 mm) or medium (i.e., diameter between 1 and 2 mm), relative to the percentage of metastatic tumors that are large or medium in patients administered a control treatment. In some embodiments, the methods reduce the percentage of metastatic tumors that are large or medium by at least about 10%, at least about 20%, at least about 30%, at least about 40%, at least about 50%, at least about 60%, at least about 70%, at least about 80%, or at least about 90%, relative to the percentage of metastatic tumors that are large or medium in patients administered a control treatment.

In particular embodiments, the methods disclosed herein reduce the number of metastatic tumors within treated patients, relative to patients administered a control treatment. Methods that may be used to measure the number of a metastatic tumor include physical examination (e.g., counting the number of tumors), IVIS imaging, and H&E staining as part of immunohistochemical analysis. In some embodiments, the methods reduce the number of metastatic tumors by at least about 10%, at least about 20%, at least about 30%, at least about 40%, at least about 50%, at least about 60%, at least about 70%, at least about 80%, or at least about 90%, relative to tumors in patients administered a control treatment.

In embodiments, the antibodies can be administered systemically, for instance, intraperitoneally, and can be in the form of an appropriate suspension, for instance an aqueous suspension, in water or another appropriate liquid such as saline solution.

For administration of the antibodies, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. In certain embodiments, the antibodies are administered at a flat or fixed dose. In embodiments, the antibodies are administered at any dosage described for the antibody in the art.

### Anti-PD-1 and Anti-PD-L1 Antibodies

As used herein, the terms “Programmed Death 1,” “Programmed Cell Death 1,” “Protein PD-1,” “PD-1,” “PD1,” “PDCD1,” “hPD-1” and “hPD-I” are used interchangeably, and include variants, isoforms, species homologs of human PD-1, and analogs having at least one common epitope with PD-1. The complete PD-1 sequence can be found under GenBank Accession No. U64863.

Programmed Cell Death 1 (PD-1) is a cell surface signaling receptor that plays a critical role in the regulation of T cell activation and tolerance (Keir M. E., et al., Annu. Rev. Immunol. 2008; 26:677-704). It is a type I transmembrane protein and together with BTLA, CTLA-4, ICOS and CD28, comprise the CD28 family of T cell co-stimulatory receptors. PD-1 is primarily expressed on activated T cells, B cells, and myeloid cells (Dong H., et al., Nat. Med. 1999; 5:1365-1369; Agata et al., supra; Okazaki et al. (2002) Curr. Opin. Immunol. 14: 391779-82; Bennett et al. (2003) J Immunol 170:711-8). It is also expressed on natural killer (NK) cells (Terme M., et al., Cancer Res. 2011; 71:5393-5399). Binding of PD-1 by its ligands, PD-L1 and PD-L2, results in phosphorylation of the tyrosine residue in the proximal intracellular immune receptor tyrosine inhibitory domain, followed by recruitment of the phosphatase SHP-2, eventually resulting in down-regulation of T cell activation. One important role of PD-1 is to limit the activity of T cells in peripheral tissues at the time of an inflammatory response to infection, thus limiting the development of autoimmunity (Pardoll D. M., Nat. Rev. Cancer 2012; 12:252-264). Evidence of this negative regulatory role comes from the finding that PD-1-deficient mice develop lupus-like autoimmune diseases including arthritis and nephritis, along with cardiomyopathy (Nishimura H., et al., Immunity, 1999; 11:141-151; and Nishimura H., et al., Science, 2001; 291:319-322). In the tumor setting, the consequence is the development of immune resistance within the tumor microenvironment. PD-1 is highly expressed on tumor-infiltrating lymphocytes, and its ligands are up-regulated on the cell surface of many different tumors (Dong H., et al., Nat. Med. 2002; 8:793-800). Multiple murine cancer models have demonstrated that binding of ligand to PD-1 results in immune evasion. In addition, blockade of this interaction results in anti-tumor activity (Topalian S. L., et al. NEJM 2012; 366(26):2443-2454; Hamid O., et al., NEJM 2013; 369:134-144). Moreover, it has been shown that inhibition of the PD-1/PD-L1 interaction mediates potent antitumor activity in preclinical models (U.S. Pat. Nos. 8,008,449 and 7,943,743).

The initial members of the PD-1 family, CD28 and ICOS, were discovered by functional effects on augmenting T cell proliferation following the addition of monoclonal antibodies (Hutloff et al. Nature (1999); 397:263-266; Hansen et al. Immunogenics (1980); 10:247-260). PD-1 was discovered through screening for differential expression in apoptotic cells (Ishida et al. EMBO J(1992); 11:3887-95). The other members of the family, CTLA-4 and BTLA, were discovered through screening for differential expression in cytotoxic T lymphocytes and TH1 cells, respectively. CD28, ICOS and CTLA-4 all have an unpaired cysteine residue allowing for homodimerization. In contrast, PD-1 is suggested to exist as a monomer, lacking the unpaired cysteine residue characteristic in other CD28 family members.

The PD-1 gene is a 55 kDa type I transmembrane protein that is part of the Ig gene superfamily (Agata et al. (1996) Int Immunol 8:765-72). PD-1 contains a membrane proximal immunoreceptor tyrosine inhibitory motif (ITIM) and a membrane distal tyrosine-based switch motif (ITSM) (Thomas, M. L. (1995) J Exp Med 181:1953-6; Vivier, E and Daeron, M (1997) Immunol Today 18:286-91). Although structurally similar to CTLA-4, PD-1 lacks the MYPPPY motif (SEQ ID NO: 239) that is critical for B7-1 and B7-2 binding. Two ligands for PD-1 have been identified, PD-L1 and PD-L2, that have been shown to downregulate T cell activation upon binding to PD-1 (Freeman et al. (2000) J Exp Med 192:1027-34; Latchman et al. (2001) Nat Immunol 2:261-8; Carter et al. (2002) Eur J Immunol 32:634-43). Both PD-L1 and PD-L2 are B7 homologs that bind to PD-1, but do not bind to other CD28 family members. PD-L1 is abundant in a variety of human cancers (Dong et al. (2002) Nat. Med. 8:787-9). The interaction between PD-1 and PD-L1 results in a decrease in tumor infiltrating lymphocytes, a decrease in T-cell receptor mediated proliferation, and immune evasion by the cancerous cells (Dong et al. (2003) J. Mol. Med 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1, and the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66).

Consistent with PD-1 being an inhibitory member of the CD28 family, PD-1 deficient animals develop various autoimmune phenotypes, including autoimmune cardiomyopathy and a lupus-like syndrome with arthritis and nephritis (Nishimura et al. (1999) Immunity 11:141-51; Nishimura et al. (2001) Science 291:319-22). Additionally, PD-1 has been found to play a role in autoimmune encephalomyelitis, systemic lupus erythematosus, graft-versus-host disease (GVHD), type I diabetes, and rheumatoid arthritis (Salama et al. (2003) J Exp Med 198:71-78; Prokunina and Alarcon-Riquelme (2004) Hum Mol Genet 13:R143; Nielsen et al. (2004) Lupus 13:510). In a murine B cell tumor line, the ITSM of PD-1 was shown to be essential to block BCR-mediated Ca²⁺-flux and tyrosine phosphorylation of downstream effector molecules (Okazaki et al. (2001) PNAS 98:13866-71).

“Programmed Death Ligand-1 (PD-L1)” is one of two cell surface glycoprotein ligands for PD-1 (the other being PD-L2) that down-regulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

Some embodiments of the invention include an anti-PD-1 antibody, or an anti-PD-L1 antibody, in combination with an anti-CD36 antibody. PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to down regulate T cell activation and cytokine secretion upon binding to PD-1. Inhibition of the PD-1/PD-L1 interaction mediates potent antitumor activity in preclinical models.

Human monoclonal antibodies (HuMAbs) that bind specifically to PD-1 with high affinity have been disclosed in U.S. Pat. Nos. 8,008,449 and 8,779,105. Other anti-PD-1 mAbs have been described in, for example, U.S. Pat. Nos. 6,808,710, 7,488,802, 8,168,757 and 8,354,509, and PCT Publication Nos. WO2012/145493 and WO2016/168716. Each of the anti-PD-1 HuMAbs disclosed in U.S. Pat. No. 8,008,449 has been demonstrated to exhibit one or more of the following characteristics: (a) binds to human PD-1 with a K_{D} of 1×10⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-7 production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates Ab responses; and (j) inhibits tumor cell growth in vivo. Anti-PD-1 antibodies useful for the present invention include mAbs that bind specifically to human PD-1 and exhibit at least one, preferably at least five, of the preceding characteristics.

Anti-human-PD-1 antibodies (or VH and/or VL domains derived therefrom) suitable for use in the invention can be generated using methods well known in the art. Alternatively, art recognized anti-PD-1 antibodies can be used. For example, monoclonal antibodies 5C4 (referred to herein as Nivolumab or BMS-936558), 17D8, 2D3, 4H1, 4A11, 7D3, and 5F4, described in WO 2006/121168, the teachings of which are hereby incorporated by reference, can be used. Other known PD-1 antibodies include lambrolizumab (MK-3475) described in WO 2008/156712, and AMP-514 described in WO 2012/145493. Further known anti-PD-1 antibodies and other PD-1 inhibitors include those described in WO 2009/014708, WO 03/099196, WO 2009/114335 and WO 2011/161699. Another known anti-PD-1 antibody is pidilizumab (CT-011). Antibodies that compete with any of these antibodies or inhibitors for binding to PD-1 also can be used.

In one embodiment, the anti-PD-1 antibody is nivolumab. Nivolumab (also known as “OPDIVO®”; BMS-936558; formerly designated 5C4, BMS-936558, MDX-1i06, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56). In another embodiment, the anti-PD-1 antibody or fragment thereof cross-competes with nivolumab. In other embodiments, the anti-PD-1 antibody or fragment thereof binds to the same epitope as nivolumab. In certain embodiments, the anti-PD-1 antibody has the same CDRs as nivolumab.

In another embodiment, the anti-PD-1 antibody is pembrolizumab. Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587.

In another embodiment, the anti-PD-1 antibody cross-competes with pembrolizumab. In some embodiments, the anti-PD-1 antibody binds to the same epitope as pembrolizumab. In certain embodiments, the anti-PD-1 antibody has the same CDRs as pembrolizumab. In another embodiment, the anti-PD-1 antibody is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587 (incorporated herein by reference in their entirety); see also http://www.cancer.gov/drugdictionary?cdrid=695789 (last accessed: May 25, 2017). Pembrolizumab has been approved by the FDA for the treatment of relapsed or refractory melanoma.

In other embodiments, the anti-PD-1 antibody thereof cross-competes with MEDI0608. In still other embodiments, the anti-PD-1 antibody binds to the same epitope as MEDI0608. In certain embodiments, the anti-PD-1 antibody has the same CDRs as MEDI0608. In other embodiments, the anti-PD-1 antibody is MEDI0608 (formerly AMP-514), which is a monoclonal antibody. MEDI0608 is described, for example, in U.S. Pat. No. 8,609,089 or in http://www.cancer.gov/drugdictionary?cdrid=756047 (last accessed May 25, 2017).

In other embodiments, the anti-PD-1 antibody cross-competes with BGB-A317. In some embodiments, the anti-PD-1 antibody binds the same epitope as BGB-A317. In certain embodiments, the anti-PD-1 antibody has the same CDRs as BGB-A317. In certain embodiments, the anti-PD-1 antibody is BGB-A317, which is a humanized monoclonal antibody. BGB-A317 is described in U.S. Publ. No. 2015/0079109.

Anti-PD-1 antibodies useful for the disclosed compositions also include isolated antibodies that bind specifically to human PD-1 and cross-compete for binding to human PD-1 with nivolumab (see, e.g., U.S. Pat. Nos. 8,008,449 and 8,779,105; Int'l Pub. No. WO 2013/173223). The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar to those of nivolumab by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., Int'l Pub. No. WO 2013/173223).

In certain embodiments, antibodies that cross-compete for binding to human PD-1 with, or bind to the same epitope region of human PD-1 as, nivolumab are mAbs. For administration to human subjects, these cross-competing antibodies can be chimeric antibodies, or humanized or human antibodies. Such chimeric, humanized or human mAbs can be prepared and isolated by methods well known in the art.

Anti-PD-1 antibodies useful for the compositions of the disclosed invention also include antigen-binding portions of the above antibodies. It has been amply demonstrated that the antigen-binding function of an antibody can be performed by fragments of a full length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; and (iv) a Fv fragment consisting of the VL and VH domains of a single arm of an antibody.

Anti-PD-1 antibodies suitable for use in the disclosed compositions are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and upregulating the immune system. In certain embodiments, the anti-PD-1 antibody cross-competes with nivolumab for binding to human PD-1. In other embodiments, the anti-PD-1 antibody is a chimeric, humanized or human monoclonal antibody or a portion thereof. In certain embodiments, the antibody is a humanized antibody. In other embodiments, the antibody is a human antibody. Antibodies of an IgG1, IgG2, IgG3 or IgG4 isotype can be used.

In certain embodiments, the anti-PD-1 antibody comprises a heavy chain constant region which is of a human IgG1 or IgG4 isotype. In certain other embodiments, the sequence of the IgG4 heavy chain constant region of the anti-PD-1 antibody contains an S228P mutation which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies (Wang et al., 2014). In yet other embodiments, the antibody comprises a light chain constant region which is a human kappa or lambda constant region. In other embodiments, the anti-PD-1 antibody is a mAb or an antigen-binding portion thereof. In certain embodiments of any of the therapeutic methods described herein comprising administration of an anti-PD-1 antibody, the anti-PD-1 antibody is nivolumab. In other embodiments, the anti-PD-1 antibody is pembrolizumab. In other embodiments, the anti-PD-1 antibody is chosen from the human antibodies 17D8, 2D3, 4H1, 4A11, 7D3 and 5F4 described in U.S. Pat. No. 8,008,449. In still other embodiments, the anti-PD-1 antibody is MEDI0608 (formerly AMP-514), AMP-224, or Pidilizumab (CT-011). Other known PD-1 antibodies include lambrolizumab (MK-3475) described in, for example, WO 2008/156712, and AMP-514 described in, for example, WO 2012/145493. Further known anti-PD-1 antibodies and other PD-1 inhibitors include those described in, for example, WO 2009/014708, WO 03/099196, WO 2009/114335 and WO 2011/161699. In one embodiment, the anti-PD-1 antibody is REGN2810. In one embodiment, the anti-PD-1 antibody is PDR001. Another known anti-PD-1 antibody is pidilizumab (CT-011). Each of the above references are incorporated by reference. Antibodies that compete with any of these antibodies or inhibitors for binding to PD-1 also can be used.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540, each of which are herein incorporated by reference.

In some embodiments, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; see Si-Yang Liu et al., J. Hematol. Oncol. 10:136 (2017)), BGB-A317 (Beigene; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANBO11; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), and IBI308 (Innovent; see WO 2017/024465, WO 2017/025016, WO 2017/132825, and WO 2017/133540). Each of the above references are herein incorporated by reference.

In embodiments, the anti-PD-1 antibody is a bispecific antibody. In embodiments, the second therapy is a PD-1 inhibitor. In embodiments, the PD-1 inhibitor is a small molecule.

Because anti-PD-1 antibodies and anti-PD-L1 antibodies target the same signaling pathway and have been shown in clinical trials to exhibit similar levels of efficacy in a variety of cancers, an anti-PD-L1 antibody can be substituted for an anti-PD-1 antibody in any of the therapeutic methods or compositions disclosed herein.

Anti-human-PD-L1 antibodies (or VH and/or VL domains derived therefrom) suitable for use in the invention can be generated using methods well known in the art. Alternatively, art recognized anti-PD-L1 antibodies can be used. For example, human anti-PD-L1 antibodies disclosed in U.S. Pat. No. 7,943,743, the contents of which are hereby incorporated by reference, can be used. Such anti-PD-L1 antibodies include 3G10, 12A4 (also referred to as BMS-936559), 10A5, 5F8, 10H10, 1B12, 7H1, 11E6, 12B7, and 13G4. Other art recognized anti-PD-L1 antibodies which can be used include those described in, for example, U.S. Pat. Nos. 7,635,757 and 8,217,149, U.S. Publication No. 2009/0317368, and PCT Publication Nos. WO 2011/066389 and WO 2012/145493, each of which are herein incorporated by reference. Other examples of an anti-PD-L1 antibody include atezolizumab (TECENTRIQ; RG7446), or durvalumab (IMFINZI; MEDI4736). Antibodies that compete with any of these art-recognized antibodies or inhibitors for binding to PD-L1 also can be used.

Examples of anti-PD-L1 antibodies useful in the methods of the present disclosure include the antibodies disclosed in U.S. Pat. No. 9,580,507, which is herein incorporated by reference. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present invention include monoclonal antibodies that bind specifically to human PD-L1 and exhibit at least one, in some embodiments, at least five, of the preceding characteristics.

In certain embodiments, the anti-PD-L1 antibody is BMS-936559 (formerly 12A4 or MDX-1105) (see, e.g., U.S. Pat. No. 7,943,743; WO 2013/173223). In other embodiments, the anti-PD-L1 antibody is MPDL3280A (also known as RG7446 and atezolizumab) (see, e.g., Herbst et al. 2013 J Clin Oncol 31(suppl):3000; U.S. Pat. No. 8,217,149), MEDI4736 (Khleif, 2013, In: Proceedings from the European Cancer Congress 2013; Sep. 27-Oct. 1, 2013; Amsterdam, The Netherlands. Abstract 802), or MSB0010718C (also called Avelumab; see US 2014/0341917). In certain embodiments, antibodies that cross-compete for binding to human PD-L1 with, or bind to the same epitope region of human PD-L1 as the above-references PD-L1 antibodies are mAbs. For administration to human subjects, these cross-competing antibodies can be chimeric antibodies, or can be humanized or human antibodies. Such chimeric, humanized or human mAbs can be prepared and isolated by methods well known in the art. In certain embodiments, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see, e.g., WO 2011/066389), avelumab (Pfizer; also known as BAVENCIO®, MSB-0010718C; see, e.g., WO 2013/079174), STI-1014 (Sorrento; see, e.g., WO2013/181634), CX-072 (Cytomx; see, e.g., WO2016/149201), KN035 (3D Med/Alphamab; see Zhang et al., Cell Discov. 7:3 (March 2017), LY3300054 (Eli Lilly Co.; see, e.g., WO 2017/034916), and CK-301 (Checkpoint Therapeutics; see Gorelik et al., AACR:Abstract 4606 (April 2016)). The above references are herein incorporated by reference.

In certain embodiments, the PD-L1 antibody is atezolizumab (TECENTRIQ®). Atezolizumab is a fully humanized IgG1 monoclonal anti-PD-L1 antibody.

In certain embodiments, the PD-L1 antibody is durvalumab (IIFINZI™) Durvalumab is a human IgG1 kappa monoclonal anti-PD-L1 antibody.

In certain embodiments, the PD-L1 antibody is avelumab (BAVENCIO®). Avelumab is a human IgG1 lambda monoclonal anti-PD-L1 antibody.

In other embodiments, the anti-PD-L1 monoclonal antibody is selected from the group consisting of 28-8, 28-1, 28-12, 29-8, 5H1, and any combination thereof.

Anti-PD-L1 antibodies usable in the disclosed methods also include isolated antibodies that bind specifically to human PD-L1 and cross-compete for binding to human PD-L1 with any anti-PD-L1 antibody disclosed herein, e.g., atezolizumab, durvalumab, and/or avelumab. In some embodiments, the anti-PD-L1 antibody binds the same epitope as any of the anti-PD-L1 antibodies described herein, e.g., atezolizumab, durvalumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., atezolizumab and/or avelumab, by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In certain embodiments, the antibodies that cross-compete for binding to human PD-L1 with, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the methods of the disclosed invention also include antigen-binding portions of the above antibodies. It has been amply demonstrated that the antigen-binding function of an antibody can be performed by fragments of a full length antibody.

Anti-PD-L1 antibodies suitable for use in the disclosed methods or compositions are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain embodiments, the anti-PD-L1 antibody cross-competes with atezolizumab, durvalumab, and/or avelumab for binding to human PD-L1.

### Anti-CTLA-4 Antibodies

In certain embodiments, an embodiment encompasses use of an anti-CTLA-4 antibody. In one embodiment, the anti-CTLA-4 antibody binds to and inhibits CTLA-4. In some embodiments, the anti-CTLA-4 antibody is ipilimumab (YERVOY), tremelimumab (ticilimumab; CP-675,206), AGEN-1884, or ATOR-1015.

### Further Embodiments

1. An isolated antibody that binds to CD36, which comprises a light chain CDR1 region, a light chain CDR2 region, a light chain CDR3 region, a heavy chain CDR1 region, a heavy chain CDR2 region, and a heavy chain CDR3 region, wherein:
   the heavy chain CDR1 region comprises a sequence selected from the group consisting of SEQ ID NOs: 85-105;
   the heavy chain CDR2 region comprises a sequence selected from the group consisting of SEQ ID NOs: 106-132, or 248;
   the heavy chain CDR3 region comprises a sequence selected from the group consisting of SEQ ID NOs: 133-158;
   the light chain CDR1 region comprises a sequence selected from the group consisting of SEQ ID NOs: 159-172;
   the light chain CDR2 region comprises a sequence selected from the group consisting of SEQ ID NOs: 173-185, or 246; and
   the light chain CDR3 region comprises a sequence selected from the group consisting of SEQ ID NOs: 186-206, or 247.
2. The isolated antibody of embodiment 1, wherein the antibody is a chimeric antibody.
3. The isolated antibody of embodiment 1, wherein the antibody is a humanized antibody.
4. The antibody of any one of embodiments 1-3, wherein the heavy chain CDR1 region comprises SEQ ID NO: 85, the heavy chain CDR2 region comprises SEQ ID NO: 106, the heavy chain CDR3 region comprises SEQ ID NO: 133, the light chain CDR1 region comprises SEQ ID NO: 159, the light chain CDR2 region comprises SEQ ID NO: 173, and the light chain CDR3 region comprises SEQ ID NO: 186.
5. The antibody of any one of embodiments 1-3, wherein the heavy chain CDR1 region comprises SEQ ID NO: 86, the heavy chain CDR2 region comprises SEQ ID NO: 107, the heavy chain CDR3 region comprises SEQ ID NO: 134, the light chain CDR1 region comprises SEQ ID NO: 160, the light chain CDR2 region comprises SEQ ID NO: 174, and the light chain CDR3 region comprises SEQ ID NO: 187.
6. The antibody of any one of embodiments 1-3, wherein the heavy chain CDR1 region comprises SEQ ID NO: 86, the heavy chain CDR2 region comprises SEQ ID NO: 108, the heavy chain CDR3 region comprises SEQ ID NO: 135, the light chain CDR1 region comprises SEQ ID NO: 160, the light chain CDR2 region comprises SEQ ID NO: 174, and the light chain CDR3 region comprises SEQ ID NO: 188.
7. The antibody of any one of embodiments 1-3, wherein the heavy chain CDR1 region comprises SEQ ID NO: 87, the heavy chain CDR2 region comprises SEQ ID NO: 109, the heavy chain CDR3 region comprises SEQ ID NO: 136, the light chain CDR1 region comprises SEQ ID NO: 160, the light chain CDR2 region comprises SEQ ID NO: 174, and the light chain CDR3 region comprises SEQ ID NO: 187.
8. The antibody of any one of embodiments 1-3, wherein the heavy chain CDR1 region comprises SEQ ID NO: 88, the heavy chain CDR2 region comprises SEQ ID NO: 110, the heavy chain CDR3 region comprises SEQ ID NO: 137, the light chain CDR1 region comprises SEQ ID NO: 160, the light chain CDR2 region comprises SEQ ID NO: 174, and the light chain CDR3 region comprises SEQ ID NO: 187.
9. The antibody of any one of embodiments 1-3, wherein the heavy chain CDR1 region comprises SEQ ID NO: 89, the heavy chain CDR2 region comprises SEQ ID NO: 111, the heavy chain CDR3 region comprises SEQ ID NO: 138, the light chain CDR1 region comprises SEQ ID NO: 161, the light chain CDR2 region comprises SEQ ID NO: 175, and the light chain CDR3 region comprises SEQ ID NO: 189.
10. The antibody of any one of embodiments 1-3, wherein the heavy chain CDR1 region comprises SEQ ID NO: 90, the heavy chain CDR2 region comprises SEQ ID NO: 112, the heavy chain CDR3 region comprises SEQ ID NO: 139, the light chain CDR1 region comprises SEQ ID NO: 160, the light chain CDR2 region comprises SEQ ID NO: 174, and the light chain CDR3 region comprises SEQ ID NO: 190.
11. The antibody of any one of embodiments 1-3, wherein the heavy chain CDR1 region comprises SEQ ID NO: 91, the heavy chain CDR2 region comprises SEQ ID NO: 113, the heavy chain CDR3 region comprises SEQ ID NO: 140, the light chain CDR1 region comprises SEQ ID NO: 160, the light chain CDR2 region comprises SEQ ID NO: 174, and the light chain CDR3 region comprises SEQ ID NO: 187.
12. The antibody of any one of embodiments 1-3, wherein the heavy chain CDR1 region comprises SEQ ID NO: 92, the heavy chain CDR2 region comprises SEQ ID NO: 114, the heavy chain CDR3 region comprises SEQ ID NO: 141, the light chain CDR1 region comprises SEQ ID NO: 160, the light chain CDR2 region comprises SEQ ID NO: 174, and the light chain CDR3 region comprises SEQ ID NO: 191.
13. The antibody of any one of embodiments 1-3, wherein the heavy chain CDR1 region comprises SEQ ID NO: 93, the heavy chain CDR2 region comprises SEQ ID NO: 115, the heavy chain CDR3 region comprises SEQ ID NO: 142, the light chain CDR1 region comprises SEQ ID NO: 163, the light chain CDR2 region comprises SEQ ID NO: 177, and the light chain CDR3 region comprises SEQ ID NO: 192.
14. The antibody of any one of embodiments 1-3, wherein the heavy chain CDR1 region comprises SEQ ID NO: 94, the heavy chain CDR2 region comprises SEQ ID NO: 116, the heavy chain CDR3 region comprises SEQ ID NO: 143, the light chain CDR1 region comprises SEQ ID NO: 164, the light chain CDR2 region comprises SEQ ID NO: 175, and the light chain CDR3 region comprises SEQ ID NO: 193.
15. The antibody of any one of embodiments 1-3, wherein the heavy chain CDR1 region comprises SEQ ID NO: 95, the heavy chain CDR2 region comprises SEQ ID NO: 117, the heavy chain CDR3 region comprises SEQ ID NO: 144, the light chain CDR1 region comprises SEQ ID NO: 160, the light chain CDR2 region comprises SEQ ID NO: 174, and the light chain CDR3 region comprises SEQ ID NO: 190.
16. The antibody of any one of embodiments 1-3, wherein the heavy chain CDR1 region comprises SEQ ID NO: 95, the heavy chain CDR2 region comprises SEQ ID NO: 118, the heavy chain CDR3 region comprises SEQ ID NO: 145, the light chain CDR1 region comprises SEQ ID NO: 160, the light chain CDR2 region comprises SEQ ID NO: 174, and the light chain CDR3 region comprises SEQ ID NO: 187.
17. The antibody of any one of embodiments 1-3, wherein the heavy chain CDR1 region comprises SEQ ID NO: 95, the heavy chain CDR2 region comprises SEQ ID NO: 119, the heavy chain CDR3 region comprises SEQ ID NO: 146, the light chain CDR1 region comprises SEQ ID NO: 160, the light chain CDR2 region comprises SEQ ID NO: 174, and the light chain CDR3 region comprises SEQ ID NO: 190.
18. The antibody of any one of embodiments 1-3, wherein the heavy chain CDR1 region comprises SEQ ID NO: 96, the heavy chain CDR2 region comprises SEQ ID NO: 120, the heavy chain CDR3 region comprises SEQ ID NO: 147, the light chain CDR1 region comprises SEQ ID NO: 159, the light chain CDR2 region comprises SEQ ID NO: 173, and the light chain CDR3 region comprises SEQ ID NO: 194.
19. The antibody of any one of embodiments 1-3, wherein the heavy chain CDR1 region comprises SEQ ID NO: 96, the heavy chain CDR2 region comprises SEQ ID NO: 121, the heavy chain CDR3 region comprises SEQ ID NO: 148, the light chain CDR1 region comprises SEQ ID NO: 159, the light chain CDR2 region comprises SEQ ID NO: 173, and the light chain CDR3 region comprises SEQ ID NO: 195.
20. The antibody of any one of embodiments 1-3, wherein the heavy chain CDR1 region comprises SEQ ID NO: 96, the heavy chain CDR2 region comprises SEQ ID NO: 120, the heavy chain CDR3 region comprises SEQ ID NO: 147, the light chain CDR1 region comprises SEQ ID NO: 159, the light chain CDR2 region comprises SEQ ID NO: 173, and the light chain CDR3 region comprises SEQ ID NO: 186.
21. The antibody of any one of embodiments 1-3, wherein the heavy chain CDR1 region comprises SEQ ID NO: 96, the heavy chain CDR2 region comprises SEQ ID NO: 121, the heavy chain CDR3 region comprises SEQ ID NO: 147, the light chain CDR1 region comprises SEQ ID NO: 159, the light chain CDR2 region comprises SEQ ID NO: 173, and the light chain CDR3 region comprises SEQ ID NO: 196.
22. The antibody of any one of embodiments 1-3, wherein the heavy chain CDR1 region comprises SEQ ID NO: 96, the heavy chain CDR2 region comprises SEQ ID NO: 121, the heavy chain CDR3 region comprises SEQ ID NO: 147, the light chain CDR1 region comprises SEQ ID NO: 159, the light chain CDR2 region comprises SEQ ID NO: 173, and the light chain CDR3 region comprises SEQ ID NO: 195.
23. The antibody of any one of embodiments 1-3, wherein the heavy chain CDR1 region comprises SEQ ID NO: 97, the heavy chain CDR2 region comprises SEQ ID NO: 122, the heavy chain CDR3 region comprises SEQ ID NO: 149, the light chain CDR1 region comprises SEQ ID NO: 165, the light chain CDR2 region comprises SEQ ID NO: 178, and the light chain CDR3 region comprises SEQ ID NO: 197.
24. The antibody of any one of embodiments 1-3, wherein the heavy chain CDR1 region comprises SEQ ID NO: 97, the heavy chain CDR2 region comprises SEQ ID NO: 123, the heavy chain CDR3 region comprises SEQ ID NO: 150, the light chain CDR1 region comprises SEQ ID NO: 165, the light chain CDR2 region comprises SEQ ID NO: 178, and the light chain CDR3 region comprises SEQ ID NO: 197.
25. The antibody of any one of embodiments 1-3, wherein the heavy chain CDR1 region comprises SEQ ID NO: 98, the heavy chain CDR2 region comprises SEQ ID NO: 124, the heavy chain CDR3 region comprises SEQ ID NO: 151, the light chain CDR1 region comprises SEQ ID NO: 160, the light chain CDR2 region comprises SEQ ID NO: 174, and the light chain CDR3 region comprises SEQ ID NO: 198.
26. The antibody of any one of embodiments 1-3, wherein the heavy chain CDR1 region comprises SEQ ID NO: 96, the heavy chain CDR2 region comprises SEQ ID NO: 120, the heavy chain CDR3 region comprises SEQ ID NO: 147, the light chain CDR1 region comprises SEQ ID NO: 159, the light chain CDR2 region comprises SEQ ID NO: 173, and the light chain CDR3 region comprises SEQ ID NO: 195.
27. The antibody of any one of embodiments 1-3, wherein the heavy chain CDR1 region comprises SEQ ID NO: 91, the heavy chain CDR2 region comprises SEQ ID NO: 125, the heavy chain CDR3 region comprises SEQ ID NO: 152, the light chain CDR1 region comprises SEQ ID NO: 160, the light chain CDR2 region comprises SEQ ID NO: 174, and the light chain CDR3 region comprises SEQ ID NO: 190.
28. The antibody of any one of embodiments 1-3, wherein the heavy chain CDR1 region comprises SEQ ID NO: 99, the heavy chain CDR2 region comprises SEQ ID NO: 126, the heavy chain CDR3 region comprises SEQ ID NO: 133, the light chain CDR1 region comprises SEQ ID NO: 162, the light chain CDR2 region comprises SEQ ID NO: 176, and the light chain CDR3 region comprises SEQ ID NO: 199.
29. The antibody of any one of embodiments 1-3, wherein the heavy chain CDR1 region comprises SEQ ID NO: 100, the heavy chain CDR2 region comprises SEQ ID NO: 127, the heavy chain CDR3 region comprises SEQ ID NO: 153, the light chain CDR1 region comprises SEQ ID NO: 166, the light chain CDR2 region comprises SEQ ID NO: 179, and the light chain CDR3 region comprises SEQ ID NO: 200.
30. The antibody of any one of embodiments 1-3, wherein the heavy chain CDR1 region comprises SEQ ID NO: 101, the heavy chain CDR2 region comprises SEQ ID NO: 128, the heavy chain CDR3 region comprises SEQ ID NO: 154, the light chain CDR1 region comprises SEQ ID NO: 167, the light chain CDR2 region comprises SEQ ID NO: 180, and the light chain CDR3 region comprises SEQ ID NO: 201.
31. The antibody of any one of embodiments 1-3, wherein the heavy chain CDR1 region comprises SEQ ID NO: 101, the heavy chain CDR2 region comprises SEQ ID NO: 128, the heavy chain CDR3 region comprises SEQ ID NO: 154, the light chain CDR1 region comprises SEQ ID NO: 168, the light chain CDR2 region comprises SEQ ID NO: 181, and the light chain CDR3 region comprises SEQ ID NO: 202.
32. The antibody of any one of embodiments 1-3, wherein the heavy chain CDR1 region comprises SEQ ID NO: 102, the heavy chain CDR2 region comprises SEQ ID NO: 129, the heavy chain CDR3 region comprises SEQ ID NO: 155, the light chain CDR1 region comprises SEQ ID NO: 169, the light chain CDR2 region comprises SEQ ID NO: 182, and the light chain CDR3 region comprises SEQ ID NO: 203.
33. The antibody of any one of embodiments 1-3, wherein the heavy chain CDR1 region comprises SEQ ID NO: 103, the heavy chain CDR2 region comprises SEQ ID NO: 130, the heavy chain CDR3 region comprises SEQ ID NO: 156, the light chain CDR1 region comprises SEQ ID NO: 170, the light chain CDR2 region comprises SEQ ID NO: 183, and the light chain CDR3 region comprises SEQ ID NO: 204.
34. The antibody of any one of embodiments 1-3, wherein the heavy chain CDR1 region comprises SEQ ID NO: 104, the heavy chain CDR2 region comprises SEQ ID NO: 131, the heavy chain CDR3 region comprises SEQ ID NO: 157, the light chain CDR1 region comprises SEQ ID NO: 171, the light chain CDR2 region comprises SEQ ID NO: 184, and the light chain CDR3 region comprises SEQ ID NO: 205.
35. The antibody of any one of embodiments 1-3, wherein the heavy chain CDR1 region comprises SEQ ID NO: 105, the heavy chain CDR2 region comprises SEQ ID NO: 132, the heavy chain CDR3 region comprises SEQ ID NO: 158, the light chain CDR1 region comprises SEQ ID NO: 172, the light chain CDR2 region comprises SEQ ID NO: 185, and the light chain CDR3 region comprises SEQ ID NO: 206.
36. The antibody of any one of embodiments 1-3, wherein the heavy chain CDR1 region comprises SEQ ID NO: 90, the heavy chain CDR2 region comprises SEQ ID NO: 248, the heavy chain CDR3 region comprises SEQ ID NO: 139, the light chain CDR1 region comprises SEQ ID NO:160, the light chain CDR2 region comprises SEQ ID NO: 174, and the light chain CDR3 region comprises SEQ ID NO: 247.
37. The antibody of any one of embodiments 1-3, wherein the heavy chain CDR1 region comprises SEQ ID NO: 90, the heavy chain CDR2 region comprises SEQ ID NO: 248, the heavy chain CDR3 region comprises SEQ ID NO: 139, the light chain CDR1 region comprises SEQ ID NO:160, the light chain CDR2 region comprises SEQ ID NO: 246, and the light chain CDR3 region comprises SEQ ID NO: 247.
38. The antibody of embodiment 1 or embodiment 2, wherein the antibody comprises a heavy chain variable region and a light chain variable region,
   wherein the heavy chain variable region has at least 80%, at least 85%, at least 90%, at least 95%, at least 96%, at least 97%, at least 98%, at least 99%, or 100% identity with the amino acid sequence of at least one of SEQ ID NOs: 13-44, 241, or 243; and
   wherein the light chain variable region has at least 80%, at least 85%, at least 90%, at least 95%, at least 96%, at least 97%, at least 98%, at least 99%, or 100% identity with the amino acid sequence of at least one of SEQ ID NOs: 45-78, 240, 242, 244, or 245.
39. The antibody of embodiment 38, wherein the antibody comprises a heavy chain variable region and a light chain variable region,
   wherein the heavy chain variable region comprises a sequence selected from the group consisting of SEQ ID NOs: 13-44, 241 and 243 and
   wherein the light chain variable region comprises a sequence selected from the group consisting of SEQ ID NOs: 45-78, 240, 242, 244, and 245.
40. The antibody of embodiment 39, wherein:
   i. the heavy chain variable region comprises SEQ ID NO: 13 and the light chain variable region comprises SEQ ID NO: 45;
   ii. the heavy chain variable region comprises SEQ ID NO: 14 and the light chain variable region comprises SEQ ID NO: 46;
   iii. the heavy chain variable region comprises SEQ ID NO: 15 and the light chain variable region comprises SEQ ID NO: 47;
   iv. the heavy chain variable region comprises SEQ ID NO: 16 and the light chain variable region comprises SEQ ID NO: 48;
   v. the heavy chain variable region comprises SEQ ID NO: 17 and the light chain variable region comprises SEQ ID NO: 49;
   vi. the heavy chain variable region comprises SEQ ID NO: 18 and the light chain variable region comprises SEQ ID NO: 50;
   vii. the heavy chain variable region comprises SEQ ID NO: 19 and the light chain variable region comprises SEQ ID NO: 51;
   viii. the heavy chain variable region comprises SEQ ID NO: 20 and the light chain variable region comprises SEQ ID NO: 52;
   ix. the heavy chain variable region comprises SEQ ID NO: 21 and the light chain variable region comprises SEQ ID NO: 53;
   x. the heavy chain variable region comprises SEQ ID NO: 22 and the light chain variable region comprises SEQ ID NO: 54;
   xi. the heavy chain variable region comprises SEQ ID NO: 23 and the light chain variable region comprises SEQ ID NO: 55;
   xii. the heavy chain variable region comprises SEQ ID NO: 24 and the light chain variable region comprises SEQ ID NO: 56;
   xiii. the heavy chain variable region comprises SEQ ID NO: 25 and the light chain variable region comprises SEQ ID NO: 57;
   xiv. the heavy chain variable region comprises SEQ ID NO: 26 and the light chain variable region comprises SEQ ID NO: 58;
   xv. the heavy chain variable region comprises SEQ ID NO: 26 and the light chain variable region comprises SEQ ID NO: 59;
   xvi. the heavy chain variable region comprises SEQ ID NO: 27 and the light chain variable region comprises SEQ ID NO: 60;
   xvii. the heavy chain variable region comprises SEQ ID NO: 28 and the light chain variable region comprises SEQ ID NO: 61;
   xviii. the heavy chain variable region comprises SEQ ID NO: 29 and the light chain variable region comprises SEQ ID NO: 62;
   xix. the heavy chain variable region comprises SEQ ID NO: 30 and the light chain variable region comprises SEQ ID NO: 63;
   xx. the heavy chain variable region comprises SEQ ID NO: 31 and the light chain variable region comprises SEQ ID NO: 64;
   xxi. the heavy chain variable region comprises SEQ ID NO: 32 and the light chain variable region comprises SEQ ID NO: 65;
   xxii. the heavy chain variable region comprises SEQ ID NO: 33 and the light chain variable region comprises SEQ ID NO: 66;
   xxiii. the heavy chain variable region comprises SEQ ID NO: 34 and the light chain variable region comprises SEQ ID NO: 67;
   xxiv. the heavy chain variable region comprises SEQ ID NO: 35 and the light chain variable region comprises SEQ ID NO: 68;
   xxv. the heavy chain variable region comprises SEQ ID NO: 36 and the light chain variable region comprises SEQ ID NO: 69;
   xxvi. the heavy chain variable region comprises SEQ ID NO: 37 and the light chain variable region comprises SEQ ID NO: 70;
   xxvii. the heavy chain variable region comprises SEQ ID NO: 38 and the light chain variable region comprises SEQ ID NO: 71;
   xxviii. the heavy chain variable region comprises SEQ ID NO: 39 and the light chain variable region comprises SEQ ID NO: 72;
   xxix. the heavy chain variable region comprises SEQ ID NO: 40 and the light chain variable region comprises SEQ ID NO: 73;
   xxx. the heavy chain variable region comprises SEQ ID NO: 40 and the light chain variable region comprises SEQ ID NO: 74;
   xxxi. the heavy chain variable region comprises SEQ ID NO: 41 and the light chain variable region comprises SEQ ID NO: 75;
   xxxii. the heavy chain variable region comprises SEQ ID NO: 42 and the light chain variable region comprises SEQ ID NO: 76;
   xxxiii. the heavy chain variable region comprises SEQ ID NO: 43 and the light chain variable region comprises SEQ ID NO: 77;
   xxxiv. the heavy chain variable region comprises SEQ ID NO: 44 and the light chain variable region comprises SEQ ID NO: 78;
   xxxv. the heavy chain variable region comprises SEQ NO: 241 and the light chain variable region comprises SEQ ID NO: 240;
   xxxvi. the heavy chain variable region comprises SEQ NO: 243 and the light chain variable region comprises SEQ ID NO: 240;
   xxxvii. the heavy chain variable region comprises SEQ NO: 241 and the light chain variable region comprises SEQ ID NO: 242;
   xxxviii. the heavy chain variable region comprises SEQ NO: 243 and the light chain variable region comprises SEQ ID NO: 242;
   xxxix. the heavy chain variable region comprises SEQ NO: 241 and the light chain variable region comprises SEQ ID NO: 244;
   xl. the heavy chain variable region comprises SEQ NO: 243 and the light chain variable region comprises SEQ ID NO: 244;
   xli. the heavy chain variable region comprises SEQ NO: 241 and the light chain variable region comprises SEQ ID NO: 245; or
   xlii. the heavy chain variable region comprises SEQ NO: 243 and the light chain variable region comprises SEQ ID NO: 245.
41. The antibody of any one of embodiments 1 to 40, wherein the antibody binds to human CD36.
42. The antibody of embodiment 41, wherein the antibody specifically binds to human CD36.
43. The antibody of embodiment 41, wherein the antibody has cross-reactivity to human CD36 and non-human CD36.
44. The antibody of embodiment 43, wherein the antibody has cross-reactivity to human CD36 and non-human primate CD36.
45. The antibody of embodiment 44, wherein the non-human primate CD36 is cynomomolgus CD36 or rhesus macaque CD36.
46. The antibody of embodiment 44, wherein the antibody has cross-reactivity to human CD36, non-human primate CD36, and rodent CD36.
47. The antibody of embodiment 46, wherein the rodent CD36 is mouse CD36 or rat CD36.
48. The antibody of embodiment 41, wherein the antibody binds to an epitope in human CD36 comprising at least one amino acid selected from the group consisting of 145A, 146S, 147H, 148I, 149Y, 150Q, 151N, 152Q, 153F, 154V, 155Q, 156M, 1571, 158L, 159N, 160S, 185P, 186F, 187L, 188S, 189L, 190V, 191P, 192Y, 193P, 194V, 195T, 196T, 197T, 198V, 199G, 398K, 399I, 400Q, 401V, 402L, 403K, 404N, 405L, 406K, 407R, 408N, 409Y, 4101, 411V, 412P, 413I, and 414L.
49. The antibody of embodiment 48, wherein the antibody binds to an epitope in human CD36 comprising at least one amino acid selected from the group consisting of 149Y, 150Q, 151N, 152Q, 153F, 154V, 155Q, 156M, 188S, 189L, 190V, 191P, 192Y, 193P, 194V, 195T, 196T, 400Q, 401V, 402L, and 403K.
50. The antibody of embodiment 49, wherein the antibody binds to an epitope in human CD36 comprising at least one amino acid selected from the group consisting of 152Q, 192Y, and 406K.
51. The antibody of embodiment 49, wherein the antibody binds to an epitope comprising 149Y, 150Q, 151N, 152Q, 153F, 154V, and 155Q and 156M.
52. The antibody of embodiment 48, wherein the antibody binds to an epitope comprising 188S, 189L, 190V, 191P, 192Y, 193P, 194V, 195T, 196T.
53. The antibody of embodiment 48, wherein the antibody binds to an epitope comprising 400Q, 401V, 402L, and 403K.
54. The antibody of embodiment 41, wherein the antibody binds to an epitope in human CD36 comprising at least one amino acid selected from the group consisting of 280E, 281S, 282D, 283V, 284N, 285L, 286K, 287G, 2881, 289P, 290V, 291Y, 292R, 293F, 294V, 295L, 296P, 297S, 298K, 341I, 342S, 343L, 344P, 345H, 346F, 347L, 348Y, 349A, 350S, 351P, 352D, 353V, 354S, 355E, 356P, 3571, 358D, 359G, 360L, 361N, 362P, 363N, 364E, and 365E.
55. The antibody of embodiment 54, wherein the antibody binds to an epitope in human CD36 comprising at least one amino acid selected from the group consisting of 286K, 287G, 2881, 289P, 290V, 291Y, 292R, 341I, 342S, 343L, 344P, 345H, 346F, 347L, 348Y, 349A, and 350S.
56. The antibody of embodiment 55, wherein the antibody binds to an epitope in human CD36 comprising at least one amino acid selected from the group consisting of 2881, 289P, 290V, 402D, 403V, and 404S.
57. The antibody of embodiment 54, wherein the antibody binds to an epitope in human CD36 comprising at least one amino acid selected from the group consisting of 286K, 287G, 2881, 289P, 290V, 291Y, and 292R.
58. The antibody of embodiment 54, wherein the antibody binds to an epitope in human CD36 comprising at least one amino acid selected from the group consisting of: 400S, 401P, 402D, and 403V.
59. The antibody of any one of embodiments 1 to 58, wherein the antibody binds to human CD36 with a K_{D} of less than 20 nM, as measured using surface plasmon resonance with a bivalent model.
60. The antibody of embodiment 59, wherein the antibody binds to human CD36 with a K_{D} of less than 10 nM, as measured using surface plasmon resonance with a bivalent model.
61. The antibody of any one of embodiments 1 to 60, which further comprises a heavy chain constant region.
62. The antibody of embodiment 61, wherein the heavy chain constant region is selected from the group consisting of human immunoglobulin IgA1, IgA2, IgG1, IgG2, IgG3, or IgG4 heavy chain constant regions.
63. The antibody of embodiment 62, which comprises an IgG1 heavy chain constant region.
64. The antibody of embodiment 63, wherein the heavy chain constant region comprises an IgG constant region containing at least one amino acid substitution, wherein the at least one amino acid substitution results in reduced Fc binding to at least one Fcgamma receptor and reduced Fc effector function.
65. The antibody of embodiment 64, wherein the at least one Fc silencing mutation includes the amino acid substitutions L234A and L235A (“LALA”).
66. The antibody of embodiment 64, wherein the at least one Fc silencing mutation includes a set of amino acid substitutions selected from the group consisting of L234G, L235S, and G236R; L234S, L235T, and G236R; L234S, L235V, and G236R; L234T, L235Q, and G236R; L234T, L235T, and G236R; L234A, L235S, and G236R; L234Q, L235S, and G236R; L234S, L235G, and G236R; L234T, L235S, and G236R; L234Q, L235S, and G236R; L234A and L235A; and L234A, L235A, and P329G.
67. The antibody of embodiment 62, which comprises an IgG4 heavy chain constant region.
68. The antibody of embodiment 66, wherein the heavy chain constant region comprises an IgG constant region containing the amino acid substitution S228P.
69. The antibody of any one of embodiments 1 to 68, wherein the antibody further comprises a light chain constant region.
70. The antibody of embodiment 69, wherein the light chain constant region is selected from the group consisting of human immunoglobulins κ and λ light chain constant regions.
71. The antibody of any one of embodiments 1 to 70, wherein the antibody further comprises a heavy chain constant region and a light chain constant region, wherein the heavy chain constant region is a human IgG1 heavy chain constant region, and wherein the light chain constant region is a human κ light chain constant region.
72. The antibody of any one of embodiments 1 to 71, wherein the antibody is a bispecific antibody.
73. The antibody of embodiment 72, comprising a first antigen-binding region that specifically binds to CD36.
74. The antibody of embodiment 73, comprising a second antigen-binding region that specifically binds to an immune cell antigen.
75. The antibody of embodiment 74, wherein the immune cell antigen is selected from the group consisting of PD-1, PD-L1, CTLA4, CD3, LAG3, OX40, CD28, CD33, B7H3, CD47, TIM3, ICOS, LGR5, 4-1BB, CD40, CD40-L, and TIGIT.
76. The antibody of embodiment 73, comprising a second antigen-binding region that specifically binds to a tumor-specific antigen.
77. The antibody of embodiment 76, wherein the tumor-specific antigen is selected from the group consisting of HER2, HER3, EGFR, VEGF, IGF-1, IGF-2, ANG2, DLL1, IGF-1R, cMET, DLL4, FAP, DR5, IL15, IL15Ra, CD3, CEA, EPCAM, HER3, PSMA, PMEL, and GPC3.
78. The antibody of embodiment 74 to 76, wherein the immune cell antigen or tumor-specific antigen is CD3.
79. The antibody of embodiment 73, wherein the antibody is a biparatopic antibody.
80. The antibody of embodiment 79, comprising two antigen-binding regions, wherein each antigen-binding region specifically binds to a unique, non-overlapping CD36 epitope.
81. The antibody of embodiment 80, comprising a first antigen-binding domain, which comprises the antigen-binding domain of 1G04.
82. The antibody of embodiment 81, further comprising a second antigen-binding domain, which comprises an antigen-binding domain of an antibody selected from the group consisting of 10G04, 11G04, 19G04, 20G04, and 30G04.
83. The antibody of any of embodiments 80-82, comprising a first antigen-binding domain, which comprises the antigen-binding domain of 1G04, and a second antigen-binding domain, which comprises an antigen-binding domain of 11G04.
84. The antibody of any one of embodiments 1-71, which is an antigen binding fragment.
85. The antigen binding fragment of embodiment 84, wherein the antigen binding fragment comprises a Fab, Fab′, F(ab′)₂, single chain Fv (scFv), disulfide linked Fv, V-NAR domain, IgNar, intrabody, IgGΔCH2, minibody, F(ab′)₃, tetrabody, triabody, diabody, single-domain antibody, DVD-Ig, Fcab, mAb2, (scFv)₂, or scFv-Fc.
86. A pharmaceutical composition comprising the antibody of any one of embodiments 1 to 87 and a pharmaceutically acceptable excipient.
87. The pharmaceutical composition of embodiment 86, wherein at least 95% of the antibodies in the composition are afucosylated.
88. The pharmaceutical composition of embodiment 86 or embodiment 87, which further comprises a PD-1 inhibitor.
89. The pharmaceutical composition of embodiment 88, wherein the PD-1 inhibitor is an anti-PD-1 antibody.
90. The pharmaceutical composition of embodiment 89, wherein the anti-PD-1 antibody is pembrolizumab, pidilizumab, or nivolumab.
91. The pharmaceutical composition of any one of embodiments 86 to 90, which further comprises a PD-L1 inhibitor
92. The pharmaceutical composition of embodiment 91, wherein the PD-L1 inhibitor is an anti-PD-L1 antibody.
93. The pharmaceutical composition of embodiment 92, wherein the anti-PD-L1 antibody is atezolizumab, durvalumab, avelumab, or BMS-936559.
94. The pharmaceutical composition of any one of embodiments 86 to 93, which further comprises a CTLA-4 inhibitor.
95. The pharmaceutical composition of embodiment 94, wherein the CTLA-4 inhibitor is an anti-CTLA-4 antibody.
96. The pharmaceutical composition of embodiment 95, wherein the anti-CTLA-4 antibody is ipilimumab.
97. The pharmaceutical composition of any one of embodiments 86 to 96, wherein the composition further comprises a chemotherapeutic agent.
98. The pharmaceutical composition of embodiment 87, wherein the chemotherapeutic agent is cisplatin.
99. A method of treating cancer in a patient comprising administering to a subject in need thereof a therapeutically effective amount of an anti-CD36 antibody, wherein the antibody inhibits fatty acid uptake in HEK 293 cells expressing CD36 with an IC₅₀ of less than 20 nM, as measured by FACS assay.
100. A method of treating cancer in a patient comprising administering to a subject in need thereof a therapeutically effective amount of an anti-CD36 antibody, wherein the anti-CD36 antibody inhibits oxLDL uptake with an IC₅₀ of less than 10 nM, as measured by the anti-CD36 antibody's ability to inhibit uptake of oxLDL linked to a fluorophore into SCC cells stably expressing human CD36.
101. The method of embodiment 99 or embodiment 100, wherein the cancer is oral squamous cell carcinoma, head and neck cancer, esophageal cancer, gastric cancer, ovarian cancer, cervical cancer, lung cancer, breast cancer, colon cancer, renal cancer, prostate cancer, sarcoma, melanoma, leukemia, or lymphoma.
102. A method of treating one or more metastatic tumors in a patient comprising administering to a subject in need thereof a therapeutically effective amount of an anti-CD36 antibody, wherein the antibody inhibits fatty acid uptake in HEK 293 cells expressing CD36 with an IC₅₀ of less than 20 nM, as measured by FACS assay.
103. A method of treating one or more metastatic tumors in a patient comprising administering to a subject in need thereof a therapeutically effective amount of an anti-CD36 antibody, wherein the anti-CD36 antibody inhibits oxLDL uptake with an IC₅₀ of less than 10 nM, as measured by the anti-CD36 antibody's ability to inhibit uptake of oxLDL linked to a fluorophore into SCC cells stably expressing human CD36.
104. The method of any one of embodiments 99 to 103, wherein the anti-CD36 antibody is the antibody of any one of embodiments 1 to 85.
105. A method of treating cancer in a patient comprising administering to a subject in need thereof a therapeutically effective amount of the antibody of any one of embodiments 1 to 85, or a therapeutically effective amount of the pharmaceutical composition of any one of embodiments 86 to 98.
106. The method of embodiment 105, wherein the cancer is oral squamous cell carcinoma, head and neck cancer, esophageal cancer, gastric cancer, ovarian cancer, cervical cancer, lung cancer, breast cancer, colon cancer, renal cancer, prostate cancer, sarcoma, liposarcoma, melanoma, leukemia, or lymphoma.
107. A method of treating one or more metastatic tumors in a patient comprising administering to a subject in need thereof a therapeutically effective amount of the antibody of any one of embodiments 1 to 85, or a therapeutically effective amount of the pharmaceutical composition of any one of embodiments 86 to 98.
108. The method of embodiment 102 or embodiment 107, wherein the metastatic tumors are metastatic oral squamous cell carcinoma, metastatic head and neck cancer, metastatic esophageal cancer, metastatic gastric cancer, metastatic ovarian cancer, metastatic cervical cancer, metastatic lung cancer, metastatic breast cancer, metastatic colon cancer, metastatic renal cancer, metastatic prostate cancer, metastatic sarcoma, metastatic melanoma, metastatic leukemia, or metastatic lymphoma.
109. The method of any one of embodiments 99 to 108, wherein the treatment reduces the size of metastatic tumors, as measured by IVIS imaging or H&E staining.
110. The method of any one of embodiments 99 to 109, wherein the treatment inhibits the formation or development of metastatic tumors, as measured by IVIS imaging or H&E staining.
111. The method of any one of embodiments 99 to 110, wherein the anti-CD36 antibody blocks the CD36-mediated uptake of fatty acids and/or oxLDL, while blocking less than 50% of CD36's binding to TSP-1 as measured by surface plasmon resonance.
112. The method of any one of embodiments 99 to 111, wherein the patient is a human patient.
113. The method of any one of embodiments 99 to 112, wherein the anti-CD36 antibody is a full length antibody, a single chain antibody, a scFv, a Fab fragment, or a F(ab′)₂ fragment.
114. The method of any one of embodiments 99 to 113, wherein the anti-CD36 antibody is a full length antibody.
115. The method of embodiment 114, wherein the anti-CD36 antibody comprises the antibody of any one of embodiments 38 to 40.
116. The method of any one of embodiments 99-115, wherein the method further comprises administering a second therapy.
117. The method of embodiment 116, wherein the second therapy is an immunotherapy.
118. The method of embodiment 117, wherein the immunotherapy is a PD-1 inhibitor.
119. The method of embodiment 118, wherein the PD-1 inhibitor is an anti-PD-1 antibody.
120. The method of embodiment 119, wherein the anti-PD-1 antibody is pembrolizumab, pidilizumab, or nivolumab.
121. The method of embodiment 117, wherein the immunotherapy is a PD-L1 inhibitor.
122. The method of embodiment 121, wherein the PD-L1 inhibitor is an anti-PD-L1 antibody.
123. The method of embodiment 122, wherein the anti-PD-L1 antibody is atezolizumab, durvalumab, avelumab, or BMS-936559.
124. The method of embodiment 117, wherein the immunotherapy is a CTLA-4 inhibitor.
125. The method of embodiment 124, wherein the CTLA-4 inhibitor is an anti-CTLA-4 antibody.
126. The method of embodiment 125, wherein the anti-CTLA-4 antibody is ipilimumab.
127. The method embodiment 116, wherein the second therapy is a chemotherapeutic agent.
128. The method of embodiment 127, wherein the chemotherapeutic agent is cisplatin.
129. The method of any one of embodiments 99-128, wherein metastasis is reduced or inhibited in the subject.
130. The method of any one of embodiments 116-129, wherein the two therapies are administered sequentially.
131. The method of any one of embodiments 116-129, wherein the two therapies are administered simultaneously.
132. The antibody of any one of embodiments 1 to 85, for use in a method of treating a subject having a cancer that expresses CD36, the method comprising administering to the subject a therapeutically effective amount of the anti-CD36 antibody according to the invention.
133. The antibody for use of embodiment 132, wherein the cancer is oral squamous cell carcinoma, head and neck cancer, esophageal cancer, gastric cancer, ovarian cancer, cervical cancer, lung cancer, breast cancer, colon cancer, renal cancer, prostate cancer, sarcoma, melanoma, leukemia, or lymphoma.
134. The antibody for use of embodiment 132 or embodiment 133, wherein the cancer is a metastatic cancer.
135. The antibody for use of any one of embodiments 132 to 134, wherein the treatment reduces the size of metastatic tumors, as measured by IVIS imaging or H&E staining.
136. The antibody for use of any one of embodiments 132 to 135, wherein the treatment inhibits the formation or development of metastatic tumors, as measured by IVIS imaging or H&E staining.
137. The antibody for use of any one of embodiments 132 to 136, wherein the anti-CD36 antibody blocks the CD36-mediated uptake of fatty acids and/or oxLDL while having little to no effect on CD36's binding to TSP-1.
138. The antibody for use of any one of embodiments 132-137, wherein the antibody inhibits fatty acid uptake in HEK 293 cells expressing CD36 with an IC₅₀ of less than 20 nM, as measured by FACS assay.
139. The antibody for use of any one of embodiments 132-138, wherein the anti-CD36 antibody inhibits oxLDL uptake with an IC₅₀ of less than 10 nM, as measured by the anti-CD36 antibody's ability to inhibit uptake of oxLDL linked to a fluorophore into SCC cells stably expressing human CD36.
140. The antibody for use of any one of embodiments 132 to 139, wherein the use is in combination with a second therapy.
141. The antibody for use of embodiment 140, wherein the second therapy is an immunotherapy.
142. The antibody for use of embodiment 141, wherein the immunotherapy is an anti-PD-1 antibody, an anti-PL-L1 antibody, or an anti-CTLA-4 antibody.
143. The antibody for use of embodiment 140, wherein the second therapy is a chemotherapeutic agent.
144. The antibody for use of embodiment 143, wherein the chemotherapeutic agent is cisplatin.
145. Use of the antibody of any one of embodiments 1 to 85 in the manufacture of a medicament for treating a subject having a cancer that expresses CD36.
146. The use of the antibody according to embodiment 145, wherein the cancer is oral squamous cell carcinoma, head and neck cancer, esophageal cancer, gastric cancer, ovarian cancer, cervical cancer, lung cancer, breast cancer, colon cancer, renal cancer, prostate cancer, sarcoma, melanoma, leukemia, or lymphoma.
147. The use of the antibody according to embodiment 145 or embodiment 146, wherein the cancer is a metastatic cancer.
148. The use of the antibody according to any one of embodiments 145 to 147, wherein the treatment reduces the size of metastatic tumors, as measured by IVIS imaging or H&E staining.
149. The use of the antibody according to any one of embodiments 145 to 148, wherein the treatment inhibits the formation or development of metastatic tumors, as measured by IVIS imaging or H&E staining.
150. The use of the antibody according to any one of embodiments 145 to 149, wherein the anti-CD36 antibody blocks the CD36-mediated uptake of fatty acids and/or oxLDL while having little to no effect on CD36's binding to TSP-1.
151. The use of the antibody according to any one of embodiments 145-150, wherein the antibody inhibits fatty acid uptake in HEK 293 cells expressing CD36 with an IC₅₀ of less than 20 nM, as measured by FACS assay.
152. The use of the antibody according to any one of embodiments 145-151, wherein the anti-CD36 antibody inhibits oxLDL uptake with an IC₅₀ of less than 10 nM, as measured by the anti-CD36 antibody's ability to inhibit uptake of oxLDL linked to a fluorophore into SCC cells stably expressing human CD36.
153. The use of the antibody according to any one of embodiments 145 to 152, wherein the use is in combination with a second therapy.
154. The use of the antibody according to embodiment 153, wherein the second therapy is an immunotherapy.
155. The use of the antibody according to embodiment 154, wherein the immunotherapy is an anti-PD-1 antibody, an anti-PL-L1 antibody, or an anti-CTLA-4 antibody.
156. The use of the antibody according to embodiment 153, wherein the second therapy is a chemotherapeutic agent.
157. The use of the antibody according to embodiment 156, wherein the chemotherapeutic agent is cisplatin.
158. An isolated polynucleotide that encodes the antibody of any one of embodiments 1 to 85.
159. The isolated polynucleotide of embodiment 158, which encodes the light chain variable region and the heavy chain variable region of embodiment 40.
160. The isolated polynucleotide of embodiment 158 or 159, which comprises at least one polynucleotide encoding a heavy chain selected from the group consisting of SEQ ID NOs: 226, 228, 230, 232, 234, 236, 257, and 258.
161. The isolated polynucleotide of any one of embodiments 158 to 160, which comprises at least one polynucleotide encoding a light chain selected from the group consisting of SEQ ID NOs: 227, 229, 231, 233, 235, 237, and 253-256.
162. The isolated polynucleotide of any one of embodiments 158 to 161, wherein the isolated polynucleotide comprises SEQ ID NOs: 226 and 227, SEQ ID NOs: 228 and 229, SEQ ID NOs: 230 and 231, SEQ ID NOs: 232 and 233, SEQ ID NOs: 234 and 235, SEQ ID NOs: 236 and 237, SEQ ID NOs: 253 and 257, SEQ ID NOs: 253 and 258, SEQ ID NOs: 254 and 257, SEQ ID NOs: 254 and 258, SEQ ID NOs: 255 and 257, SEQ ID NOs: 255 and 258, SEQ ID NOs: 256 and 257, or SEQ ID NOs: 256 and 258.
163. A vector comprising the isolated polynucleotide of any one of embodiments 158 to 162.
164. A cell comprising the isolated polynucleotide of any one of embodiments 158 to 162 or the vector of embodiment 163.
165. The cell of embodiment 164, which is selected from the group consisting of E. coli, Pseudomonas, Bacillus, Streptomyces, yeast, CHO, YB/20, NS0, PER-C6, HEK 293, HEK 293T, NIH 3T3, HeLa, BHK, Hep G2, SP2/0, R1.1, B-W, L-M, COS 1, COS 7, BSC1, BSC40, BMT10 cell, plant cell, insect cell, and human cell in tissue culture.
166. The cell of embodiment 164 or 165, wherein the cell lacks a functional alpha-1,6-fucosyltransferase gene (FUT8) gene.
167. A method of making an antibody that is capable of specifically binding CD36, comprising culturing the cell of any one of embodiments 164 to 166 under conditions suitable for expression of the antibody and isolating the antibody expressed therein.
168. A method of embodiment 167, wherein the the antibody is secreted from the cell and isolated from media in which the cell has been cultured.
169. The use of an antibody of any one of embodiments 1 to 85, for the manufacture of a pharmaceutical composition.
170. The use of an antibody of any one of embodiments 1 to 85 and a pharmaceutically acceptable excipient or carrier for the manufacture of a pharmaceutical composition.
171. The method of any one of embodiments 99 to 131, wherein the metastatic tumors are present in one or more of the liver, lung, spleen, kidney, cervical lymph nodes, or peritoneal wall.
172. The antibody for use of any one of embodiments 132 to 144, wherein the metastatic cancer comprises metastatic tumors in one or more of the liver, lung, spleen, kidney, cervical lymph nodes, or peritoneal wall.
173. The use of the antibody of any one of embodiments 145-157, wherein the metastatic cancer comprises metastatic tumors in one or more of the liver, lung, spleen, kidney, cervical lymph nodes, or peritoneal wall.
174. A method of treating both a primary tumor and metastatic tumors in a patient comprising administering to a subject in need thereof a therapeutically effective amount of the antibody of any one of embodiments 1 to 85 or a therapeutically effective amount of the pharmaceutical composition of any one of embodiments 86 to 98.
175. The method of embodiment 174, wherein the cancer is oral squamous cell carcinoma, head and neck cancer, esophageal cancer, gastric cancer, ovarian cancer, cervical cancer, lung cancer, breast cancer, colon cancer, renal cancer, prostate cancer, sarcoma, melanoma, leukemia, or lymphoma.
176. The method of embodiment 174 or 175, wherein the metastatic tumors are metastatic oral squamous cell carcinoma, metastatic head and neck cancer, metastatic esophageal cancer, metastatic gastric cancer, metastatic ovarian cancer, metastatic cervical cancer, metastatic lung cancer, metastatic breast cancer, metastatic colon cancer, metastatic renal cancer, metastatic prostate cancer, metastatic sarcoma, metastatic melanoma, metastatic leukemia, or metastatic lymphoma.
177. The method of any one of embodiments 174 to 176, wherein the treatment reduces the size of metastatic tumors, as measured by IVIS imaging or H&E staining.
178. The method of any one of embodiments 174 to 177 wherein the treatment reduces the size of a primary tumor.
179. The method of any one of embodiments 174 to 178, wherein the treatment inhibits the formation or development of metastatic tumors, as measured by IVIS imaging or H&E staining.
180. The method of any one of embodiments 174 to 179, wherein the anti-CD36 antibody blocks the CD36-mediated uptake of fatty acids and/or oxLDL while blocking less than 50% of CD36's binding to TSP-1 as measured by surface plasmon resonance.
181. The method of any one of embodiments 174-180, wherein the antibody inhibits fatty acid uptake in HEK 293 cells expressing CD36 with an IC₅₀ of less than 20 nM, as measured by FACS assay.
182. The method of any one of embodiments 174-181, wherein the anti-CD36 antibody inhibits oxLDL uptake with an IC₅₀ of less than 10 nM, as measured by the anti-CD36 antibody's ability to inhibit uptake of oxLDL linked to a fluorophore into SCC cells stably expressing human CD36.
183. The method of any one of embodiments 174 to 182, wherein the patient is a human patient.
184. The method of any one of embodiments 174 to 183, wherein the anti-CD36 antibody is a full length antibody, a single chain antibody, a scFv, a Fab fragment, or a F(ab′)₂ fragment.
185. The method of any one of embodiments 174 to 184, wherein the anti-CD36 antibody is a full length antibody.
186. The method of embodiment 185, wherein the anti-CD36 antibody comprises the antibody of any one of embodiments 38 to 40.
187. The method of any one of embodiments 174 to 186, wherein the method further comprises administering a second therapy.
188. The method of embodiment 187, wherein the second therapy is an immunotherapy.
189. The method of embodiment 188, wherein the immunotherapy is a PD-1 inhibitor.
190. The method of embodiment 189, wherein the PD-1 inhibitor is an anti-PD-1 antibody.
191. The method of embodiment 190, wherein the anti-PD-1 antibody is pembrolizumab, pidilizumab, or nivolumab.
192. The method of embodiment 188, wherein the immunotherapy is a PD-L1 inhibitor.
193. The method of embodiment 192, wherein the PD-L1 inhibitor is an anti-PD-L1 antibody.
194. The method of embodiment 193, wherein the anti-PD-L1 antibody is atezolizumab, durvalumab, avelumab, or BMS-936559.
195. The method of embodiment 188, wherein the immunotherapy is a CTLA-4 inhibitor.
196. The method of embodiment 195, wherein the CTLA-4 inhibitor is an anti-CTLA-4 antibody.
197. The method of embodiment 196, wherein the anti-CTLA-4 antibody is ipilimumab.
198. The method embodiment 187, wherein the second therapy is a chemotherapeutic agent.
199. The method of embodiment 198, wherein the chemotherapeutic agent is cisplatin.
200. The method of any one of embodiments 174 to 199, wherein metastasis is reduced or inhibited in the subject.
201. The method of any one of embodiments 187-200, wherein the two therapies are administered sequentially.
202. The method of any one of embodiments 187-200, wherein the two therapies are administered simultaneously.
203. An antibody that binds to human CD36, wherein the epitope comprises or consists of amino acid residues 149-156 and/or amino acid residues 188-196 and/or amino acid residues 400-403 within human CD36 defined by SEQ ID NO: 1; preferably all, more preferably wherein determination of the epitope is conducted by hydrogen-deuterium exchange mass spectrometry.
204. The antibody of embodiment 203, wherein the epitope comprises or consists of amino acid residues 145-160 and/or amino acid residues 185-199 and/or amino acid residues 398-414 within human CD36 defined by SEQ ID NO: 1, preferably the epitope comprises or consists of all said amino acid residues.
205. An antibody that specifically binds to human CD36, that binds to the same epitope on human CD36 as antibody 1G04 or competes with antibody 1G04 for binding to human CD36.
206. The antibody of any of embodiments 203 to 205, wherein said antibody is not 1G04 or wherein said antibody is 1G06.
207. The antibody according to any of the embodiments 203 to 206, wherein said antibody is an antibody having the HCDR3 of SEQ ID NO: 136 or an HCDR3 with at least a 70% identity thereto with at least a 70% identity thereto.
208. The antibody according to embodiment 207, wherein said antibody is selected from the group consisting of:
   a) an antibody wherein the heavy chain CDR1 region comprises or consists of SEQ ID NO: 87, the heavy chain CDR2 region comprises or consists of SEQ ID NO: 109, the heavy chain CDR3 region comprises or consists of SEQ ID NO: 136, the light chain CDR1 region comprises or consists of SEQ ID NO: 160, the light chain CDR2 region comprises or consists of SEQ ID NO: 174, and the light chain CDR3 region comprises or consists of SEQ ID NO: 187; preferably wherein the heavy chain variable region comprises or consists of SEQ ID NO: 16 and the light chain variable region comprises or consists of SEQ ID NO: 48; more preferably wherein said antibody is 7G04;
   b) an antibody wherein the heavy chain CDR1 region comprises or consists of SEQ ID NO: 88, the heavy chain CDR2 region comprises or consists of SEQ ID NO: 110, the heavy chain CDR3 region comprises or consists of SEQ ID NO: 137, the light chain CDR1 region comprises or consists of SEQ ID NO: 160, the light chain CDR2 region comprises or consists of SEQ ID NO: 174, and the light chain CDR3 region comprises or consists of SEQ ID NO: 187; preferably wherein the heavy chain variable region comprises or consists of SEQ ID NO: 17 and the light chain variable region comprises or consists of SEQ ID NO: 49; more preferably wherein said antibody is 09G04.
209. The antibody according to any of the embodiments 203 to 206, wherein said antibody is an antibody having the HCDR3 of SEQ ID NO: 142 or an HCDR3 with at least a 70% identity thereto.
210. The antibody according to embodiment 209, wherein the heavy chain CDR1 region comprises or consists of SEQ ID NO: 93, the heavy chain CDR2 region comprises or consists of SEQ ID NO: 115, the heavy chain CDR3 region comprises or consists of SEQ ID NO: 142, the light chain CDR1 region comprises or consists of SEQ ID NO: 163, the light chain CDR2 region comprises or consists of SEQ ID NO: 177, and the light chain CDR3 region comprises or consists of SEQ ID NO: 192; preferably wherein the heavy chain variable region comprises or consists of SEQ ID NO: 22 and the light chain variable region comprises or consists of SEQ ID NO: 54; more preferably wherein said antibody is 14G04.
211. The antibody according to any of the embodiments 203 to 206, wherein said antibody is an antibody having the HCDR3 of SEQ ID NO:133 or an HCDR3 with at least a 70% identity thereto.
212. The antibody according to embodiment 211, wherein said antibody is selected from the group consisting of:
   i. an antibody wherein the heavy chain CDR1 region comprises or consists of SEQ ID NO: 85, the heavy chain CDR2 region comprises or consists of SEQ ID NO: 106, the heavy chain CDR3 region comprises or consists of SEQ ID NO: 133, the light chain CDR1 region comprises or consists of SEQ ID NO: 159, the light chain CDR2 region comprises or consists of SEQ ID NO: 173, and the light chain CDR3 region comprises or consists of SEQ ID NO: 186; preferably wherein the heavy chain variable region comprises or consists of SEQ ID NO: 13 and the light chain variable region comprises or consists of SEQ ID NO: 45; more preferably wherein said antibody is 04G04
   ii. an antibody wherein the heavy chain CDR1 region comprises or consists of SEQ ID NO: 99, the heavy chain CDR2 region comprises or consists of SEQ ID NO: 126, the heavy chain CDR3 region comprises or consists of SEQ ID NO: 133, the light chain CDR1 region comprises or consists of SEQ ID NO: 162, the light chain CDR2 region comprises or consists of SEQ ID NO: 176, and the light chain CDR3 region comprises or consists of SEQ ID NO: 199; preferably wherein the heavy chain variable region comprises or consists of SEQ ID NO: 38 and the light chain variable region comprises or consists of SEQ ID NO: 71; more preferably wherein said antibody is 31G04.
213. The antibody according to any of the embodiments 203 to 206, wherein said antibody is an antibody having the HCDR3 of SEQ ID NO: 141 or an HCDR3 with at least a 70% identity thereto.
214. The antibody according to embodiment 213, wherein the heavy chain CDR1 region comprises or consists of SEQ ID NO: 92, the heavy chain CDR2 region comprises or consists of SEQ ID NO: 114, the heavy chain CDR3 region comprises or consists of SEQ ID NO: 141, the light chain CDR1 region comprises or consists of SEQ ID NO: 162, the light chain CDR2 region comprises or consists of SEQ ID NO: 176, and the light chain CDR3 region comprises or consists of SEQ ID NO: 191; preferably wherein the heavy chain variable region comprises or consists of SEQ ID NO: 21 and the light chain variable region comprises or consists of SEQ ID NO: 53; more preferably, wherein said antibody is 13G04.
215. The antibody according to any of the embodiments 203 to 206, wherein said antibody is an antibody having the HCDR3 of SEQ ID NO:147 or an HCDR3 with at least a 70% identity thereto.
216. The antibody according to embodiment 215, wherein said antibody is selected from the group consisting of:
   a. an antibody wherein the heavy chain CDR1 region comprises or consists of SEQ ID NO: 96, the heavy chain CDR2 region comprises or consists of SEQ ID NO: 120, the heavy chain CDR3 region comprises or consists of SEQ ID NO: 147, the light chain CDR1 region comprises or consists of SEQ ID NO: 159, the light chain CDR2 region comprises or consists of SEQ ID NO: 173, and the light chain CDR3 region comprises or consists of SEQ ID NO: 194; preferably wherein the heavy chain variable region comprises or consists of SEQ ID NO: 28 and the light chain variable region comprises or consists of SEQ ID NO: 61; more preferably wherein said antibody is 21G04;
   b. an antibody wherein the heavy chain CDR1 region comprises or consists of SEQ ID NO: 96, the heavy chain CDR2 region comprises or consists of SEQ ID NO: 121, the heavy chain CDR3 region comprises or consists of SEQ ID NO: 148, the light chain CDR1 region comprises or consists of SEQ ID NO: 159, the light chain CDR2 region comprises or consists of SEQ ID NO: 173, and the light chain CDR3 region comprises or consists of SEQ ID NO: 195; preferably wherein the heavy chain variable region comprises or consists of SEQ ID NO: 29 and the light chain variable region comprises or consists of SEQ ID NO: 62; preferably wherein said antibody is 22G04;
   c. an antibody wherein the heavy chain CDR1 region comprises or consists of SEQ ID NO: 96, the heavy chain CDR2 region comprises or consists of SEQ ID NO: 120, the heavy chain CDR3 region comprises or consists of SEQ ID NO: 147, the light chain CDR1 region comprises or consists of SEQ ID NO: 159, the light chain CDR2 region comprises or consists of SEQ ID NO: 173, and the light chain CDR3 region comprises or consists of SEQ ID NO: 186; preferably wherein the heavy chain variable region comprises or consists of SEQ ID NO: 30 and the light chain variable region comprises or consists of SEQ ID NO: 63; more preferably wherein said antibody is 23G04;
   d. an antibody wherein the heavy chain CDR1 region comprises or consists of SEQ ID NO: 96, the heavy chain CDR2 region comprises or consists of SEQ ID NO: 121, the heavy chain CDR3 region comprises or consists of SEQ ID NO: 147, the light chain CDR1 region comprises or consists of SEQ ID NO: 159, the light chain CDR2 region comprises or consists of SEQ ID NO: 173, and the light chain CDR3 region comprises or consists of SEQ ID NO: 196; preferably wherein the heavy chain variable region comprises or consists of SEQ ID NO: 31 and the light chain variable region comprises or consists of SEQ ID NO: 64; more preferably wherein said antibody is 24G04;
   e. an antibody wherein the heavy chain CDR1 region comprises or consists of SEQ ID NO: 96, the heavy chain CDR2 region comprises or consists of SEQ ID NO: 121, the heavy chain CDR3 region comprises or consists of SEQ ID NO: 147, the light chain CDR1 region comprises or consists of SEQ ID NO: 159, the light chain CDR2 region comprises or consists of SEQ ID NO: 173, and the light chain CDR3 region comprises or consists of SEQ ID NO: 195; preferably wherein the heavy chain variable region comprises or consists of SEQ ID NO: 32 and the light chain variable region comprises or consists of SEQ ID NO: 65; more preferably wherein said antibody is 25G04;
   f. an antib'ody wherein the heavy chain CDR1 region comprises or consists of SEQ ID NO: 96, the heavy chain CDR2 region comprises or consists of SEQ ID NO: 120, the heavy chain CDR3 region comprises or consists of SEQ ID NO: 147, the light chain CDR1 region comprises or consists of SEQ ID NO: 159, the light chain CDR2 region comprises or consists of SEQ ID NO: 173, and the light chain CDR3 region comprises or consists of SEQ ID NO: 195; preferably wherein the heavy chain variable region comprises or consists of SEQ ID NO: 36 and the light chain variable region comprises or consists of SEQ ID NO: 69; more preferably wherein said antibody is 29G04.
217. The antibody according to any of the embodiments 203 to 206, wherein said antibody is an antibody having the HCDR3 of SEQ ID NO: 143 or an HCDR3 with at least a 70% identity thereto.
218. The antibody according to embodiment 217, wherein the heavy chain CDR1 region comprises or consists of SEQ ID NO: 94, the heavy chain CDR2 region comprises or consists of SEQ ID NO: 116, the heavy chain CDR3 region comprises or consists of SEQ ID NO: 143, the light chain CDR1 region comprises or consists of SEQ ID NO: 164, the light chain CDR2 region comprises or consists of SEQ ID NO: 175, and the light chain CDR3 region comprises or consists of SEQ ID NO: 193; preferably wherein the heavy chain variable region comprises or consists of SEQ ID NO: 23 and the light chain variable region comprises or consists of SEQ ID NO: 55; more preferably wherein said antibody is 15G04.
219. The antibody according to any of the embodiments 203 to 206, wherein said antibody is an antibody having the HCDR3 of SEQ ID NO: 150 or an HCDR3 with at least a 70% identity thereto.
220. The antibody according to embodiment 219, wherein the heavy chain CDR1 region comprises or consists of SEQ ID NO: 97, the heavy chain CDR2 region comprises or consists of SEQ ID NO: 123, the heavy chain CDR3 region comprises or consists of SEQ ID NO: 150, the light chain CDR1 region comprises or consists of SEQ ID NO: 165, the light chain CDR2 region comprises or consists of SEQ ID NO: 178, and the light chain CDR3 region comprises or consists of SEQ ID NO: 197; preferably wherein the heavy chain variable region comprises or consists of SEQ ID NO: 34 and the light chain variable region comprises or consists of SEQ ID NO: 67; more preferably wherein said antibody is 27G04.
221. The antibody according to any of the embodiments 203 to 206, wherein said antibody is an antibody having the HCDR3 of SEQ ID NO: 149 or an HCDR3 with at least a 70% identity thereto.
222. The antibody according to the embodiment 221, wherein the heavy chain CDR1 region comprises or consists of SEQ ID NO: 97, the heavy chain CDR2 region comprises or consists of SEQ ID NO: 122, the heavy chain CDR3 region comprises or consists of SEQ ID NO: 149, the light chain CDR1 region comprises or consists of SEQ ID NO: 165, the light chain CDR2 region comprises or consists of SEQ ID NO: 178, and the light chain CDR3 region comprises or consists of SEQ ID NO: 197; preferably wherein the heavy chain variable region comprises or consists of SEQ ID NO: 33 and the light chain variable region comprises or consists of SEQ ID NO: 66; more preferably wherein said antibody is 26G04.
223. The antibody according to any of the embodiments 203 to 206, wherein said antibody is an antibody having the HCDR3 of SEQ ID NO: 151 or an HCDR3 with at least a 70% identity thereto.
224. The antibody according to the embodiment 223, wherein the heavy chain CDR1 region comprises or consists of SEQ ID NO: 98, the heavy chain CDR2 region comprises or consists of SEQ ID NO: 124, the heavy chain CDR3 region comprises or consists of SEQ ID NO: 151, the light chain CDR1 region comprises or consists of SEQ ID NO: 160, the light chain CDR2 region comprises or consists of SEQ ID NO: 174, and the light chain CDR3 region comprises or consists of SEQ ID NO: 198; preferably wherein the heavy chain variable region comprises or consists of SEQ ID NO: 35 and the light chain variable region comprises or consists of SEQ ID NO: 68; more preferably wherein said antibody is 28G04.
225. The antibody according to any of the embodiments 203 to 206, wherein said antibody is an antibody having the HCDR3 of SEQ ID NO: 146 or with an HCDR3 at least a 70% identity thereto.
226. The antibody according to the embodiment 225, wherein the heavy chain CDR1 region comprises or consists of SEQ ID NO: 95, the heavy chain CDR2 region comprises or consists of SEQ ID NO: 119, the heavy chain CDR3 region comprises or consists of SEQ ID NO: 146, the light chain CDR1 region comprises or consists of SEQ ID NO: 160, the light chain CDR2 region comprises or consists of SEQ ID NO: 174, and the light chain CDR3 region comprises or consists of SEQ ID NO: 190; preferably wherein the heavy chain variable region comprises or consists of SEQ ID NO: 26 and the light chain variable region comprises or consists of SEQ ID NO: 58; more preferably wherein said antibody is 18G04.
227. The antibody according to any of the embodiments 203 to 206, wherein said antibody is an antibody having the HCDR3 of SEQ ID NO:144 or an HCDR3 with at least a 70% identity thereto.
228. The antibody according to the embodiment 227, wherein said antibody is selected from the group consisting of:
   a. an antibody wherein the heavy chain CDR1 region comprises or consists of SEQ ID NO: 95, the heavy chain CDR2 region comprises or consists of SEQ ID NO: 117, the heavy chain CDR3 region comprises or consists of SEQ ID NO: 144, the light chain CDR1 region comprises or consists of SEQ ID NO: 160, the light chain CDR2 region comprises or consists of SEQ ID NO: 174, and the light chain CDR3 region comprises or consists of SEQ ID NO: 190; preferably wherein the heavy chain variable region comprises or consists of SEQ ID NO: 24 and the light chain variable region comprises or consists of SEQ ID NO: 56; more preferably where said antibody is 16G04;
   b. an an'tibody wherein the heavy chain CDR1 region comprises or consists of SEQ ID NO: 95, the heavy chain CDR2 region comprises or consists of SEQ ID NO: 118, the heavy chain CDR3 region comprises or consists of SEQ ID NO: 145, the light chain CDR1 region comprises or consists of SEQ ID NO: 160, the light chain CDR2 region comprises or consists of SEQ ID NO: 174, and the light chain CDR3 region comprises or consists of SEQ ID NO: 187 preferably wherein the heavy chain variable region comprises or consists of SEQ ID NO: 25 and the light chain variable region comprises or consists of SEQ ID NO: 57; more preferably wherein said antibody is 17G04.
229. The antibody according to any of the embodiments 203 to 206, wherein said antibody is an antibody having the HCDR3 of SEQ ID NO:134 or an HCDR3 with at least a 70% identity thereto.
230. The antibody according to embodiment 229, wherein said antibody is selected from the group consisting of:
   a. an antibody wherein the heavy chain CDR1 region comprises or consists of SEQ ID NO: 86, the heavy chain CDR2 region comprises or consists of SEQ ID NO: 107, the heavy chain CDR3 region comprises or consists of SEQ ID NO: 134, the light chain CDR1 region comprises or consists of SEQ ID NO: 160, the light chain CDR2 region comprises or consists of SEQ ID NO: 174, and the light chain CDR3 region comprises or consists of SEQ ID NO: 187; preferably wherein the heavy chain variable region comprises or consists of SEQ ID NO: 14 and the light chain variable region comprises or consists of SEQ ID NO: 4; more preferably wherein said antibody is 05G04;
   b. an antibody wherein the heavy chain CDR1 region comprises or consists of SEQ ID NO: 86, the heavy chain CDR2 region comprises or consists of SEQ ID NO: 108, the heavy chain CDR3 region comprises or consists of SEQ ID NO: 135, the light chain CDR1 region comprises or consists of SEQ ID NO: 160, the light chain CDR2 region comprises or consists of SEQ ID NO: 174, and the light chain CDR3 region comprises or consists of SEQ ID NO: 188; preferably wherein the heavy chain variable region comprises or consists of SEQ ID NO: 15 and the light chain variable region comprises or consists of SEQ ID NO: 47; more preferably wherein said antibody is 06G04.
231. The antibody according to any of the embodiments 203 to 206, wherein said antibody is an antibody having the HCDR3 of SEQ ID NO: 140 or an HCDR3 with at least a 70% identity thereto.
232. The antibody according to embodiment 231, wherein the heavy chain CDR1 region comprises or consists of SEQ ID NO: 91, the heavy chain CDR2 region comprises or consists of SEQ ID NO: 113, the heavy chain CDR3 region comprises or consists of SEQ ID NO: 140, the light chain CDR1 region comprises or consists of SEQ ID NO: 160, the light chain CDR2 region comprises or consists of SEQ ID NO: 174, and the light chain CDR3 region comprises or consists of SEQ ID NO: 187; preferably wherein the heavy chain variable region comprises or consists of SEQ ID NO: 20 and the light chain variable region comprises or consists of SEQ ID NO: 52; more preferably wherein said antibody is 12G04.
233. The antibody according to any of the embodiments 203 to 206, wherein said antibody is an antibody having the HCDR3 of SEQ ID NO: 152 or an HCDR3 with at least a 70% identity thereto.
234. The antibody according to embodiment 233, wherein the heavy chain CDR1 region comprises or consists of SEQ ID NO: 91, the heavy chain CDR2 region comprises or consists of SEQ ID NO: 125, the heavy chain CDR3 region comprises or consists of SEQ ID NO: 152, the light chain CDR1 region comprises or consists of SEQ ID NO: 160, the light chain CDR2 region comprises or consists of SEQ ID NO: 174, and the light chain CDR3 region comprises or consists of SEQ ID NO: 190; preferably the heavy chain variable region comprises or consists of SEQ ID NO: 37 and the light chain variable region comprises or consists of SEQ ID NO: 70; more preferably wherein said antibody is 30G04.
235. The antibody according to any of the embodiments 203 to 206, wherein said antibody is an antibody having the HCDR3 of SEQ ID NO: 153 or an HCDR3 with at least a 70% identity thereto.
236. The antibody according to embodiment 235, wherein the heavy chain CDR1 region comprises or consists of SEQ ID NO: 100, the heavy chain CDR2 region comprises or consists of SEQ ID NO: 127, the heavy chain CDR3 region comprises or consists of SEQ ID NO: 153, the light chain CDR1 region comprises or consists of SEQ ID NO: 166, the light chain CDR2 region comprises or consists of SEQ ID NO: 179, and the light chain CDR3 region comprises or consists of SEQ ID NO: 200; preferably wherein the heavy chain variable region comprises or consists of SEQ ID NO: 39 and the light chain variable region comprises or consists of SEQ ID NO: 72; more preferably wherein said antibody is 32G04.
237. The antibody according to any of the embodiments 203 to 206, wherein said antibody is an antibody having the HCDR3 of SEQ ID NO:154 or an HCDR3 with at least a 70% identity thereto.
238. The antibody according to embodiment 237, preferably wherein said antibody is selected from the group consisting of:
   a. an antibody wherein the heavy chain CDR1 region comprises or consists of SEQ ID NO: 101, the heavy chain CDR2 region comprises or consists of SEQ ID NO: 128, the heavy chain CDR3 region comprises or consists of SEQ ID NO: 154, the light chain CDR1 region comprises or consists of SEQ ID NO: 167, the light chain CDR2 region comprises or consists of SEQ ID NO: 180, and the light chain CDR3 region comprises or consists of SEQ ID NO: 201; preferably wherein the heavy chain variable region comprises or consists of SEQ ID NO: 40 and the light chain variable region comprises or consists of SEQ ID NO: 73; more preferably wherein said antibody is 33G04;
   b. an antibody wherein the heavy chain CDR1 region comprises or consists of SEQ ID NO: 101, the heavy chain CDR2 region comprises or consists of SEQ ID NO: 128, the heavy chain CDR3 region comprises or consists of SEQ ID NO: 154, the light chain CDR1 region comprises or consists of SEQ ID NO: 168, the light chain CDR2 region comprises or consists of SEQ ID NO: 181, and the light chain CDR3 region comprises or consists of SEQ ID NO: 202; preferably wherein the heavy chain variable region comprises or consists of SEQ ID NO: 40 and the light chain variable region comprises or consists of SEQ ID NO: 74; more preferably wherein said antibody is 34G04.
239. The antibody according to any of the embodiments 203 to 206, wherein said antibody is an antibody having the HCDR3 of SEQ ID NO:155 or an HCDR3 with at least a 70% identity thereto.
240. The antibody according to embodiment 239, wherein the heavy chain CDR1 region comprises or consists of SEQ ID NO: 102, the heavy chain CDR2 region comprises or consists of SEQ ID NO: 129, the heavy chain CDR3 region comprises or consists of SEQ ID NO: 155, the light chain CDR1 region comprises or consists of SEQ ID NO: 169, the light chain CDR2 region comprises or consists of SEQ ID NO: 182, and the light chain CDR3 region comprises or consists of SEQ ID NO: 203; preferably wherein the heavy chain variable region comprises or consists of SEQ ID NO: 41 and the light chain variable region comprises or consists of SEQ ID NO: 75; more preferably wherein said antibody is 35G04.
241. The antibody according to any of the embodiments 203 to 206, wherein said antibody is an antibody having the HCDR3 of SEQ ID NO:156 or an HCDR3 with at least a 70% identity thereto.
242. The antibody according to embodiment 241, wherein the heavy chain CDR1 region comprises or consists of SEQ ID NO: 103, the heavy chain CDR2 region comprises or consists of SEQ ID NO: 130, the heavy chain CDR3 region comprises or consists of SEQ ID NO: 156, the light chain CDR1 region comprises or consists of SEQ ID NO: 170, the light chain CDR2 region comprises or consists of SEQ ID NO: 183, and the light chain CDR3 region comprises or consists of SEQ ID NO: 204; preferably wherein the heavy chain variable region comprises or consists of SEQ ID NO: 42 and the light chain variable region comprises or consists of SEQ ID NO: 76; more preferably wherein said antibody is 36G04.
243. The antibody according to any of the embodiments 203 to 206, wherein said antibody is an antibody having the HCDR3 of SEQ ID NO:157 or an HCDR3 with at least a 70% identity thereto.
244. The antibody according to embodiment 243, wherein the heavy chain CDR1 region comprises or consists of SEQ ID NO: 104, the heavy chain CDR2 region comprises or consists of SEQ ID NO: 131, the heavy chain CDR3 region comprises or consists of SEQ ID NO: 157, the light chain CDR1 region comprises or consists of SEQ ID NO: 171, the light chain CDR2 region comprises or consists of SEQ ID NO: 184, and the light chain CDR3 region comprises or consists of SEQ ID NO: 205; preferably wherein the heavy chain variable region comprises or consists of SEQ ID NO: 43 and the light chain variable region comprises or consists of SEQ ID NO: 77; more preferably wherein said antibody is 37G04.
245. An antibody that binds to human CD36, wherein the epitope comprises or consists of or consists of amino acid residues 286-292 and/or amino acid residues 341-350 within human CD36 defined by SEQ ID NO: 1, preferably all the residues; more preferably wherein determination of the epitope is conducted by hydrogen-deuterium exchange mass spectrometry.
246. The antibody of embodiment 245, wherein the epitope comprises or consists of amino acid residues 280-298 and/or amino acid residues 341-365 within human CD36 defined by SEQ ID NO: 1, preferably comprises or consists of all the residues.
247. An antibody that specifically binds to human CD36, that binds to the same epitope on human CD36 as antibody 11G04 or competes with antibody 11G04 for binding to human CD36.
248. The antibody according to any of embodiments 245-247, wherein said antibody is an antibody having the HCDR3 of SEQ ID NO: 146 or an HCDR3 with at least a 70% identity thereto.
249. The antibody according to embodiment 248, wherein the heavy chain CDR1 region comprises or consists of or consists of SEQ ID NO: 95, the heavy chain CDR2 region comprises or consists of SEQ ID NO: 119, the heavy chain CDR3 region comprises or consists of SEQ ID NO: 146, the light chain CDR1 region comprises or consists of SEQ ID NO: 160, the light chain CDR2 region comprises or consists of SEQ ID NO: 174, and the light chain CDR3 region comprises or consists of SEQ ID NO: 190; preferably wherein said antibody is selected from the group consisting of:
   a) an antibody wherein the heavy chain variable region comprises or consists of SEQ ID NO: 26 and the light chain variable region comprises or consists of SEQ ID NO: 58; more preferably wherein said antibody is 18G04;
   b) an antibody wherein the heavy chain variable region comprises or consists of SEQ ID NO: 26 and the light chain variable region comprises or consists of SEQ ID NO: 59; more preferably wherein said antibody is 19G04;
   c) an antibody wherein the heavy chain variable region comprises or consists of SEQ ID NO: 27 and the light chain variable region comprises or consists of SEQ ID NO: 60; more preferably wherein said antibody is 20G04.
250. The antibody according to any of embodiments 245-247, wherein said antibody is an antibody having the HCDR3 of SEQ ID NO: 144 or an HCDR3 with at least a 70% identity thereto.
251. The antibody according to embodiment 250, wherein the heavy chain CDR1 region comprises or consists of SEQ ID NO: 95, the heavy chain CDR2 region comprises or consists of SEQ ID NO: 117, the heavy chain CDR3 region comprises or consists of SEQ ID NO: 144, the light chain CDR1 region comprises or consists of SEQ ID NO: 160, the light chain CDR2 region comprises or consists of SEQ ID NO: 174, and the light chain CDR3 region comprises or consists of SEQ ID NO: 190; preferably wherein the heavy chain variable region comprises or consists of SEQ ID NO: 24 and the light chain variable region comprises or consists of SEQ ID NO: 56; more preferably wherein said antibody is 16G04.
252. The antibody according to any of embodiments 245-247, wherein said antibody is an antibody having the HCDR3 of SEQ ID NO: 152 or an HCDR3 with at least a 70% identity thereto.
253. The antibody according to embodiment 252, wherein the heavy chain CDR1 region comprises or consists of SEQ ID NO: 91, the heavy chain CDR2 region comprises or consists of SEQ ID NO: 125, the heavy chain CDR3 region comprises or consists of SEQ ID NO: 152, the light chain CDR1 region comprises or consists of SEQ ID NO: 160, the light chain CDR2 region comprises or consists of SEQ ID NO: 174, and the light chain CDR3 region comprises or consists of SEQ ID NO: 190; preferably wherein the heavy chain variable region comprises or consists of SEQ ID NO: 37 and the light chain variable region comprises or consists of SEQ ID NO: 70; more preferably wherein said antibody is 30G04.
254. The antibody according to any of embodiments 245-247, wherein said antibody is an antibody having the HCDR3 of SEQ ID NO: 154 or an HCDR3 with at least a 70% identity thereto.
255. The antibody according to embodiment 254, wherein said antibody is selected from the group consisting of:
   a. an antibody wherein the heavy chain CDR1 region comprises or consists of SEQ ID NO: 101, the heavy chain CDR2 region comprises or consists of SEQ ID NO: 128, the heavy chain CDR3 region comprises or consists of SEQ ID NO: 154, the light chain CDR1 region comprises or consists of SEQ ID NO: 167, the light chain CDR2 region comprises or consists of SEQ ID NO: 180, and the light chain CDR3 region comprises or consists of SEQ ID NO: 201; preferably wherein the heavy chain variable region comprises or consists of SEQ ID NO: 40 and the light chain variable region comprises or consists of SEQ ID NO: 73; more preferably wherein said antibody is 33G04;
   b. an antibody wherein the heavy chain CDR1 region comprises or consists of SEQ ID NO: 101, the heavy chain CDR2 region comprises or consists of SEQ ID NO: 128, the heavy chain CDR3 region comprises or consists of SEQ ID NO: 154, the light chain CDR1 region comprises or consists of SEQ ID NO: 168, the light chain CDR2 region comprises or consists of SEQ ID NO: 181, and the light chain CDR3 region comprises or consists of SEQ ID NO: 202; preferably wherein the heavy chain variable region comprises or consists of SEQ ID NO: 40 and the light chain variable region comprises or consists of SEQ ID NO: 74; more preferably wherein said antibody is 34G04.
256. The antibody according to any of embodiments 245-247, wherein said antibody is an antibody having the HCDR3 of SEQ ID NO: 155 or an HCDR3 with at least a 70% identity thereto.
257. The antibody according to embodiment 256, wherein the heavy chain CDR1 region comprises or consists of SEQ ID NO: 102, the heavy chain CDR2 region comprises or consists of SEQ ID NO: 129, the heavy chain CDR3 region comprises or consists of SEQ ID NO: 155, the light chain CDR1 region comprises or consists of SEQ ID NO: 169, the light chain CDR2 region comprises or consists of SEQ ID NO: 182, and the light chain CDR3 region comprises or consists of SEQ ID NO: 203; preferably wherein the heavy chain variable region comprises or consists of SEQ ID NO: 41 and the light chain variable region comprises or consists of SEQ ID NO: 75; more preferably wherein said antibody is 35G04.
258. The antibody according to any of embodiments 245-247, wherein said antibody is an antibody having the HCDR3 of SEQ ID NO: 156 or an HCDR3 with at least a 70% identity thereto.
259. The antibody according to embodiment 258, wherein the heavy chain CDR1 region comprises or consists of SEQ ID NO: 103, the heavy chain CDR2 region comprises or consists of SEQ ID NO: 130, the heavy chain CDR3 region comprises or consists of SEQ ID NO: 156, the light chain CDR1 region comprises or consists of SEQ ID NO: 170, the light chain CDR2 region comprises or consists of SEQ ID NO: 183, and the light chain CDR3 region comprises or consists of SEQ ID NO: 204; preferably wherein the heavy chain variable region comprises or consists of SEQ ID NO: 42 and the light chain variable region comprises or consists of SEQ ID NO: 76; more preferably wherein said antibody is 36G04.
260. The antibody according to any of embodiments 245-247, wherein said antibody is an antibody having the HCDR3 of SEQ ID NO: 157 or an HCDR3 with at least a 70% identity thereto.
261. The antibody according to embodiment 260, wherein the heavy chain CDR1 region comprises or consists of SEQ ID NO: 104, the heavy chain CDR2 region comprises or consists of SEQ ID NO: 131, the heavy chain CDR3 region comprises or consists of SEQ ID NO: 157, the light chain CDR1 region comprises or consists of SEQ ID NO: 171, the light chain CDR2 region comprises or consists of SEQ ID NO: 184, and the light chain CDR3 region comprises or consists of SEQ ID NO: 205; preferably wherein the heavy chain variable region comprises or consists of SEQ ID NO: 43 and the light chain variable region comprises or consists of SEQ ID NO: 77; more preferably wherein said antibody is 37G04.
262. The antibody according to any of embodiments 245-247, wherein said antibody is an antibody having the HCDR3 of SEQ ID NO: 158 or an HCDR3 with at least a 70% identity thereto.
263. The antibody according to embodiment 262, wherein the heavy chain CDR1 region comprises or consists of SEQ ID NO: 105, the heavy chain CDR2 region comprises or consists of SEQ ID NO: 132, the heavy chain CDR3 region comprises or consists of SEQ ID NO: 158, the light chain CDR1 region comprises or consists of SEQ ID NO: 172, the light chain CDR2 region comprises or consists of SEQ ID NO: 185, and the light chain CDR3 region comprises or consists of SEQ ID NO: 206; preferably wherein the heavy chain variable region comprises or consists of SEQ ID NO: 44 and the light chain variable region comprises or consists of SEQ ID NO: 78; more preferably wherein said antibody is 38G04.
264. The antibody according to any of embodiments 245-247, wherein said antibody is an antibody having the HCDR3 of SEQ ID NO: 139 or an HCDR3 with at least a 70% identity thereto.
265. The antibody according to embodiment 262, wherein said antibody is selected from the group consisting of:
   a) an antibody wherein the heavy chain CDR1 region comprises or consists of SEQ ID NO: 90, the heavy chain CDR2 region comprises or consists of SEQ ID NO: 112, the heavy chain CDR3 region comprises or consists of SEQ ID NO: 139, the light chain CDR1 region comprises or consists of SEQ ID NO: 160, the light chain CDR2 region comprises or consists of SEQ ID NO: 174, and the light chain CDR3 region comprises or consists of SEQ ID NO: 190; preferably wherein the heavy chain variable region comprises or consists of SEQ ID NO: 19 and the light chain variable region comprises or consists of SEQ ID NO: 51; more preferably wherein said antibody is 11G04 or 11G06;
   b) an antibody wherein the heavy chain CDR1 region comprises or consists of SEQ ID NO: 90, the heavy chain CDR2 region comprises or consists of SEQ ID NO: 248, the heavy chain CDR3 region comprises or consists of SEQ ID NO: 139, the light chain CDR1 region comprises or consists of SEQ ID NO:160, the light chain CDR2 region comprises or consists of SEQ ID NO: 174, and the light chain CDR3 region comprises or consists of SEQ ID NO: 247; preferably wherein said antibody is selected from the group consisting of:
      i. an antibody wherein the heavy chain variable region comprises or consists of SEQ NO: 241 and the light chain variable region comprises or consists of SEQ ID NO: 240; preferably wherein said antibody is 73G06;
      ii. an antibody wherein the heavy chain variable region comprises or consists of SEQ NO: 243 and the light chain variable region comprises or consists of SEQ ID NO: 240; preferably wherein said antibody is 74G06;
      iii. an antibody wherein the heavy chain variable region comprises or consists of SEQ NO: 241 and the light chain variable region comprises or consists of SEQ ID NO: 242; preferably wherein said antibody is 75G06; and
      iv. an antibody wherein the heavy chain variable region comprises or consists of SEQ NO: 243 and the light chain variable region comprises or consists of SEQ ID NO: 242; preferably wherein said antibody is 76G06;
   c) wherein the heavy chain CDR1 region comprises or consists of SEQ ID NO: 90, the heavy chain CDR2 region comprises or consists of SEQ ID NO: 248, the heavy chain CDR3 region comprises or consists of SEQ ID NO: 139, the light chain CDR1 region comprises or consists of SEQ ID NO:160, the light chain CDR2 region comprises or consists of SEQ ID NO: 246, and the light chain CDR3 region comprises or consists of SEQ ID NO: 247; preferably wherein said antibody is selected from the group consisting of:
      i. the heavy chain variable region comprises or consists of SEQ NO: 241 and the light chain variable region comprises or consists of SEQ ID NO: 244; preferably wherein said antibody is 77G06;
      ii. the heavy chain variable region comprises or consists of SEQ NO: 243 and the light chain variable region comprises or consists of SEQ ID NO: 244; preferably wherein said antibody is 78G06;
      iii. the heavy chain variable region comprises or consists of SEQ NO: 241 and the light chain variable region comprises or consists of SEQ ID NO: 245; preferably wherein said antibody is 79G06; and
      iv. the heavy chain variable region comprises or consists of SEQ NO: 243 and the light chain variable region comprises or consists of SEQ ID NO: 245; preferably wherein said antibody is 80G06.
266. The antibody of any one of the preceding embodiments, wherein the antibody specifically binds to human CD36.
267. The antibody of embodiment 266, wherein the antibody has cross-reactivity to human CD36 and non-human CD36.
268. The antibody of embodiment 267, wherein the antibody has cross-reactivity to human CD36 and non-human primate CD36.
269. The antibody of embodiment 268, wherein the non-human primate CD36 is cynomomolgus CD36 or rhesus macaque CD36.
270. The antibody of embodiment 267, wherein the antibody has cross-reactivity to human CD36, non-human primate CD36, and rodent CD36.
271. The antibody of embodiment 270, wherein the rodent CD36 is mouse CD36 or rat CD36.
272. The antibody of any one of the preceding embodiments, wherein the antibody binds to human CD36 with a K_{D} of less than 20 nM, as measured using surface plasmon resonance with a bivalent model.
273. The antibody of embodiment 272, wherein the antibody binds to human CD36 with a K_{D} of less than 10 nM, as measured using surface plasmon resonance with a bivalent model.
274. The antibody of any of the preceding embodiments, wherein the antibody inhibits fatty acid uptake in HEK 293 cells expressing CD36 with an IC₅₀ of less than 20 nM, as measured by FACS assay.
275. The antibody of any of the preceding embodiments, wherein the anti-CD36 antibody inhibits oxLDL uptake with an IC₅₀ of less than 10 nM, as measured by the anti-CD36 antibody's ability to inhibit uptake of oxLDL linked to a fluorophore into SCC cells stably expressing human CD36.
276. The antibody of any one of the preceding embodiments, which comprises a heavy chain constant region.
277. The antibody of embodiment 276, wherein the heavy chain constant region is selected from the group consisting of human immunoglobulin IgA1, IgA2, IgG1, IgG2, IgG3, or IgG4 heavy chain constant regions.
278. The antibody of embodiment 277, which comprises an IgG1 heavy chain constant region.
279. The antibody of embodiment 277 or 278, wherein the heavy chain constant region comprises an IgG constant region containing at least one amino acid substitution, wherein the at least one amino acid substitution results in reduced Fc binding to at least one Fcgamma receptor and reduced Fc effector function.
280. The antibody of embodiment 277 or 278, wherein the at least one Fc silencing mutation includes the amino acid substitutions L234A and L235A (“LALA”).
281. The antibody of embodiment 279, wherein the at least one Fc silencing mutation includes a set of amino acid substitutions selected from the group consisting of L234G, L235S, and G236R; L234S, L235T, and G236R; L234S, L235V, and G236R; L234T, L235Q, and G236R; L234T, L235T, and G236R; L234A, L235S, and G236R; L234Q, L235S, and G236R; L234S, L235G, and G236R; L234T, L235S, and G236R; L234Q, L235S, and G236R; L234A and L235A; and L234A, L235A, and P329G.
282. The antibody of embodiment 277, which comprises an IgG4 heavy chain constant region.
283. The antibody of any of embodiments 281 or 282, wherein the heavy chain constant region comprises an IgG constant region containing the amino acid substitution S228P; preferably wherein said heavy chain constant region is a human IgG4 constant region containing the amino acid substitutions S228P and L235E.
284. The antibody of any one of the preceding embodiments, wherein the antibody comprises a light chain constant region.
285. The antibody of embodiment 284, wherein the light chain constant region is selected from the group consisting of human immunoglobulins κ and λ light chain constant regions.
286. The antibody of any one of the preceding embodiments, wherein the antibody further comprises a heavy chain constant region and a light chain constant region, wherein the heavy chain constant region is a human IgG1 heavy chain constant region, and wherein the light chain constant region is a human κ light chain constant region.
287. The antibody of any one of the preceding embodiments, wherein the antibody is a bispecific antibody.
288. The antibody of embodiment 287, comprising a first antigen-binding region that specifically binds to CD36.
289. The antibody of embodiment 288, comprising a second antigen-binding region that specifically binds to an immune cell antigen.
290. The antibody of embodiment 289, wherein the immune cell antigen is selected from the group consisting of PD-1, PD-L1, CTLA4, CD3, LAG3, OX40, CD28, CD33, B7H3, CD47, TIM3, ICOS, LGR5, 4-1BB, CD40, CD40-L, and TIGIT.
291. The antibody of embodiment 288, comprising a second antigen-binding region that specifically binds to a tumor-specific antigen.
292. The antibody of embodiment 291, wherein the tumor-specific antigen is selected from the group consisting of HER2, HER3, EGFR, VEGF, IGF-1, IGF-2, ANG2, DLL1, IGF-1R, cMET, DLL4, FAP, DR5, IL15, IL15Ra, CD3, CEA, EPCAM, HER3, PSMA, PMEL, and GPC3.
293. The antibody of embodiment 289 to 292, wherein the immune cell antigen or tumor-specific antigen is CD3.
294. The antibody of embodiment 288, wherein the antibody is a biparatopic antibody.
295. The antibody of embodiment 294, comprising two antigen-binding regions, wherein each antigen-binding region specifically binds to a unique, non-overlapping CD36 epitope.
296. The antibody of embodiment 295, comprising a first antigen-binding domain, which comprises the antigen-binding domain of 1G04.
297. The antibody of embodiment 296, further comprising a second antigen-binding domain, which comprises an antigen-binding domain of an antibody selected from the group consisting of 10G04, 11G04, 19G04, 20G04, and 30G04.
298. The antibody of any of embodiments 294 to 297, comprising a first antigen-binding domain, which comprises the antigen-binding domain of 1G04, and a second antigen-binding domain, which comprises an antigen-binding domain of 11G04.
299. The antibody of any one of the preceding embodiments, which is an antigen binding fragment.
300. The antigen binding fragment of embodiment 299, wherein the antigen binding fragment comprises a Fab, Fab′, F(ab′)₂, single chain Fv (scFv), disulfide linked Fv, V-NAR domain, IgNar, intrabody, IgGΔCH2, minibody, F(ab′)₃, tetrabody, triabody, diabody, single-domain antibody, DVD-Ig, Fcab, mAb2, (scFv)₂, or scFv-Fc.
301. A pharmaceutical composition comprising the antibody of any one of the preceding embodiments and a pharmaceutically acceptable excipient.
302. The pharmaceutical composition of embodiment 301, wherein at least 95% of the antibodies in the composition are afucosylated.
303. An antibody or pharmaceutical composition as described in any one of the preceding embodiments for use as a medicament.
304. An antibody or pharmaceutical composition as described in any one of the preceding embodiments for use in a method of treating cancer as described in the present application, optionally in a combination treatment as described herein.
305. An antibody or pharmaceutical composition according to embodiment 304, wherein the cancer is oral squamous cell carcinoma, head and neck cancer, esophageal cancer, gastric cancer, ovarian cancer, cervical cancer, lung cancer, breast cancer, colon cancer, renal cancer, prostate cancer, sarcoma, melanoma, leukemia, or lymphoma.
306. An antibody or pharmaceutical composition according to embodiment 304, wherein the treatment reduces the size of metastatic tumors, as measured by IVIS imaging or H&E staining.
307. An antibody or pharmaceutical composition according to embodiment 302, wherein the treatment inhibits the formation or development of metastatic tumors, as measured by IVIS imaging or H&E staining.

### EXAMPLES

### Example 1: Animal Studies Materials and Methods

Unless otherwise indicated, the animal studies disclosed in the Examples below were carried out using the following materials and methodologies.

NOD scid gamma (NSG) (NOD.Cg-Prkd^{scid}II2rg^{tm1Wj1}/SzJ) mice were purchased from Charles River and crossed in-house. All mice were housed under a regimen of 12h light/12h dark cycles and SPF conditions, and all procedures were evaluated and approved by the CEEA (Ethical Committee for Animal Experimentation) from the Government of Catalunya. SCC intra-tongue injection was performed as previously described (Oskarsson et al., 2014; Nieman et al., 2011). Briefly, mice were anesthetized by intraperitoneal injection with a mixture of 50 mg per kg of ketamine and 0.5 mg per kg of medetomidin, and SCC cells resuspended in 30 l PBS were injected into each mouse tongue with a BD ultra-fine 6 mm needle. Mice were monitored for the luciferase bioluminescent signal immediately after injection (T0) and once weekly thereafter with a Xenogen IVIS Imaging System-100 (Caliper Life Sciences). Briefly, animals were injected by retro-orbital injection with 50 μl of D-luciferin (Promega) diluted in 1×PBS at 5 mg ml⁻¹. Continuous administration of isofluorane gas was provided to ensure anesthetizing animals during imaging. Data was quantified with the Living Image software version 4.4 (Caliper Life Sciences). Quantifications were calculated with unsaturated pixels. Color scale minimum and maximum values are shown in pictures.

To treat mice in vivo with neutralizing anti-CD36 antibodies, mice were injected intraperitoneally with 100 μl of physiological serum containing 5 μg, 10 μg or 20 μg of the neutralizing monoclonal anti-CD36 antibody JC63.1 (CAYMAN, CAY-10009893-500); 5 μg, 10 μg or 20 μg of neutralizing monoclonal anti-CD36 ONA-0-v1 (either IgA or IgG isotype); or 5 μg, 10 μg or 20 μg of the corresponding control IgA (mouse IgA, kappa [S107], Abcam, ab37322) or IgG antibody. These doses corresponded to 0.25, 0.5, and 1 mg/kg, respectively. All antibodies were azide-free with no added preservative compound.

For each experiment, mice were sacrificed at the same time, once an experimental group reached the humane endpoint according to the approved CEEA protocol (4-6 weeks after the orthotopic injection as soon as mice started to lose weight due to the growth of the oral lesion), and subsequent cell analysis was performed.

Total blood samples from mice were collected from the inferior vena cava and then processed in the Experimental Toxicology and Ecotoxicology Unit (PCB) following standard procedures.

Animal tissue was collected and fixed with 4% paraformaldehyde (PFA) for overnight at room temperature (RT) and then either embedded in OCT and frozen at −80° C. or dehydrated and embedded in paraffin. Toxicological study was performed at the Histopathology Facility according to standard procedures.

Histological Analysis. For analysis, cryo- or de-paraffinized antigen retrieved sections (10 min in boiling 0.01M citric acid, pH 6.0) of 8 m were permeabilized for 25 min in 0.25% Triton X-100/PBS and blocked for 90 min in 0.25% gelatin/PBS. Hematoxilin and eosin (H&E) staining was done according to the standard protocol. Images were acquired using a Nikon E600+Olympus DP72, Leica SPE and a Leica TCS SP5 confocal microscope. Representative pictures were selected in each case.

For all the experiments, adequate sample size was determined based on results of pilot studies. No statistical method was used to determine sample size. All the animals that fulfilled proper experimental conditions during the experimental procedures were included in the analysis. Based on results of pilot studies, homogeneous groups of males and females between 8 and 12 weeks and their control littermates were used for the experimental studies. Animals were randomized at day 7 post-injection based on luminescence intensity of the primary tumours or of the cervical lymph nodes metastasis. Data are generally shown as the mean±s.e.m. Statistical significance was analyzed using Prism 6 software (GraphPad) by using a two-tailed t-test, Mann-Whitney U test, Fisher exact test or hypergeometric test. Significance was considered at P< or equal 0.05.

### Example 2: Treatment of Cancer Using an Anti-CD36 Antibody, with or without Cisplatin, in a Mouse Model of Oral Cancer

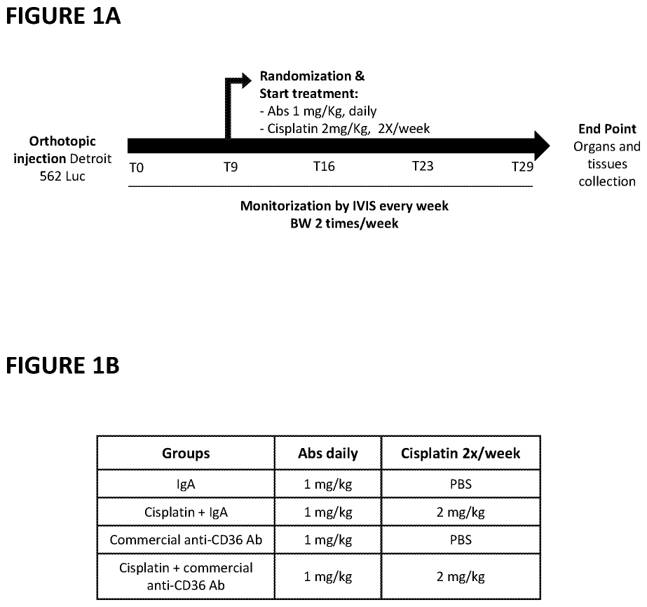

Studies of the effects of an anti-CD36 antibody, both with and without cisplatin, were performed in NSG mice (immuno-deficient). An experimental overview of these studies is provided in FIG. 1A. The studies included only male mice, though similar trends (data not reported) were observed using female mice. All mice were inoculated with commercially available Detroit 562 (ATCC) cancer cells, transduced with a retroviral vector expressing luciferase and the green fluorescent protein (Luc-GFP). Detroit 562 cells were derived from the metastatic site of a pharyngeal carcinoma (i.e., from an oral cancer). Prior to inoculation, the Detroit 562 cells were cultured in a humidified incubator at 37° C. with 5% CO₂, and were grown in EMEM (LONZA) supplemented with 5 μg ml⁻¹ penicillin/streptomycin and 10% FBS (GIBCO).

For each mouse, 50,000 Detroit 562 cells were inoculated via orthotopic injection. Previous testing revealed that, in untreated NSG mice, 100% of mice inoculated with Detroit 562 cells formed a large primary tumor and 81% of inoculated mice were observed to develop lymph node metastases within one week of inoculation.

Treatment of the inoculated mice began nine days after inoculation with the cancer cells. Inoculated mice were divided into four distinct treatment groups. As can be seen in FIG. 1B, the treatment groups were:
Group 1: IgA isotype control (n=9 on days 1 through 23; n=6 on day 29);
Group 2: IgA isotype control plus cisplatin (n=5);
Group 3: commercial anti-CD36 antibody (JC63.1) (n=6 on days 1 through 23; n=4 on day 29);
Group 4: commercial anti-CD36 antibody (JC63.1) plus cisplatin (n=5).

Antibody treatments were administered via intraperitoneal (i.p.) injection daily at a dose of 1 mg/kg. Cisplatin was administered twice weekly at a dose of 2 mg/kg (Groups 2 and 4). Mice that did not receive cisplatin (Groups 1 and 3) instead received a volume equivalent injection of PBS. During the course of treatment, mice were observed once weekly using an in vivo imaging system (IVIS). Further, mouse body weight was measured twice weekly to update appropriate dosage amounts. Mice were sacrificed either when their bodyweight dropped under the ethical approved guidelines or at the end of the treatment period. Upon sacrifice, organs and tissues were collected for performance of immunohistochemistry analysis.

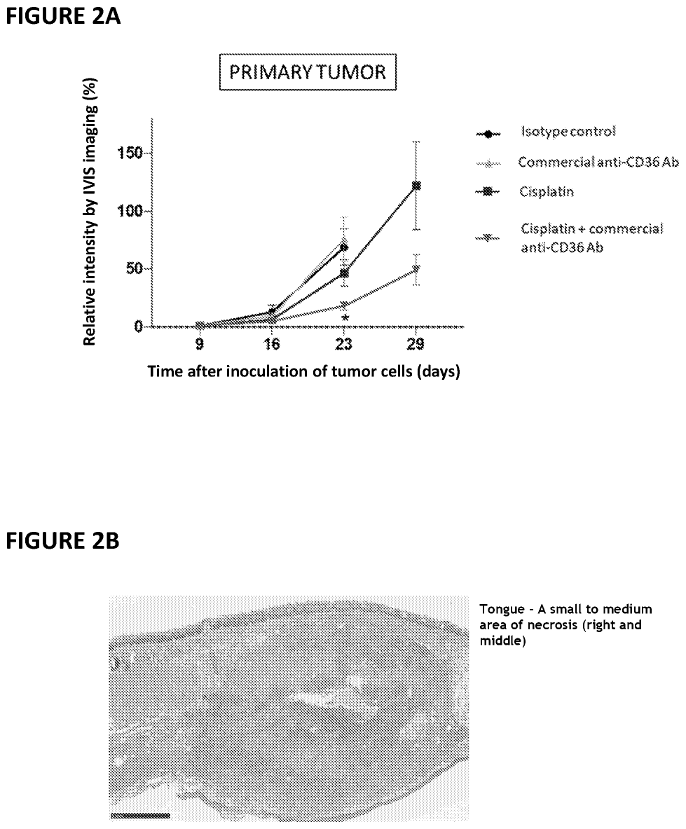

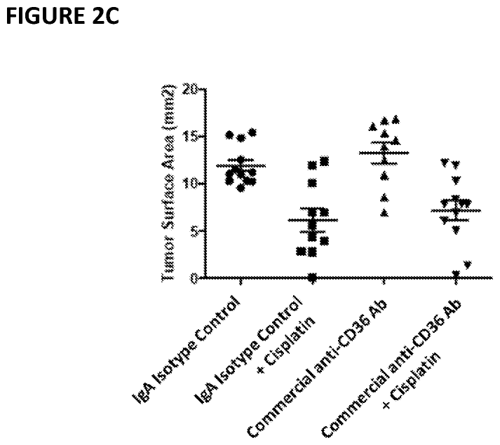

As can be seen in FIGS. 2A-2C, the tested anti-CD36 Ab treatment has at least additive anti-tumor activity with cisplatin on suppressing the growth of a primary tumor in oral cancer. FIG. 2A shows that mice treated with both anti-CD36 antibody and cisplatin were better able to suppress tumor growth than mice treated with control antibody (IgA) and cisplatin, as measured by the relative intensity of luciferase-induced luminescence in treated mice relative to control mice. FIG. 2B shows a representative image of a primary tumor developed in the tongue after orthotopic injection of the Detroit 562 cells. FIG. 2C shows that mice treated with both anti-CD36 antibody or with control antibody (IgA) and cisplatin had primary tumors with reduced tumor surface area.

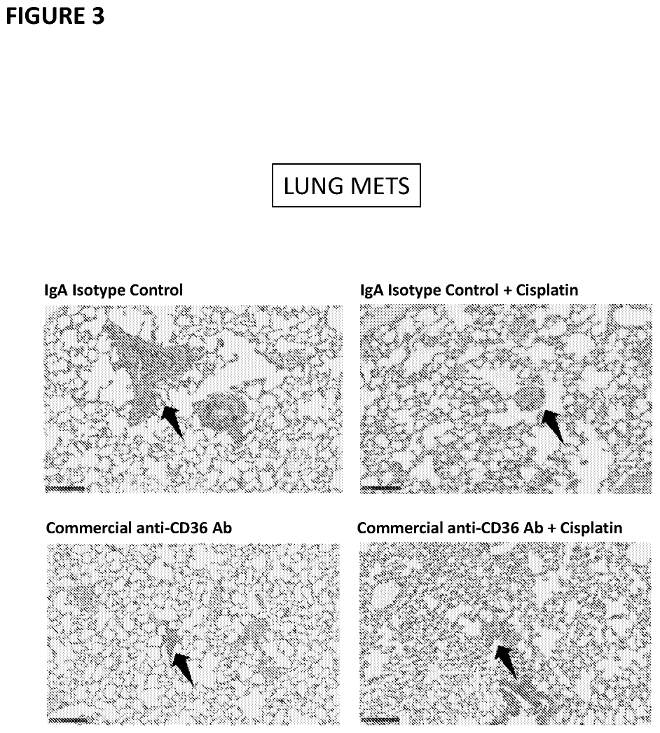

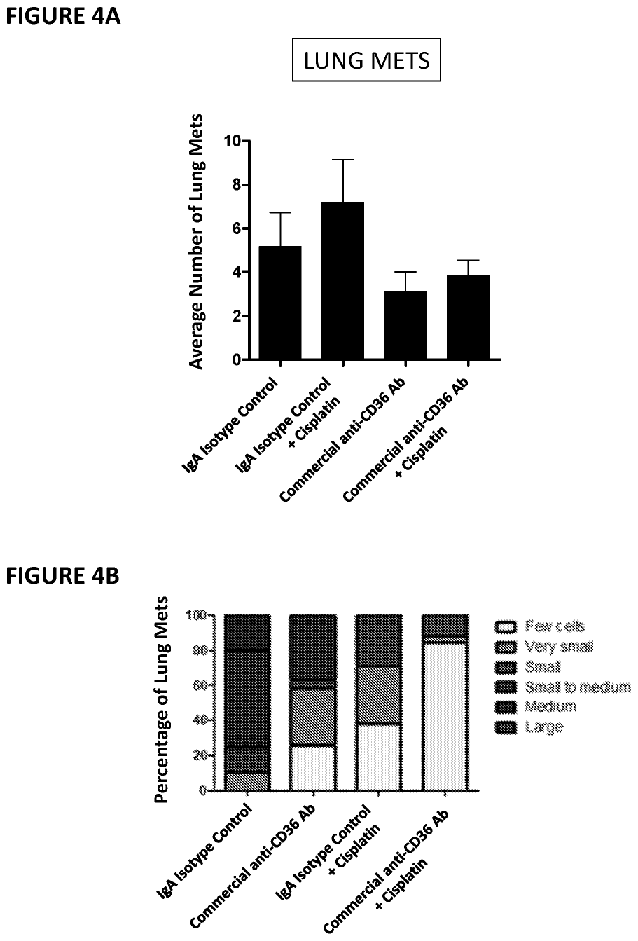

FIG. 3 shows representative images of lung metastases present in mice inoculated with Detroit 562 cancer cells and treated as described above. These images illustrate that mice treated with cisplatin (top right), a commercial anti-CD36 antibody (JC63.1; bottom left), or cisplatin and JC63.1 (bottom right) have fewer and smaller metastases than control treated mice (top left). Further, the quantitation of the number (FIG. 4A) and size (FIG. 4B) of lung metastases shows that mice treated with JC63.1 alone had smaller and fewer metastases than control-treated mice. Mice treated with cisplatin alone had similar numbers of metastases to control cells, though cisplatin did reduce the size of the metastatic tumors. Treatment with both JC63.1 and cisplatin resulted in mice with similar numbers of metastases as treatment with JC63.1 alone. However, treatment with both JC63.1 and cisplatin resulted in reduction of metastatic tumor size to a greater extent than either JC63.1 or cisplatin alone. For example, anti-CD36 antibody treatment alone, and cisplatin in combination with an isotype control antibody, inhibited limited the size of metastatic tumors such that 25-40% of the metastatic tumors were only a few cells, as measured using immunohistochemistry analysis. Treatment with anti-CD36 antibody in combination with cisplatin limited the size of metastatic tumors such that over 80% of the metastatic tumors were only a few cells, as measured using immunohistochemistry analysis.

### Example 3: Treatment of Cancer Using the ONA-0-v1 Anti-CD36 Antibody, with or without Cisplatin, in a Mouse Model of Oral Cancer

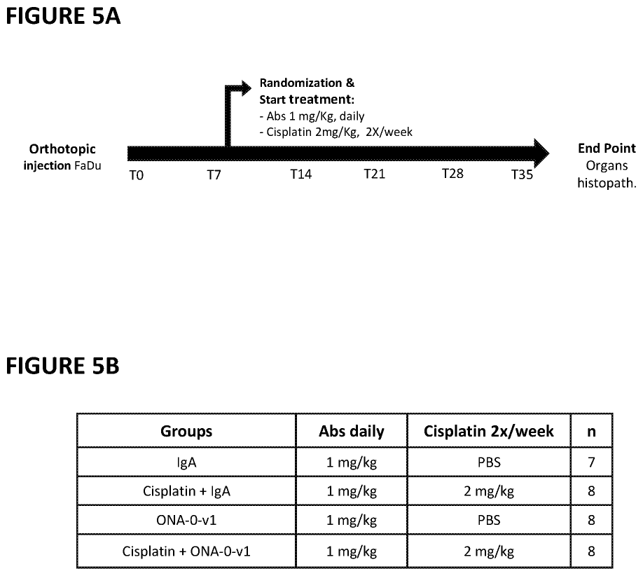

Studies on the combination of the effects of the ONA-0-v1 anti-CD36 antibody, both with and without cisplatin, were performed in NSG mice (immuno-deficient). An experimental overview of these studies is provided in FIG. 5A. The studies included both male and female mice. All mice were inoculated with commercially available FaDu (ATCC) cancer cells, transduced with a retroviral vector expressing luciferase and the green fluorescent protein (Luc-GFP). FaDu cells were derived from a squamous cell carcinoma (i.e., from an oral cancer). Prior to inoculation, the FaDu cells were cultured in a humidified incubator at 37° C. with 5% CO₂, and were grown in EMEM (LONZA) supplemented with 5 μg ml⁻¹ penicillin/streptomycin and 10% FBS (GIBCO).

For each mouse, 100,000 FaDu cells were inoculated via orthotopic injection. Previous testing revealed that, in untreated NSG mice, 100% of mice inoculated with FaDu cells formed a large primary tumor and 91% of inoculated mice were observed to develop lymph node metastases within one week of inoculation.

Treatment of the inoculated mice began nine days after inoculation with the cancer cells. Inoculated mice were divided into four distinct treatment groups. As can be seen in FIG. 5B, the treatment groups were:
Group 1: IgA isotype control (n=7);
Group 2: IgA isotype control plus cisplatin (n=8);
Group 3: anti-CD36 antibody ONA-0-v1 (n=8);
Group 4: anti-CD36 antibody ONA-0-v1 plus cisplatin (n=8).

Antibody treatments were administered via intraperitoneal (i.p.) injection daily at a dose of 1 mg/kg. Cisplatin was administered twice weekly at a dose of 2 mg/kg (Groups 2 and 4). Mice that did not receive cisplatin (Groups 1 and 3) instead received a volume equivalent injection of PBS. During the course of treatment, mice were observed once weekly using an in vivo imaging system (IVIS). Further, mouse body weight was measured twice weekly to update appropriate dosage amounts. At the end of the treatment period, the mice were sacrificed, and organs and tissues were collected for performance of immunohistochemistry analysis.

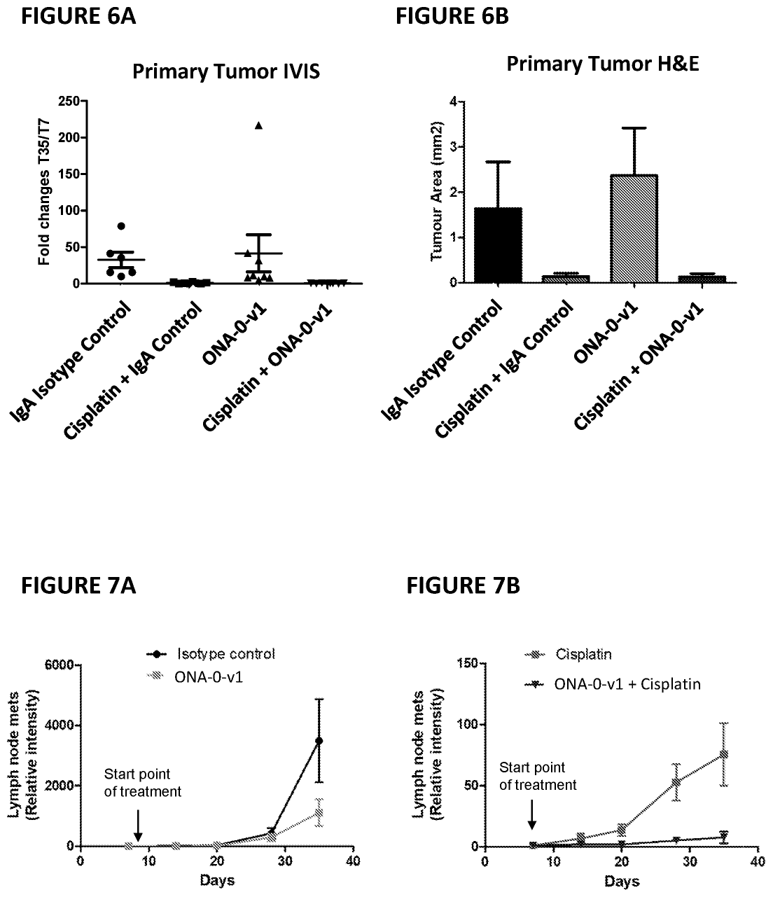

As can be seen in FIGS. 6A and 6B, treating with the anti-CD36 antibody ONA-0-v1 in combination with cisplatin had similar effects to treating with cisplatin alone as measured by IVIS imaging and H&E staining of the primary tumor. Treatment with ONA-0-v1 alone at a 1 mg/kg dose in this model did not have a statistically significant effect on the primary tumor relative to treatment with an isotype control antibody. In contrast, FIGS. 7A and 7B show that treatment with ONA-0-v1 alone was able to inhibit growth of lymph node metastases, as measured by relative intensity in IVIS imaging. Moreover, treatment with ONA-0-v1 in combination with cisplatin resulted in almost complete inhibition of lymph node metastasis growth, as measured by relative intensity in IVIS imaging.

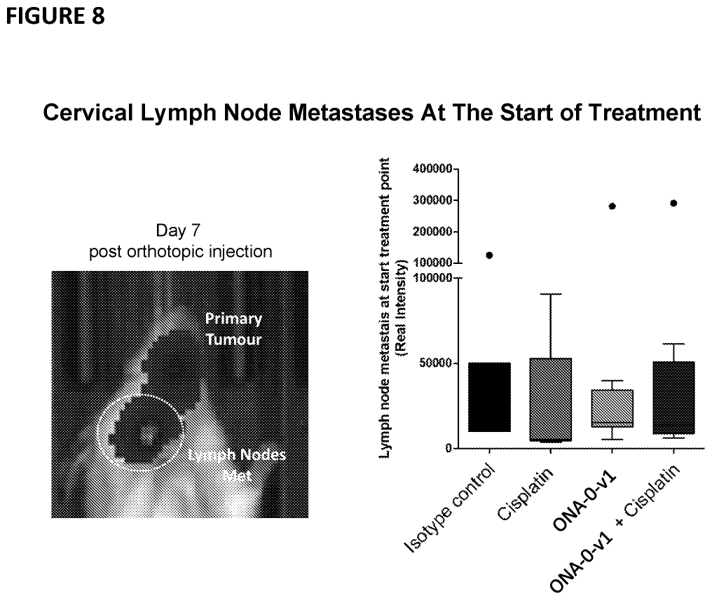

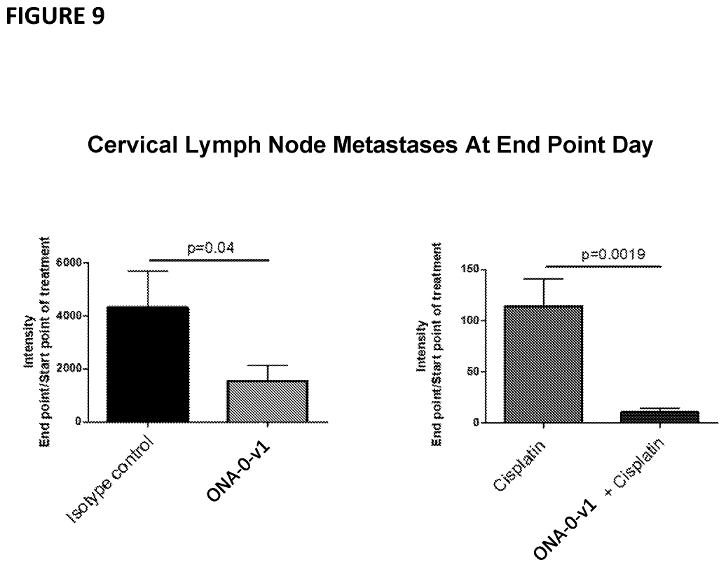

Treatment with the ONA-0-v1 antibody inhibited growth of lymph node metastases. FIG. 8 shows a representative IVIS image of an inoculated NSG mouse on day 7 post-orthotopic injection of FaDu cells, immediately prior to the start of treatment. The lymph node metastasis in that mouse is indicated by the circled area, with the intensity of the luciferase signalling indicated by the heat map. FIG. 8 also shows the quantitation of the lymph node metastases present in all groups of mice on day 7. That initial intensity was the same in all groups. Further IVIS imaging was performed at the end-point of treatment, As shown in FIG. 9 (left panel), treatment with ONA-0-v1 antibody inhibited metastatic tumor growth by greater than 50% relative to the IgA isotype control, as measured by the ratio of IVIS imaging intensity between the ending and starting points of treatment. Further, also as shown in FIG. 9 (right panel), addition of ONA-0-v1 to cisplatin enhanced cisplatin's ability to inhibit metastatic tumor growth. The ONA-0-v1 and cisplatin combination resulted in almost complete inhibition of tumor growth in lymph node metastases.

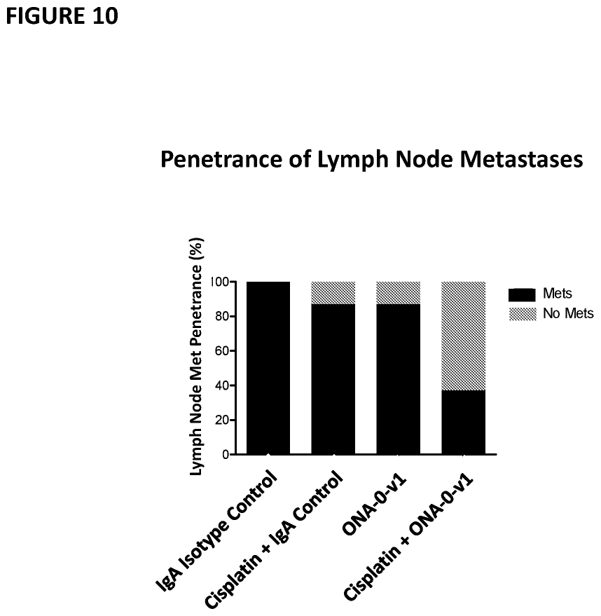

Treatment with the ONA-0-v1 antibody also inhibited penetrance of metastases into lymph nodes, as shown in FIG. 10. All control mice presented with lymph node metastases. Treatment with either cisplatin or ONA-0-v1 prevented metastasis into the lymph nodes in one of the eight tested mice in each respective treatment group (i.e., 12.5%). Moreover, ONA-0-v1's inhibition of penetrance was synergistic with that of cisplatin, as the combination of cisplatin and ONA-0-v1 prevented any metastasis in five of the eight tested mice (i.e., 62.5%).

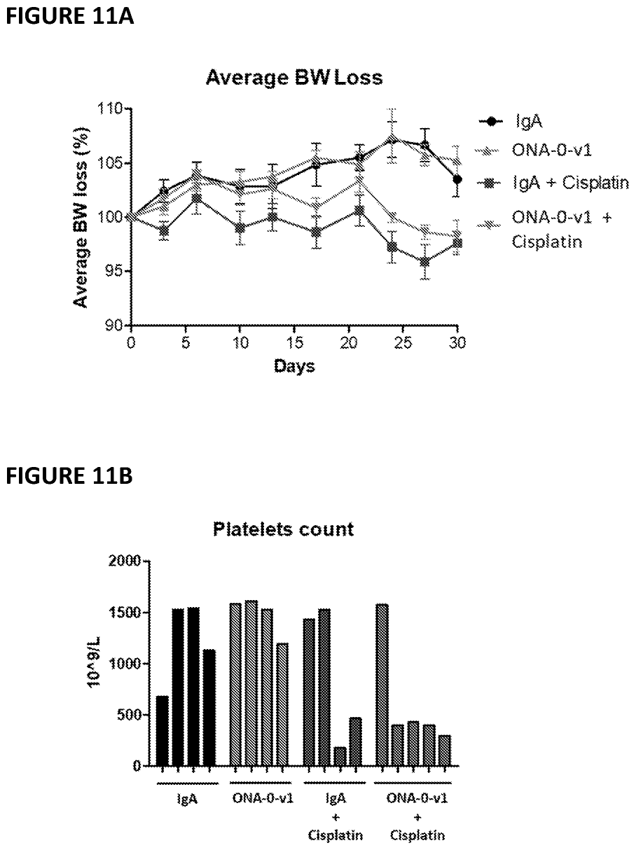

Treatment with the ONA-0-v1 antibody was well-tolerated by NSG mice over the course of treatment. As shown in FIG. 11A and FIG. 11B, ONA-0-v1 treatment alone did not have any effects on mouse body weight or platelet count relative to isotype control-treated mice. ONA-0-v1 treatment also did not significantly enhance cisplatin-mediated weight loss or the cisplatin-mediated decrease in platelet count.

### Example 4: Antitumor Efficacy of Anti-CD36 Antibodies in Combination with PD1 Inhibition in C57B16/J Mice Bearing YUMM1.7 Cells-Derived Melanoma Tumors

250,000 YUMM1.7 cells are suspended in PBS and are injected subcutaneously in the flank of 8-12 week-old C57B16/J mice. When tumors reach a mean volume of 50-100 mm³, mice are randomized and the treatment is started.

The experimental groups are as shown in Table 6 below.

TABLE 6
Treatment Groups for Treating with
anti-CD36 and anti-PD-1 Antibodies

| Group | No. Mice | Treatment |
|---|---|---|
| 1 | 10 | anti-PD1 isotype control |
| | | (rat IgG2a, clone 2A3) |
| 2 | 10 | anti-mouse PD-1 |
| | | (clone RMP1-14) |
| 3 | 10 | anti-CD36 isotype control |
| 4 | 10 | anti-CD36 |
| 5 | 10 | anti-mouse PD-1 + anti-CD36 |

All antibodies are injected IP at the concentration of 10 mg/kg, 3 times/week. Mice are monitored three times per week for body weight and tumour volume and daily for behaviour and survival. When tumour reaches a maximum volume of 1.500 mm³, mice are euthanized and tissues collected. Primary tumours are weighted and measured again with a caliper. Lung and liver are embedded in paraffin for H&E staining and a blinded analysis for metastatic lesions. Results of the study will show that anti-CD36 antibodies (e.g., 1G04) and anti-PD-1 antibodies have additive or synergistic effects in treating cancer in the YUMM1.7 mouse model of melanoma.

### Example 5: Treatment of Ovarian Cancer Using the ONA-0-v1 Anti-CD36 Antibody

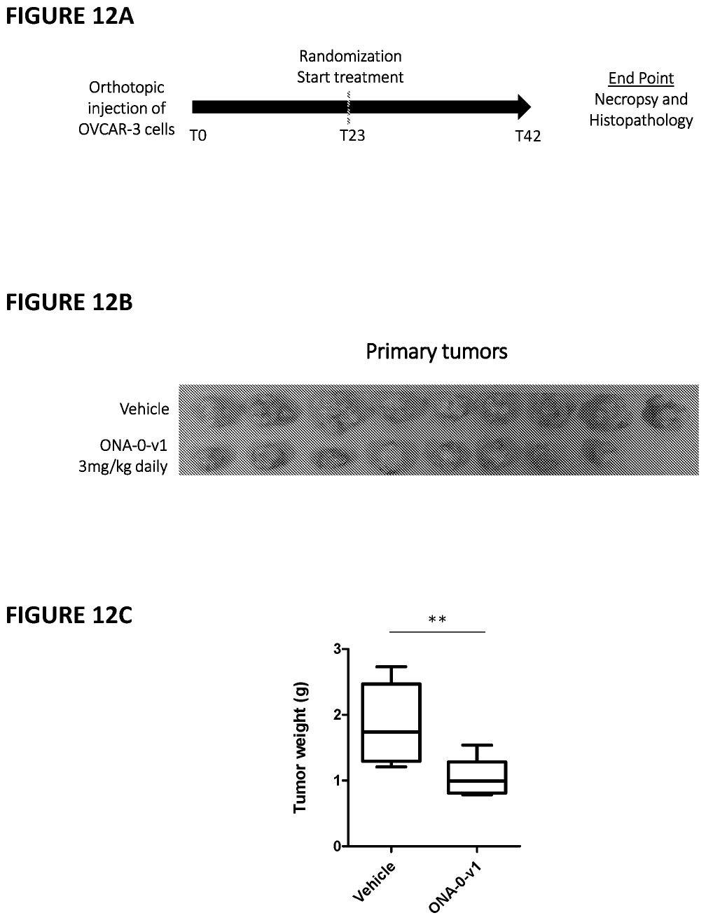

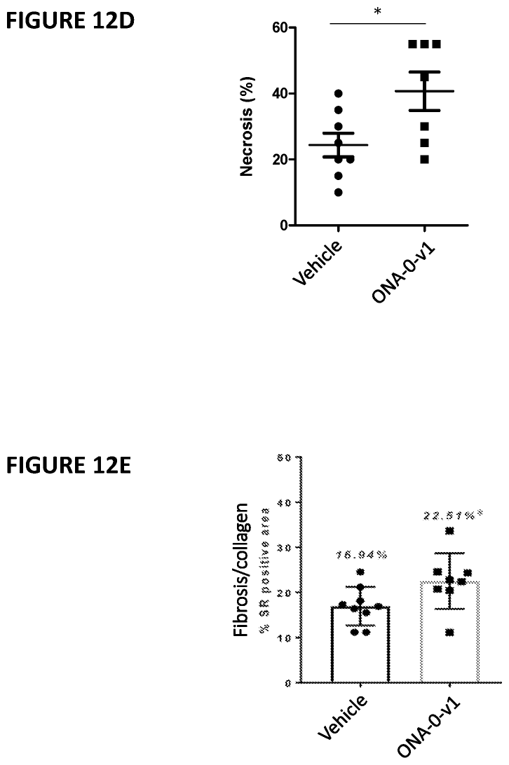

Studies of the effects of the ONA-0-v1 anti-CD36 antibody on ovarian cancer were performed in NSG mice (immuno-deficient). An experimental overview of these studies is provided in FIG. 12A. The studies included only female mice. All mice were inoculated with commercially available OVCAR-3 (ATCC) cancer cells. OVCAR-3 cells were derived from a human progressive adenocarcinoma of the ovary (i.e., from an ovarian cancer). Prior to inoculation, the OVCAR-3 cells were cultured in a humidified incubator at 37° C. with 5% CO₂, and were grown in RPMI-1640 supplemented with 5 μg ml⁻¹ penicillin/streptomycin, 0.01 mg/ml bovine insulin and 20% FBS (GIBCO).

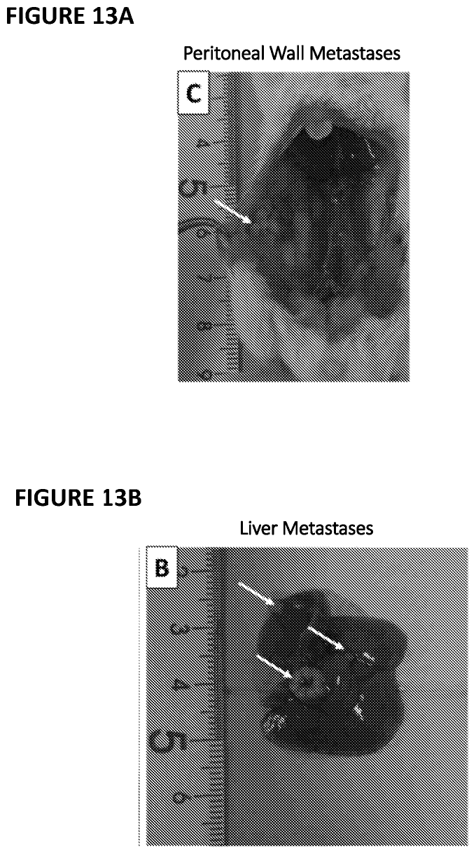

For each mouse, a piece of an OVCAR-3 xenograft was implanted orthotopically. As shown in FIG. 12B, NSG mice implanted with OVCAR-3 cells form a large primary tumor. Mice implanted with OVCAR-3 also develop metastases in both the peritoneal wall and liver. Exemplary metastases from inoculated mice are shown in FIGS. 13A and 13B.

Treatment of the implanted mice began 23 days after implantation with the OVCAR-3 tumor pieces. Inoculated mice were divided into one of two treatment groups: vehicle injection control (n=9) or ONA-0-v1 treatment (n=9). Antibody treatments were administered via intraperitoneal (i.p.) injection daily at a dose of 3 mg/kg, while control mice received an equal volume of vehicle on the same schedule. Mice were sacrificed at the end of the treatment period. Upon sacrifice, organs and tissues were collected for performance of immunohistochemistry analysis.

As can be seen in FIGS. 12B and 12C, treatment with ONA-0-v1 results in smaller tumors in the OVCAR-3 mouse model of ovarian cancer. The quantification of this effect in FIG. 12C shows that treatment with OVCAR-3 reduced tumor weight from an average of 1.844 g to an average of 1.058 g, a decrease of 43% percent. These data indicate that ONA-0-v1 inhibited tumor growth and/or promoted tumor cell destruction during the treatment period.

Histological analysis of the primary tumors in vehicle-treated and ONA-0-v1-treated mice was also performed. First, the tumors were analyzed to determine percent necrosis by visual inspection and quantification of a pathologist. The results of this analysis are shown in FIG. 12D, which shows that ONA-0-v1 increased necrosis from approximately 24.4% to approximately 40.71% (*=p value of 0.0287). This increase indicates that treated tumors present higher cell death. The primary tumors of treated and ONA-0-v1-treated mice were also analyzed to determine the percent of collagenous and fibrotic areas by Sirius red staining. The results of this analysis are shown in FIG. 12E, which shows that ONA-0-v1 increased the SR positive area from 16.9% to 22.5% (*=p value of 0.0457). This increase indicates that treatment with ONA-0-v1 increases fibrosis and, together with the increased necrosis, indicates that the treated tumors are not only smaller, but also they are composed of fewer tumoral cells.

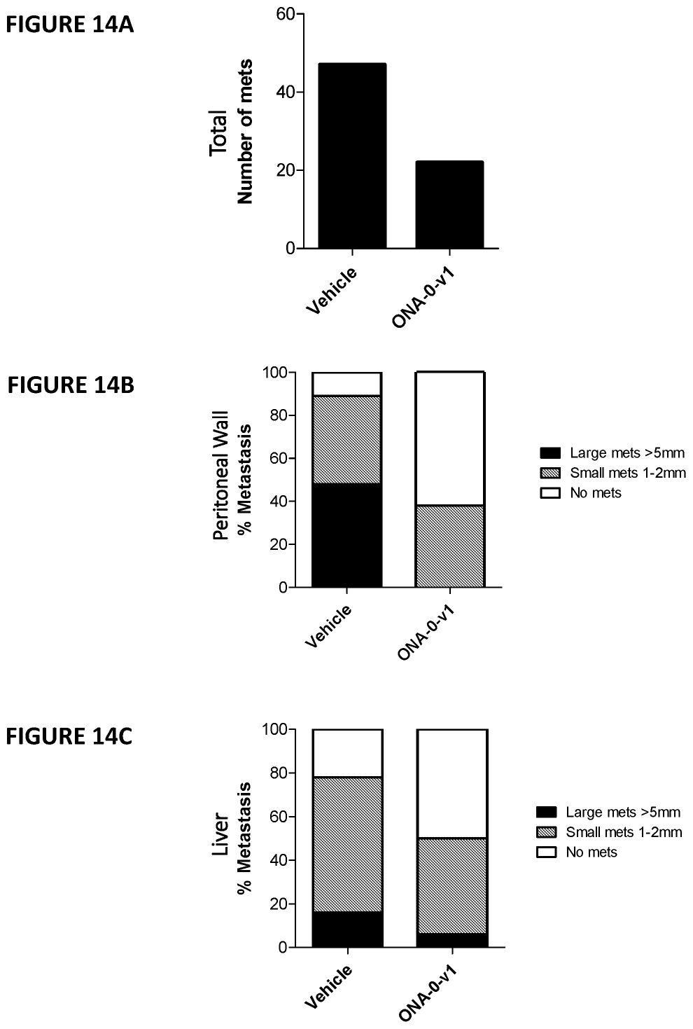

FIGS. 14A, 14B, and 14C show the results of quantifying metastatic tumors in ONA-0-v1 treated mice. FIG. 14A shows that the total number of metastases decreased by over 50% in the ONA-0-v1-treated mice relative to vehicle-treated mice. The total number of metastases was determined by visual inspection of the organs. FIG. 14B and FIG. 14C show the results of macroscopic analysis of the size of metastases in the peritoneal wall and liver, respectively. The size of the metastases was measured by visual inspection. In the vehicle-treated group, 48% of the animals had large metastasis (>5 mm), 41% small metastasis (1-2 mm), and 11% no metastasis in the peritoneal wall. In the ONA-0-v1 treated animals, no large metastasis were detected, 38% of the animals had small metastasis, and 63% presented no metastasis. In the liver, the percentage of mice without metastasis increased from 22% in the vehicle group to 50% in the treated group. Among the animals with liver metastasis, the number of large ones was reduced from 16% to 6% and small ones from 62% to 44%. Treating with ONA-0-v1 shifted the size of peritoneal wall metastases such that large metastases disappeared entirely, and more mice did not have peritoneal metastases at all (FIG. 14B). Similarly, treating with ONA-0-v1 shifted the size of liver metastases such that fewer large metastases were found, and more mice did not have liver metastases at all (FIG. 14C). Collectively, FIGS. 14A, 14B, and 14C show that ONA-0-v1 is effective at reducing the formation and growth of metastases from ovarian cancer.

### Example 6: Treatment of Ovarian Cancer Using the ONA-0-v1 and 1G04 Anti-CD36 Antibodies

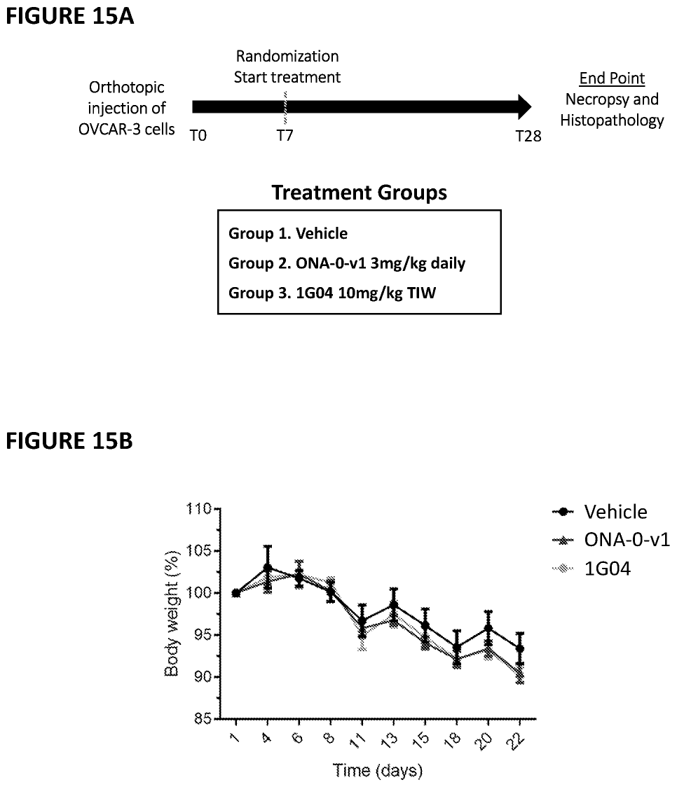

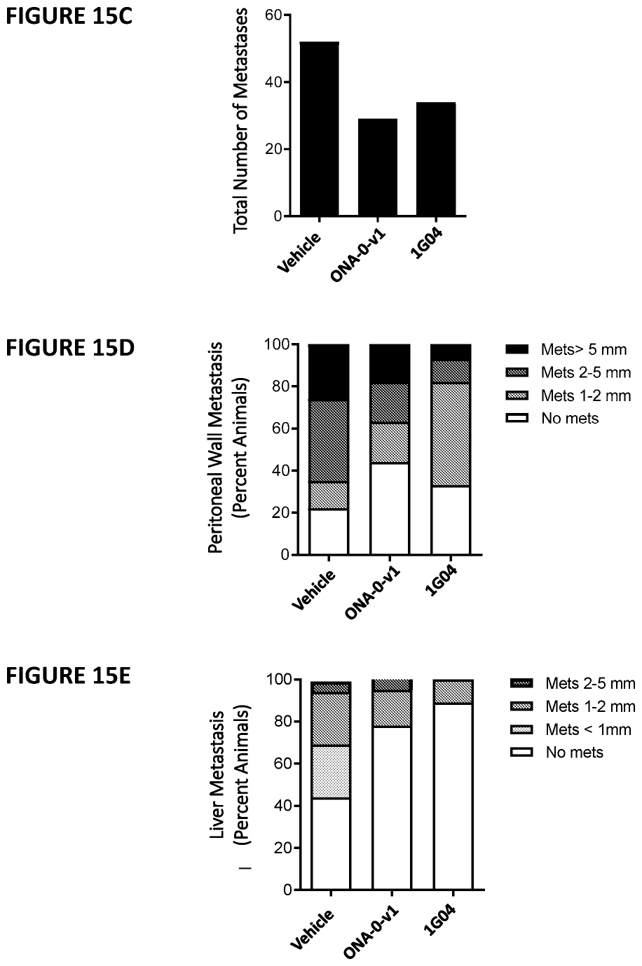

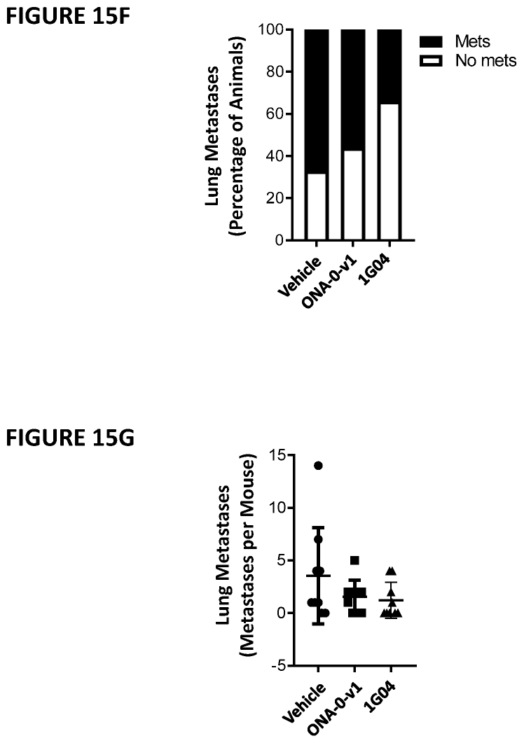

Studies of the effects of the ONA-0-v1 and 1G04 anti-CD36 antibodies on ovarian cancer were performed in NSG mice (immuno-deficient). An experimental overview of these studies is provided in FIG. 15A. The studies included only female mice. All mice were inoculated with commercially available OVCAR-3 (ATCC) cancer cells. OVCAR-3 cells were derived from a human progressive adenocarcinoma of the ovary (i.e., from an ovarian cancer). For each mouse, a piece of an OVCAR-3 xenograft was implanted orthotopically. Prior to inoculation, the OVCAR-3 cells were cultured in a humidified incubator at 37° C. with 5% CO₂, and were grown in RPMI-1640 supplemented with 5 g ml⁻¹ penicillin/streptomycin, 0.01 mg/ml bovine insulin and 20% FBS (GIBCO).

Treatment of the implanted mice began 7 days after implantation with the OVCAR-3 tumor pieces. Inoculated mice were divided into one of three treatment groups: vehicle injection control (n=9), ONA-0-v1 treatment (n=9), or 1G04 treatment (n=9). ONA-0-v1 antibody treatments were administered via intraperitoneal (i.p.) injection daily at a dose of 3 mg/kg. 1G04 antibody treatments were administered via i.p. injection TIW (three-times weekly) at a dose of 10 mg/kg. Control mice received an equal volume of vehicle daily. As can be seen in FIG. 15B, the weight of mice in all three treatment groups remained the same throughout the treatment period. Mice were sacrificed at the end of the treatment period. Upon sacrifice, organs and tissues were collected for performance of necropsy and histopathology analysis.

FIGS. 15C-15G show the results of quantifying metastatic tumors in treated mice. FIG. 15C shows the total number of metastases for each treatment condition. The total number of metastases was determined by visual inspection of the organs. This analysis revealed that the number of metastases decreased by approximately 45% in the ONA-0-v1-treated mice, relative to vehicle-treated mice (52 metastasis counted in vehicle and 29 in treated group). The total number of metastases also decreased by approximately 35% in the 1G04-treated mice, relative to vehicle-treated mice (52 metastasis counted in vehicle and 34 in treated group).

FIG. 15D and FIG. 15E show the results of macroscopic analysis of the size of metastases in the peritoneal wall and liver, respectively. The size of the metastases was measured by visual inspection. Treating with either ONA-0-v1 or 1G04 reduced the observed size of metastases such that fewer large (>5 mm) and medium (1-2 mm) sized metastases were observed. Vehicle-treated animals presented with 26% of the mice having >5 mm metastasis, 39% having 2-5 mm metastasis, and 13% having 1-2 mm metastasis in the peritoneal wall. ONA-0-v1 treated animals presented with 19% of mice having >5 mm metastasis, 19% having 2-5 mm metastasis, and 19% having 1-2 mm metastasis in the peritoneal wall. 1G04-treated animals presented with 7% of mice having >5 mm metastasis, 11% with 2-5 mm metastasis, and 49% with 1-2 mm metastasis in the peritoneal wall. In addition, the livers of treated mice showed an analogous pattern. Vehicle-treated animals presented with 5% of the mice having 2-5 mm metastasis, 25% having 1-2 mm metastasis, and 25% having <1 mm metastasis in the liver. ONA-0-v1 treated animals presented with 6% of the mice having 2-5 mm metastasis, 17% having 1-2 mm metastasis, and none having <1 mm metastasis in the liver. 1G04-treated none with 2-5 mm metastasis, 11% with 1-2 mm metastasis and none with <1 mm metastasis. Moreover, treatment with either ONA-0-v1 or 1G04 increased the percentage of animals that were free of metastases in the peritoneal wall and liver. 22% of the vehicle-treated mice, 44% of the ONA-0-v1-treated mice, and 33% of the 1G04-treated mice were metastasis-free in the peritoneal wall. 44% of vehicle-treated mice, 78% of the ONA-O-v1-treated mice, and 89% of the 1G04-treated mice were free of metastasis in the liver.

FIG. 15F shows the results of microscopic analysis of the penetrance of metastases in the lung. As with the peritoneal wall and liver, treatment with either ONA-0-v1 or 1G04 increased the percentage of animals that were free of metastases in the lung (from 33% in vehicle to 44% and 66% in ONA-0-v1 and 1G04 groups respectively). Moreover, as quantified in FIG. 15G, treatment with either ONA-0-v1 or 1G04 reduced the number of metastases in the lungs per mouse (mean metastasis number 3.6 in vehicle-treated, 1.6 in ONA-0-v1 and 1.2 in 1G04 groups).

Collectively, FIGS. 15C-15G show that both ONA-0-v1 (a murine IgA antibody) and 1G04 (a chimeric IgG1 antibody) are effective at reducing the formation and growth of metastases from ovarian cancer.

### Example 7: Treatment of Colon Cancer Using the 1G04 Anti-CD36 Antibody

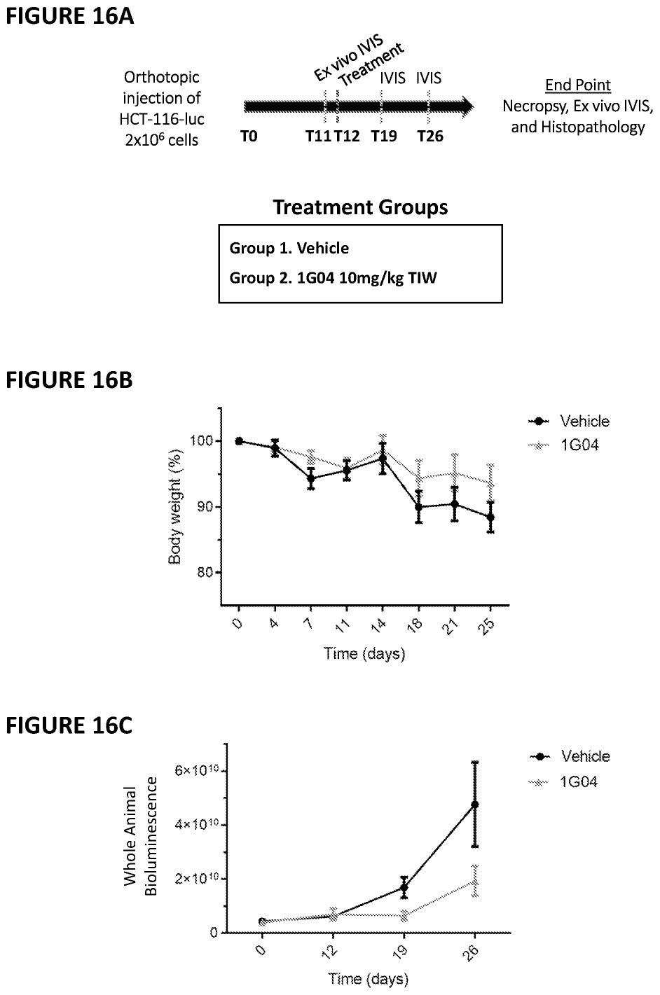

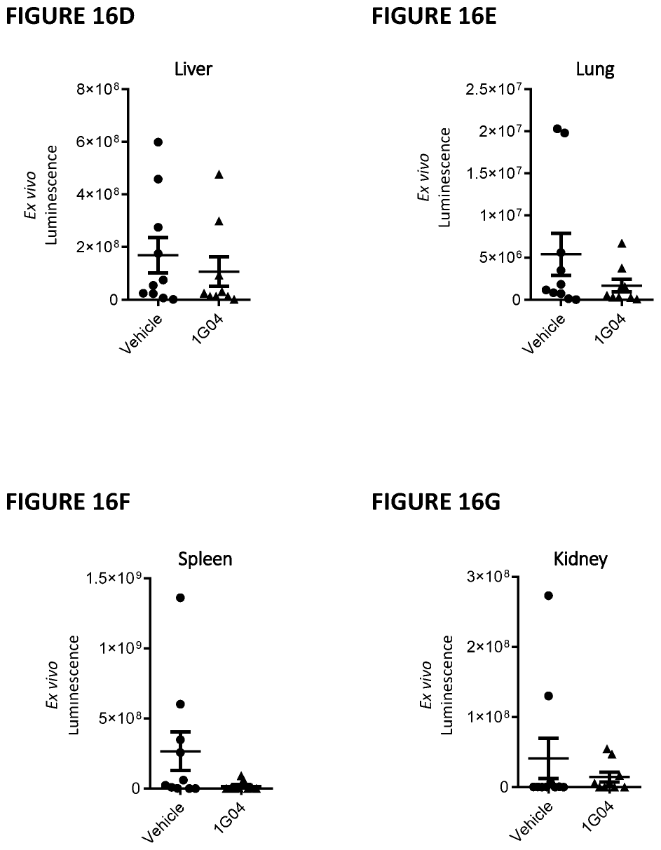

Studies of the effects of the 1G04 anti-CD36 antibody on colon cancer were performed in BALB/c nude mice (immuno-deficient). An experimental overview of these studies is provided in FIG. 16A. The studies included only female mice. All mice were inoculated with commercially available HCT-116 (ATCC) cancer cells, transduced with a retroviral vector expressing luciferase. HCT-116 cells were derived from a human colorectal carcinoma (i.e., from a colon cancer). Prior to inoculation, the HCT-116 cells were cultured in a humidified incubator at 37° C. with 5% CO₂, and were grown in McCoy's 5A medium supplemented with 5 g ml⁻¹ penicillin/streptomycin and 10% FBS (GIBCO).

For each mouse, 2×10⁶ HCT-116 cells were inoculated via orthotopic injection. Each mouse was imaged after inoculation and one week later and liver metastasis were confirmed by ex vivo luminescence prior to start of treatment. Treatment began 12 days after inoculation with the HCT-116 cells. Inoculated mice were divided into one of two treatment groups: vehicle injection control (n=10) or 1G04 treatment (n=10). Antibody treatments were administered via intraperitoneal (i.p.) injection at a dose of 10 mg/kg three times per week, while control mice received an equal volume of vehicle on the same schedule. One day prior to the start of treatment, and at 7, 14, and 21 days after the start of treatment, all mice were imaged via IVIS. Mice were sacrificed at the end of the treatment period (day 25). Upon sacrifice, organs and tissues were collected for performance of necropsy, ex-vivo IVIS, and histopathology.

As can be seen in FIG. 16B, mice treated with 1G04 were better able to maintain weight during the course of treatment. FIG. 16C shows the results of whole-animal bioluminescence imaging over time, which is a readout for the growth of luciferase-containing tumor cells in the mouse. The bioluminescence imaging showed that 1G04 decreased whole animal luminescence, and thus slowed the growth of the injected HCT-116 tumor cells in vivo.

FIGS. 16D, 16E, 16F, and 16G show the results of quantifying metastatic tumors in the 1G04 and vehicle treated mice. After the organs to be examined were removed from the mice, the luminescence of the metastatic tumors in the liver (FIG. 16D), lung (FIG. 16E), spleen (FIG. 16F), and kidney (FIG. 16G) was quantified by ex vivo luminescence using IVIS. In each organ, 1G04 treatment decreases the luminescence, reflecting a reduction in the size and/or number of metastases. The observed mean luminiscence values for liver, lung, spleen and kidney of vehicle-treated mice were 1.69*10⁸, 5.38*10⁶, 2.66*10⁸, and 4.11*10⁷, respectively. The observed mean luminiscence values for liver, lung, spleen and kidney of 1G04-treated mice were 1.07*10⁸, 1.68*10⁶, 1.83*10⁷, and 1.46*10⁷, respectively. These data indicate that 1G04 is a potent inhibitor of metastasis spread and growth in colon cancer.

Collectively, FIGS. 16D-16G show that 1G04 is effective at reducing the formation and growth of metastases from colon cancer.

### Example 8: Treatment of Lung Cancer Using the 1G04 Anti-CD36 Antibody

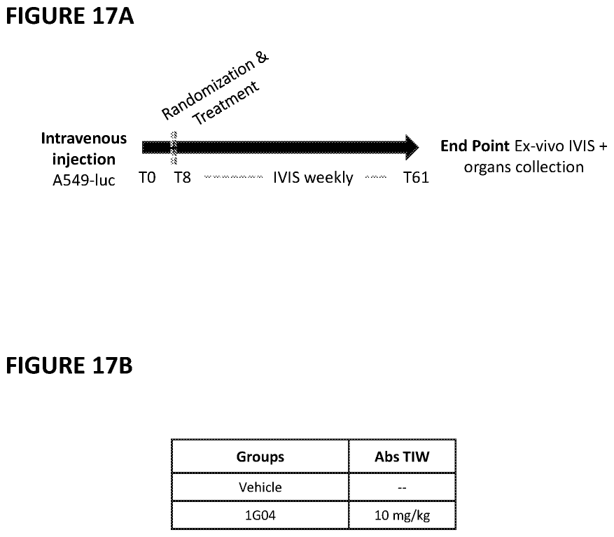

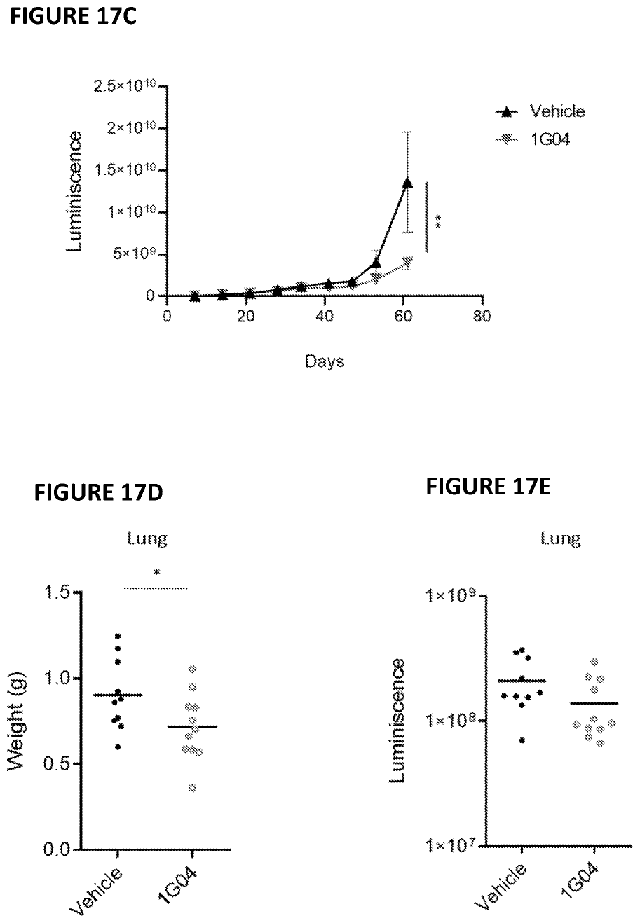

Studies of the effects of the 1G04 anti-CD36 antibody on lung cancer were performed in NSG mice (immuno-deficient). An experimental overview of these studies is provided in FIG. 17A. The studies included only female mice. All mice were inoculated with commercially available A549-luc2 (ATCC) cancer cells, a modified version of A549 cells generated by stable transduction with a lentiviral vector expressing luciferase. A549 cells are cells derived from a lung carcinoma (i.e. from a lung cancer), and therefore were used as part of a mouse model of lung cancer. Prior to inoculation, the A549 cells were cultured in a humidified incubator at 37° C. with 5% CO₂, and were grown in F-12K medium supplemented with 5 g ml⁻¹ penicillin/streptomycin and 10% FBS (GIBCO).

For each mouse, 1×10⁶ A549 cells were inoculated intravenously via tail vein injection. Each mouse was imaged after inoculation and one week later and lung metastasis was confirmed by luminescence prior to start of treatment. Treatment began 8 days after inoculation with the A549 cells. As detailed in FIG. 17B, inoculated mice were divided into one of two treatment groups: vehicle injection control (n=11) or 1G04 treatment (n=11). Antibody treatments were administered via intraperitoneal (i.p.) injection at a dose of 10 mg/kg three times per week, while control mice received an equal volume of vehicle on the same schedule. One day prior to the start of treatment, and once weekly after the start of treatment, all mice were imaged via IVIS. Mice were sacrificed at the end of the treatment period (day 61). Upon sacrifice, organs and tissues were collected for performance of necropsy and ex-vivo IVIS.

FIG. 17C shows the results of imaging whole-animal bioluminescence over time, with decreased fluorescence observed in 1G04 treated mice. This indicates that 1G04 treatment reduced the growth of the injected A549 tumor cells in vivo (**=p value of p=0.0002). At endpoint, lungs of mice treated with 1G04 antibody were smaller than lungs from control vehicle-treated mice (FIG. 17D), indicating that less tumor growth occurred. The observed mean lung weight was 0.90 g in vehicle-treated mice and 0.72 g in 1G04-treated mice (20% reduction). Animals treated with 1G04 also presented less luminescence in the lung at endpoint (2.11*108 to 1.39*108) as presented in FIG. 17E. These results indicate that 1G04 inhibits metastasis growth in lung cancer.

### Example 9: Treatment of Colon Cancer Using the 1G04 Anti-CD36 Antibody

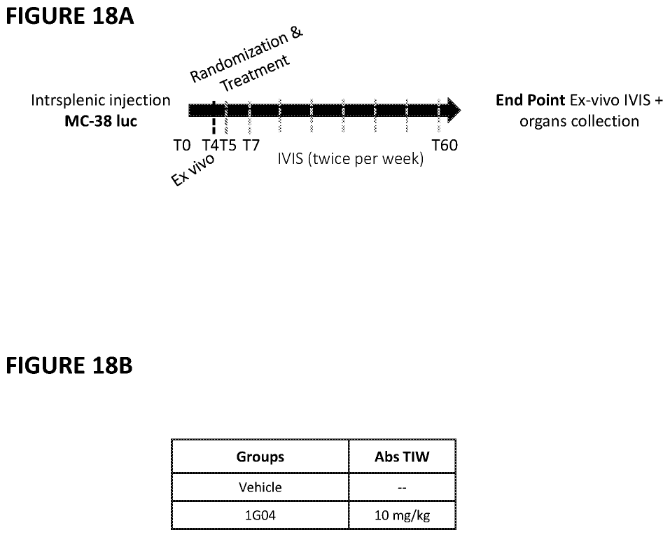

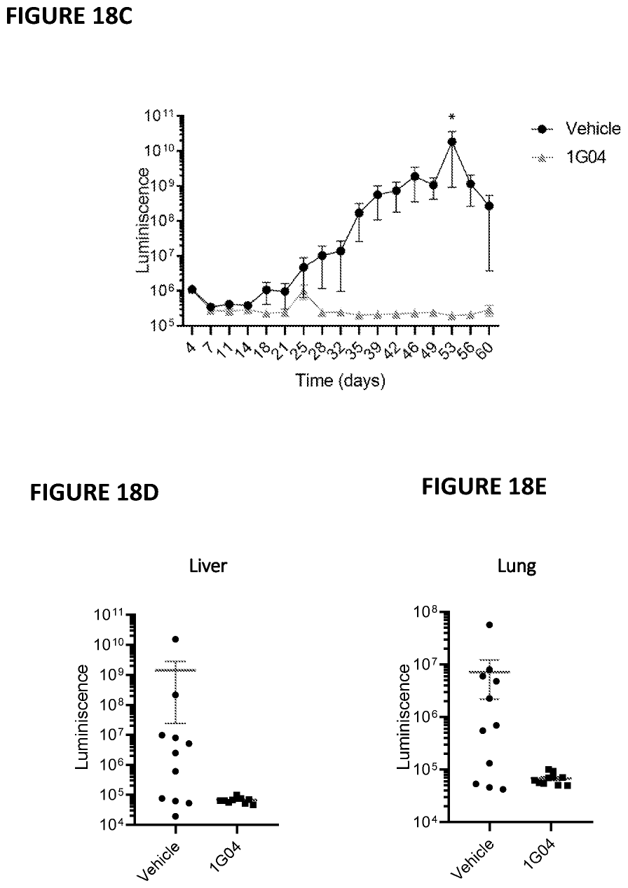

Studies of the effects of the 1G04 anti-CD36 antibody on colon cancer were performed in C57BL/6 mice (immuno-competent). An experimental overview of these studies is provided in FIG. 18A. The studies included only female mice. All mice were inoculated with commercially available MC-38 cancer cells, transduced with a vector expressing luciferase. MC-38 cells are cells derived from a mouse colon adenocarcinoma (i.e. from a colon cancer). Prior to inoculation, MC-38 cells were cultured in a humidified incubator at 37° C. with 5% CO₂ and were grown in DMEM medium supplemented with 5 μg ml⁻¹ penicillin/streptomycin and 10% FBS (GIBCO).

For each mouse, 1×10⁶ MC-38 cells were inoculated intrasplenically. Each mouse was imaged 4 days later, and liver metastasis was confirmed by ex vivo luminescence prior to start of treatment on day 5 after inoculation. As detailed in FIG. 18B, inoculated mice were divided into one of two treatment groups: vehicle injection control (n=13) or 1G04 treatment (n=10). Antibody treatments were administered via intraperitoneal (i.p.) injection at a dose of 10 mg/kg three times per week, while control mice received an equal volume of vehicle on the same schedule. One day prior to the start of treatment, and twice weekly after the start of treatment, all mice were imaged via IVIS. Mice were sacrificed at the end of the treatment period (day 60). Upon sacrifice, organs and tissues were collected for performance of necropsy and ex-vivo IVIS.

Whole-animal bioluminescence imaging during the study showed that 1G04-treatment decreases luminescence, indicating a reduction in tumoral growth (*=p value of 0.003, FIG. 18C). Ex vivo analysis of luminescence showed that mice treated with 1G04 present lower luminescence in both liver (1.41*10⁹ to 6.67*10⁴; i.e., greater than 99.99% reduction) and lungs (7.23*10⁶ to 6.78*10⁴; i.e., greater than 99% reduction) (FIGS. 18D and 18E, respectively). In conclusion, 1G04 showed efficacy decreasing metastasis size in colon cancer.

### Example 10: Treatment of Breast Cancer Using the 1G04 Anti-CD36 Antibody

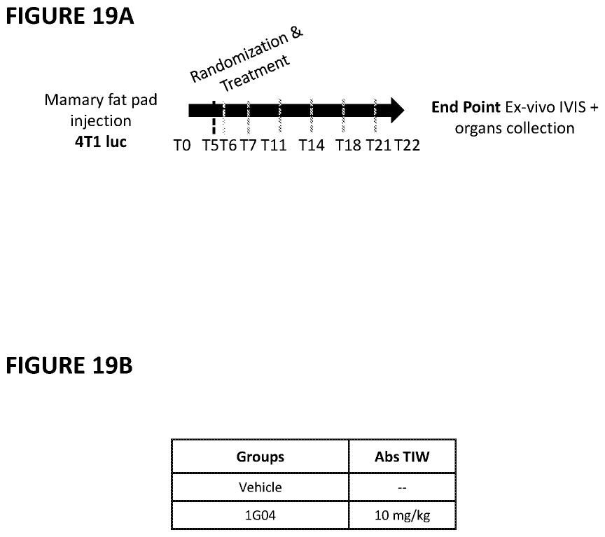

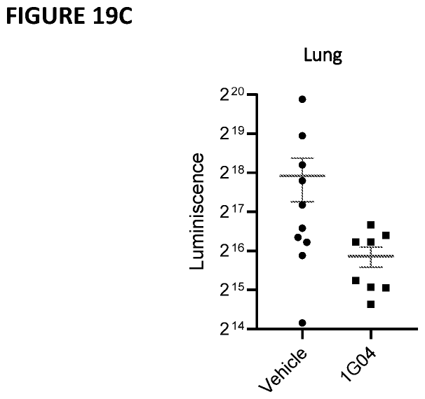

Studies of the effects of the 1G04 anti-CD36 antibody on breast cancer were performed in BALB/c mice (immuno-competent). An experimental overview of these studies is provided in FIG. 19A. The studies included only female mice. All mice were inoculated with commercially available 4T1 cancer cells (ATCC), transduced with a vector expressing luciferase. 4T1 cells were derived from murine mammary gland tissue (i.e. from a breast cancer). Prior to inoculation, 4T1 cells were cultured in a humidified incubator at 37° C. with 5% CO₂, and were grown in RPMI medium supplemented with 5 μg ml⁻¹ penicillin/streptomycin, 2 mM L-Glutamine and 10% FBS (GIBCO).

For each mouse, 4×10⁴ 4T1 cells were inoculated orthotopically in the mammary fat pad. Treatment began 5 days after inoculation with 4T1 cells. Mice were divided into one of two treatment groups: vehicle injection control (n=10) or 1G04 treatment (n=10). Antibody treatments were administered via intraperitoneal (i.p.) injection at a dose of 10 mg/kg three times per week, while control mice received an equal volume of vehicle on the same schedule (FIG. 19B). Mice were sacrificed at the end of the treatment period (day 22). Upon sacrifice, organs and tissues were collected for performance of necropsy and ex-vivo IVIS.

Luminescence in the lungs was reduced in 1G04-treated mice compared to vehicle-treated ones (2.49*10⁵ to 5.96*10⁴, FIG. 19C), indicating that anti-CD36 treatment reduces the size of metastasis and/or metastatic spread to distant organs.

### Example 11: Characterization of Anti-CD36 Antibodies

Novel anti-CD36 antibodies were generated via immunization of mice or chickens with recombinant human CD36. Mice were immunized with five injections of human CD36 protein, followed by a sixth injection of human-CD36-overexpressing CHO cells. Other mice were immunized with four injections of human-CD36-overexpressing CHO cells. Chickens were immunized with four injections of human CD36 protein. These immunization resulted in generation of Fab fragments, and the variable regions from these Fab fragments were then grafted onto a human IgG scaffold with the LALA alteration to form chimeric antibodies. The murine immunization process resulted in generation of the 4G04, 5G04, 6G04, 7G04, 9G04, 10G04, 11G04, 12G04, 13G04, 14G04, 15G04, 16G04, 17G04, 18G04, 19G04, 20G04, 21G04, 22G04, 23G04, 24G04, 25G04, 26G04, 27G04, 28G04, 29G04, 30G04, 31G04, and 32G04 chimeric antibodies. The chicken immunization process resulted in generation of the 33G04, 34G04, 35G04, 36G04, 37G04, and 38G04 chimeric antibodies. Humanization of 11G04 resulted in the generation of, 73G06, 74G06, 75G06, 76G06, 77G06, 78G06, 79G06, or 80G06. Amino acid sequences relating to these antibodies are provided in Table 2 and Table 3.

The murine-derived chimeric antibodies were screened for their ability to bind to HEK 293 cells transiently overexpressing human CD36 (SEQ ID NO: 1), nonhuman primate CD36 (SEQ ID NO: 2), or mouse CD36 (SEQ ID NO: 3). After transiently transfecting the HEK 293 cells with a CD36 expression construct, cells were treated with three-fold serial dilution of each candidate antibody (10 dilutions starting at 500 nM). The antibody 1G04 (SEQ ID NOs: 9 and 10; see PCT/IB2021/051881, which is herein incorporated by reference) was used as a positive control. After incubation with the cells, an anti-human fluorescently labelled antibody was added to the cell media, allowed to incubate further, and the unbound antibody was washed off of the cells. Subsequently, the cells were subjected to FACS analysis to determine antibody binding to the cells. The results were fitted to a sigmoidal titration curve. Using a nonlinear regression model, the EC₅₀ values for each antibody were calculated from the GeoMFI and MFI values using the log(agonist) versus response (four parameters) fitting on the data transformed according to X=Log[X] in GraphPad PRISM. The results of this analysis are provided in Table 7 below as the EC₅₀ of each antibody's ability to bind human CD36, non-human primate CD36 (cynomolgus), and mouse CD36 on the HEK 293 cells. All antibodies bound human CD36, but some were classified as not determined (N.D.) because they did not present a proper titration curve that fits the nonlinear regression model used to calculate EC₅₀. Most of the antibodies were measured to have a similar EC₅₀ as 1G04 against human CD36. Two antibodies (31G04 and 4G04) showed low affinity for the three CD36 species tested, and these antibodies' EC₅₀ value could not be unambiguously calculated by nonlinear regression. Two antibodies (26G04 and 31G04) did not bind nonhuman primate CD36, while one antibody (4G04) bound to nonhuman primate CD36 with significantly less affinity than the other antibodies. Eleven antibodies did not bind mouse CD36 or bound to it with significantly less affinity than the other antibodies.

TABLE 7
Antibody Binding to HEK 293 Cells Overexpressing CD36

| | | Non-Human | |
|---|---|---|---|
| | Human CD36 | Primate CD36 | Mouse CD36 |
| Antibody | (nM) | (nM) | (nM) |
| 24G04 | 0.26 | 0.81 | N.D. |
| 23G04 | 0.30 | 1.69 | N.D. |
| 13G04 | 0.31 | 1.06 | 5.93 |
| 12G04 | 0.31 | 0.67 | 1.20 |
| 30G04 | 0.32 | 1.63 | 0.96 |
| 21G04 | 0.36 | 6.38 | N.D. |
| 29G04 | 0.36 | 0.32 | N.D. |
| 11G04 | 0.45 | 8.93 | 11.23 |
| 7G04 | 0.48 | 4.16 | 3.73 |
| 9G04 | 0.50 | 13.92 | 11.97 |
| 6G04 | 0.53 | 4.85 | 1.51 |
| 28G04 | 0.60 | 7.77 | 8.50 |
| 26G04 | 0.72 | N.D. | 158.20 |
| 25G04 | 0.74 | 2.19 | N.D. |
| 10G04 | 0.77 | 1.48 | 15.99 |
| 27G04 | 0.91 | 0.63 | N.D. |
| 32G04 | 0.94 | 6.52 | 5.14 |
| 17G04 | 1.25 | 4.03 | 9.31 |
| 22G04 | 1.25 | 1.68 | N.D. |
| 16G04 | 1.36 | 5.40 | 18.16 |
| 14G04 | 1.47 | 0.88 | 1.57 |
| 19G04 | 1.92 | 5.45 | 22.47 |
| 1G04 | 2.07 | 1.23 | 1.994 |
| 31G04 | N.D. | N.D. | N.D. |
| 20G04 | N.D. | 5.03 | 18.23 |
| 18G04 | N.D. | 6.60 | 14.66 |
| 15G04 | N.D. | 2.20 | N.D. |
| 5G04 | N.D. | 2.43 | 5.03 |
| 4G04 | N.D. | 27.69 | N.D. |
| 33G04 | 0.31 | 0.52 | 4.33 |
| 34G04 | 0.56 | 1.40 | 6.75 |
| 35G04 | 0.63 | 0.59 | 2.25 |
| 36G04 | 0.33 | 0.29 | 0.66 |
| 37G04 | 0.62 | 1.45 | 2.29 |
| 38G04 | 0.35 | 0.33 | 0.69 |

A subset of humanized antibodies were also screened for their ability to bind to HEK 293 cells transiently overexpressing human CD36 (SEQ ID NO: 1). After transiently transfecting the HEK 293 cells with a CD36 expression construct, cells were treated with three-fold serial dilution of each candidate antibody (10 dilutions starting at 500 nM). The antibodies 11G04 (SEQ ID NOs: 19 and 51) and 11G06 (SEQ ID NOs: 16 and 51) were used as a positive control. Antibody binding to cells was measured by FACS as above. The results of this analysis are provided in Table 8 below as the EC₅₀ of each antibody's ability to bind human CD36. All tested antibodies bound human CD36 and have a similar EC₅₀ as 11G04 and 11G06 against human CD36.

TABLE 8
Humanized Antibody Binding to HEK293
Cells Overexpressing CD36

| | Human CD36 |
|---|---|
| Antibody | (nM) |
| 11G04 | 0.49 ± 0.16 |
| 11G06 | 0.76 ± 0.29 |
| 73G06 | 0.87 ± 0.35 |
| 74G06 | 1.10 ± 0.49 |
| 75G06 | 0.98 ± 0.54 |
| 76G06 | 0.88 ± 0.50 |
| 77G06 | 0.81 ± 0.38 |
| 78G06 | 1.15 ± 0.70 |
| 79G06 | 0.65 ± 0.32 |
| 80G06 | 0.65 ± 0.37 |

A subset of the murine-derived chimeric antibodies were also screened for their ability to bind to SCC25 cells stably overexpressing human CD36 (SEQ ID NO: 1). Using the same FACS-based titration assay as used for the HEK 293 cells, the tested antibodies all had similar EC₅₀'s with SCC25 cells as they did with HEK 293 cells. These results are shown in Table 9 below.

TABLE 9
Antibody Binding to SCC25 Cells Overexpressing CD36

| | Human CD36 |
|---|---|
| Antibody | (nM) |
| 13G04 | 0.26 |
| 12G04 | 0.39 |
| 30G04 | 0.22 |
| 11G04 | 0.46 |
| 7G04 | 0.68 |
| 9G04 | 0.41 |
| 6G04 | 0.56 |
| 28G04 | 0.95 |
| 10G04 | 3.30 |
| 32G04 | 1.88 |
| 16G04 | 0.57 |
| 14G04 | 0.41 |
| 19G04 | 0.75 |
| 1G04 | 0.34 |

The binding affinity of the reported antibodies for human CD36 was also measured by surface plasmon resonance (SPR) using a Biacore T200. The anti-histidine antibody provided in His Capture Kit (Cytiva #28-9950-56) was diluted to 50 g/ml in immobilization buffer and covalently coupled to a CM5 Sensor Chip by standard amine coupling to a level of approximately 13,000 RU. For each single-cycle experiment, human CD36 (His-tagged) was captured at low density (approximately 40 RU) and a titration of five antibody concentrations (diluted in running buffer) were injected over the captured antigen and then the dissociation of the complex was measured. A double referencing method was employed in which data from the reference surfaces where no antigen was captured (fc 1) were subtracted from the antigen bound capture surface (fc 2). Blank injections of buffer were run for every antibody titration cycle and then subtracted from analyte injection cycles, to correct for small changes in the capture surface density. Surface regeneration between cycles was performed by injecting Glycine pH 1.5 for 60 s. All analysis was performed at 20° C. in HBS-P running buffer (flow 30 μL/min), and the sample rack was kept at 6° C. during experimental runs. Data was fitted to a bivalent analyte model from which the kinetic and thermodynamic binding constants were derived. Data from this analysis is provided in Table 10 below.

TABLE 10
Mean Kinetic Data for the SPR-Measured
Interaction with CD36 (Bivalent Model)

| Antibody | kₐ₁ (1/Ms) | K_{d1} (1/s) | K_{D1} (M) |
|---|---|---|---|
| 1G04 | 2.9E+06 | 9.4E−03 | 3.2E−09 |
| 6G04 | 3.9E+05 | 2.0E−04 | 5.1E−10 |
| 7G04 | 7.1E+05 | 3.5E−03 | 4.9E−09 |
| 11G04 | 1.2E+06 | 2.2E−02 | 1.9E−08 |
| 13G04 | 2.4E+06 | 3.7E−03 | 1.5E−09 |
| 14G04 | 3.1E+06 | 7.2E−03 | 2.3E−09 |
| 28G04 | 8.7E+05 | 1.3E−02 | 1.4E−08 |

To further assess properties of the anti-CD36 antibodies, the melting temperature of the antibodies was measured. Each tested antibody was mixed with a dye that strongly fluoresces when it binds to hydrophobic regions of a protein (Sypro orange), and slowly heated to 100° C. The melting temperature was identified as the point of maximum slope in a plot of the fluorescence over time, representing when the protein unfolds and exposes its hydrophobic regions to the dye. The results of this assay are shown in Table 11 below.

TABLE 11
Melting Temperature of Anti-CD36 Antibodies

| Antibody | Melting Temp (° C.) |
|---|---|
| 01G04 | 69.8 |
| 04G04 | 69.5 |
| 05G04 | 70.5 |
| 06G04 | 68.5 |
| 07G04 | 68.8 |
| 09G04 | 70.5 |
| 10G04 | 67.5 |
| 11G04 | 67.0 |
| 12G04 | 67.1 |
| 13G04 | 67.8 |
| 14G04 | 69.5 |
| 15G04 | 67.4 |
| 16G04 | 66.5 |
| 17G04 | n.d. |
| 18G04 | 70.1 |
| 19G04 | 66.8 |
| 20G04 | 62.8 |
| 21G04 | 67.5 |
| 22G04 | 69.4 |
| 23G04 | 68.1 |
| 24G04 | 67.7 |
| 25G04 | 70.0 |
| 26G04 | 70.6 |
| 27G04 | 66.7 |
| 28G04 | 70.4 |
| 29G04 | 68.2 |
| 30G04 | 55.9 |
| 31G04 | 56.8 |
| 32G04 | 70.6 |

A set of anti-CD36 antibodies were evaluated for their ability to compete with either the 1G04 antibody, the FA6-152 anti-CD36 antibody (Abcam), and/or the 11G04 antibody for binding to human CD36. This was done using the AlphaScreen® bead-based microplate assay. The reference antibody (1G04, FA6-152, or 11G04) (20 g/mL) was conjugated to AlphaLISA® acceptor beads and incubated with purified biotin-tagged human CD36 (12.5 nM) and the test anti-CD36 antibody (two-fold serial dilutions from 500 to 0.10 nM). AlphaLISA® Streptavidin Donor Beads were then added and the mix incubated for 30 min at 25° C. in the dark. The donor bead contains a photosensitizer, which upon excitation by light at 680 nm, converts oxygen (O₂) into an excited form, singlet oxygen. Singlet oxygen molecules have a reduced lifetime (4 microseconds half-life) and can diffuse approximately 200 nm in solution before falling back to ground state. In the absence of acceptor beads, the singlet oxygen molecules fall back to ground state without producing any light signal. In case an acceptor bead is within 200 nm, energy is transferred from the singlet oxygens to the bead, resulting in light production at 615 nm. Accordingly, the degree of binding between the reference antibody and CD36 was measured as the degree of fluorescence at 615 nm observed in the presence of a test anti-CD36 antibody, with decreased fluorescence relative to controls expected if the antibody is capable of disrupting the bead-to-bead interaction by competing for binding to the bound CD36. The results of the competition assay are summarized in Table 12 below. Most of the tested antibodies compete solely with 1G04 (05G04, 17G04, 21G04, 22G04, 23G04, 32G04, 12G04, 7G04, 9G04, 28G04 and 14G04), or with both 1G04 and 11G04 (16G04, 18G04, 30G04, 33G04, 34G04, 35G04, 36G04, 37G04, and 38G04). The 19G04 and 20G04 antibodies were observed to compete solely with 11G04. The 4G04, 6G04, 13G04, 15G04, 24G04, 25G04, 26G04, 27G04, 29G04, and 31G04 antibodies were observed to compete with both 1G04 and FA6-152. The 10G04 and 11G04 and 19G04 antibodies did not compete with either 1G04 or FA6-152.

TABLE 12
Anti-CD36 Antibody Competition

| | Competes with | Competes with | Competes with |
|---|---|---|---|
| Antibody | 1G04 | FA6-152 | 11G04 |
| 13G04 | YES (EC₅₀ = 0.17 | YES (EC₅₀ = 46.80 | N.D. |
| | nM) | nM) | |
| 12G04 | YES (EC₅₀ = 0.78 | NO | N.D. |
| | nM) | | |
| 11G04 | NO | NO | N.D. |
| 7G04 | YES (EC₅₀ = 0.95 | NO | N.D. |
| | nM) | | |
| 9G04 | YES (EC₅₀ = 2.06 | NO | N.D. |
| | nM) | | |
| 6G04 | YES (EC₅₀ = 1.15 | YES (EC₅₀ = 1044 | N.D. |
| | nM) | nM) | |
| 28G04 | YES (EC₅₀ = 4.69 | NO | N.D. |
| | nM) | | |
| 16G04 | YES (EC₅₀ = 9.19 | NO | YES (EC₅₀ = 3.73 |
| | nM) | | nM) |
| 14G04 | YES (EC₅₀ = 0.31 | NO | N.D. |
| | nM) | | |
| 19G04 | NO | NO | YES (EC₅₀ = 5.23) |
| 04G04 | YES (EC₅₀ = 0.11 | YES (EC₅₀ = 9.89 | N.D. |
| | nM) | nM) | |
| 05G04 | YES (EC₅₀ = 0.02 | NO | N.D. |
| | nM) | | |
| 18G04 | YES (EC₅₀ = 15.61 | NO | YES (EC₅₀ = 4.54) |
| | nM) | | |
| 20G04 | NO | NO | YES (EC₅₀ = 4.69) |
| 21G04 | YES (EC₅₀ = 0.45 | NO | N.D. |
| | nM) | | |
| 22G04 | YES (EC₅₀ = 0.53 | NO | N.D. |
| | nM) | | |
| 23G04 | YES (EC₅₀ = 0.28 | NO | N.D. |
| | nM) | | |
| 24G04 | YES (EC₅₀ = 0.29 | YES (EC₅₀ = n.d.) | N.D. |
| | nM) | | |
| 25G04 | YES (EC₅₀ = 0.29 | YES (EC₅₀ = n.d.) | N.D. |
| | nM) | | |
| 26G04 | YES (EC₅₀ = 0.46 | YES (EC₅₀ = 2.66 | N.D. |
| | nM) | nM) | |
| 27G04 | YES (EC₅₀ = 0.15 | YES (EC₅₀ = 1.8 | N.D. |
| | nM) | nM) | |
| 29G04 | YES (EC₅₀ = 0.37 | YES (EC₅₀ = 33.8 | N.D. |
| | nM) | nM) | |
| 31G04 | YES (EC₅₀ = 0.27 | YES (EC₅₀ = 2.50 | N.D. |
| | nM) | nM) | |
| 32G04 | YES (EC₅₀ = 1.46 | NO | N.D. |
| | nM) | | |
| 08G04 | YES (EC₅₀ = 0.68 | NO | N.D. |
| | nM) | | |
| 10G04 | NO | NO | N.D. |
| 15G04 | YES (EC₅₀ = 0.41 | YES (EC₅₀ = 5.65 | N.D. |
| | nM) | nM) | |
| 17G04 | YES (EC₅₀ = 0.52 | NO | N.D. |
| | nM) | | |
| 28G04 | YES (EC₅₀ = 4.69 | NO | N.D. |
| | nM) | | |
| 30G04 | YES (EC₅₀ = n.d.) | NO | YES (EC₅₀ = 0.65 |
| | | | nM) |
| 33G04 | YES (EC₅₀ = 1.58 | N.D. | YES (EC₅₀ = 3.68 |
| | nM) | | nM) |
| 34G04 | YES (EC₅₀ = 2.47 | N.D. | YES (EC₅₀ = 4.82 |
| | nM) | | nM) |
| 35G04 | YES (EC₅₀ = 0.66 | N.D. | YES (EC₅₀ = 1.35 |
| | nM) | | nM) |
| 36G04 | YES (EC₅₀ = 0.57 | N.D. | YES (EC₅₀ = 1.04 |
| | nM) | | nM) |
| 37G04 | YES (EC₅₀ = 9.91 | N.D. | YES (EC₅₀ = 3.30 |
| | nM) | | nM) |
| 38G04 | YES (EC₅₀ = 1.45 | N.D. | YES (EC₅₀ = 1.05 |
| | nM) | | nM) |

### Example 12: Testing Effects of Anti-CD36 Antibodies on CD36's Interaction with its Ligands

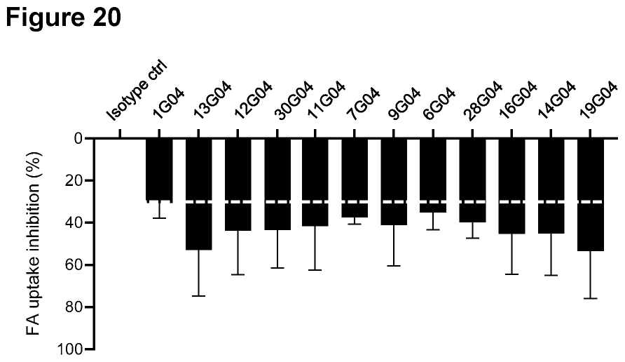

The ability of anti-CD36 antibodies to affect CD36-mediated fatty acid uptake was measured using a cell-based assay adapted from prior publications. See Feng, W. et al., Cell/Reports 29(11):3405-3420 (2019); Henkin, A. et al., ACS Chem. Biol. 7(11):1884-91 (2012). Briefly, SCC25 cells were engineered to overexpress both luciferase and human CD36 and plated in a 96-well plate. Palmitic acid coupled to luciferin was then added to the cell media, and incubated to allow CD36-mediated uptake and the generation of luminescence by reaction of luciferase with its substrate luciferin. The resultant luminescence was measured after incubation either with a control isotype antibody or an anti-CD36 antibody present in the cell media. As shown in FIG. 20, each anti-CD36 antibody tested inhibited fatty acid uptake relative to the uptake observed after treatment with an isotype control antibody. The mean percent inhibition of fatty acid uptake observed is also shown in Table 13.

TABLE 13
Anti-CD36 Antibody Inhibition of Fatty Acid Uptake

| | Percent Fatty Acid Uptake |
|---|---|
| Antibody | Inhibition |
| 13G04 | 52.8 |
| 12G04 | 43.8 |
| 30G04 | 43.5 |
| 11G04 | 41.6 |
| 7G04 | 37.4 |
| 9G04 | 41.1 |
| 6G04 | 35.1 |
| 28G04 | 39.7 |
| 16G04 | 45.2 |
| 14G04 | 45.1 |
| 19G04 | 53.3 |
| 1G04 | 30.5 |

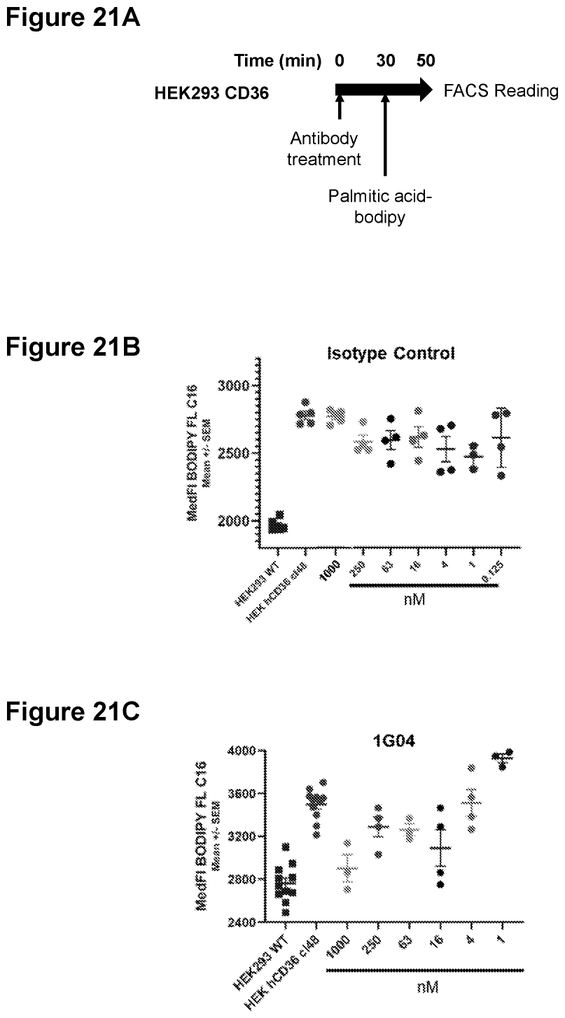

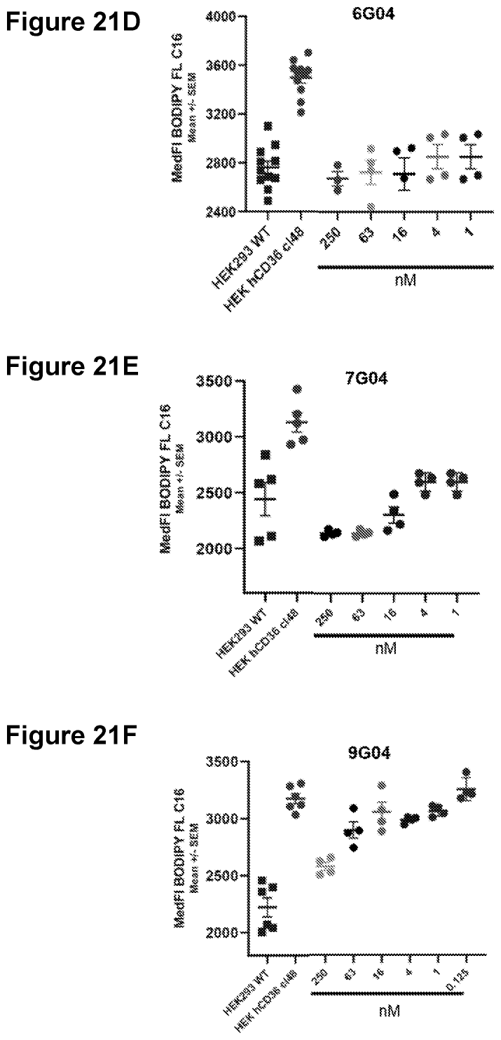

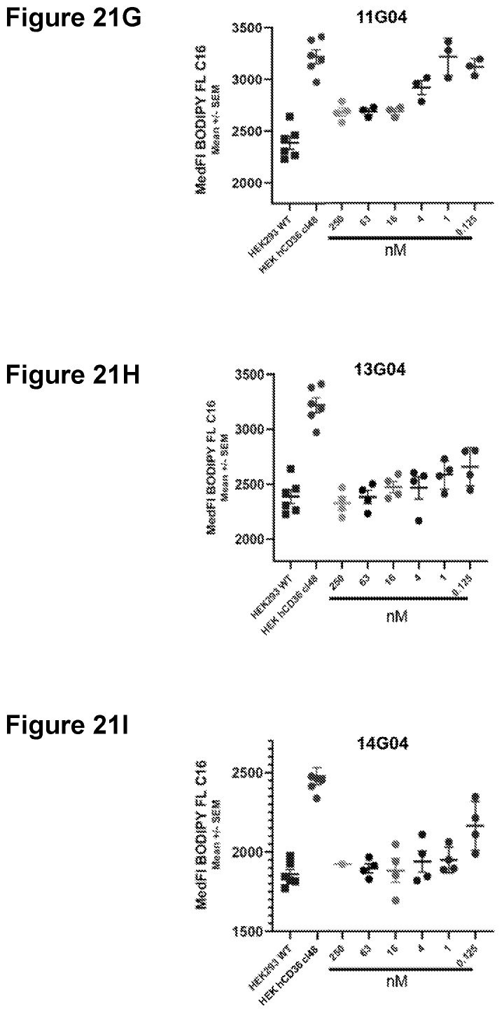

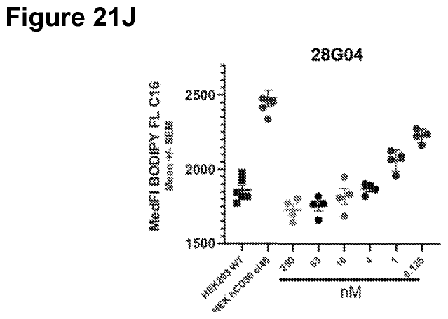

The ability of anti-CD36 antibodies to inhibit CD36-mediated fatty acid uptake was also measured by their ability to inhibit uptake of palmitic acid linked to a fluorophore (BODIPY FL C16). The basic experimental protocol is provided in FIG. 21A. HEK 293 cells were stably transfected with a vector to overexpress human CD36 to generate a stable cell line (HEK hCD36 c148), which enhanced fatty acid uptake. Both control untransfected HEK 293 cells and the HEK hCD36 c148 cells were used in each assay as reference controls (FIGS. 21B-21J). Either an anti-CD36 antibody or an isotype control were then added to the cell media of a well containing HEK hCD36 c148 cells, and incubated for 30 minutes at 37° C. Bodipy-labelled palmitic acid was then added and incubated with the cells for 20 minutes at 37° C., at which point FACS analysis was performed to detect intracellular fluorescence. Each antibody was titrated to test its effects at a range of concentrations from 0.125 nM to 1000 nM. The results of this assay are plotted in FIGS. 21B-21J. Each of 6G04 (FIG. 21D), 7G04 (FIG. 21E), 9G04 (FIG. 21F), 11G04 (FIG. 21G), 13G04 (FIG. 21H), 14G04 (FIG. 21I), and 28G04 (FIG. 21J) inhibited palmitic acid uptake into HEK 293 cells, relative to treatment with isotype control antibody (FIG. 21B). In addition, each of 6G04, 7G04, 9G04, 11G04, 13G04, 14G04, and 28G04 inhibited palmitic acid uptake more effectively than 1G04 (FIG. 21C). The IC₅₀ for inhibition of fatty acid uptake for each tested antibody is provided in Table 14.

TABLE 14
Anti-CD36 inhibition (IC₅₀ calculation) of fatty acid uptake

| Antibody | IC₅₀ (nM) |
|---|---|
| 001G04 | 1.0 |
| 004G04 | 483.0 |
| 005G04 | >1000 |
| 006G04 | 0.1 |
| 007G04 | 1.0 |
| 009G04 | 170.0 |
| 010G04 | 33.0 |
| 011G04 | 0.07 |
| 012G04 | 3.90 |
| 013G04 | 0.20 |
| 014G04 | 0.13 |
| 015G04 | >1000 |
| 016G04 | 0.15 |
| 018G04 | 2.60 |
| 019G04 | 0.05 |
| 023G04 | 1.00 |
| 028G04 | 0.09 |
| 029G04 | 0.08 |
| 030G04 | 0.80 |
| 032G04 | 42.0 |
| 033G04 | 0.1 |
| 034G04 | 0.2 |
| 035G04 | 0.04 |
| 036G04 | 0.81 |
| 037G04 | 0.3 |
| 038G04 | 0.18 |

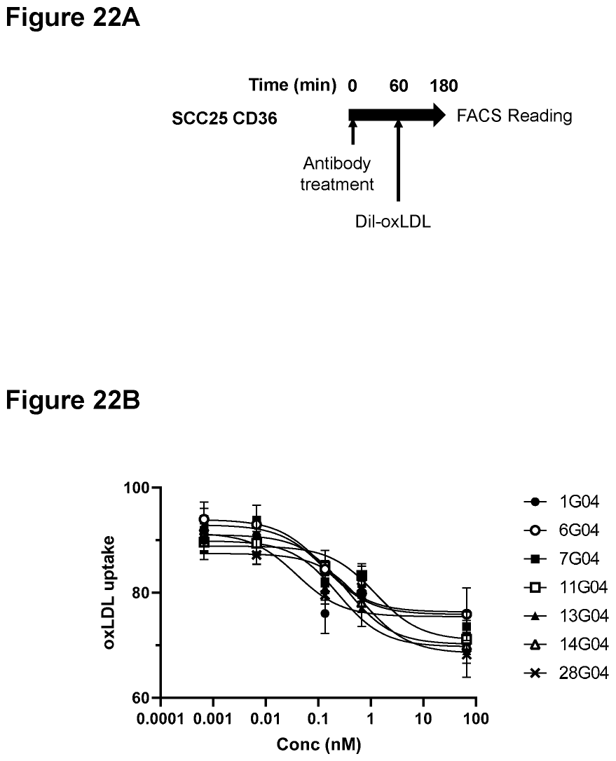

In addition to facilitating fatty acid uptake, CD36 facilitates internalization of oxidized LDL (“oxLDL”). The ability of anti-CD36 antibodies to inhibit CD36-mediated oxLDL uptake was measured by their ability to inhibit uptake of oxLDL linked to a fluorophore (DiI). The basic experimental protocol is provided in FIG. 22A. SCC25 cells stably expressing human CD36 were plated and treated with either an anti-CD36 antibody or an isotype control in the cell media and incubated for 60 minutes at 37° C. DiI-labelled oxLDL was then added and incubated with the cells for 120 minutes at 37° C., at which point FACS analysis was performed to detect intracellular fluorescence. Each antibody was titrated to test its effects at a range of concentrations from 335 nM to 0.00067 nM. The results of this assay are plotted in FIG. 22B. Each of 1G04, 6G04, 7G04, 11G04, 13G04, 14G04, and 28G04 inhibited oxLDL uptake into SCC25 cells, relative to treatment with isotype control antibody. The IC₅₀ for inhibition of oxLDL uptake for each tested antibody is provided in Table 15.

TABLE 15
Anti-CD36 inhibition of oxLDL uptake

| Antibody | IC₅₀ (nM) |
|---|---|
| 1G04 | 0.36 |
| 6G04 | 0.27 |
| 7G04 | 0.27 |
| 11G04 | 1.17 |
| 13G04 | 0.26 |
| 14G04 | 0.35 |
| 28G04 | 1.67 |
| 33G04 | 0.28 |
| 34G04 | 1.06 |
| 35G04 | 0.11 |
| 36G04 | 0.09 |
| 37G04 | 1.14 |
| 38G04 | 0.35 |

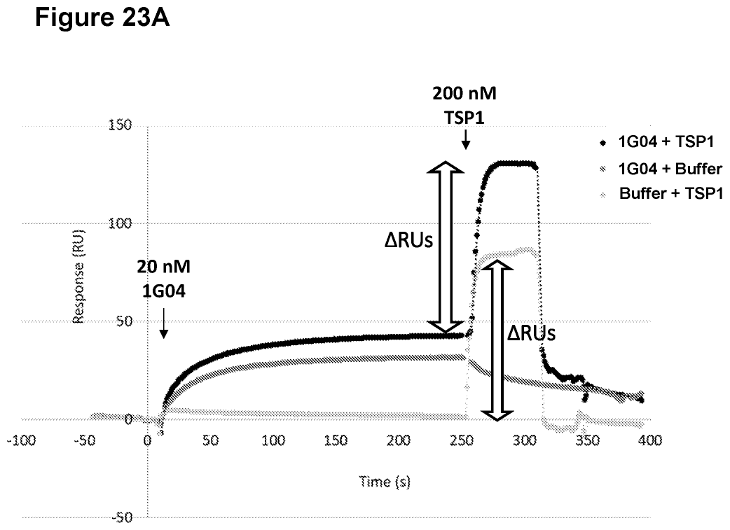

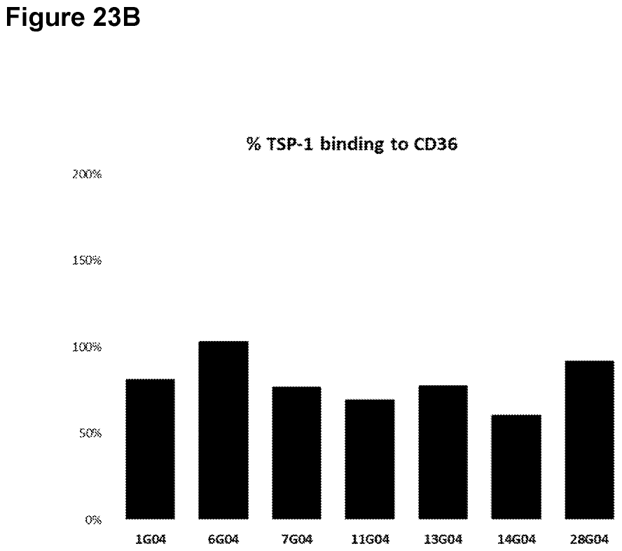

To evaluate if the binding of the Abs to CD36 interferes with the CD36's interaction with TSP1, anti-CD36 antibodies were tested in an SPR competition experiment. After immobilizing CD36 on the SPR surface, 20 nM anti-CD36 antibody was injected to reach binding saturation on CD36. After binding saturation is reached (approximately 250 seconds), a short pulse of 200 nM TSP1 was injected. An exemplary plot of the data obtained using 1G04, with the protocol steps annotated, is provides in FIG. 23A. To determine whether anti-CD36 antibody interferes with TSP-1 interaction, the change in response units (RUs) after TSP-1 injection was compared for injections containing anti-CD36 antibody and injections containing buffer only (see FIG. 23A). As shown in FIG. 23B, none of the tested antibodies reduced the TSP-1/CD36 interaction by more than 50%.

### Example 13—Engineering the Fc Region of Anti-CD36 Antibodies

The variable regions of the 1G04 anti-CD36 antibody were cloned to be attached to different constant regions, each of which contained a different Fc silencing alteration. The constant regions tested were: (1) the human IgG1 wild type sequence; (2) human IgG1 with the L234A and L235A (“LALA”) alteration; (3) human IgG1 with the amino acid mutations L234A, L235A and P329G (“P329G LALA”); human IgG1 with the amino acid mutations L234S, L235T, and G236R (“STR”); and human IgG4 with the amino acid mutations S228P and L235E (“SPLE”). The antibodies generated in this way were the 1G03 antibody (WT IgG1), the 1G05 antibody (IgG1 with P329G LALA); the 1G06 antibody (IgG1 with STR); and the 1G07 antibody (IgG4 with SPLE).

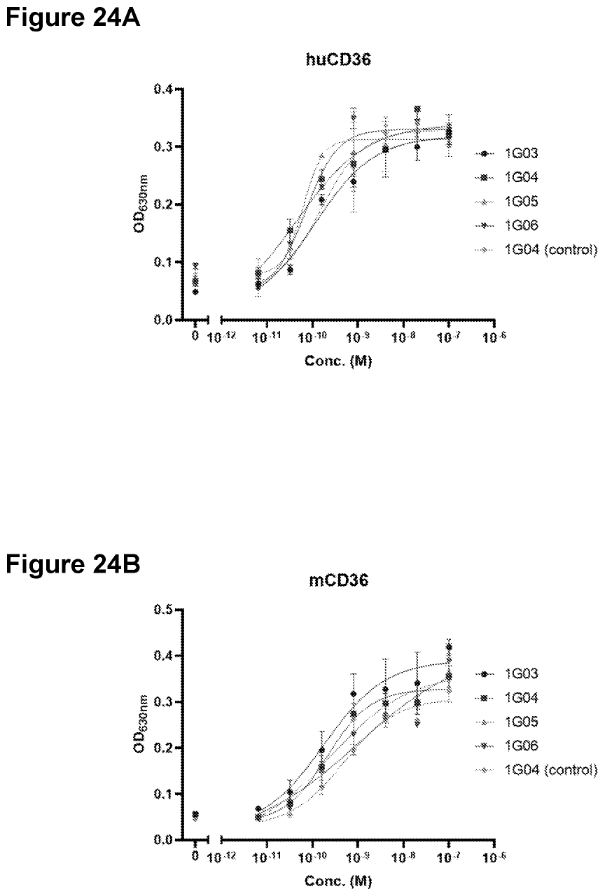

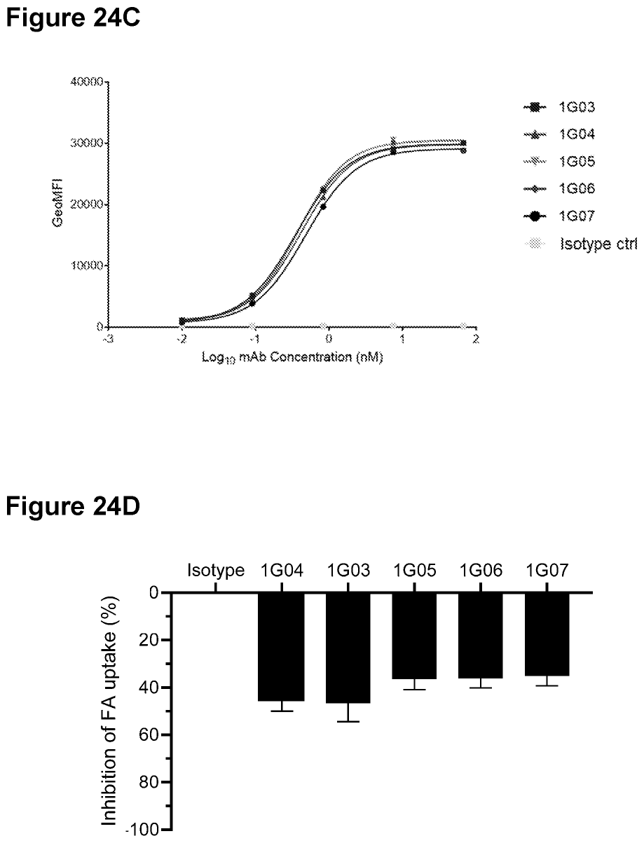

Each of the resultant chimeric antibodies derived from 1G04 was then tested for its ability to bind to human CD36 and mouse CD36. Binding was first tested using an ELISA assay in which 96-well ELISA plates were coated with human CD36 (Sino Biological) or mouse CD36 protein. As shown in FIG. 24A (binding to human CD36) and FIG. 24B (binding to mouse CD36), no binding differences were observed for the different Fc-formatted antibodies.

The different Fc-formatted anti-CD36 antibodies were also tested for their ability to bind to SCC25 cells stably expressing human CD36 (SEQ ID NO: 1). SCC25 cells were treated with 10-fold serial dilution of each antibody (5 dilutions starting at 100 nM). After incubation with the cells, an anti-human fluorescently labelled antibody was added to the cell media, allowed to incubate further, and the unbound antibody was washed off of the cells. Subsequently, the cells were subjected to FACS analysis to determine antibody binding to the cells. The results were fitted to a sigmoidal titration curve. Using a nonlinear regression model, the EC₅₀ values for each antibody were calculated from the GeoMFI and MFI values using the log(agonist) versus response (four parameters) fitting on the data transformed according to X=Log[X] in GraphPad PRISM. The results of this analysis are provided in Table 16 below, and plotted in FIG. 24C. These data further confirm that Fc engineering does not affect binding to CD36.

TABLE 16
Antibody Binding to SCC25 Cells Overexpressing CD36

| Antibody | EC₅₀ huCD36 (nM) |
|---|---|
| 1G03 | 0.38 |
| 1G04 | 0.42 |
| 1G05 | 0.38 |
| 1G06 | 0.38 |
| 1G07 | 0.47 |

The ability of the different Fc-formatted anti-CD36 antibodies to affect CD36-mediated fatty acid uptake was measured using a cell-based assay adapted from prior publications. See Feng et al., Cell Reports 2019; Henkin et al., ACS Chem. Biol. 2012. Briefly, SCC25 cells were engineered to overexpress both luciferase and human CD36 and plated in a 96-well plate. Palmitic acid coupled to luciferin was then added to the cell media, and incubated to allow CD36-mediated uptake and the generation of luminescence by reaction of luciferase with its substrate luciferin. The resultant luminescence was measured after incubation either with a control isotype antibody or an anti-CD36 antibody present in the cell media. As shown in FIG. 24D, each of the different Fc variants inhibited fatty acid uptake at similar levels, which showed that Fc engineering does not affect CD36-mediated fatty acid uptake inhibition by anti-CD36 antibodies.

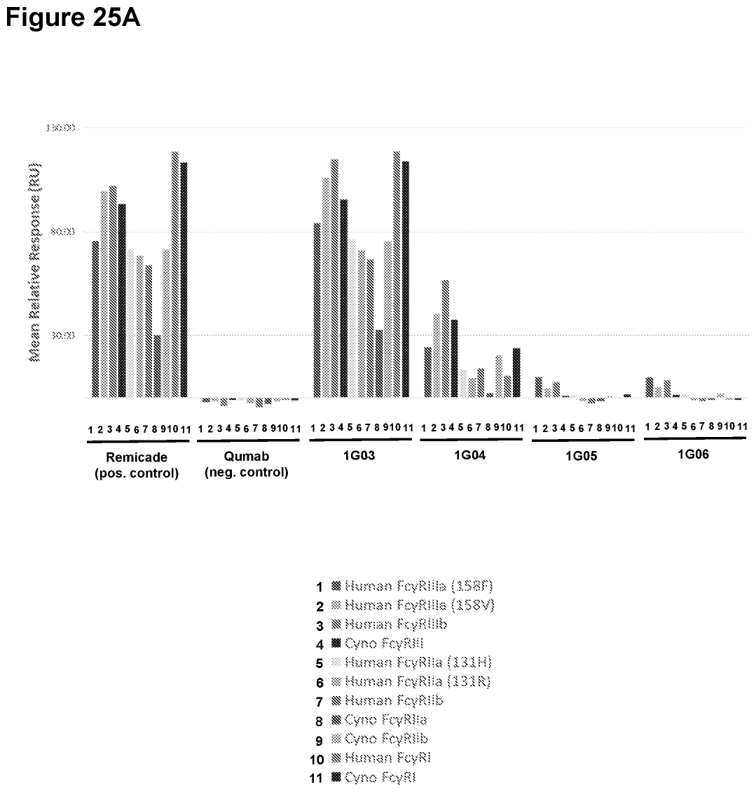

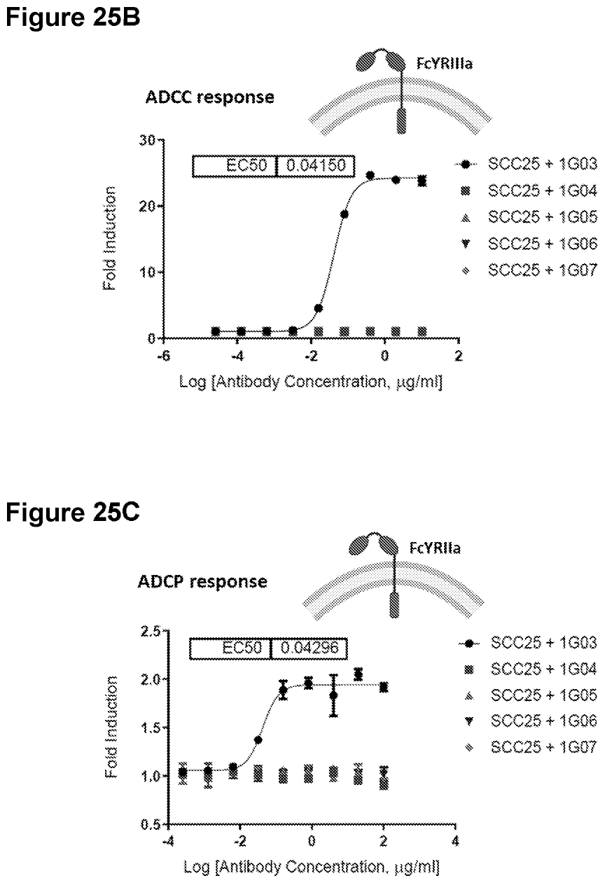

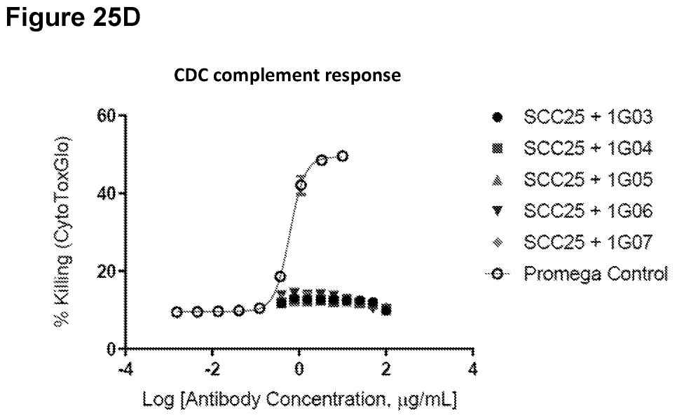

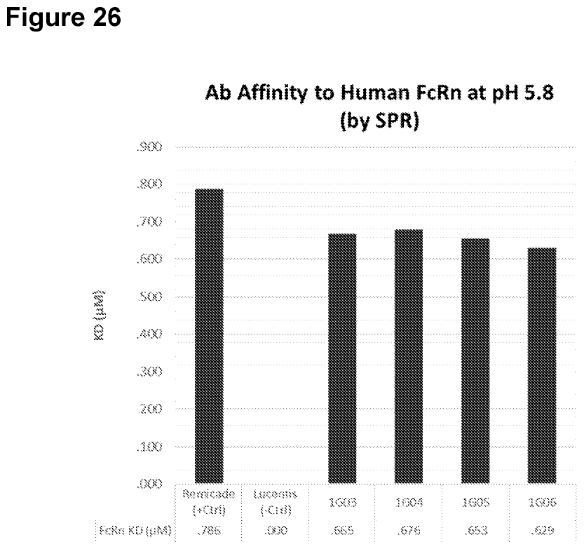

The binding of the different Fc-formatted anti-CD36 antibodies to FcγRs and FcRn was measured by SPR by immobilizing the antibodies through their light chains. The setup for these assays orients the antibody such that the Fc domain is solvent-exposed, allowing quantification of the interaction with soluble receptor proteins. Antibodies with well-defined binding properties were included as positive/negative controls in all the assays. As shown in FIG. 25A, each of the tested Fc alterations reduced interaction with human and cynomolgus FcγRs. Antibodies containing the LALAPG (1G05) and STR alterations were the most inactive toward human and cynomolgus FcγRs. In contrast, none of the alterations altered the affinity of the interaction (K_{D}) with human FcRn at pH 5.8—as shown in FIG. 26. This indicates that all Fc variants maintain FcRn affinity at endosomal pH, while dissociation at neutral pH is fast, thus ensuring an efficient Fc-mediated cellular recycling of the Ab in vivo.

The different Fc-formatted anti-CD36 antibodies were also tested using Promega's ADCC, ADCP, and CDC Reporter Bioassays according to the manufacturer's instructions. As shown in FIG. 25B, the FcγRIIIa-mediated ADCC response is completed abrogated (i.e., undetectable) in cells exposed to 1G04, 1G05, 1G06, and 1G07 antibodies. Similarly, as shown in FIG. 25C, the FcγRIIa-mediated ADCP response is completely abrogated (i.e., undetectable) in cells exposed to 1G04, 1G05, 1G06, and 1G07 antibodies. And finally, as shown in FIG. 25D, the CDC complement response is undetectable in cells exposed to 1G04, 1G05, 1G06, and 1G07 antibodies. In each of these assays, a significant response was observed after exposure to the 1G03 antibody, which contains an unmodified IgG1 Fc region.

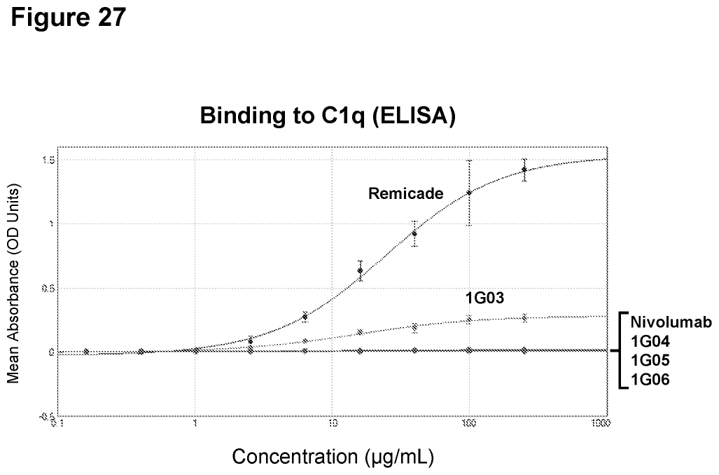

Binding to complement C1q protein was also measured, using a standard ELISA assay in which 96-well ELISA plates were coated with the tested antibodies and C1q human protein was added (2.5-fold serial dilution starting at 250 g/mL). As shown in FIG. 27, no binding was observed for any of the Fc-engineered samples, while the wild type Fc antibodies showed binding to C1q human protein.

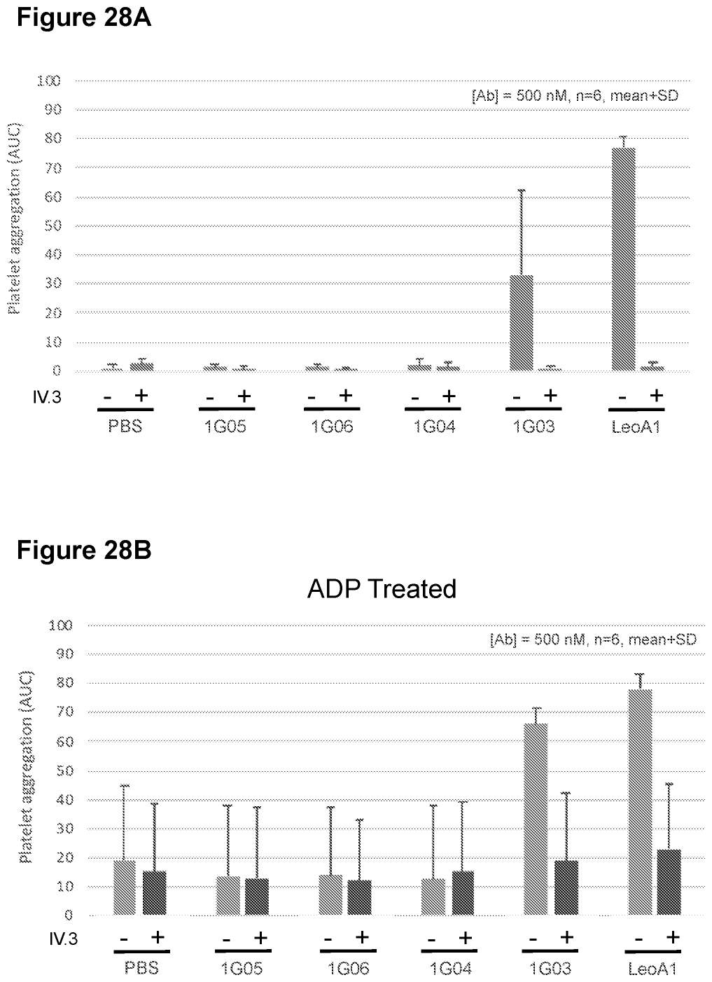

The different Fc formatted anti-CD36 antibodies were also tested in an in vitro antibody-induced platelet aggregation assay. Platelet aggregation responses were measured by light transmission aggregometry (LTA) as an increase in light transmission through the sample using a specialised aggregometer, AggRAM (Helena Biosciences), in platelet rich plasma (PRP) prepared from whole blood samples from six healthy volunteers. Antibodies were tested in the presence and absence of the platelet agonist adenosine diphosphate (ADP). ADP was titrated to produce a partial aggregation response in PRP from all volunteers (thereby confirming the viability of the PRP samples). An anti-CD226/DNAM-1 (LeoA1) antibody with a known effect on platelets via FcγRIIa receptor was included as a positive control. Possible direct and potentiation effects of the test antibodies were also assessed in the presence of a blocking anti-CD32 antibody (IV.3) to confirm if any observed effects were mediated via the FcγRIIa receptor. Each anti-CD36 antibody was tested at a 500 nM concentration (with n=6). As shown in FIGS. 28A and 28B, each of the Fc alterations eliminated antibody-induced platelet aggregation, even in the presence of a platelet aggregation agonist (ADP).

### Example 14—Epitope Analysis of the 1G04 and 11G04 Anti-CD36 Antibodies

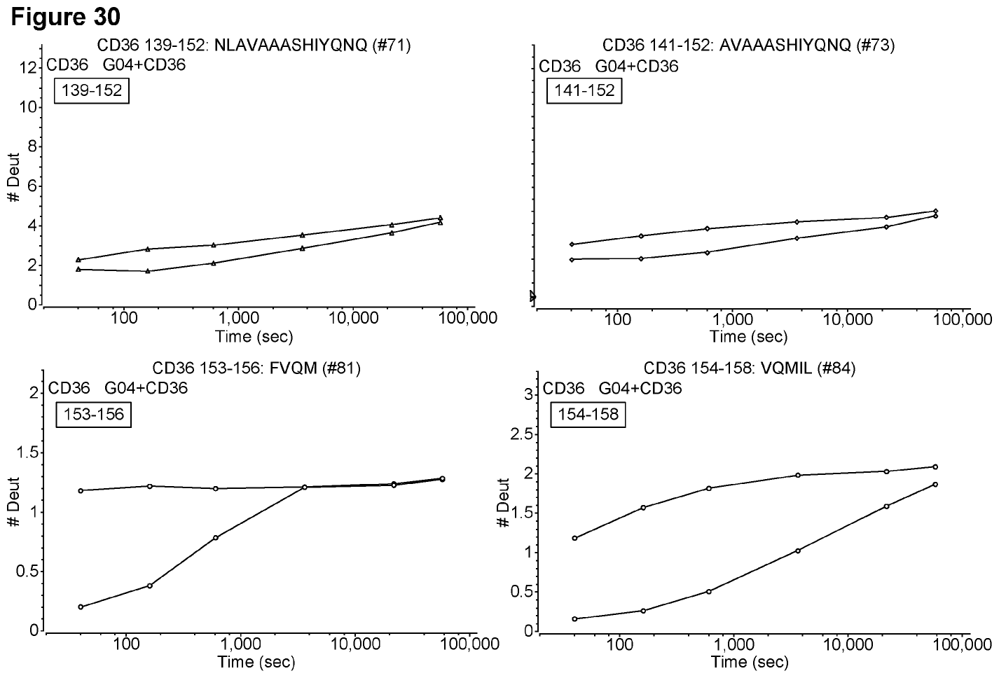

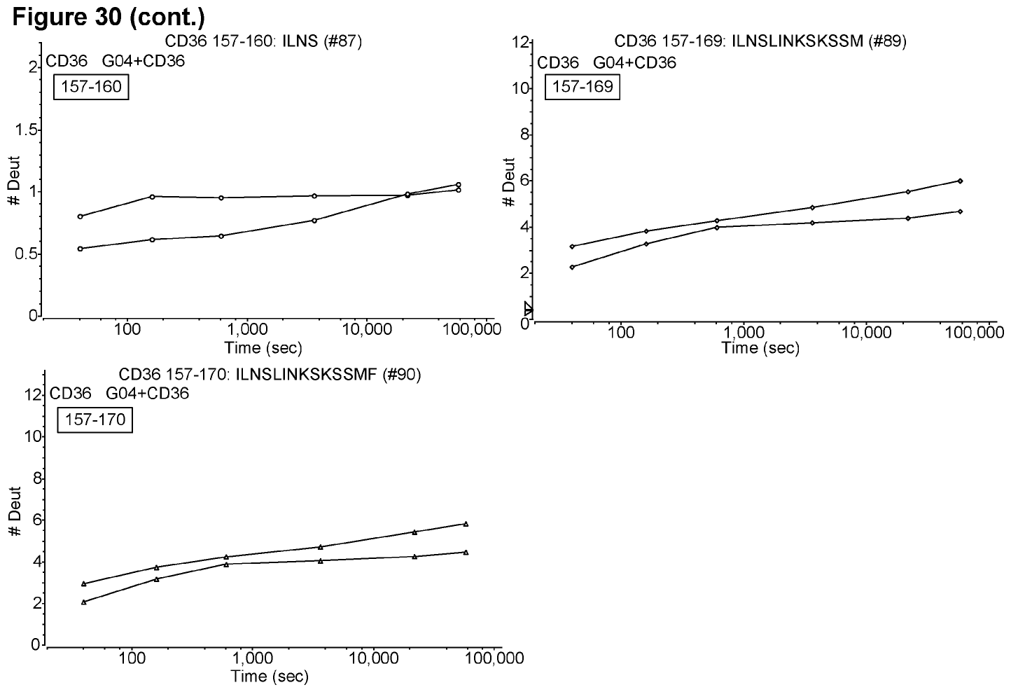

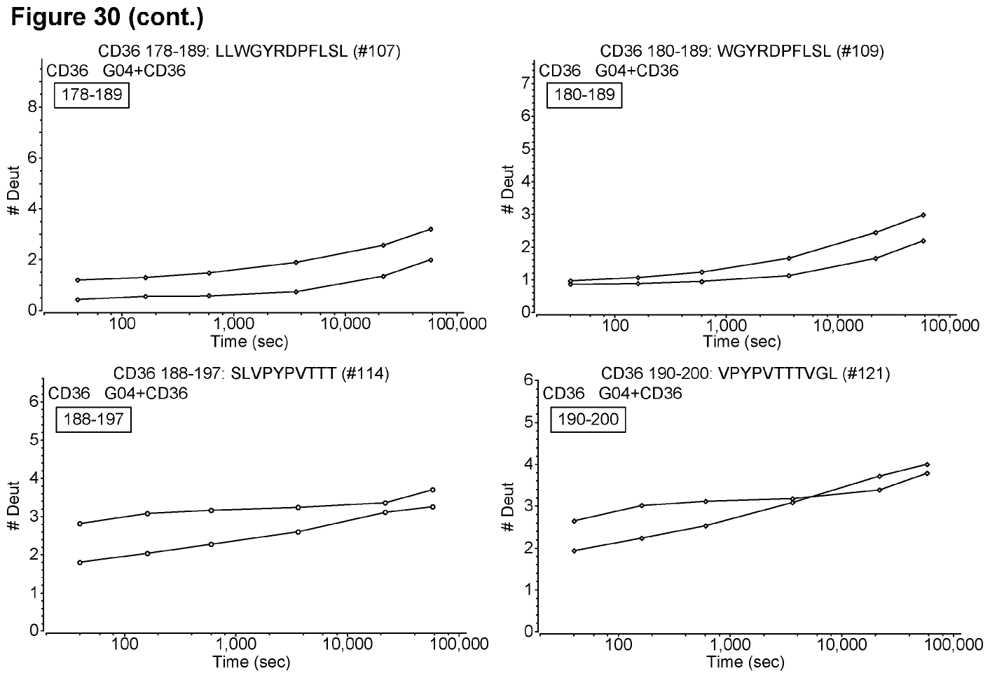

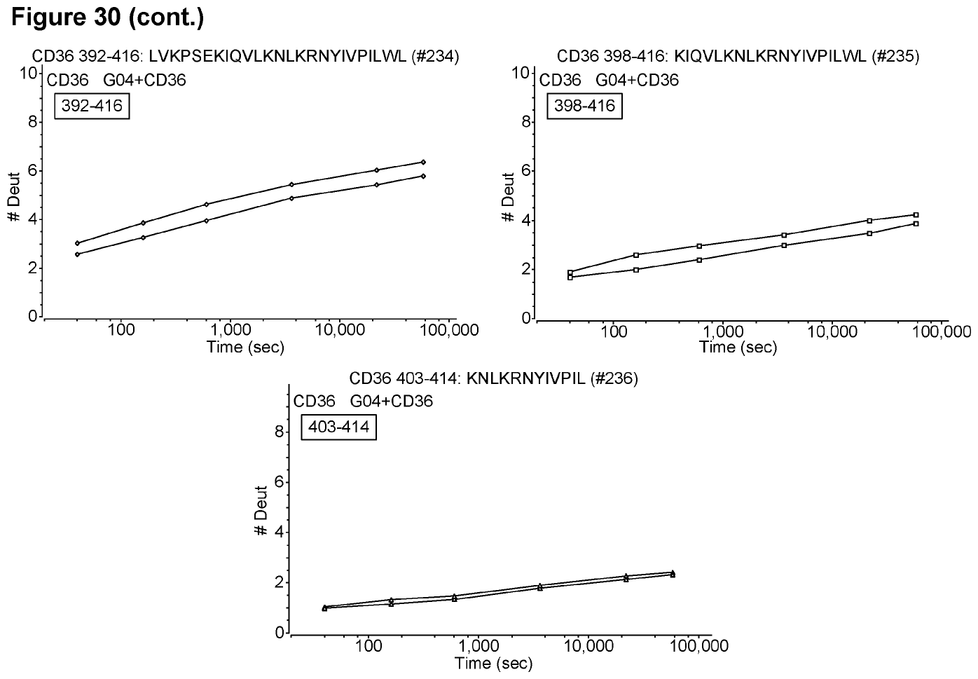

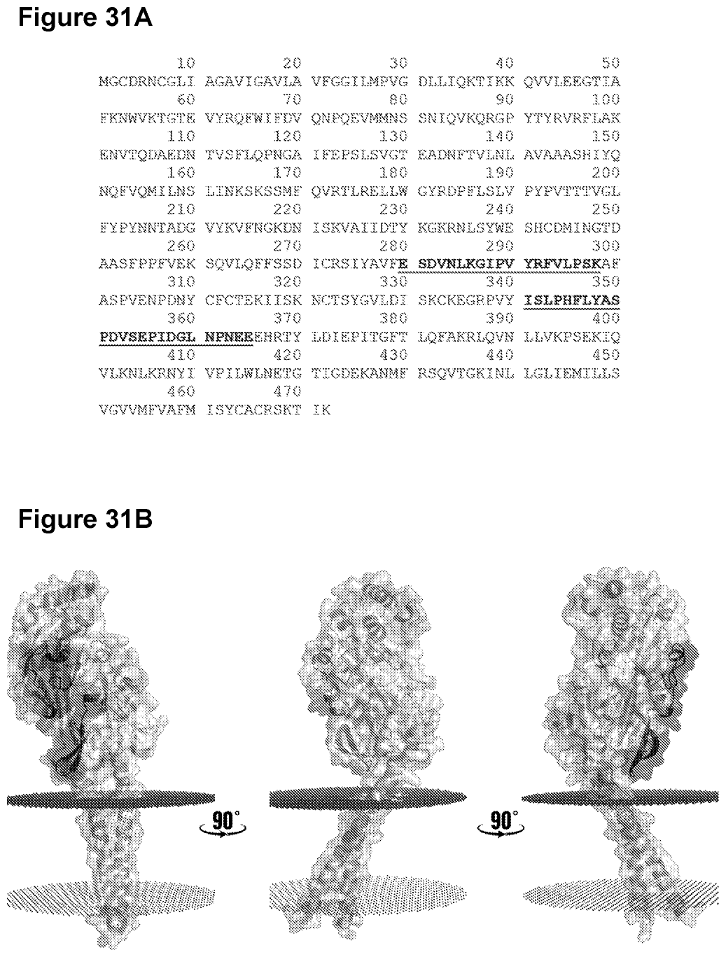

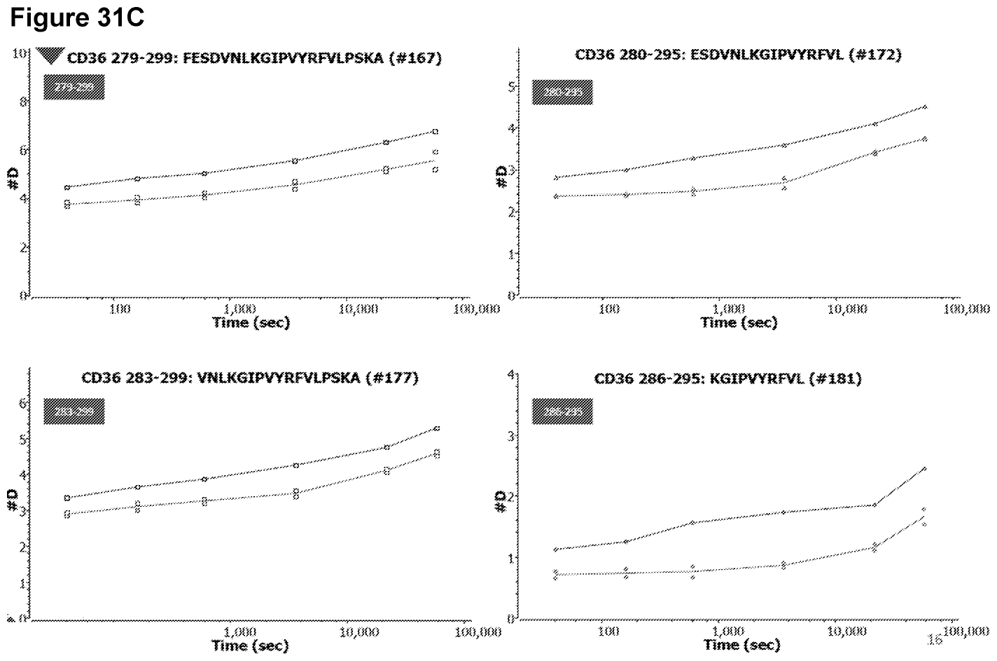

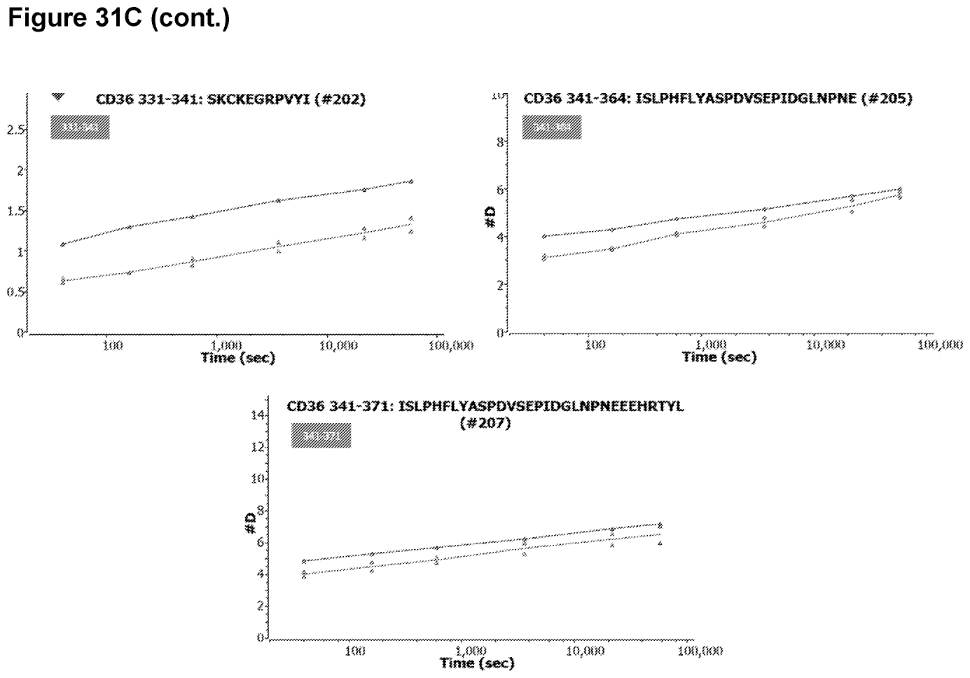

For deuterium labeling, the 1G04 and 11G04 antibodies were each incubated with recombinant human CD36 at an optimal stoichiometric ratio of 1:1.5 in D20-labeling solution and concentrated to 10 mg/mL via membrane filtration. All samples were buffer exchanged in PBS pH 7.4 and incubated for various time points at room temperature. The deuteration was quenched by transferring 30 μL of the sample to 30 μL of prechilled 1 M tris(2-carboxyethyl)phosphine (TCEP) in 8 M guanidine chloride, pH 2.5 (quench buffer), and the mixed sample was incubated at 1.0° C. for 2 min. The quenched sample was then subjected to online digestion using an immobilized pepsin/protease column. The digested peptides were trapped onto a C18 precolumn at 0° C. and eluted to an analytical C18 column for chromatographic separation using a 9 min gradient separation of 3-40% 5 (mobile phase A: 0.1% formic acid in water; mobile phase B: 0.1% formic acid in acetonitrile). For identification of CD36 peptides, LC/MSE data from an undeuterated sample were processed and searched against a database including human CD36. The deuterium uptake of each peptide in CD36 alone and CD36 with mAb was calculated based on centroid mass value at each time point with aligned retention time and high mass accuracy (<10 ppm) from triplicates. Any region of CD36 with significant protection upon mAb binding was defined as a component of the mAb's epitope. FIG. 30 shows the deuterium uptake for each CD36 peptide from 1G04 alone and with mAb. FIG. 31C shows the deuterium uptake for each CD36 peptide from 11G04 alone and with mAb.

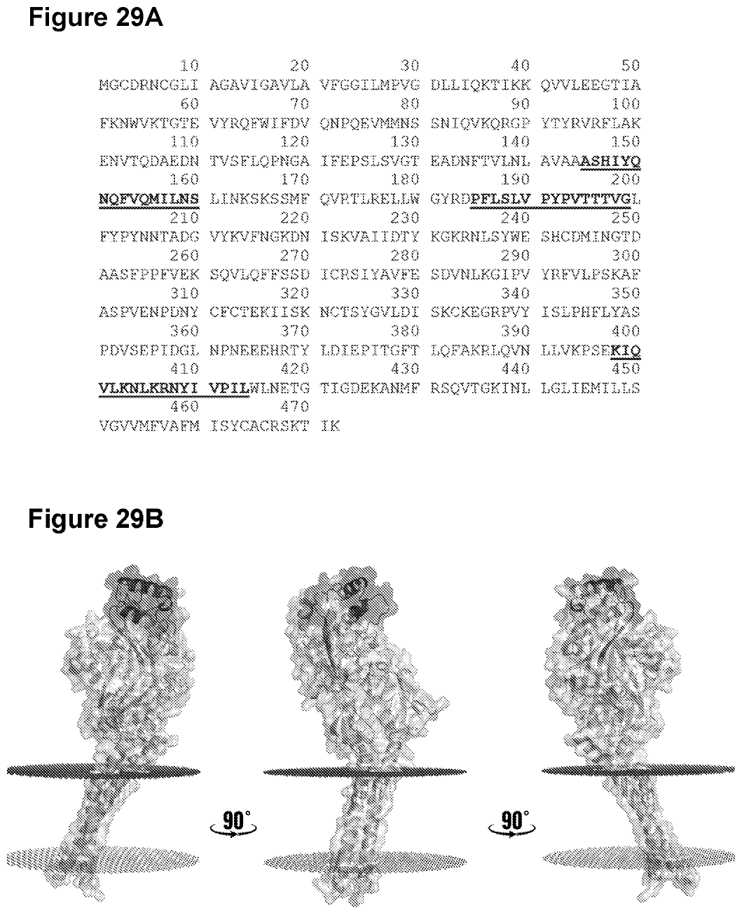

The residues that were identified as constituting the main 1G04 binding epitope are shown underlined in bold in the human CD36 sequence (SEQ ID NO: 1) in FIG. 29A. A combined ribbon and space filling model of CD36 is shown in FIG. 29B. Residues identified by hydrogen-deuterium exchange experiments to constitute the main binding epitope for 1G04 are highlighted in bold (145A, 146S, 147H, 148I, 149Y, 150Q, 151N, 152Q, 153F, 154V, 155Q, 156M, 1571, 158L, 159N, 160S, 185P, 186F, 187L, 188S, 189L, 190V, 191P, 192Y, 193P, 194V, 195T, 196T, 197T, 198V, 199G, 398K, 399I, 400Q, 401V, 402L, 403K, 404N, 405L, 406K, 407R, 408N, 409Y, 4101, 411V, 412P, 4131, and 414L).

The residues that were identified as constituting the main 11G04 binding epitope are shown underlined in bold in the human CD36 sequence (SEQ ID NO: 1) in FIG. 31A. A combined ribbon and space filling model of CD36 is shown in FIG. 31B. Residues identified by hydrogen-deuterium exchange experiments to constitute the main binding epitope for 11G04 are highlighted in bold (280E, 281S, 282D, 283V, 284N, 285L, 286K, 287G, 2881, 289P, 290V, 291Y, 292R, 293F, 294V, 295L, 296P, 297S, 298K, 341I, 342S, 343L, 344P, 345H, 346F, 347L, 348Y, 349A, 350S, 351P, 352D, 353V, 354S, 355E, 356P, 3571, 358D, 359G, 360L 361N, 362D, 363N, 364E, 365E).

### Example 15: Treatment of Colon Cancer Using Anti-CD36 Antibodies

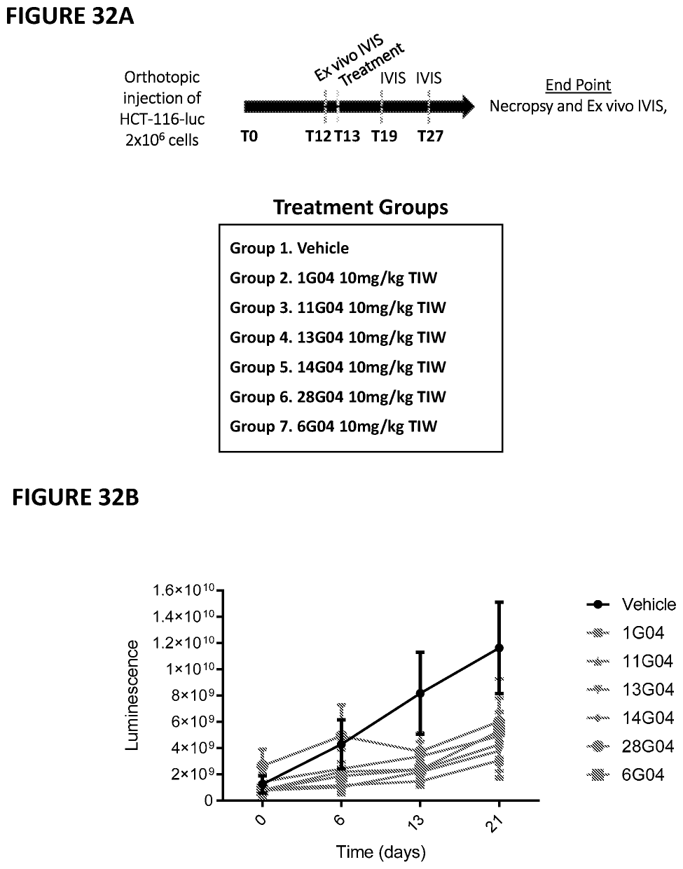

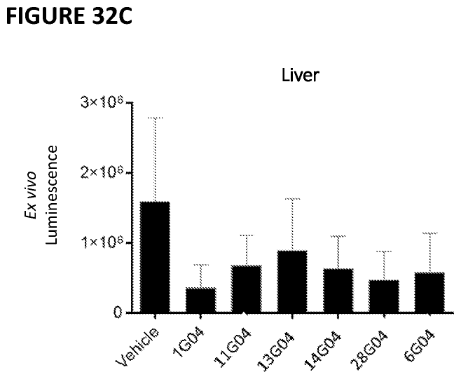

Studies of the effects of the anti-CD36 antibodies on colon cancer were performed in BALB/c nude mice (immuno-deficient). An experimental overview of these studies is provided in FIG. 32A. The studies included only female mice. All mice were inoculated with commercially available HCT-116 (ATCC) cancer cells, transduced with a retroviral vector expressing luciferase. HCT-116 cells were derived from a human colorectal carcinoma (i.e., from a colon cancer). Prior to inoculation, the HCT-116 cells were cultured in a humidified incubator at 37° C. with 5% CO₂, and were grown in McCoy's 5A medium supplemented with 5 g ml⁻¹ penicillin/streptomycin and 10% FBS (GIBCO).

For each mouse, 2×10⁶ HCT-116 cells were inoculated via orthotopic injection. Each mouse was imaged after inoculation and one week later and liver metastasis were confirmed by ex vivo luminescence prior to start of treatment. Treatment began 13 days after inoculation with the HCT-116 cells. Inoculated mice were divided into one of seven treatment groups: vehicle injection control (n=8), 1G04 treatment (n=8), 11G04 treatment (n=9), 13G04 treatment (n=6), 14G04 treatment (n=7), 28G04 treatment (n=9) and 6G04 treatment (n=8). Antibody treatments were administered via intraperitoneal (i.p.) injection at a dose of 10 mg/kg three times per week, while control mice received an equal volume of vehicle on the same schedule. One day prior to the start of treatment, and at 6, 13, and 21 days after the start of treatment, all mice were imaged via IVIS. Mice were sacrificed at the end of the treatment period (day 23). Upon sacrifice, organs and tissues were collected for performance of necropsy and ex-vivo IVIS.

FIG. 32B shows the results of whole-animal bioluminescence imaging over time, which is a readout for the growth of luciferase-containing tumor cells in the mouse. The bioluminescence imaging showed that all anti-CD36 antibodies decreased whole animal luminescence, and thus slowed the growth of the injected HCT-116 tumor cells in vivo.

After the liver was removed from the mice, the luminescence of the metastatic tumors in the liver (FIG. 32C) was quantified by ex vivo luminescence using IVIS. The observed mean luminescence values for vehicle-treated mice were 1.60*10⁸. The observed mean luminescence values for the groups of mice treated with anti-CD36 antibodies were 3.61*10⁷ (1G04), 6.85*10⁷ (11G04), 8.99*10⁷ (13G04), 6.38*10⁷ (14G04), 4.76*10⁷ (28G04) and 5.84*10⁷ (6G04). Thus, anti-CD36 treatment decreases the luminescence, reflecting a reduction in the size and/or number of metastases. These data indicate that all tested antibodies were potent inhibitors of metastasis spread and growth in colon cancer.

Collectively, FIGS. 32B and 32C show that the tested anti-CD36 antibodies are effective at reducing the formation and growth of metastases from colon cancer.

### Example 16: Antibody Clustering According to Similarity of HCDR3

HCDR3 clustering of some of the anti-CD36 antibodies was performed based on the mutual similarity in the amino acid sequence across HCDR3, which constitutes the main hypervariable loop in the paratope.

The results are shown in Table 17 below:

TABLE 17
HCDR3 clustering

| mAb ID | | | |
|---|---|---|---|
| (lgG | | | HCDR3 |
| LALA) | HCDR3 (Kabat) | HCDR3 nr. | Cluster |
| 11G04 | KLDFDY | 42 | 1 |
| 07G04 | GGGYDGAWFAY | 27 | 2 |
| 09G04 | GYGNYGAWFAY | 36 | 2 |
| 14G04 | WLLSGNGMDY | 55 | 3 |
| 04G04 | DYYGSSYGYFDV | 15 | 4 |
| 31G04 | DYYGSSYGYFDV | 15 | 4 |
| 13G04 | SDYGNGYSFYLDV | 49 | 5 |
| 21G04 | IYYGLDN | 41 | 6 |
| 22G04 | IHYGLDN | 40 | 6 |
| 23G04 | IYYGLDN | 41 | 6 |
| 24G04 | IYYGLDN | 41 | 6 |
| 25G04 | IYYGLDN | 41 | 6 |
| 29G04 | IYYGLDN | 41 | 6 |
| 15G04 | EVYYGGYEDYGMDY | 18 | 7 |
| 27G04 | FILENYFDY | 19 | 8 |
| 26G04 | FTVEVTDAMDY | 20 | 9 |
| 28G04 | AGDYAFDY | 1 | 10 |
| 18G04 | DRYDVWFAY | 10 | 11 |
| 19G04 | DRYDVWFAY | 10 | 11 |
| 20G04 | DRYDVWFAY | 10 | 11 |
| 16G04 | DYFYGDANPWFAY | 11 | 12 |
| 17G04 | DYFYGDGYPWFTY | 12 | 12 |
| 10G04 | DGGYDVYFDY | 4 | 13 |
| 05G04 | DYYGSSTAWFAY | 14 | 14 |
| 06G04 | GEDYEGTWFAY | 23 | 14 |
| 12G04 | HGYDYDEEGAWFAY | 38 | 15 |
| 30G04 | HGQTGTWFAY | 37 | 16 |
| 32G04 | LLLR | 62 | 17 |
| 33G04 | SAYGDWFYGSSGPYADSIDA | 72 | 18 |
| 34G04 | SAYGDWFYGSSGPYADSIDA | 72 | 18 |
| 35G04 | AADSGYLYITDSIDA | 66 | 19 |
| 36G04 | SADTGYCSWSACIADSIDA | 70 | 20 |
| 37G04 | SSYGGGWSDVGSIDA | 77 | 21 |
| 38G04 | DSGAVSIDA | 68 | 22 |

1. An isolated antibody that binds to CD36, preferably wherein the antibody is a chimeric or humanized antibody, which comprises a light chain CDR1 region, a light chain CDR2 region, a light chain CDR3 region, a heavy chain CDR1 region, a heavy chain CDR2 region, and a heavy chain CDR3 region, wherein
(a) the heavy chain CDR1 region comprises SEQ ID NO: 85, the heavy chain CDR2 region comprises SEQ ID NO: 106, the heavy chain CDR3 region comprises SEQ ID NO: 133, the light chain CDR1 region comprises SEQ ID NO: 159, the light chain CDR2 region comprises SEQ ID NO: 173, and the light chain CDR3 region comprises SEQ ID NO: 186;
(b) the heavy chain CDR1 region comprises SEQ ID NO: 86, the heavy chain CDR2 region comprises SEQ ID NO: 107, the heavy chain CDR3 region comprises SEQ ID NO: 134, the light chain CDR1 region comprises SEQ ID NO: 160, the light chain CDR2 region comprises SEQ ID NO: 174, and the light chain CDR3 region comprises SEQ ID NO: 187;
(c) the heavy chain CDR1 region comprises SEQ ID NO: 86, the heavy chain CDR2 region comprises SEQ ID NO: 108, the heavy chain CDR3 region comprises SEQ ID NO: 135, the light chain CDR1 region comprises SEQ ID NO: 160, the light chain CDR2 region comprises SEQ ID NO: 174, and the light chain CDR3 region comprises SEQ ID NO: 188;
(d) the heavy chain CDR1 region comprises SEQ ID NO: 87, the heavy chain CDR2 region comprises SEQ ID NO: 109, the heavy chain CDR3 region comprises SEQ ID NO: 136, the light chain CDR1 region comprises SEQ ID NO: 160, the light chain CDR2 region comprises SEQ ID NO: 174, and the light chain CDR3 region comprises SEQ ID NO: 187;
(e) the heavy chain CDR1 region comprises SEQ ID NO: 88, the heavy chain CDR2 region comprises SEQ ID NO: 110, the heavy chain CDR3 region comprises SEQ ID NO: 137, the light chain CDR1 region comprises SEQ ID NO: 160, the light chain CDR2 region comprises SEQ ID NO: 174, and the light chain CDR3 region comprises SEQ ID NO: 187;
(f) the heavy chain CDR1 region comprises SEQ ID NO: 89, the heavy chain CDR2 region comprises SEQ ID NO: 111, the heavy chain CDR3 region comprises SEQ ID NO: 138, the light chain CDR1 region comprises SEQ ID NO: 161, the light chain CDR2 region comprises SEQ ID NO: 175, and the light chain CDR3 region comprises SEQ ID NO: 189;
(g) the heavy chain CDR1 region comprises SEQ ID NO: 90, the heavy chain CDR2 region comprises SEQ ID NO: 112, the heavy chain CDR3 region comprises SEQ ID NO: 139, the light chain CDR1 region comprises SEQ ID NO: 160, the light chain CDR2 region comprises SEQ ID NO: 174, and the light chain CDR3 region comprises SEQ ID NO: 190;
(h) the heavy chain CDR1 region comprises SEQ ID NO: 91, the heavy chain CDR2 region comprises SEQ ID NO: 113, the heavy chain CDR3 region comprises SEQ ID NO: 140, the light chain CDR1 region comprises SEQ ID NO: 160, the light chain CDR2 region comprises SEQ ID NO: 174, and the light chain CDR3 region comprises SEQ ID NO: 187;
(i) the heavy chain CDR1 region comprises SEQ ID NO: 92, the heavy chain CDR2 region comprises SEQ ID NO: 114, the heavy chain CDR3 region comprises SEQ ID NO: 141, the light chain CDR1 region comprises SEQ ID NO: 162, the light chain CDR2 region comprises SEQ ID NO: 176, and the light chain CDR3 region comprises SEQ ID NO: 191;
(j) the heavy chain CDR1 region comprises SEQ ID NO: 93, the heavy chain CDR2 region comprises SEQ ID NO: 115, the heavy chain CDR3 region comprises SEQ ID NO: 142, the light chain CDR1 region comprises SEQ ID NO: 163, the light chain CDR2 region comprises SEQ ID NO: 177, and the light chain CDR3 region comprises SEQ ID NO: 192;
(k) the heavy chain CDR1 region comprises SEQ ID NO: 94, the heavy chain CDR2 region comprises SEQ ID NO: 116, the heavy chain CDR3 region comprises SEQ ID NO: 143, the light chain CDR1 region comprises SEQ ID NO: 164, the light chain CDR2 region comprises SEQ ID NO: 175, and the light chain CDR3 region comprises SEQ ID NO: 193;
(l) the heavy chain CDR1 region comprises SEQ ID NO: 95, the heavy chain CDR2 region comprises SEQ ID NO: 117, the heavy chain CDR3 region comprises SEQ ID NO: 144, the light chain CDR1 region comprises SEQ ID NO: 160, the light chain CDR2 region comprises SEQ ID NO: 174, and the light chain CDR3 region comprises SEQ ID NO: 190;
(m) the heavy chain CDR1 region comprises SEQ ID NO: 95, the heavy chain CDR2 region comprises SEQ ID NO: 118, the heavy chain CDR3 region comprises SEQ ID NO: 145, the light chain CDR1 region comprises SEQ ID NO: 160, the light chain CDR2 region comprises SEQ ID NO: 174, and the light chain CDR3 region comprises SEQ ID NO: 187;
(n) the heavy chain CDR1 region comprises SEQ ID NO: 95, the heavy chain CDR2 region comprises SEQ ID NO: 119, the heavy chain CDR3 region comprises SEQ ID NO: 146, the light chain CDR1 region comprises SEQ ID NO: 160, the light chain CDR2 region comprises SEQ ID NO: 174, and the light chain CDR3 region comprises SEQ ID NO: 190;
(o) the heavy chain CDR1 region comprises SEQ ID NO: 96, the heavy chain CDR2 region comprises SEQ ID NO: 120, the heavy chain CDR3 region comprises SEQ ID NO: 147, the light chain CDR1 region comprises SEQ ID NO: 159, the light chain CDR2 region comprises SEQ ID NO: 173, and the light chain CDR3 region comprises SEQ ID NO: 194;
(p) the heavy chain CDR1 region comprises SEQ ID NO: 96, the heavy chain CDR2 region comprises SEQ ID NO: 121, the heavy chain CDR3 region comprises SEQ ID NO: 148, the light chain CDR1 region comprises SEQ ID NO: 159, the light chain CDR2 region comprises SEQ ID NO: 173, and the light chain CDR3 region comprises SEQ ID NO: 195;
(q) the heavy chain CDR1 region comprises SEQ ID NO: 96, the heavy chain CDR2 region comprises SEQ ID NO: 120, the heavy chain CDR3 region comprises SEQ ID NO: 147, the light chain CDR1 region comprises SEQ ID NO: 159, the light chain CDR2 region comprises SEQ ID NO: 173, and the light chain CDR3 region comprises SEQ ID NO: 186;
(r) the heavy chain CDR1 region comprises SEQ ID NO: 96, the heavy chain CDR2 region comprises SEQ ID NO: 121, the heavy chain CDR3 region comprises SEQ ID NO: 147, the light chain CDR1 region comprises SEQ ID NO: 159, the light chain CDR2 region comprises SEQ ID NO: 173, and the light chain CDR3 region comprises SEQ ID NO: 196;
(s) the heavy chain CDR1 region comprises SEQ ID NO: 96, the heavy chain CDR2 region comprises SEQ ID NO: 121, the heavy chain CDR3 region comprises SEQ ID NO: 147, the light chain CDR1 region comprises SEQ ID NO: 159, the light chain CDR2 region comprises SEQ ID NO: 173, and the light chain CDR3 region comprises SEQ ID NO: 195;
(t) the heavy chain CDR1 region comprises SEQ ID NO: 97, the heavy chain CDR2 region comprises SEQ ID NO: 122, the heavy chain CDR3 region comprises SEQ ID NO: 149, the light chain CDR1 region comprises SEQ ID NO: 165, the light chain CDR2 region comprises SEQ ID NO: 178, and the light chain CDR3 region comprises SEQ ID NO: 197;
(u) the heavy chain CDR1 region comprises SEQ ID NO: 97, the heavy chain CDR2 region comprises SEQ ID NO: 123, the heavy chain CDR3 region comprises SEQ ID NO: 150, the light chain CDR1 region comprises SEQ ID NO: 165, the light chain CDR2 region comprises SEQ ID NO: 178, and the light chain CDR3 region comprises SEQ ID NO: 197;
(v) the heavy chain CDR1 region comprises SEQ ID NO: 98, the heavy chain CDR2 region comprises SEQ ID NO: 124, the heavy chain CDR3 region comprises SEQ ID NO: 151, the light chain CDR1 region comprises SEQ ID NO: 160, the light chain CDR2 region comprises SEQ ID NO: 174, and the light chain CDR3 region comprises SEQ ID NO: 198;
(w) the heavy chain CDR1 region comprises SEQ ID NO: 96, the heavy chain CDR2 region comprises SEQ ID NO: 120, the heavy chain CDR3 region comprises SEQ ID NO: 147, the light chain CDR1 region comprises SEQ ID NO: 159, the light chain CDR2 region comprises SEQ ID NO: 173, and the light chain CDR3 region comprises SEQ ID NO: 195;
(x) the heavy chain CDR1 region comprises SEQ ID NO: 91, the heavy chain CDR2 region comprises SEQ ID NO: 125, the heavy chain CDR3 region comprises SEQ ID NO: 152, the light chain CDR1 region comprises SEQ ID NO: 160, the light chain CDR2 region comprises SEQ ID NO: 174, and the light chain CDR3 region comprises SEQ ID NO: 190;
(y) the heavy chain CDR1 region comprises SEQ ID NO: 99, the heavy chain CDR2 region comprises SEQ ID NO: 126, the heavy chain CDR3 region comprises SEQ ID NO: 133, the light chain CDR1 region comprises SEQ ID NO: 162, the light chain CDR2 region comprises SEQ ID NO: 176, and the light chain CDR3 region comprises SEQ ID NO: 199;
(z) the heavy chain CDR1 region comprises SEQ ID NO: 100, the heavy chain CDR2 region comprises SEQ ID NO: 127, the heavy chain CDR3 region comprises SEQ ID NO: 153, the light chain CDR1 region comprises SEQ ID NO: 166, the light chain CDR2 region comprises SEQ ID NO: 179, and the light chain CDR3 region comprises SEQ ID NO: 200;
(aa) the heavy chain CDR1 region comprises SEQ ID NO: 101, the heavy chain CDR2 region comprises SEQ ID NO: 128, the heavy chain CDR3 region comprises SEQ ID NO: 154, the light chain CDR1 region comprises SEQ ID NO: 167, the light chain CDR2 region comprises SEQ ID NO: 180, and the light chain CDR3 region comprises SEQ ID NO: 201;
(bb) the heavy chain CDR1 region comprises SEQ ID NO: 101, the heavy chain CDR2 region comprises SEQ ID NO: 128, the heavy chain CDR3 region comprises SEQ ID NO: 154, the light chain CDR1 region comprises SEQ ID NO: 168, the light chain CDR2 region comprises SEQ ID NO: 181, and the light chain CDR3 region comprises SEQ ID NO: 202;
(cc) the heavy chain CDR1 region comprises SEQ ID NO: 102, the heavy chain CDR2 region comprises SEQ ID NO: 129, the heavy chain CDR3 region comprises SEQ ID NO: 155, the light chain CDR1 region comprises SEQ ID NO: 169, the light chain CDR2 region comprises SEQ ID NO: 182, and the light chain CDR3 region comprises SEQ ID NO: 203;
(dd) the heavy chain CDR1 region comprises SEQ ID NO: 103, the heavy chain CDR2 region comprises SEQ ID NO: 130, the heavy chain CDR3 region comprises SEQ ID NO: 156, the light chain CDR1 region comprises SEQ ID NO: 170, the light chain CDR2 region comprises SEQ ID NO: 183, and the light chain CDR3 region comprises SEQ ID NO: 204;
(ee) the heavy chain CDR1 region comprises SEQ ID NO: 104, the heavy chain CDR2 region comprises SEQ ID NO: 131, the heavy chain CDR3 region comprises SEQ ID NO: 157, the light chain CDR1 region comprises SEQ ID NO: 171, the light chain CDR2 region comprises SEQ ID NO: 184, and the light chain CDR3 region comprises SEQ ID NO: 205;
(ff) the heavy chain CDR1 region comprises SEQ ID NO: 105, the heavy chain CDR2 region comprises SEQ ID NO: 132, the heavy chain CDR3 region comprises SEQ ID NO: 158, the light chain CDR1 region comprises SEQ ID NO: 172, the light chain CDR2 region comprises SEQ ID NO: 185, and the light chain CDR3 region comprises SEQ ID NO: 206;
(gg) the heavy chain CDR1 region comprises SEQ ID NO: 90, the heavy chain CDR2 region comprises SEQ ID NO: 248, the heavy chain CDR3 region comprises SEQ ID NO: 139, the light chain CDR1 region comprises SEQ ID NO:160, the light chain CDR2 region comprises SEQ ID NO: 174, and the light chain CDR3 region comprises SEQ ID NO: 247: or
(hh) the heavy chain CDR1 region comprises SEQ ID NO: 90, the heavy chain CDR2 region comprises SEQ ID NO: 248, the heavy chain CDR3 region comprises SEQ ID NO: 139, the light chain CDR1 region comprises SEQ ID NO:160, the light chain CDR2 region comprises SEQ ID NO: 246, and the light chain CDR3 region comprises SEQ ID NO: 247.

2-38. (canceled)

39. The antibody of claim 1, wherein the antibody comprises a heavy chain variable region and a light chain variable region, wherein:
i. the heavy chain variable region comprises SEQ ID NO: 13 and the light chain variable region comprises SEQ ID NO: 45;
ii. the heavy chain variable region comprises SEQ ID NO: 14 and the light chain variable region comprises SEQ ID NO: 46;
iv. the heavy chain variable region comprises SEQ ID NO: 15 and the light chain variable region comprises SEQ ID NO: 47;
iv. the heavy chain variable region comprises SEQ ID NO: 16 and the light chain variable region comprises SEQ ID NO: 48;
v. the heavy chain variable region comprises SEQ ID NO: 17 and the light chain variable region comprises SEQ ID NO: 49,
vi. the heavy chain variable region comprises SEQ ID NO: 18 and the light chain variable region comprises SEQ ID NO: 50;
vii. the heavy chain variable region comprises SEQ ID NO: 19 and the light chain variable region comprises SEQ ID NO: 51;
viii. the heavy chain variable region comprises SEQ ID NO: 20 and the light chain variable region comprises SEQ ID NO: 52;
ix. the heavy chain variable region comprises SEQ ID NO: 21 and the light chain variable region comprises SEQ ID NO: 53;
x. the heavy chain variable region comprises SEQ ID NO: 22 and the light chain variable region comprises SEQ ID NO: 54;
xi. the heavy chain variable region comprises SEQ ID NO: 23 and the light chain variable region comprises SEQ ID NO: 55;
xii. the heavy chain variable region comprises SEQ ID NO: 24 and the light chain variable region comprises SEQ ID NO: 56;
xiii. the heavy chain variable region comprises SEQ ID NO: 25 and the light chain variable region comprises SEQ ID NO: 57;
xiv. the heavy chain variable region comprises SEQ ID NO: 26 and the light chain variable region comprises SEQ ID NO: 58;
xv. the heavy chain variable region comprises SEQ ID NO: 26 and the light chain variable region comprises SEQ ID NO: 59;
xvi. the heavy chain variable region comprises SEQ ID NO: 27 and the light chain variable region comprises SEQ ID NO: 60;
xvii. the heavy chain variable region comprises SEQ ID NO: 28 and the light chain variable region comprises SEQ ID NO: 61;
xviii. the heavy chain variable region comprises SEQ ID NO: 29 and the light chain variable region comprises SEQ ID NO: 62;
xix. the heavy chain variable region comprises SEQ ID NO: 30 and the light chain variable region comprises SEQ ID NO: 63;
xx. the heavy chain variable region comprises SEQ ID NO: 31 and the light chain variable region comprises SEQ ID NO: 64;
xxi. the heavy chain variable region comprises SEQ ID NO: 32 and the light chain variable region comprises SEQ ID NO: 65;
xxii. the heavy chain variable region comprises SEQ ID NO: 33 and the light chain variable region comprises SEQ ID NO: 66;
xxiii. the heavy chain variable region comprises SEQ ID NO: 34 and the light chain variable region comprises SEQ ID NO: 67;
xxiv. the heavy chain variable region comprises SEQ ID NO: 35 and the light chain variable region comprises SEQ ID NO: 68;
xxv. the heavy chain variable region comprises SEQ ID NO: 36 and the light chain variable region comprises SEQ ID NO: 69;
xxvi. the heavy chain variable region comprises SEQ ID NO: 37 and the light chain variable region comprises SEQ ID NO: 70;
xxvii. the heavy chain variable region comprises SEQ ID NO: 38 and the light chain variable region comprises SEQ ID NO: 71;
xxviii. the heavy chain variable region comprises SEQ ID NO: 39 and the light chain variable region comprises SEQ ID NO: 72;
xxix. the heavy chain variable region comprises SEQ ID NO: 40 and the light chain variable region comprises SEQ ID NO: 73;
xxx. the heavy chain variable region comprises SEQ ID NO: 40 and the light chain variable region comprises SEQ ID NO: 74;
xxxi. the heavy chain variable region comprises SEQ ID NO: 41 and the light chain variable region comprises SEQ ID NO: 75;
xxxii. the heavy chain variable region comprises SEQ ID NO: 42 and the light chain variable region comprises SEQ ID NO: 76;
xxxiii. the heavy chain variable region comprises SEQ ID NO: 43 and the light chain variable region comprises SEQ ID NO: 77;
xxxiv. the heavy chain variable region comprises SEQ ID NO: 44 and the light chain variable region comprises SEQ ID NO: 78;
xxxv. the heavy chain variable region comprises SEQ NO: 241 and the light chain variable region comprises SEQ ID NO: 240;
xxxvi. the heavy chain variable region comprises SEQ NO: 243 and the light chain variable region comprises SEQ ID NO: 240;
xxxvii. the heavy chain variable region comprises SEQ NO: 241 and the light chain variable region comprises SEQ ID NO: 242;
xxxviii. the heavy chain variable region comprises SEQ NO: 243 and the light chain variable region comprises SEQ ID NO: 242;
xxxix. the heavy chain variable region comprises SEQ NO: 241 and the light chain variable region comprises SEQ ID NO: 244;
xl. the heavy chain variable region comprises SEQ NO: 243 and the light chain variable region comprises SEQ ID NO: 244;
xli. the heavy chain variable region comprises SEQ NO: 241 and the light chain variable region comprises SEQ ID NO: 245; or
xlii. the heavy chain variable region comprises SEQ NO: 243 and the light chain variable region comprises SEQ ID NO: 245.

40-47. (canceled)

48. The antibody of claim 1, wherein the antibody binds to an epitope in human CD36 comprising at least one amino acid selected from the group consisting of 145A, 146S, 147H, 148I, 149Y, 150Q, 151N, 152Q, 153F, 154V, 155Q, 156M, 157I, 158L, 159N, 160S, 185P, 186F, 187L, 188S, 189L, 190V, 191P, 192Y, 193P, 194V, 195T, 196T, 197T, 198V, 199G, 398K, 399I, 400Q, 401V, 402L, 403K, 404N, 405L, 406K, 407R, 408N, 409Y, 410I, 411V, 412P, 413I, and 414L;
preferably wherein the antibody binds to an epitope in human CD36 comprising at least one amino acid selected from the group consisting of 149Y, 1500, 151N, 152Q, 153F, 154V, 155Q, 156M, 188S, 189L, 190V, 191P, 192Y, 193P, 194V, 195T, 196T, 400Q, 401V, 402L, 403K; or
wherein the antibody binds to an epitope in human CD36 comprising at least one amino acid selected from the group consisting of 152Q, 192Y, and 406K.

49-60. (canceled)

61. The antibody of claim 1, which further comprises a heavy chain constant region selected from the group consisting of human immunoglobulin IgA1, IgA2, IgG1, IgG2, IgG3, or IgG4 heavy chain constant regions;
preferably wherein the heavy chain constant region comprises an IgG constant region containing at least one amino acid substitution, wherein the at least one amino acid substitution results in reduced binding to at least one Fcγ receptor and reduced or dampened Fc effector function; more preferably wherein
(i) the at least one Fc silencing mutation includes the amino acid substitutions L234A and L235A (LALA); or
(ii) the at least one Fc silencing mutation includes a set of amino acid substitutions selected from the group consisting of L234G, L235S, and G236R; L234S, L235T, and G236R; L234S, L235V, and G236R; L234T, L235Q, and G236R; L234T, L235T, and G236R; L234A and L235A; and L234A, L235A, and P329G.

62-68. (canceled)

69. The antibody of claim 1, wherein the antibody further comprises a light chain constant region,
preferably wherein the light chain constant region is selected from the group consisting of human immunoglobulins κ and λ light chain constant regions.

70. (canceled)

71. The antibody of claim 1, wherein the antibody further comprises a heavy chain constant region and a light chain constant region, wherein the heavy chain constant region is a human IgG1 heavy chain constant region, and wherein the light chain constant region is a human κ light chain constant region.

72. The antibody of claim 1, wherein the antibody is a bispecific antibody or a biparatopic antibody, preferably wherein when the antibody is a bispecific antibody, it is characterized by:
comprising a first antigen-binding region that specifically binds to CD36; and
comprising a second antigen-binding region that specifically binds to an immune cell antigen, preferably wherein the immune cell antigen is selected from the group consisting of PD-1, PD-L1, CTLA4, CD3, LAG3, OX40, CD28, CD33, B7H3, CD47, TIM3, ICOS, LGR5, 4-1BB, CD40, CD40-L, and TIGIT, more preferably, wherein the immune cell antigen is CD3; or
comprising a second antigen-binding region that specifically binds to a tumor-specific antigen, preferably wherein the tumor-specific antigen is selected from the group consisting of HER2, HER3, EGFR, VEGF, IGF-1, IGF-2, ANG2, DLL1, IGF-1R, cMET, DLL4, FAP, DR5, IL15, IL15Ra, CD3, CEA, EPCAM, HER3, PSMA, PMEL, and GPC3, more preferably wherein the tumor-specific antigen is CD3.

73-83. (canceled)

84. The antibody of claim 1, which is an antigen binding fragment, preferably wherein the antigen binding fragment comprises a Fab, Fab′, F(ab′)₂, single chain Fv (scFv), disulfide linked Fv, V-NAR domain, IgNar, intrabody, IgGΔCH2, minibody, F(ab′)₃, tetrabody, triabody, diabody, single-domain antibody, DVD-Ig, Fcab, mAb2, (scFv)₂, or scFv-Fc.

85. (canceled)

86. A pharmaceutical composition comprising the antibody of claim 1 and a pharmaceutically acceptable excipient, preferably wherein at least 95% of the antibodies in the composition are afucosylated.

87. (canceled)

88. The pharmaceutical composition of claim 86, which further comprises:
(a) a PD-1 inhibitor, preferably wherein the PD-1 inhibitor is an anti-PD-1 antibody, more preferably wherein the anti-PD-1 antibody is pembrolizumab, pidilizumab, or nivolumab;
(b) a PD-L1 inhibitor, preferably wherein the PD-L1 inhibitor is an anti-PD-L1 antibody, more preferably wherein the anti-PD-L1 antibody is atezolizumab, durvalumab, avelumab, or BMS-936559;
(c) a CTLA-4 inhibitor, preferably wherein the CTLA-4 inhibitor is an anti-CTLA-4 antibody, more preferably wherein the anti-CTLA-4 antibody is ipilimumab; or
(d) a chemotherapeutic agent, preferably wherein the chemotherapeutic agent is cisplatin.

89-104. (canceled)

105. A method of treating cancer in a patient comprising administering to a subject in need thereof a therapeutically effective amount of the antibody of claim 1, preferably wherein the patient is a human patient.

106. The method of claim 105, wherein the cancer is oral squamous cell carcinoma, head and neck cancer, esophageal cancer, gastric cancer, ovarian cancer, cervical cancer, lung cancer, breast cancer, colon cancer, renal cancer, prostate cancer, sarcoma, liposarcoma, melanoma, leukemia, or lymphoma.

107. A method of treating one or more metastatic tumors in a patient comprising administering to a subject in need thereof a therapeutically effective amount of the antibody of claim 1 preferably wherein the patient is a human patient.

108. The method of claim 107, wherein the metastatic tumors are metastatic oral squamous cell carcinoma, metastatic head and neck cancer, metastatic esophageal cancer, metastatic gastric cancer, metastatic ovarian cancer, metastatic cervical cancer, metastatic lung cancer, metastatic breast cancer, metastatic colon cancer, metastatic renal cancer, metastatic prostate cancer, metastatic sarcoma, metastatic liposarcoma, metastatic melanoma, metastatic leukemia, or metastatic lymphoma.

109-157. (canceled)

158. An isolated polynucleotide that encodes the antibody of claim 1.

159-162. (canceled)

163. A vector comprising the isolated polynucleotide of claim 158.

164. A cell comprising the isolated polynucleotide of claim 158, preferably wherein the cell is selected from the group consisting of E. coli, Pseudomonas, Bacillus, Streptomyces, yeast, CHO, YB/20, NS0, PER-C6, HEK 293, HEK 293T, NIH 3T3, HeLa, BHK, Hep G2, SP2/0, R1.1, B-W, L-M, COS 1, COS 7, BSC1, BSC40, BMT10 cell, plant cell, insect cell, and human cell in tissue culture, more preferably wherein the cell lacks a functional alpha-1,6-fucosyltransferase gene (FUT8) gene.

165-166. (canceled)

167. A method of making an antibody that is capable of specifically binding CD36, comprising culturing the cell of claim 164 under conditions suitable for expression of the antibody and isolating the antibody expressed therein, preferably wherein the antibody is secreted from the cell and isolated from media in which the cell has been cultured.

168-202. (canceled)
